(19) United States
(12) Patent Application Publication
(10) Pub. No.: US 2024/0263191 A1
(43) Pub. Date: Aug. 8, 2024

(54) ECTOPICALLY EXPRESSED TRANSCRIPTION FACTORS AND USES THEREOF

(71) Applicant: UNIVERSITA DI NAPOLI FEDERICO II, Napoli (IT)

(72) Inventors: Enrico Maria SURACE, Napoli (NA) (IT); Salvatore BOTTA, Pozzuoli (NA) (IT); Elena MARROCCO, Napoli (NA) (IT); Nicola DE PRISCO, Pozzuoli (NA) (IT)

(73) Assignee: UNIVERSITA DI NAPOLI FEDERICO II, Napoli (IT)

(21) Appl. No.: 16/772,901

(22) PCT Filed: Dec. 21, 2018
(86) PCT No.: PCT/EP2018/086782

§ 371 Date: Jun. 15, 2020

(30) Foreign Application Priority Data
Dec. 21, 2017 (EP) 17209892.3

(51) Int. Cl. C12N 15/86

(52) U.S. Cl. CPC C12N 15/86; A61K 48/0058; C07K 14/4703; C07K 14/723; C12N 5/062; C12N 15/63; C12N 2710/10041; C12N 2830/008

(57) ABSTRACT

The present invention relates to a nucleic acid construct allowing to drives the expression of a transcription factor in rod cells or cone cells thereby silencing the expression of a gene which mutated form is responsible for a retinal dystrophy and its medical use, relative expression vector, host cell, viral particle and pharmaceutical composition.

### TECHNICAL FIELD

The present invention relates to a nucleic acid construct allowing to drive the expression of a transcription factor in rod cells or cone cells thereby silencing the expression of a gene which mutated form is responsible for a retinal dystrophy and its medical use, relative expression vector, host cell, viral particle and pharmaceutical composition.

### BACKGROUND

Transcription factors (TFs) control space- and time-dependent activation or repression of genes to control biological functions (1). They regulate these genetic programs by genome-wide scanning of DNA sequences and eventually binding to discrete motifs present in gene regulatory regions (promoters and enhancers) (2, 3). TFs have an intrinsic ability to recognize primary nucleotide DNA sequence motifs (a base readout (4) of typically 5-15 bp). The principles of TF protein-DNA recognition have enabled the determination of their DNA binding preferences and the design of synthetic TFs directed to specific genomic DNA sequences (5, 6). However, individual TFs and TF family members show differential DNA binding preferences indicating that the TF-DNA recognition code is far from being fully elucidated (7), particularly in vivo. Local and distal chromosomal features, protein-protein interactions, and nuclear topography are emerging as determinants conditioning the DNA accessibility, binding and ultimately activity of TFs (8-10). These features are inherent to cell-specific composition and may be envisaged as extrinsic co-factors that complement the intrinsic TF recognition properties for DNA base readout: somatic cells of an individual organism have the same DNA sequence (syngeneic) while expressing cell-specific factors.

The retina is a layered structure composed of six neuronal and one glial cell type, which are organized in three cellular layers: the ganglion cell layer, comprising retinal ganglion (RGC) and displaced amacrine cells, the inner nuclear layer (INL), which contains bipolar, horizontal and amacrine interneurons and Müller glial cells, and the outer nuclear layer (ONL), where rod and cone photoreceptors are located. The retina is immediately adjacent to the retinal pigment epithelium (RPE), a pigmented cell layer that nourishes retinal visual cells, and is firmly attached to the underlying choroid and overlying retinal visual cells.

Rod and cone photoreceptors are the first and key transducer of light in electrical responses thus are essential for vision. Rod and cone photoreceptors display similar phenotypic features to capture and transduce light stimuli. Cones show high sensitivity for bright light, while rods show sensitivity for dim light. Rod and cone photoreceptors are anatomically located next one another and biochemically share several proteins of phototransduction cascade while others are cone and rod specific. Mutation affecting cone-specific genes typically generate cone dystrophies (COD) and cone-rod dystrophies (CORD). Mutation affecting rod-specific genes typically generate Retinitis Pigmentosa (RP), Leber Congenital Amaurosis (LCA) or rod-cone dystrophy (RCD).

Inherited retinal dystrophies (IRDs) represent one of the most frequent causes of genetic blindness in the western world. The primary condition that underlies this group of diseases is the degeneration of photoreceptors, i.e., the cells that convert the light information into chemical and electrical signals that are then transmitted to the brain through the visual circuits. There are two types of photoreceptor cells in the human retina: rods and cones. Rods represent about 95% of photoreceptor cells in the human retina and are responsible for sensing contrast, brightness and motion, whereas fine resolution, spatial resolution and color vision are perceived by cones.

IRDs can be subdivided into different groups of diseases, namely Retinitis Pigmentosa (RP), Leber Congenital Amaurosis (LCA), cone-rod dystrophies (CORD) and cone dystrophies (COD), rod-cone dystrophy (RCD).

RP is the most frequent form of inherited retinal dystrophy with an approximate frequency of about 1 in 4,000 individuals (E. L. Berson, Invest Ophtalmol Vis Sci 34, 1659 (1993)). At its clinical onset, RP is characterized by night blindness and progressive degeneration of photoreceptors accompanied by bone spicule-like pigmentary deposits and a reduced or absent electroretinogram (ERG). RP is characterized by primary loss in rod photoreceptors, later followed by the secondary loss in cone photoreceptors; it can be either isolated or syndromic, i.e., associated with extraocular manifestations such as in Usher syndrome or in Bardet-Biedle syndrome. From a genetic point of view, RP is highly heterogeneous, with autosomal dominant, autosomal recessive and X-linked patterns of inheritance. A significant percentage of RP patients, however, are apparently sporadic. To date, around 50 causative genes/loci have been found to be responsible for non-syndromic forms of RP and over 25 for syndromic RPs (RETnet web site: http://www.sph.uth.tmc.edu/RetNet/).

LCA has a prevalence of about 2-3 in 100,000 individuals and is characterized by a severe visual impairment that starts in the first months/years of life (F. P. Cremers, et al., Hum. Mol. Genet. 11, 1169 (May 15, 2002). LCA has retinal, ocular as well as extraocular features, and occasionally systemic associations. LCA is genetically heterogeneous. The autosomal dominant Leber congenital amaurosis, is due to mutations in the Inosine-5′-monophosphate dehydrogenase 1 (IMPDH1), OTX2 and CRX genes. While IMPDH1 is ubiquitously expressed, OTX2 and CRX are mainly retinal-specific and affect primarily photoreceptors.

IRDs of interest for the present invention are due to the degeneration and subsequent death of photoreceptor cells, primarily rod photoreceptors, followed by a secondary degeneration of cones. Genes responsible for IRDs of interest to the present inventions are expressed predominantly in photoreceptors, particularly in rods the main consequence that derives from the dysfunction of these genes is a damage of photoreceptor function, which then translate into photoreceptor degeneration and death. For most forms of the above-mentioned diseases an effective therapy is currently unavailable.

IRDs of interest for the present invention are due to the degeneration and subsequent death of photoreceptor cells, primarily rod photoreceptors, followed by a secondary degeneration of cones. Genes responsible for IRDs of interest to the present inventions are expressed predominantly in photoreceptors, particularly in rods the main consequence that derives from the dysfunction of these genes is a damage of photoreceptor function, which then translate into photoreceptor degeneration and death. For most forms of the above-mentioned diseases an effective therapy is currently unavailable.

IRDs with dominant pattern of inheritance have been associated to genes expressed predominantly in the retina; of particular interest to the present invention are the Rhodopsin (RHO), Peripherin 2 (PRPH2), Retinitis Pigmentosa 1 protein (RP1), Cone-Rod homeobox (CRX) nuclear receptor subfamily 2 group E3 (NR2E3), neural retina leucine zipper (NRL), retinal outer segment membrane protein 1 (ROM1).

Known genes causing autosomal dominant IRDs and associated proteins names are listed in Table 1.

TABLE 1
Known genes causing autosomal dominant IRDs and associated proteins
names. References are at RetNet: https://sph.uth.edu/retnet/.

| Protein | Disease Gene |
|---|---|
| Cone-Rod Homeobox | CRX |
| Guanylate cyclase activator 1B | GUCA1B, RP48 |
| Nuclear receptor subfamily 2 group E | NR2E3 |
| member 3 | |
| Neural retina leucine zipper | NRL, RP27 |
| Peripherin 2 | PRPH2, RDS, RP7 |
| Rhodopsin | RHO |
| Retinal outer segment membrane protein 1 | ROM1 |
| Retinitis pigmentosa 1 protein | RP1, L1 |
| retinol dehydrogenase 12 | RDH12, LCA13, RP53 |

Currently, there are no effective treatments for IRDs. Nutritional therapy featuring vitamin A or vitamin A plus docosahexaenoic acid reduces the rate of degeneration in some patients. Retinal analogs and pharmaceuticals functioning as chaperones show some progress in protecting the retina in animal models, and several antioxidant studies have shown lipophilic antioxidant taurousodeoxycholic acid (TUDCA), metallocomplex zinc desferrioxamine, N-acetyl-cysteine, and a mixture of antioxidants slow retinal degeneration in rodent rd1, rd10, and Q344ter models. A clinical trial is under way to test the efficacy of the protein deacetylase inhibitor valproic acid as a treatment for retinitis pigmentosa. Valproic acid blocks T-type calcium channels and voltage-gated sodium channels and is associated with significant side effects such as hearing loss and diarrhea. Thus, the use of valproic acid as a treatment for retinitis pigmentosa has been questioned (Rossmiller et al. Molecular Vision 2012; 18:2479-2496).

Therefore, there is still the need for a treatment of retinal dystrophies that is efficient and selective.

Cone-rod dystrophies (CRDs) have a prevalence of 1/40,000 individuals and are characterized by retinal pigment deposits visible upon fundus examination, predominantly localized to the macular region. In contrast to typical RP, which is characterized by primary loss in rod photoreceptors, later followed by the secondary loss in cone photoreceptors, CRDs reflect the opposite sequence of events. CRD is characterized by a primary cone involvement, that explains the predominant symptoms of CRDs: decreased visual acuity, color vision defects, photo-aversion and decreased sensitivity in the central visual field, later followed by progressive loss in peripheral vision and night blindness (C. P. Hamel, Orphanet J Rare Dis 2, 7 (2007). Mutations in at least 20 different genes have been associated with CRD (RETnet web site: http://www.sph.uth.tmc.edu/RetNet/).

Cone dystrophies (CD) are conditions in which cone photoreceptors display a selective dysfunction that does not extend to rods. They are characterized by visual deficit, abnormalities of color vision, visual field loss, and a variable degree of nystagmus and photophobia. In CDs, cone function is absent or severely impaired on electroretinography (ERG) and psychophysical testing (M. Michaelides, et al. Surv. Ophthalmol. 51, 232 (May-June, 2006). Similar to the other forms of inherited retinal dystrophies, CDs are heterogeneous conditions that can be caused by mutations in at least 10 different genes (RETnet web site: http://www.sph.uth.tmc.edu/RetNet/).

Cone dystrophies and cone-rod dystrophies have been associated to genes expressed predominantly in the retina; of particular interest to the present invention are retinal guanylate cyclase 2D (GUCY2D), and, guanylate cyclase activator 1A (GUCA1A)

### SUMMARY OF THE INVENTION

The genome-wide activity of transcription factors (TFs) on multiple regulatory elements precludes their use as gene specific regulators. The present inventors surprisingly show that ectopic expression of a TF in a cell-specific context can be used to silence the expression of a specific gene as a therapeutic approach to regulate gene expression in human disease.

Surprisingly, the present inventors found that cell-specific context conditioning of the activity of a TF can be successfully applied to somatic gene-targeted manipulation and gene therapy of retinal diseases, particularly inherited retinal dystrophies, more particularly retinal dystrophies wherein the primary disease is a rod disease or a cone disease, eg a disease affecting primarily rod or cone photoreceptors.

DNA constructs of the present invention therefore comprise a nucleotide sequence encoding a first promoter which is operably linked to and drives the expression of a transcription factor to rod cells or cone cells in the retina, where said transcription factor is not physiologically expressed. Further, the transcription factor of the constructs of the invention recognizes at least one nucleotide sequence of a gene which mutation is responsible for a retinal dystrophy, preferably selected from retinitis pigmentosa or Leber's congenital amaurosis, cone dystrophy or cone-rod dystrophy, thereby silencing the expression of said gene.

Furthermore, the same construct or alternatively a second construct may deliver a replacement cDNA for the mutated gene, eg a nucleotide sequence coding for a wild-type form of a mutated coding sequence, wherein said mutated coding sequence is responsible for the retinal dystrophy.

Ectopic expression of a gene is an abnormal gene expression in a cell type, tissue type, or developmental stage in which said gene is not usually expressed.

The invention relies on the use of ectopic expression of endogenous transcription factors (TFs) in rod photoreceptors or in cone cells. Said TFs, which are not physiologically expressed in rod photoreceptors or in cone photoreceptors, are used to repress genes expression of retinal diseases genes affecting the retina and preferably rod photoreceptors or cone photoreceptors. Repression of diseases gene expression by ectopic TFs is expected to prevent the toxic effect causing said retinal diseases.

In a preferred embodiment, the retinal dystrophy is characterized by photoreceptor degeneration, preferably rod cells degeneration or cone cells degeneration. Preferably, the retinal dystrophy is an inherited retinal dystrophy. Still preferably the inherited retinal degeneration is selected from the group consisting of dominant forms of: Retinitis Pigmentosa (RP), and Leber Congenital Amaurosis (LCA) with rod primary disease; alternatively, the retinal degeneration is a cone dystrophy or a cone-rod dystrophy.

Preferably, one or more wild-type forms of the coding sequence responsible for the retinal dystrophy is selected from the group consisting of any one of SEQ ID NO: 416 to SEQ ID No. 427. Any combination of SEQ ID NO: 416 to SEQ ID No. 427 is suitable for the present invention.

It is contemplated that the therapeutic methods of the present invention may be used in combination with another method of treating a retinal dystrophy. Additional therapeutic agents may include a neuroprotective molecule such as: growth factors such as ciliary neurotrophic factor (CNTF), glial-derived neurotrophic factor (GDNF), cardiotrophin-1, brain-derived neurotrophic factor (BDNF) and basic fibroblast growth factor (bFGF) or the rod-derived cone viability factors such as RdCVF and RdCVF2.

In the present invention the wild-type form of the coding sequence responsible for the retinal dystrophy, in particular characterized by photoreceptor degeneration, in particular inherited retinal dystrophy are selected from the group consisting of the following genes: RHO, PRPH2, CRX, RP1, GUCA1B, RDH12, NR2E3, NRLROM1, GUCY2D, CUGA1A.

In an embodiment of the invention the promoter is a rod specific promoter, in a still preferred embodiment the promoter is selected from: hGNAT1 (SEQ ID No. 12), or any one of SEQ ID No. 13 to 23.

In an alternative embodiment of the invention the promoter is a cone specific promoter, preferably the red opsin gene promoter.

The compositions of the present invention may be in form of a solution, e.g. an injectable solution, a cream, ointment, tablet, suspension or the like. The composition may be administered in any suitable way, e.g. by injection, particularly by intraocular injection, preferably by subretinal injection, by oral, topical, nasal, rectal application etc. The carrier may be any suitable pharmaceutical carrier. Preferably, a carrier is used, which is capable of increasing the efficacy of the DNA molecules to enter the target-cells. Suitable examples of such carriers are liposomes, particularly cationic liposomes.

By “biologically compatible form suitable for administration in vivo” is meant a form of the substance to be administered in which any toxic effects are outweighed by the therapeutic effects. Administration of a therapeutically active amount of the pharmaceutical compositions of the present invention, or an “effective amount”, is defined as an amount effective at dosages and for periods of time, necessary to achieve the desired result of increasing/decreasing the production of proteins. A therapeutically effective amount of a substance may vary according to factors such as the disease state/health, age, sex, and weight of the recipient, and the inherent ability of the particular polypeptide, nucleic acid coding therefore, or recombinant virus to elicit the desired response. Dosage regimen may be adjusted to provide the optimum therapeutic response. For example, several divided doses may be administered daily or at periodic intervals, and/or the dose may be proportionally reduced as indicated by the exigencies of the therapeutic situation. Suitable administration routes are intramuscular injections, subcutaneous injections, intravenous injections or intraperitoneal injections, oral and intranasal administration. In the case of IRD, injecting the constructs of the invention into the retina of the subject may be preferred. The composition of the invention may also be provided via implants, which can be used for slow release of the composition over time.

In the case of photoreceptor degeneration, such as in IRDs (in particular, Retinitis Pigmentosa (RP), Leber Congenital Amaurosis (LCA), cone-rod dystrophies and cone dystrophies), the compositions of the invention may be administered topically to the eye in effective volumes of from about 5 microliters to about 75 microliters, for example from about 7 microliters to about 50 microliters, preferably from about 10 microliters to about 30 microliters. The constructs of the invention may be highly soluble in aqueous solutions. Topical instillation in the eye of compositions of the invention in volumes greater than 75 microliters may result in loss of composition from the eye through spillage and drainage. Thus, it is preferred to administer a high concentration of composition (e.g., from 1 nM to 100 μM, with a preferred range between 10 and 1000 nM) by topical instillation to the eye in volumes of from about 5 microliters to about 75 microliters.

In one aspect, the parenteral administration route may be intraocular administration. Intraocular administration of the present composition can be accomplished by injection or direct (e.g., topical) administration to the eye, as long as the administration route allows the miRNA to enter the eye. In addition to the topical routes of administration to the eye described above, suitable intraocular routes of administration include intravitreal, intraretinal, subretinal, subtenon, peri- and retro-orbital, trans-corneal and trans-scleral administration. Such intraocular administration routes are within the skill in the art (Acheampong A A et al, 2002, Drug Metabol. and Disposition 30: 421-429; Bennett J, Pakola S, Zeng Y, Maguire A M. Hum Gene Ther. 1996; 7:1763-1769; Ambatia, J., and Adamis, A. P., Progress in Retinal and Eye Res. 2002; 21: 145-151 and Cheng Y, Ji R, Yue J, et al. Am J Pathol 2007; 170: 1831-1840).

The inventors have selected transcription factors based on their ability to recognize specific DNA sequence motifs present in the promoter of certain genes responsible for autosomal dominant forms of retinal dystrophies, their lack of expression in terminally differentiated rod photoreceptors or cone photoreceptors and their ability to silence said genes.

In an example, the inventors have selected the TF Kruppel-like factor 15 (KLF15) based on its putative ability to recognize a specific DNA sequence motif present in the RHODOPSIN (RHO) promoter and its lack of expression in terminally differentiated rod photoreceptors (the RHO-expressing cells). The inventors have surprisingly found that adeno-associated virus (AAV) vector-mediated ectopic expression of KLF15 in rod photoreceptors enables Rho silencing with limited genome-wide transcriptional perturbations. Suppression of a RHO mutant allele by KLF15 corrects the phenotype of a mouse model of retinitis pigmentosa (RP) with no observed toxicity.

The invention will be now illustrated by means of non-limiting examples referring to the following figures.

### BRIEF DESCRIPTION OF THE DRAWINGS

FIG. 1. KLF15 is not expressed in rods and binds the human Rhodopsin promoter.
(A) Transfac® analysis of the human rhodopsin promoter identifies TFs predicted to bind the Rhodopsin regulatory motif hRHOcis (−88 to −58 from the Transcription Start Site, TSS; FIG. 2A, (12, 13)) including KLF15 TF (orange arrow, minus strand).
(B) Immunofluorescence analysis of Klf15 in C57Bl6/J retina shows its absence in photoreceptors in the outer nuclear layer (ONL) and expression in the inner nuclear layer (INL) and in the ganglion cell layer (GCL); scale bar 50 μm.
(C) qReal Time PCR of mRNA (2-ΔCt) shows that Klf15 is not expressed in porcine rods. Porcine rodstransduced with AAV8-hGNAT1-eGFP (1×1012, vectorgenomes, GC) and FACS sorted show lack of expression of Klf15. For comparison the retinal-specific Cone-Rod Homeobox (Crx) and rod-specific Neural Retina Leucine Zipper (Nrl) TFs are shown.
(D) Gel mobility shift titrations of hKLF15 and artificial ZF6-DB transcription factor with the hRHO 65 bp oligonucleotide. In the saturation binding experiments the nanomolar concentration of specific binding data were plotted against nanomolar increasing concentration of DNA ligand. KLF15 and the synthetic-TF ZF6-DB show similar binding affinity for the target sequence (12, 13).
(E) qReal Time PCR ChIP analysis of the human rhodopsin TSS region, after the transfection of hKLF15 in HEK293 cells, shows enrichment of binding in the Rho promoter region compared with eGFP transfected cells; Error bars, means+/−s.e.m. **p<0.01; two-tailed Student's t test. n=3 independent experiments.

FIG. 2. KLF15 ectopically expressed in porcine rod photoreceptors represses Rho expression with limited off-targeting.
(A) Alignment of human, porcine and murine Rho proximal promoter around the hRHOcis. In red, the sequence recognized by KLF15 retrieved by Transfac analysis (FIG. 1A-Table 1).
(B) qReal Time PCR of mRNA levels (2-ΔΔCt) of adult porcine retina injected subretinally with AAV8-hGNAT1-hKLF15 (n=6) or AAV8-hGNAT1-eGFP (n=6) at a vector dose of 2×1010 genome copies (gc) 15 days after vector delivery shows significant repression of the Rho transcript; Rho, Rhodopsin; Gnat1, Guanine Nucleotide Binding Protein1; Arr3, Arrestin 3. Error bars, means+/−s.e.m. ***p<0.001; two-tailed Student's t test.
(C) Western Blot analysis of porcine retinae injected with AAV8-hGNAT1-hKLF15 and AAV8-hGNAT1-eGFP shows the decrease in Rho protein consequent to KLF15 expression.
(D) Rho (cyan) and KLF15 (red) immunofluorescence confocal analysis shows expression of hKLF15 in the ONL of injected retina (co-injected with AAV8-hGNAT1-eGFP, green) toward the nuclear interior of rod photoreceptor nuclei (euchromatin, (33)), the collapse of the Rho-deprived outer-segment (OS) and partial retention of Rho in the cytoplasm.
(E) Histological confocal immunofluorescence analysis of Gnat1 (red), which marks the soma of rods, confirmed rod-specific expression of hKLF15 upon transduction with AAV8-hGNAT1-hKLF15. Scale bar, 50 μm.

FIG. 3. KLF15 ectopic expression preserves retinal function in adRP transgenic RHO-P347S mice.
(A) Electroretinography (ERG) traces from a representative mouse injected with AAV carrying hKLF15, mKlf15 or eGFP measured at increasing luminances (cd·s/m2).
(B) ERG analysis on P347S mice subretinally injected at postnatal day 14 (P14) with AAV8-hGNAT1-hKLF15 (n=12), AAV8-hGNAT1-mKlf15 (n=9), AAV8-hGNAT1-eGFP (n=14) or not injected (n=6) and analysed at P30. Retinal responses in both scotopic (dim light) and photopic (bright light) showed that both A- and B-wave amplitudes, evoked by increasing light intensities, were more preserved in hKLF15 and mKlf15 injected eyes compared to eGFP control eyes.
(C) Immunofluorescence staining of P347S mouse retina, injected at P14 with AAV8-hGNAT1-hKLF15, AAV8-hGNAT1-mKlf15 or AAV8-hGNAT1-eGFP and analysed at P30. hKLF15 and mKlf15 treated retina show KLF15 positive expression toward the periphery of rod photoreceptor nuclei, an inverted pattern compared with pig (FIG. 2D, (33)), and higher preservation of the ONL compared with eGFP controls. ONL, outer nuclear layer; INL, inner nuclear layer.
(D) qReal Time PCR of mRNA levels (2-ΔCt normalized on mGnat1 gene) demonstrates that hKLF15 and mKLF15 down-regulate human P347S RHO expression without changing the endogenous wild type murine Rhodopsin transcript.

FIG. 4. Klf15 is not expressed in rods.
(A-B) Immunofluorescences of Klf15 in murine, porcine and human retina show the absence of endogenous Klf15 in rods. Scale bar 50 μm. (C) Co-immunofluorescence confocal analysis of porcine retina injected with AAV8-hGNAT1-eGFP to mark rods (green), shows the presence of Klf15 expression, in grey, in the inner nuclear layer, (INL) in the ganglion cell layer (GCL) and in cones, as revealed by co-expression with arrestin 3, (Arr3) in red, whereas eGFP shows no co-localization with Klf15 staining. OS, outer segment; ONL, outer nuclear layer; INL, inner nuclear layer. Scale bar 25 μm.

FIG. 5. hKLF15 and mKlf15 preserve retinal morphology in adRP transgenic RHO-P347S mice. Heamatoxylin and eosin (H&E) staining of P347S mouse retinae shows the preservation of Outer Nuclear Layer (ONL) morphology in the eyes injected with AAV8 driving hKLF15 or mKlf15 compared with eGFP treated eyes. RPE, retinal pigment epithelium; ONL, outer nuclear layer; INL, inner nuclear layer; IPL, inner plexiform layer.

FIG. 6. Human hKLF15 and murine mKlf15 ectopic expression in wild type mouse retina do not exert detrimental effects.
(A) Electroretinography (ERG) analysis on wild-type C57Bl6/J mice subretinally injected at postnatal day 60 (PD60) with AAV8-CMV-hKLF15 (n=5) AAV8-CMV-mKlf15 (n=5) or AAV8-hGNAT1-eGFP (n=5) and analysed after 80 days. Retinal responses in both scotopic (dim light) and photopic (bright light) show no differences in A- (left panel) and B-waves (right panel) amplitudes, evoked by increasing light intensities.
(B) qReal Time PCR of murine Rho expression mRNA levels (2-ΔΔCt) show no differences upon injection of AAV8-CMV-hKLF15, AAV8-CMV-mKlf15 or AAV8-CMV-eGFP. Error bars, means+/−s.e.m. *p<0.05, ***p<0.001; two-tailed Student's t test.

FIG. 7. Immunofluorescence analysis of C57Bl6/J wild-type mice subretinally injected with KLF15.
(A) Klf15 staining of retina following administration at postnatal day 60 (P60) of AAV8-hGNAT1-eGFP (n=5), AAV8-CMV-hKLF15 (n=5) or AAV8-CMV-mKlf15 (n=5) and analysed after 80 days post injection (P140). Transduced retinae show expression and maintenance of ONL integrity upon human and murine KLF15 expression (red) in the ONL;
(B) rhodopsin localization and expression in the correspondent transduced areas is unaltered upon human and murine Klf15 expression in rods (A). Outer nuclear layer, ONL, inner nuclear layer, INL, and ganglion cells, GC.

FIG. 8. Transfac® analysis of the human PRPH2 promoter identifies TFs predicted to bind the PRPH2 regulatory region

FIG. 9. Transfac® analysis of the human CRX promoter identifies TFs predicted to bind the CRX regulatory region

FIG. 10. Transfac® analysis of the human RP1 promoter identifies TFs predicted to bind the RP1 regulatory region

FIG. 11. Transfac® analysis of the human GUCA1B Promoter identifies TFs predicted to bind the GUCA1B regulatory region

FIG. 12. Transfac® analysis of the human RDH12 Promoter identifies TFs predicted to bind the RDH12 regulatory region

FIG. 13. Transfac® analysis of the human GUCA1A, guanylate cyclase activator 1A Promoter identifies TFs predicted to bind the GUCA1A regulatory region

FIG. 14. Transfac® analysis of the human GUCY2D, guanylate cyclase 2D, retinal Promoter identifies TFs predicted to bind the GUCY2D regulatory region

FIG. 15. Transfac® analysis of the human N2RE3 Promoter identifies TFs predicted to bind the N2RE3 regulatory region

FIG. 16. Transfac® analysis of the human N2RL Promoter identifies TFs predicted to bind the NRL regulatory region

FIG. 17. Transfac® analysis of the human OTX2, Promoter identifies TFs predicted to bind the OTX2 regulatory region

FIG. 18. Transfac® analysis of the human ROM1 Promoter identifies TFs predicted to bind the ROM1 regulatory region

Brief Description of the Sequences in the Sequence listing

Promoters:
hGNAT1
(SEQ ID NO: 12)
TCCCTGCAGGTCATAAAATCCCAGTCCAGAGTCACCAGCCCTTCTTAACCACTTCCTACTGTGTGACCCT
TTCAGCCTTTACTTCCTCATCAGTAAAATGAGGCTGATGATATGGGCATCCATACTCCAGGGCCAGTGT
GAGCTTACAACAAGATAAGGAGTGGTGCTGAGCCTGGTGCCGGGCAGGCAGCAGGCATGTTTCTCCC
AATTATGCCCTCTCACTGCCAGCCCCACCTCCATTGTCCTCACCCCCAGGGCTCAAGGTTCTGCCTTCCC
CTTTCTCAGCCCTGACCCTACTGAACATGTCTCCCCACTCCCAGGCAGTGCCAGGGCCTCTCCTGGAGG
GTTGCGGGGACAGAAGGACAGCCGGAGTGCAGAGTCAGCGGTTGAGGGATTGGGGCTATGCCAGCT
AATCCGAAGGGTTGGGGGGGCTGAGCTGGATTCACCTGTCCTTGTCTCTGATTGGCTCTTGGACACCC
CTAGCCCCCAAATCCCACTAAGCAGCCCCACCAGGGATTGCACAGGTCCGTAGAGAGCCAGTTGATTG
CAGGTCCTCCTGGGGCCAGAAGGGTGCCTGGGAGGCCAGGTTCTGGGGATCCCCTCCATCCAGAAGA
ACCACCTGCTCACTCTGTCCCTTCGCCTGCTGCTGGGACC
Rod specific promoters:
Nucleotide sequence of Prom A (SEQ ID No. 13)
TCCTCCTAGTGTCACCTTGGCCCCTCTTAGAAGCCAATTAGGCCCTCAGTTTCTGCAGCGGGGATTAAT
ATGATTATGAACACCCCCAATCGATGCTGATTCAGCCAGGAGCTTAGGAGGGGGAGGTCACTTTATAA
GGGTCTGGGGGGGTCAGAACCCAGAGTCATCCAGCTGGAGCCCTGAGTGGCTGAGCTCAGGCCTTCG
CAGCATTCTTGGGTGGGAGCAGCCACGGGTCAGCCACAAGGGCCACAGCC
Nucleotide sequence of Prom B (SEQ ID No.14)
TCCTCCTAGTGTCACCTTGGCCCCTCTTAGAAGCCAATTAGGCCCTCAGTTTCTGCAGCGGGGATTAAT
ATGATTATGAACACCCCCAATCTCAACTCGTAGGATTCAGCCAGGAGCTTAGGAGGGGGAGGTCACTT
TATAAGGGTCTGGGGGGGTCAGAACCCAGAGTCATCCAGCTGGAGCCCTGAGTGGCTGAGCTCAGGC
CTTCGCAGCATTCTTGGGTGGGAGCAGCCACGGGTCAGCCACAAGGGCCACAGCC
Nucleotide sequence of Prom C (SEQ ID No. 15)
TCCTCCTAGTGTCACCTTGGCCCCTCTTAGAAGCCAATTAGGCCCTCAGTTTCTGCAGCGGGGATTAAT
ATGATTATGAACACCCCCACGAGAAACTCTGCTGATTCAGCCAGGAGCTTAGGAGGGGGAGGTCACTT
TATAAGGGTCTGGGGGGGTCAGAACCCAGAGTCATCCAGCTGGAGCCCTGAGTGGCTGAGCTCAGGC
CTTCGCAGCATTCTTGGGTGGGAGCAGCCACGGGTCAGCCACAAGGGCCACAGCC
Nucleotide sequence of Prom D (SEQ ID No.16)
TCCTCCTAGTGTCACCTTGGCCCCTCTTAGAAGCCAATTAGGCCCTCAGTTTCTGCAGCGGGGATTAAT
ATGATTAGTCCACACCCCACGAGAAACTCTGCTGATTCAGCCAGGAGCTTAGGAGGGGGAGGTCACTT
TATAAGGGTCTGGGGGGGTCAGAACCCAGAGTCATCCAGCTGGAGCCCTGAGTGGCTGAGCTCAGGC
CTTCGCAGCATTCTTGGGTGGGAGCAGCCACGGGTCAGCCACAAGGGCCACAGCC
Nucleotide sequence of Prom E (SEQ ID No.17)
TCCTCCTAGTGTCACCTTGGCCCCTCTTAGAAGCCAATTAGGCCCTCAGTTTCTGCAGCGGGGATTAAT
ATGATTATGAACACATGATATCTCCCAGATGCTGATTCAGCCAGGAGCTTAGGAGGGGGAGGTCACTT
TATAAGGGTCTGGGGGGGTCAGAACCCAGAGTCATCCAGCTGGAGCCCTGAGTGGCTGAGCTCAGGC
CTTCGCAGCATTCTTGGGTGGGAGCAGCCACGGGTCAGCCACAAGGGCCACAGCC
Nucleotide sequence of Prom F (SEQ ID No.18)
TCCTCCTAGTGTCACCTTGGCCCCTCTTAGAAGCCAATTAGGCCCTCAGTTTCTGCAGCGGGGATTAAT
ATGATTATGAACACATCTCCCAGATGCTGATTCAGCCAGGAGCTTAGGAGGGGGAGGTCACTTTATAA
GGGTCTGGGGGGGTCAGAACCCAGAGTCATCCAGCTGGAGCCCTGAGTGGCTGAGCTCAGGCCTTCG
CAGCATTCTTGGGTGGGAGCAGCCACGGGTCAGCCACAAGGGCCACAGCC
Nucleotide sequence of Prom G (SEQ ID No.19)
TCCTCCTAGTGTCACCTTGGCCCCTCTTAGAAGCCAATTAGGCCCTCAGTTTCTGCAGCGGGGATTAAT
ATGATTAGTCCACACCCCAATCTCCCAGATGCTGATTCAGCCAGGAGCTTAGGAGGGGGAGGTCACTT
TATAAGGGTCTGGGGGGGTCAGAACCCAGAGTCATCCAGCTGGAGCCCTGAGTGGCTGAGCTCAGGC
CTTCGCAGCATTCTTGGGTGGGAGCAGCCACGGGTCAGCCACAAGGGCCACAGCC
Nucleotide sequence of Prom H (SEQ ID No.20)
TCCTCCTAGTGTCACCTTGGCCCCTCTTAGAAGCCAATTAGGCCCTCAGTTTCTGCAGCGGGGATTAAT
ATGATTACGACCGTATCGGGGTTAGGGAGTGCTGATTCAGCCAGGAGCTTAGGAGGGGGAGGTCACT
TTATAAGGGTCTGGGGGGGTCAGAACCCAGAGTCATCCAGCTGGAGCCCTGAGTGGCTGAGCTCAGG
CCTTCGCAGCATTCTTGGGTGGGAGCAGCCACGGGTCAGCCACAAGGGCCACAGCC
Nucleotide sequence of Prom I (SEQ ID No.21)
TCCTCCTAGTGTCACCTTGGCCCCTCTTAGAAGCCAATTAGGCCCTCAGTTTCTGCAGCGGGGATTAAT
ATGATTATCCCCCAATCTCCCAGATGCTGATTCAGCCAGGAGCTTAGGAGGGGGAGGTCACTTTATAA
GGGTCTGGGGGGGTCAGAACCCAGAGTCATCCAGCTGGAGCCCTGAGTGGCTGAGCTCAGGCCTTCG
CAGCATTCTTGGGTGGGAGCAGCCACGGGTCAGCCACAAGGGCCACAGCC
Nucleotide sequence of Prom L (SEQ ID No.22)
TCCTCCTAGTGTCACCTTGGCCCCTCTTAGAAGCCAATTAGGCCCTCAGTTTCTGCAGCGGGGATTAAT
ATGATTAGAGGGATTGGTGCTATGCCAGCTGCTGATTCAGCCAGGAGCTTAGGAGGGGGAGGTCACT
TTATAAGGGTCTGGGGGGGTCAGAACCCAGAGTCATCCAGCTGGAGCCCTGAGTGGCTGAGCTCAGG
CCTTCGCAGCATTCTTGGGTGGGAGCAGCCACGGGTCAGCCACAAGGGCCACAGCC
Nucleotide sequence of Prom hRHO-s-AZF6 (SEQ ID No.23)
TCCTCCTAGTGTCACCTTGGCCCCTCTTAGAAGCCAATTAGGCCCTCAGTTTCTGCAGCGGGGATTAAT
ATGATTATGAAATCTCCCAGATGCTGATTCAGCCAGGAGCTTAGGAGGGGGAGGTCACTTTATAAGGG
TCTGGGGGGGTCAGAACCCAGAGTCATCCAGCTGGAGCCCTGAGTGGCTGAGCTCAGGCCTTCGCAG
CATTCTTGGGTGGGAGCAGCCACGGGTCAGCCACAAGGGCCACAGCC
Transcription factors:
hKLF15 CDS, SEQ ID NO. 837:
ATGGTGGACCACTTACTTCCAGTGGACGAGAACTTCTCGTCGCCAAAATGCCCAGTTGGGTATCTGGGT
GATAGGCTGGTTGGCCGGCGGGCATATCACATGCTGCCCTCACCCGTCTCTGAAGATGACAGCGATGC
CTCCAGCCCCTGCTCCTGTTCCAGTCCCGACTCTCAAGCCCTCTGCTCCTGCTATGGTGGAGGCCTGGG
CACCGAGAGCCAGGACAGCATCTTGGACTTCCTATTGTCCCAGGCCACGCTGGGCAGTGGCGGGGGC
AGCGGCAGTAGCATTGGGGCCAGCAGTGGCCCCGTGGCCTGGGGGCCCTGGCGAAGGGCAGCGGCC
CCTGTGAAGGGGGAGCATTTCTGCTTGCCCGAGTTTCCTTTGGGTGATCCTGATGACGTCCCACGGCCC
TTCCAGCCTACCCTGGAGGAGATTGAAGAGTTTCTGGAGGAGAACATGGAGCCTGGAGTCAAGGAGG
TCCCTGAGGGCAACAGCAAGGACTTGGATGCCTGCAGCCAGCTCTCAGCTGGGCCACACAAGAGCCAC
CTCCATCCTGGGTCCAGCGGGAGAGAGCGCTGTTCCCCTCCACCAGGTGGTGCCAGTGCAGGAGGTG
CCCAGGGCCCAGGTGGGGGCCCCACGCCTGATGGCCCCATCCCAGTGTTGCTGCAGATCCAGCCCGTG
CCTGTGAAGCAGGAATCGGGCACAGGGCCTGCCTCCCCTGGGCAAGCCCCAGAGAATGTCAAGGTTG
CCCAGCTCCTGGTCAACATCCAGGGGCAGACCTTCGCACTCGTGCCCCAGGTGGTACCCTCCTCCAACT
TGAACCTGCCCTCCAAGTTTGTGCGCATTGCCCCTGTGCCCATTGCCGCCAAGCCTGTTGGATCGGGAC
CCCTGGGGCCTGGCCCTGCCGGTCTCCTCATGGGCCAGAAGTTCCCCAAGAACCCAGCCGCAGAACTC
ATCAAAATGCACAAATGTACTTTCCCTGGCTGCAGCAAGATGTACACCAAAAGCAGCCACCTCAAGGCC
CACCTGCGCCGGCACACGGGTGAGAAGCCCTTCGCCTGCACCTGGCCAGGCTGCGGCTGGAGGTTCTC
GCGCTCTGACGAGCTGTCGCGGCACAGGCGCTCGCACTCAGGTGTGAAGCCGTACCAGTGTCCTGTGT
GCGAGAAGAAGTTCGCGCGGAGCGACCACCTCTCCAAGCACATCAAGGTGCACCGCTTCCCGCGGAG
CAGCCGCTCCGTGCGCTCCGTGAACTGA
hKLF8 CDS, SEQ ID No. 838
ATGGTCGATATGGATAAACTCATAAACAACTTGGAGGTCCAACTTAATTCAGAAGGTGGCTCAATGCA
GGTATTCAAGCAGGTCACTGCTTCTGTTCGGAACAGAGATCCCCCTGAGATAGAATACAGAAGTAATA
TGACTTCTCCAACACTCCTGGATGCCAACCCCATGGAGAACCCAGCACTGTTTAATGACATCAAGATTG
AGCCCCCAGAAGAACTTTTGGCTAGTGATTTCAGCCTGCCCCAAGTGGAACCAGTTGACCTCTCCTTTC
ACAAGCCCAAGGCTCCTCTCCAGCCTGCTAGCATGCTACAAGCTCCAATACGTCCCCCCAAGCCACAGT
CTTCTCCCCAGACCCTTGTGGTGTCCACGTCAACATCTGACATGAGCACTTCAGCAAACATTCCTACTGT
TCTGACCCCAGGCTCTGTCCTGACCTCCTCTCAGAGCACTGGTAGCCAGCAGATCTTACATGTCATTCAC
ACTATCCCCTCAGTCAGTCTGCCAAATAAGATGGGTGGCCTGAAGACCATCCCAGTGGTAGTGCAGTCT
CTGCCCATGGTGTATACTACTTTGCCTGCAGATGGGGGCCCTGCAGCCATTACAGTCCCACTCATTGGA
GGAGATGGTAAAAATGCTGGATCAGTGAAAGTTGACCCCACCTCCATGTCTCCACTGGAAATTCCAAG
TGACAGTGAGGAGAGTACAATTGAGAGTGGATCCTCAGCCTTGCAGAGTCTGCAGGGACTACAGCAA
GAACCAGCAGCAATGGCCCAAATGCAGGGAGAAGAGTCGCTTGACTTGAAGAGAAGACGGATTCACC
AATGTGACTTTGCAGGATGCAGCAAAGTGTACACCAAAAGCTCTCACCTGAAAGCTCACCGCAGAATC
CATACAGGAGAGAAGCCTTATAAATGCACCTGGGATGGCTGCTCCTGGAAATTTGCTCGCTCAGATGA
GCTCACTCGCCATTTCCGCAAGCACACAGGCATCAAGCCTTTTCGGTGCACAGACTGCAACCGCAGCTT
TTCTCGTTCTGACCACCTGTCCCTGCATCGCCGTCGCCATGACACCATGTGA
Aminoacid sequence SEQ ID No. 839
MVDMDKLINNLEVQLNSEGGSMQVFKQVTASVRNRDPPEIEYRSNMTSPTLLDANPMENPALFNDIKIEP
PEELLASDFSLPQVEPVDLSFHKPKAPLQPASMLQAPIRPPKPQSSPQTLVVSTSTSDMSTSANIPTVLT
PGSVLTSSQSTGSQQILHVIHTIPSVSLPNKMGGLKTIPVVVQSLPMVYTTLPADGGPAAITVPLIGGDG
KNAGSVKVDPTSMSPLEIPSDSEESTIESGSSALQSLQGLQQEPAAMAQMQGEESLDLKRRRIHQCDFAG
CSKVYTKSSHLKAHRRIHTGEKPYKCTWDGCSWKFARSDELTRHFRKHTGIKPFRCTDCNRSFSRSDHLS
LHRRRHDTM
Zinc finger protein 780A (O75290)
SEQ ID No. 840
ATGGTCCATGGATCAGTGACATTCAGGGATGTGGCCATTGACTTCTCTCAGGAGGAGTGGGAGTGCCT
GCAGCCTGATCAGAGGACCTTGTACAGGGATGTGATGTTGGAGAACTACAGCCACCTGATCTCACTGG
CAGGAAGTTCCATTTCTAAACCAGATGTAATTACGTTACTAGAGCAAGAGAAAGAGCCCTGGATGGTT
GTAAGGAAAGAAACAAGCAGACGGTATCCAGATTTGGAGTTAAAATATGGACCTGAGAAAGTATCTCC
AGAAAATGATACCTCTGAAGTAAATTTACCCAAACAGGTTATAAAGCAAATAAGTACAACTCTTGGCAT
TGAGGCCTTTTATTTTAGAAATGACTCAGAATATAGACAATTTGAGGGACTACAGGGATATCAAGAAG
GAAATATCAATCAAAAGATGATCAGCTATGAAAAACTGCCTACTCATACTCCTCATGCTTCTCTTATTTG
CAATACACATAAACCGTATGAATGTAAGGAATGTGGGAAATACTTTAGTCGTAGTGCAAATCTTATTCA
GCATCAGAGTATTCATACTGGAGAGAAACCCTTTGAATGTAAGGAGTGTGGGAAAGCCTTTCGACTTC
ACATACAATTTACTCGACATCAGAAATTTCATACTGGTGAGAAACCTTTTGAATGTAACGAATGTGGAA
AGGCCTTTAGTCTTCTTACCCTGCTTAATCGCCATAAGAACATTCACACAGGTGAGAAACTGTTTGAAT
GTAAGGAATGTGGGAAGTCCTTTAATCGTAGCTCAAACCTTGTTCAACATCAGAGTATTCATTCTGGTG
TAAAACCATATGAATGTAAGGAGTGTGGGAAAGGCTTTAATCGTGGTGCACACCTTATTCAGCATCAG
AAAATTCATTCCAATGAGAAACCCTTTGTATGTAAGGAATGTGGGATGGCCTTTCGATATCATTACCAA
CTTATTGAACATTGCCAAATTCATACTGGTGAGAAACCCTTTGAATGTAAAGAATGTGGAAAGGCGTTT
ACTCTTCTGACAAAGCTTGTTCGACATCAGAAGATTCATACTGGTGAGAAACCCTTTGAATGCAGGGAA
TGTGGGAAGGCCTTTAGTCTTCTCAACCAGCTTAATCGCCATAAGAACATTCACACAGGTGAAAAACCG
TTTGAATGTAAGGAATGTGGGAAGTCCTTTAATCGTAGCTCAAACCTTGTTCAACATCAGAGTATTCAT
GCTGGTATAAAACCATATGAATGTAAGGAGTGTGGGAAAGGCTTTAATCGTGGTGCACACCTTATTCA
GCATCAGAAAATTCATTCCAATGAGAAACCTTTTGTATGTAGGGAATGTGAGATGGCCTTTAGATATCA
TTGCCAACTTATTGAACATTCTCGAATTCATACTGGTGACAAGCCATTTGAATGTCAAGACTGTGGGAA
GGCCTTCAATCGTGGCTCAAGCCTTGTTCAACATCAGAGTATTCACACTGGTGAGAAGCCCTATGAATG
TAAGGAGTGTGGGAAGGCTTTTAGACTTTACCTACAACTTTCCCAACATCAGAAAACTCACACAGGTGA
AAAACCATTTGAATGTAAGGAATGTGGGAAATTCTTTCGTCGTGGTTCAAATCTTAATCAACATCGAAG
TATTCATACTGGAAAGAAACCCTTTGAATGTAAGGAATGTGGGAAAGCCTTTCGACTTCATATGCACCT
TATTCGACATCAGAAATTGCATACTGGTGAGAAACCCTTTGAATGTAAGGAGTGTGGGAAAGCCTTTC
GACTTCATATGCAACTTATTCGACATCAGAAATTGCATACTGGTGAGAAACCCTTTGAATGTAAGGAAT
GTGGAAAGGTTTTTAGTCTTCCCACCCAGCTTAATCGCCATAAGAACATTCACACAGGTGAGAAGGCAT
CTTGA
Aminoacid sequence SEQ ID No. 841
MVHGSVTFRDVAIDFSQEEWECLQPDQRTLYRDVMLENYSHLISLAGSSISKPDVITLLEQEKEPWMVVR
KETSRRYPDLELKYGPEKVSPENDTSEVNLPKQVIKQISTTLGIEAFYFRNDSEYRQFEGLQGYQEGNIN
QKMISYEKLPTHTPHASLICNTHKPYECKECGKYFSRSANLIQHQSIHTGEKPFECKECGKAFRLHIQFT
RHQKFHTGEKPFECNECGKAFSLLTLLNRHKNIHTGEKLFECKECGKSFNRSSNLVQHQSIHSGVKPYEC
KECGKGFNRGAHLIQHQKIHSNEKPFVCKECGMAFRYHYQLIEHCQIHTGEKPFECKECGKAFTLLTKLV
RHQKIHTGEKPFECRECGKAFSLLNQLNRHKNIHTGEKPFECKECGKSFNRSSNLVQHQSIHAGIKPYEC
KECGKGFNRGAHLIQHQKIHSNEKPFVCRECEMAFRYHCQLIEHSRIHTGDKPFECQDCGKAFNRGSSLV
QHQSIHTGEKPYECKECGKAFRLYLOLSQHQKTHTGEKPFECKECGKFFRRGSNLNQHRSIHTGKKPFEC
KECGKAFRLHMHLIRHQKLHTGEKPFECKECGKAFRLHMQLIRHQKLHTGEKPFECKECGKVFSLPTQLN
RHKNIHTGEKAS
HMX1 (Q9NP08), SEQ ID No. 842
ATGCCTGACGAGCTGACGGAGCCCGGGCGCGCCACGCCGGCCCGCGCCTCCTCCTTCCTCATCGAGAA
CCTGCTGGCGGCCGAGGCCAAGGGCGCAGGGCGCGCGACCCAGGGCGACGGCAGCCGGGAGGACG
AGGAGGAGGACGACGACGACCCCGAAGACGAGGACGCCGAGCAGGCGCGGCGGCGACGGCTACAG
CGGCGGCGACAGTTGCTCGCGGGCACCGGGCCCGGCGGGGAGGCGCGGGCCCGTGCGCTGCTCGGG
CCGGGCGCGCTGGGCCTCGGTCCTCGGCCGCCCCCCGGTCCCGGGCCGCCCTTCGCTCTGGGCTGCGG
AGGCGCAGCGCGCTGGTACCCACGGGCGCACGGTGGCTATGGAGGCGGCCTCAGTCCTGACACCAGC
GACCGGGACTCACCGGAGACGGGCGAGGAGATGGGCCGTGCGGAGGGCGCCTGGCCGCGAGGCCCC
GGGCCGGGAGCGGTGCAGCGGGAGGCAGCGGAGCTGGCGGCGCGTGGCCCGGCGGCCGGCACGGA
GGAGGCGTCGGAGCTGGCCGAGGTCCCTGCGGCGGCTGGGGAGACACGCGGCGGCGTTGGCGTGG
GCGGCGGCCGAAAGAAGAAGACGCGCACAGTCTTCTCCCGCAGCCAGGTCTTCCAGCTGGAATCCACC
TTCGACCTGAAGCGCTACCTGAGCAGCGCCGAGCGCGCCGGCCTGGCCGCCTCCCTGCAGCTCACCGA
GACGCAGGTTAAGATCTGGTTCCAGAACCGCCGCAACAAGTGGAAGCGGCAGCTGGCAGCCGAGCTG
GAGGCGGCCAGCCTGTCCCCGCCGGGAGCGCAGCGCCTGGTCCGCGTGCCGGTGCTCTACCACGAAA
GCCCCCCGGCCGCAGCCGCCGCTGGGCCCCCGGCCACCCTGCCCTTCCCGCTGGCGCCCGCCGCGCCC
GCGCCGCCCCCACCGCTGCTCGGCTTCTCCGGGGCCCTCGCCTACCCGCTGGCCGCCTTCCCGGCCGCC
GCCTCCGTGCCCTTTCTGCGGGCGCAGATGCCTGGCCTGGTGTGA
Aminoacid sequence SEQ ID No. 843
MPDELTEPGRATPARASSFLIENLLAAEAKGAGRATQGDGSREDEEEDDDDPEDEDAEQARRRRLQRRRQ
LLAGTGPGGEARARALLGPGALGLGPRPPPGPGPPFALGCGGAARWYPRAHGGYGGGLSPDTSDRDSPE
TGEEMGRAEGAWPRGPGPGAVQREAAELAARGPAAGTEEASELAEVPAAAGETRGGVGVGGGRKKKTR
TVFSRSQVFQLESTFDLKRYLSSAERAGLAASLQLTETQVKIWFQNRRNKWKRQLAAELEAASLSPPGAQRL
VRVPVLYHESPPAAAAAGPPATLPFPLAPAAPAPPPPLLGFSGALAYPLAAFPAAASVPFLRAQMPGLV
MZF-1, Myeloid zinc finger 1 (P28698), SEQ ID No. 844
TGAGGCCTGCGGTGCTGGGCTCCCCAGACCGAGCACCCCCAGAAGATGAGGGGCCTGTCATGGTGAA
GCTAGAGGACTCTGAGGAGGAGGGTGAGGCTGCCTTATGGGACCCAGGCCCTGAAGCTGCACGCCTG
CGTTTCCGGTGCTTCCGCTATGAGGAGGCCACAGGGCCCCAAGAGGCCCTGGCCCAGCTCCGAGAGCT
GTGTCGCCAGTGGCTGCGTCCAGAGGTACGCTCCAAGGAGCAGATGCTGGAGCTGTTGGTGCTGGAG
CAGTTCCTGGGCGCACTGCCCCCTGAGATCCAGGCCCGTGTGCAGGGGCAGCGGCCAGGCAGCCCCG
AGGAGGCTGCTGCCCTAGTAGATGGGCTGCGCCGGGAGCCGGGCGGACCCCGGAGATGGGTCACAG
TCCAGGTGCAGGGCCAGGAGGTCCTATCAGAGAAGATGGAGCCCTCCAGTTTCCAGCCCCTACCTGAA
ACTGAGCCTCCAACTCCAGAGCCTGGGCCCAAGACACCTCCTAGGACTATGCAGGAATCACCACTGGG
CCTGCAGGTGAAAGAGGAGTCAGAGGTTACAGAGGACTCAGATTTCCTGGAGTCTGGGCCTCTAGCT
GCCACCCAGGAGTCTGTACCCACCCTCCTGCCTGAGGAGGCCCAGAGATGTGGGACCGTGCTGGACCA
GATCTTTCCCCACAGCAAGACTGGGCCTGAGGGTCCCTCATGGAGGGAGCACCCCAGGGCCCTGTGGC
ATGAGGAAGCTGGGGGCATCTTCTCCCCAGGGTTCGCGCTGCAGCTAGGCAGCATCTCCGCAGGTCCA
GGTAGTGTAAGCCCTCACCTCCACGTCCCCTGGGACCTCGGCATGGCTGGCCTTTCTGGCCAGATCCAA
TCACCCTCCCGCGAAGGTGGCTTTGCGCATGCGCTTCTGCTCCCCAGCGATCTGAGGAGTGAACAGGA
CCCCACGGACGAGGATCCCTGCCGGGGTGTGGGCCCTGCTCTGATCACCACCCGCTGGCGCTCCCCCA
GGGGCCGGAGCCGGGGCCGCCCCAGCACTGGGGGGGGGGGGTTAGGGGCGGCCGTTGCGATGTAT
GTGGCAAGGTGTTCAGCCAACGCAGCAACCTGCTGAGGCACCAGAAGATCCACACGGGTGAGCGACC
ATTCGTGTGCAGCGAGTGCGGCCGCAGCTTCAGCCGCAGCTCGCACCTGCTGCGCCACCAGCTTACGC
ACACCGAGGAGCGGCCGTTCGTGTGCGGCGACTGTGGCCAGGGCTTCGTGCGCAGCGCGCGCCTGGA
AGAGCATCGGAGAGTGCACACGGGCGAACAGCCTTTCCGTTGCGCTGAGTGCGGCCAGAGCTTCCGG
CAGCGCTCCAATCTGCTGCAGCACCAGCGCATCCACGGCGATCCCCCGGGCCCTGGCGCTAAGCCCCC
GGCCCCTCCTGGTGCGCCCGAGCCTCCCGGCCCCTTTCCGTGCAGCGAGTGCCGCGAGAGCTTCGCGC
GGCGCGCCGTGCTGCTGGAGCACCAGGCGGTACACACGGGCGACAAGTCCTTTGGCTGCGTCGAGTG
CGGCGAGCGCTTCGGCCGCCGCTCAGTGCTGCTGCAGCACCGGCGCGTGCACAGTGGCGAGCGGCCC
TTCGCCTGTGCCGAGTGCGGCCAGAGCTTCCGGCAGCGCTCCAACCTGACGCAGCACCGGCGCATCCA
CACCGGGGAGCGGCCCTTCGCCTGCGCCGAGTGTGGCAAGGCCTTCCGCCAGCGGCCTACGCTCACGC
AGCATCTCCGCGTACACACGGGCGAGAAACCCTTTGCCTGCCCCGAGTGTGGCCAGCGCTTCAGCCAG
CGCCTCAAGCTCACGCGTCATCAGAGGACACACACCGGCGAAAAGCCCTACCACTGCGGTGAGTGC
GGCCTGGGCTTCACGCAGGTCTCGCGGCTCACCGAGCACCAGCGCATCCACACGGGCGAACGGCCCTT
CGCCTGCCCCGAGTGCGGCCAGAGCTTTCGGCAGCACGCCAACCTCACCCAGCACCGGCGCATCCACA
CGGGTGAACGGCCCTACGCATGCCCTGAGTGTGGCAAGGCCTTCCGCCAGCGGCCCACGCTCACGCAG
CATCTGCGCACCCACCGACGAGAGAAGCCCTTCGCCTGCCAGGACTGTGGCCGCCGCTTCCACCAGAG
CACCAAGCTCATTCAGCACCAGCGCGTCCACAGCGCCGAGTAG
Aminoacid sequence, SEQ ID No. 845
MRPAVLGSPDRAPPEDEGPVMVKLEDSEEEGEAALWDPGPEAARLRFRCFRYEEATGPQEALAQLRELCR
QWLRPEVRSKEQMLELLVLEQFLGALPPEIQARVQGQRPGSPEEAAALVDGLRREPGGPRRWVTVQVQG
QEVLSEKMEPSSFQPLPETEPPTPEPGPKTPPRTMQESPLGLQVKEESEVTEDSDFLESGPLAATQESVPT
LLPEEAQRCGTVLDQIFPHSKTGPEGPSWREHPRALWHEEAGGIFSPGFALQLGSISAGPGSVSPHLHVP
WDLGMAGLSGQIQSPSREGGFAHALLLPSDLRSEQDPTDEDPCRGVGPALITTRWRSPRGRSRGRPSTGG
GVVRGGRCDVCGKVFSQRSNLLRHQKIHTGERPFVCSECGRSFSRSSHLLRHOLTHTEERPFVCGDCGQG
FVRSARLEEHRRVHTGEQPFRCAECGQSFRQRSNLLQHQRIHGDPPGPGAKPPAPPGAPEPPGPFPCSEC
RESFARRAVLLEHQAVHTGDKSFGCVECGERFGRRSVLLQHRRVHSGERPFACAECGQSFRQRSNLTQHR
RIHTGERPFACAECGKAFRQRPTLTQHLRVHTGEKPFACPECGQRFSQRLKLTRHQRTHTGEKPYHCGEC
GLGFTQVSRLTEHQRIHTGERPFACPECGQSFRQHANLTQHRRIHTGERPYACPECGKAFRQRPTLTQHL
RTHRREKPFACQDCGRRFHQSTKLIQHQRVHSAE
Zinc finger protein 14 (P17017), SEQ ID No. 846
ATGGACTCAGTCTCCTTTGAGGATGTGGCCGTGAACTTCACCCTGGAGGAGTGGGCTTTGCTGGATTCT
TCACAGAAAAAGCTCTATGAAGATGTGATGCAGGAGACCTTCAAAAACCTGGTTTGTCTAGGAAAAAA
GTGGGAAGACCAGGACATTGAAGATGACCACAGAAACCAGGGGAAAAATCGAAGATGTCATATGGTT
GAGAGACTCTGTGAAAGTAGAAGAGGTAGCAAATGTGGAGAAACCACTAGCCAGATGCCAAATGTTA
ATATCAACAAGGAAACTTTTACTGGAGCAAAACCACATGAATGCAGCTTTTGTGGAAGAGACTTCATTC
ATCATTCGTCCCTTAATAGGCACATGAGATCTCACACTGGACAGAAACCAAATGAGTATCAGGAATATG
AAAAGCAACCATGTAAATGTAAAGCAGTTGGGAAAACCTTCAGTTATCACCACTGCTTTCGCAAACATG
AAAGAACTCACACTGGAGTGAAGCCCTATGAATGTAAACAGTGTGGGAAAGCCTTTATATATTACCAG
CCATTTCAAAGACATGAAAGGACTCATGCTGGACAGAAACCCTATGAATGTAAGCAATGTGGAAAAAC
CTTTATATATTACCAGTCTTTTCAAAAACATGCTCATACTGGAAAGAAACCCTATGAATGTAAACAGTGT
GGGAAAGCCTTTATATGTTACCAATCTTTTCAAAGACACAAAAGGACTCACACTGGAGAGAAACCCTAT
GAATGTAAGCAATGTGGTAAGGCTTTCAGTTGTCCCACATACTTTCGAACTCATGAAAGAACTCACACT
GGAGAAAAACCCTACAAATGTAAAGAATGTGGTAAAGCCTTCAGTTTTCTCAGTTCTTTTCGAAGGCAT
AAAAGGACTCATAGTGGAGAGAAACCCTATGAATGTAAAGAATGTGGAAAAGCCTTCTTTTATTCTGC
AAGCTTTCGAGCACATGTAATAATACACACTGGGGCTCGACCTTATAAATGTAAAGAATGTGGGAAAG
CCTTCAACTCTTCTAATTCCTGTCGAGTGCATGAAAGAACTCATATTGGAGAAAAACCATATGAATGTA
AACGATGTGGCAAATCATTCAGTTGGTCCATTTCTCTTCGATTGCATGAAAGAACTCATACTGGAGAGA
AACCTTATGAGTGTAAACAGTGTCATAAAACCTTCAGTTTTTCAAGTTCCCTTCGAGAACACGAAACAA
CTCACACTGGAGAGAAACCCTATGAATGTAAACAATGTGGTAAAACCTTCAGTTTTTCAAGTTCCCTTC
AAAGACATGAAAGGACTCACAATGCAGAGAAACCCTATGAATGTAAACAGTGTGGGAAAGCCTTCAG
GTGTTCAAGTTATTTTCGAATTCATGAAAGGTCACACACTGGAGAGAAACCCTATGAATGTAAACAGTG
TGGAAAAGTTTTCATTCGTTCCAGTTCCTTTCGACTGCATGAAAGAACACACACTGGAGAGAAACCCTA
TGAATGTAAACTATGCGGTAAAACCTTCAGTTTTTCAAGTTCCCTTCGAGAACATGAAAAAATTCACACT
GGAAATAAGCCTTTTGAGTGTAAGCAATGTGGTAAGGCCTTCCTTCGTTCCAGTCAAATTCGATTGCAT
GAAAGGACTCACACTGGAGAGAAACCGTATCAATGTAAACAATGTGGAAAAGCCTTCATTTCTTCCAG
TAAATTTCGAATGCATGAGAGAACTCACACGGGAGAGAAACCCTATCGATGTAAACAATGTGGGAAA
GCCTTCAGATTTTCAAGTTCTGTTCGAATTCATGAAAGGTCTCACACTGGAGAGAAACCTTATGAATGC
AAACAATGTGGAAAAGCCTTCATTTCTTCCAGTCACTTTCGACTGCATGAAAGGACTCATATGGGAGAG
AAAGTCTAA
Aminoacid sequence, SEQ ID No. 847
MDSVSFEDVAVNFTLEEWALLDSSQKKLYEDVMQETFKNLVCLGKKWEDQDIEDDHRNQGKNRRCHMV
ERLCESRRGSKCGETTSQMPNVNINKETFTGAKPHECSFCGRDFIHHSSLNRHMRSHTGQKPNEYQEYEKQ
PCKCKAVGKTFSYHHCFRKHERTHTGVKPYECKQCGKAFIYYQPFQRHERTHAGQKPYECKQCGKTFIYYQ
SFQKHAHTGKKPYECKQCGKAFICYQSFQRHKRTHTGEKPYECKQCGKAFSCPTYFRTHERTHTGEKPYK
CKECGKAFSFLSSFRRHKRTHSGEKPYECKECGKAFFYSASFRAHVIIHTGARPYKCKECGKAFNSSNSC
RVHERTHIGEKPYECKRCGKSFSWSISLRLHERTHTGEKPYECKQCHKTFSFSSSLREHETTHTGEKPYE
CKQCGKTFSFSSSLQRHERTHNAEKPYECKQCGKAFRCSSYFRIHERSHTGEKPYECKQCGKVFIRSSSF
RLHERTHTGEKPYECKLCGKTFSFSSSLREHEKIHTGNKPFECKQCGKAFLRSSQIRLHERTHTGEKPYQ
CKQCGKAFISSSKFRMHERTHTGEKPYRCKQCGKAFRFSSSVRIHERSHTGEKPYECKQCGKAFISSSHF
RLHERTHMGEKV
Zinc finger protein 333 (Q96JL9), SEQ ID No. 848
ATGGAATCCGTCACCTTTGAGGATGTGGCCGTGGAGTTCATCCAGGAGTGGGCATTGCTGGACAGCGC
ACGGAGGAGCCTGTGCAAATACAGGATGCTTGACCAGTGCAGGACCCTGGCCTCCAGGGGAACTCCA
CCATGCAAACCCAGTTGTGTCTCCCAGCTGGGGCAAAGAGCAGAGCCAAAGGCAACAGAACGAGGGA
TTCTCCGTGCCACAGGTGTTGCCTGGGAATCTCAACTTAAACCCGAAGAGTTGCCTTCTATGCAGGATC
TTTTGGAAGAAGCATCCTCCAGGGACATGCAAATGGGGCCGGGGCTGTTCCTGAGGATGCAGCTGGT
GCCCTCCATAGAAGAGAGGGAGACACCATTGACTCGAGAGGACCGGCCAGCTCTCCAGGAGCCGCCT
TGGTCTCTGGGATGCACGGGACTGAAGGCCGCTATGCAGATTCAGAGGGTGGTGATACCAGTGCCTA
CTCTGGGCCACCGCAACCCATGGGTGGCCAGGGATTCTGCTGTGCCTGCACGTGACCCTGCCTGGCTT
CAGGAGGACAAAGTGGAGGAAGAAGCTATGGCTCCTGGGCTGCCAACCGCCTGTTCACAGGAACCAG
TCACCTTTGCAGATGTGGCTGTGGTGTTCACCCCAGAAGAATGGGTGTTTCTGGACTCTACTCAGAGGA
GCCTGTATAGAGATGTGATGCTGGAGAACTACAGGAACCTGGCCTCTGTGGCTGATCAACTGTGCAAA
CCCAATGCGTTGTCTTATTTGGAAGAAAGAGGAGAGCAGTGGACCACTGACAGGGGCGTCCTCTCAGA
CACCTGTGCAGAACCTCAGTGTCAACCCCAAGAGGCAATTCCTAGCCAAGATACTTTTACAGAGATCCT
GTCCATTGATGTGAAAGGGGAGCAACCTCAGCCTGGAGAAAAACTCTATAAATATAATGAACTTGAGA
AACCTTTTAACAGCATTGAACCACTTTTCCAGTACCAGAGAATTCATGCTGGAGAGGCATCCTGTGAAT
GTCAAGAGATTAGAAATTCCTTCTTCCAGAGTGCCCACCTAATTGTGCCCGAGAAAATCCGTAGTGGG
GATAAATCCTATGCATGTAACAAATGTGAAAAATCCTTCAGATACAGCTCTGACCTTATCAGGCATGAG
AAGACTCATACTGCAGAGAAGTGCTTTGACTGTCAAGAATGTGGGCAAGCCTTCAAATATTCCTCGAAT
CTCCGGCGACACATGAGAACCCATACCGGAGAGAAGCCATTTGAATGTAGTCAGTGTGGGAAAACCTT
CACGAGGAACTTTAACCTGATTTTGCACCAGAGAAACCACACAGGAGAGAAGCCCTACGAGTGTAAAG
ATTGTGGGAAAGCCTTCAATCAGCCATCATCCCTCAGGAGCCACGTGAGAACTCACACTGGAGAGAAG
CCCTTTGAATGCAGCCAGTGTGGGAAAGCCTTCAGGGAACACTCTTCACTGAAGACACATCTGCGAAC
CCATACCAGAGAGAAACCATATGAATGCAACCAGTGTGGCAAGCCCTTCCGGACGAGCACTCATCTGA
ACGTGCACAAGAGGATACACACAGGGGAGAAACTGTATGAGTGCGCGACTTGCGGTCAGGTCTTGAG
TCGTCTTTCAACCCTGAAGAGTCACATGCGAACTCACACTGGAGAGAAGCCCTATGTGTGCCAGGAAT
GTGGGCGAGCCTTCAGTGAGCCCTCATCCCTCAGGAAACATGCAAGGACTCACAGTGGCAAGAAGCCC
TATGCATGCCAGGAATGCGGGCGAGCCTTTGGTCAGTCTTCACATCTTATTGTACATGTGAGAACACAC
AGTGCCGGGAGACCCTATCAATGTAATCAGTGTGAGAAAGCCTTCAGGCACAGCTCCTCACTCACTGT
ACACAAAAGAACCCATGTGGGAAGAGAGACCATTAGGAATGGCAGCCTGCCTTTATCCATGTCTCATC
CATACTGTGGGCCCCTTGCTAATTAA
Aminoacid sequence, SEQ ID No. 849
MESVTFEDVAVEFIQEWALLDSARRSLCKYRMLDQCRTLASRGTPPCKPSCVSQLGQRAEPKATERGILR
ATGVAWESQLKPEELPSMQDLLEEASSRDMQMGPGLFLRMQLVPSIEERETPLTREDRPALQEPPWSLGC
TGLKAAMQIQRVVIPVPTLGHRNPWVARDSAVPARDPAWLQEDKVEEEAMAPGLPTACSQEPVTFADV
AVVFTPEEWVFLDSTQRSLYRDVMLENYRNLASVADQLCKPNALSYLEERGEQWTTDRGVLSDTCAEPQC
QPQEAIPSQDTFTEILSIDVKGEQPQPGEKLYKYNELEKPFNSIEPLFQYQRIHAGEASCECQEIRNSFFQS
AHLIVPEKIRSGDKSYACNKCEKSFRYSSDLIRHEKTHTAEKCFDCQECGQAFKYSSNLRRHMRTHTGEK
PFECSQCGKTFTRNFNLILHQRNHTGEKPYECKDCGKAFNQPSSLRSHVRTHTGEKPFECSQCGKAFREH
SSLKTHLRTHTREKPYECNQCGKPFRTSTHLNVHKRIHTGEKLYECATCGQVLSRLSTLKSHMRTHTGEK
PYVCQECGRAFSEPSSLRKHARTHSGKKPYACQECGRAFGQSSHLIVHVRTHSAGRPYQCNQCEKAFRHS
SSLTVHKRTHVGRETIRNGSLPLSMSHPYCGPLAN
Zinc finger protein 709 (Q8N972), SEQ ID No. 850
ATGGACTCAGTGGTCTTTGAGGATGTGGCTGTGAACTTCACCCAGGAGGAGTGGGCTTTGCTGGGTCC
CTCTCAGAAGAAACTCTACAGAGATGTGATGCAAGAAACCTTTGTTAACTTGGCCTCTATAGGGGAAA
ACTGGGAGGAGAAGAACATTGAAGATCACAAAAATCAGGGGAGAAAGCTAAGAAGTCATATGGTAG
AGAGGCTCTGTGAAAGGAAAGAAGGTAGTCAGTTTGGAGAAACCATCAGTCAGACTCCAAATCCTAAA
CCAAACAAGAAAACTTTTACTAGAGTAAAACCATATGAATGTAGTGTGTGTGGAAAGGACTATATGTG
TCATTCATCTCTTAATAGGCACATGAGATCTCATACTGAACATAGATCATATGAATATCACAAATATGGA
GAGAAATCATATGAATGTAAGGAATGTGGGAAAAGATTCAGCTTTCGAAGTTCATTTCGAATACATGA
AAGAACTCACACTGGAGAGAAACCCTATAAATGTAAACAGTGTGGTAAGGCTTTCAGTTGGCCCAGTT
CCTTTCAAATACATGAAAGAACTCATACTGGAGAGAAACCTTATGAATGTAAGGAATGTGGGAAGGCC
TTCATTTATCACACAACCTTTCGAGGACACATGAGAATGCACACAGGGGAGAAACCCTATAAATGTAAA
GAATGCGGGAAAACGTTCAGTCATCCCAGTTCTTTTCGAAATCATGAAAGAACTCACTCTGGAGAGAA
ACCCTATGAATGTAAACAATGTGGAAAAGCTTTCAGATATTACCAAACTTTTCAAATACATGAAAGGAC
TCACACTGGGGAAAAACCCTATCAGTGTAAGCAATGTGGTAAAGCTCTTAGTTGTCCCACATCCTTTCG
AAGTCATGAAAGGATTCACACTGGAGAAAAACCCTATAAATGTAAAAAATGTGGGAAAGCCTTCAGTT
TTCCTAGTTCCTTTAGAAAACATGAAAGAATTCATACAGGAGAGAAACCCTATGATTGTAAGGAATGTG
GGAAAGCATTCATTTCTCTTCCAAGCTATCGAAGACATATGATAATGCACACTGGAAATGGACCTTATA
AATGCAAGGAATGTGGGAAAGCCTTTGATTGTCCTAGTTCTTTTCAAATCCATGAACGAACTCACACTG
GAGAGAAACCCTATGAATGTAAACAGTGTGGTAAAGCCTTCAGTTGTTCCAGTTCCTTTCGAATGCATG
AAAGAACTCACACTGGAGAGAAACCCCATGAATGTAAACAATGTGGTAAAGCCTTCAGTTGTTCCAGT
TCTGTTCGAATACATGAAAGGACTCACACTGGAGAGAAACCCTATGAATGTAAACAGTGTGGTAAAGC
CTTCAGTTGTTCCAGTTCCTTTCGAATGCATGAAAGAATTCACACTGGAGAGAAACCCTATGAATGTAA
ACAGTGTGGTAAAGCCTTTAGTTTTTCTAGTTCCTTTCGGATGCATGAAAGGACTCACACTGGAGAGAA
ACCCTATGAATGTAAACAATGTGGTAAAGCCTTCAGTTGTTCCAGTTCCTTTCGAATGCATGAAAGGAC
TCACACTGGGGAGAAACCCTATGAATGTAAACAGTGTGGTAAGGCGTTTAGTTGTTCCAGTTCCATTCG
AATACATGAAAGGACTCACACTGGAGAGAAACCTTATGAGTGTAAACAATGTGGTAAGGCCTTCAGTT
GTTCTAGTTCTGTTCGAATGCATGAAAGGACTCACACTGGAGTGAAACCCTATGAATGTAAACAATGTG
ACAAAGCCTTCAGTTGCTCACGTTCCTTTCGAATCCATGAACGAACTCACACTGGAGAGAAACCCTATG
CATGTCAACAATGTGGTAAAGCCTTCAAGTGTTCCCGTTCCTTTCGAATACATGAAAGAGTTCATAGTG
GAGAGTAA
Aminoacid sequence, SEQ ID No. 851
MDSVVFEDVAVNFTQEEWALLGPSQKKLYRDVMQETFVNLASIGENWEEKNIEDHKNQGRKLRSHMVE
RLCERKEGSQFGETISQTPNPKPNKKTFTRVKPYECSVCGKDYMCHSSLNRHMRSHTEHRSYEYHKYGEKSY
ECKECGKRFSFRSSFRIHERTHTGEKPYKCKQCGKAFSWPSSFQIHERTHTGEKPYECKECGKAFIYHTT
FRGHMRMHTGEKPYKCKECGKTFSHPSSFRNHERTHSGEKPYECKQCGKAFRYYQTFQIHERTHTGEKPY
QCKQCGKALSCPTSFRSHERIHTGEKPYKCKKCGKAFSFPSSFRKHERIHTGEKPYDCKECGKAFISLPS
YRRHMIMHTGNGPYKCKECGKAFDCPSSFQIHERTHTGEKPYECKQCGKAFSCSSSFRMHERTHTGEKPH
ECKQCGKAFSCSSSVRIHERTHTGEKPYECKQCGKAFSCSSSFRMHERIHTGEKPYECKQCGKAFSFSSS
FRMHERTHTGEKPYECKQCGKAFSCSSSFRMHERTHTGEKPYECKQCGKAFSCSSSIRIHERTHTGEKPY
ECKQCGKAFSCSSSVRMHERTHTGVKPYECKQCDKAFSCSRSFRIHERTHTGEKPYACQQCGKAFKCSRS
FRIHERVHSGE
ZNF35, zinc finger protein 35, SEQ ID No. 852
ATGACTGCAGAATTGAGAGAAGCCATGGCCCTAGCCCCATGGGGCCCAGTGAAGGTGAAAAAGGAGGAGG
AAGAAGAAGAAAACTTCCCAGGTCAGGCATCCAGCCAACAAGTGCACTCCGAGAACATCAAAGTCTGGGC
CCCAGTGCAGGGTCTTCAGACAGGCCTTGATGGATCAGAAGAGGAAGAAAAGGGTCAGAACATATCCTGG
GATATGGCGGTAGTCCTGAAAGCAACTCAGGAGGCACCTGCTGCTTCAACCCTTGGCAGCTACTCATTAC
CAGGGACTCTGGCCAAGAGTGAGATACTGGAGACTCATGGGACCATGAACTTTCTAGGTGCTGAAACCAA
GAACCTACAGTTACTGGTTCCAAAAACTGAGATATGTGAGGAAGCTGAAAAACCCCTCATCATATCAGAA
AGAATCCAGAAAGCTGATCCTCAAGGACCTGAGTTAGGAGAAGCTTGTGAAAAGGGAAACATGTTAAAGA
GGCAGAGAATAAAGAGAGAAAAGAAAGATTTCAGACAAGTGATAGTGAATGACTGTCACTTACCTGAAAG
CTTCAAAGAAGAGGAAAACCAGAAATGTAAGAAATCTGGAGGAAAATATAGCCTTAATTCTGGCGCTGTT
AAAAATCCAAAAACCCAGCTTGGACAAAAGCCTTTTACGTGTAGCGTGTGTGGGAAAGGATTTAGTCAGA
GTGCAAACCTCGTTGTGCATCAGCGAATCCACACTGGAGAGAAACCCTTTGAATGTCATGAGTGTGGGAA
GGCCTTCATTCAGAGTGCAAACCTCGTTGTGCATCAGAGAATCCACACTGGACAGAAACCTTATGTTTGC
TCAAAATGTGGGAAAGCCTTCACTCAGAGTTCAAATCTGACTGTACATCAAAAAATCCACTCCTTAGAAA
AAACTTTTAAGTGCAATGAATGTGAGAAAGCCTTTAGTTACAGCTCACAACTTGCTCGGCACCAGAAAGT
CCACATTACGGAAAAATGCTATGAATGTAATGAATGTGGGAAAACATTTACTAGGAGCTCAAACCTCATT
GTCCACCAGAGGATCCACACTGGGGAGAAGCCCTTTGCCTGTAACGACTGTGGCAAAGCCTTTACCCAGA
GTGCAAATCTTATTGTACATCAGCGAAGCCATACTGGTGAGAAGCCATATGAGTGTAAAGAGTGTGGGAA
AGCCTTTAGTTGTTTTTCACACCTTATTGTGCACCAGAGAATTCACACTGCAGAGAAACCTTACGACTGC
AGCGAATGTGGGAAAGCCTTCAGTCAGCTCTCTTGCCTTATTGTCCACCAGAGAATTCACAGTGGAGATC
TTCCTTACGTGTGTAATGAATGTGGGAAGGCCTTCACATGTAGCTCATACCTACTTATTCATCAGAGAAT
TCATAATGGAGAAAAACCTTACACATGTAATGAGTGTGGGAAGGCCTTCAGACAGAGGTCGAGCCTCACC
GTGCACCAGAGAACCCACACTGGGGAGAAGCCCTATGAATGTGAGAAGTGTGGTGCAGCTTTCATTTCCA
ACTCACACCTCATGCGACACCATAGAACCCATCTTGTTGAATAA
Aminoacid sequence SEQ ID No. 853
MTAELREAMALAPWGPVKVKKEEEEEENFPGQASSQQVHSENIKVWAPVQGLQTGLDGSEEEEKGQNISW
DMAVVLKATQEAPAASTLGSYSLPGTLAKSEILETHGTMNFLGAETKNLQLLVPKTEICEEAEKPLIISE
RIQKADPQGPELGEACEKGNMLKRQRIKREKKDFRQVIVNDCHLPESFKEEENQKCKKSGGKYSLNSGAV
KNPKTQLGQKPFTCSVCGKGFSQSANLVVHQRIHTGEKPFECHECGKAFIQSANLVVHQRIHTGQKPYVC
SKCGKAFTQSSNLTVHQKIHSLEKTFKCNECEKAFSYSSQLARHQKVHITEKCYECNECGKTFTRSSNLI
VHQRIHTGEKPFACNDCGKAFTQSANLIVHQRSHTGEKPYECKECGKAFSCFSHLIVHQRIHTAEKPYDC
SECGKAFSQLSCLIVHORIHSGDLPYVCNECGKAFTCSSYLLIHQRIHNGEKPYTCNECGKAFRQRSSLT
VHQRTHTGEKPYECEKCGAAFISNSHLMRHHRTHLVE
Disease genes:
Rho, Rhodopsin (Ensembl: ENSG00000163914)
Nucleotide sequence SEQ ID No. 854
ATGAATGGCACAGAAGGCCCTAACTTCTACGTGCCCTTCTCCAATGCGACGGGTGTGGTACGCAGCCC
CTTCGAGTACCCACAGTACTACCTGGCTGAGCCATGGCAGTTCTCCATGCTGGCCGCCTACATGTTTCT
GCTGATCGTGCTGGGCTTCCCCATCAACTTCCTCACGCTCTACGTCACCGTCCAGCACAAGAAGCTGCG
CACGCCTCTCAACTACATCCTGCTCAACCTAGCCGTGGCTGACCTCTTCATGGTCCTAGGTGGCTTCACC
AGCACCCTCTACACCTCTCTGCATGGATACTTCGTCTTCGGGCCCACAGGATGCAATTTGGAGGGCTTC
TTTGCCACCCTGGGCGGTGAAATTGCCCTGTGGTCCTTGGTGGTCCTGGCCATCGAGCGGTACGTGGT
GGTGTGTAAGCCCATGAGCAACTTCCGCTTCGGGGAGAACCATGCCATCATGGGCGTTGCCTTCACCT
GGGTCATGGCGCTGGCCTGCGCCGCACCCCCACTCGCCGGCTGGTCCAGGTACATCCCCGAGGGCCTG
CAGTGCTCGTGTGGAATCGACTACTACACGCTCAAGCCGGAGGTCAACAACGAGTCTTTTGTCATCTAC
ATGTTCGTGGTCCACTTCACCATCCCCATGATTATCATCTTTTTCTGCTATGGGCAGCTCGTCTTCACCGT
CAAGGAGGCCGCTGCCCAGCAGCAGGAGTCAGCCACCACACAGAAGGCAGAGAAGGAGGTCACCCG
CATGGTCATCATCATGGTCATCGCTTTCCTGATCTGCTGGGTGCCCTACGCCAGCGTGGCATTCTACATC
TTCACCCACCAGGGCTCCAACTTCGGTCCCATCTTCATGACCATCCCAGCGTTCTTTGCCAAGAGCGCCG
CCATCTACAACCCTGTCATCTATATCATGATGAACAAGCAGTTCCGGAACTGCATGCTCACCACCATCTG
CTGCGGCAAGAACCCACTGGGTGACGATGAGGCCTCTGCTACCGTGTCCAAGACGGAGACGAGCCAG
GTGGCCCCGGCCTAA
Amino acid sequence SEQ ID No. 855
MNGTEGPNFYVPFSNATGVVRSPFEYPQYYLAEPWQFSMLAAYMFLLIVLGFPINFLTLYVTVQHKKLRT
PLNYILLNLAVADLFMVLGGFTSTLYTSLHGYFVFGPTGCNLEGFFATLGGEIALWSLVVLAIERYVVVC
KPMSNFRFGENHAIMGVAFTWVMALACAAPPLAGWSRYIPEGLQCSCGIDYYTLKPEVNNESFVIYMFVV
HFTIPMIIIFFCYGQLVFTVKEAAAQQQESATTQKAEKEVTRMVIIMVIAFLICWVPYASVAFYIFTHQG
SNFGPIFMTIPAFFAKSAAIYNPVIYIMMNKQFRNCMLTTICCGKNPLGDDEASATVSKTETSQVAPA
PRPH2, peripherin 2 (Ensembl: ENSG00000112619)
Nucleotide sequence SEQ ID No. 856
ATGGCGCTACTGAAAGTCAAGTTTGACCAGAAGAAGCGGGTCAAGTTGGCCCAAGGGCTCTGGCTCAT
GAACTGGTTCTCCGTGTTGGCTGGCATCATCATCTTCAGCCTAGGACTGTTCCTGAAGATTGAACTCCG
AAAGAGGAGCGATGTGATGAATAATTCTGAGAGCCATTTTGTGCCCAACTCATTGATAGGGATGGGG
GTGCTATCCTGTGTCTTCAACTCGCTGGCTGGGAAGATCTGCTACGACGCCCTGGACCCAGCCAAGTAT
GCCAGATGGAAGCCCTGGCTGAAGCCGTACCTGGCTATCTGTGTTCTCTTCAACATCATCCTCTTCCTTG
TGGCTCTCTGCTGCTTTCTGCTTCGGGGCTCGCTGGAGAACACCCTGGGCCAAGGGCTCAAGAACGGC
ATGAAGTACTACCGGGACACAGACACCCCTGGCAGGTGTTTCATGAAGAAGACCATCGACATGCTGCA
GATCGAGTTCAAATGCTGCGGCAACAACGGTTTTCGGGACTGGTTTGAGATTCAGTGGATCAGCAATC
GCTACCTGGACTTTTCCTCCAAAGAAGTCAAAGATCGAATCAAGAGCAACGTGGATGGGCGGTACCTG
GTGGACGGCGTCCCTTTCAGCTGCTGCAATCCTAGCTCGCCACGGCCCTGCATCCAGTATCAGATCACC
AACAACTCAGCACACTACAGTTACGACCACCAGACGGAGGAGCTCAACCTGTGGGTGCGTGGCTGCA
GGGCTGCCCTGCTGAGCTACTACAGCAGCCTCATGAACTCCATGGGTGTCGTCACGCTCCTCATTTGGC
TCTTCGAGGTGACCATTACAATTGGGCTGCGCTACCTACAGACGTCGCTGGATGGTGTGTCCAACCCCG
AGGAATCTGAGAGCGAGAGCCAGGGCTGGCTGCTGGAGAGGAGCGTGCCGGAGACCTGGAAGGCCT
TTCTGGAGAGTGTGAAGAAGCTGGGCAAGGGCAACCAGGTGGAAGCCGAGGGCGCAGACGCAGGCC
AGGCCCCAGAGGCTGGCTGA
Amino acid sequence SEQ ID No. 857
MALLKVKFDQKKRVKLAQGLWLMNWFSVLAGIIIFSLGLFLKIELRKRSDVMNNSESHFVPNSLIGMGVL
SCVFNSLAGKICYDALDPAKYARWKPWLKPYLAICVLFNIILFLVALCCFLLRGSLENTLGQGLKNGMKY
YRDTDTPGRCFMKKTIDMLQIEFKCCGNNGFRDWFEIQWISNRYLDFSSKEVKDRIKSNVDGRYLVDGVP
FSCCNPSSPRPCIQYQITNNSAHYSYDHQTEELNLWVRGCRAALLSYYSSLMNSMGVVTLLIWLFEVTIT
IGLRYLQTSLDGVSNPEESESESQGWLLERSVPETWKAFLESVKKLGKGNQVEAEGADAGQAPEAG
RP1, axonemal microtubule associated (Ensembl: ENSG00000104237)
Nucleotide sequence SEQ ID No. 858
ATGAGTGATACCCCTTCTACTGGTTTTTCCATCATTCATCCTACGTCTTCTGAAGGTCAAGTTCCACCCC
CTCGCCATTTGAGCCTCACTCATCCTGTTGTGGCCAAGCGAATCAGTTTCTACAAGAGCGGAGACCCCC
AATTCGGCGGGGTCAGGGTGGTGGTCAACCCTCGCTCCTTTAAGTCCTTTGATGCTCTGCTGGATAACT
TGTCCAGGAAGGTGCCCCTCCCTTTTGGAGTGAGGAACATCAGCACCCCTCGGGGCAGGCACAGCATC
ACGCGCCTGGAGGAGCTGGAGGACGGCGAGTCCTACCTATGTTCCCACGGCAGGAAGGTGCAGCCTG
TAGACCTGGACAAAGCCCGTCGGCGCCCGCGGCCCTGGCTCAGCAGCCGGGCCATTAGCGCGCACTCA
CCGCCCCACCCCGTAGCCGTCGCTGCTCCCGGCATGCCCCGCCCCCCACGGAGCCTAGTGGTCTTCAGG
AATGGCGACCCGAAGACGAGGCGTGCGGTTCTTCTGAGCAGGAGGGTCACCCAGAGCTTCGAGGCAT
TTCTACAGCACCTGACAGAGGTCATGCAGCGCCCTGTGGTCAAGCTGTACGCTACGGACGGAAGGAG
GGTTCCCAGCCTCCAGGCAGTGATCCTGAGCTCTGGAGCTGTGGTGGCGGCAGGAAGGGAGCCATTT
AAACCAGGAAATTATGACATCCAAAAATACTTGCTTCCTGCTAGATTACCAGGGATCTCTCAGCGTGTG
TACCCCAAGGGAAATGCAAAGTCAGAAAGCAGAAAGATAAGCACACATATGTCTTCAAGCTCAAGGTC
CCAGATTTATTCTGTTTCTTCTGAGAAAACACATAATAATGATTGCTACTTAGACTATTCTTTTGTTCCTG
AAAAGTACTTGGCCTTAGAAAAGAATGATTCTCAGAATTTACCAATATATCCTTCTGAAGATGATATTG
AGAAATCAATTATTTTTAATCAAGACGGCACTATGACAGTTGAGATGAAAGTTCGATTCAGAATAAAA
GAGGAAGAAACCATAAAATGGACAACTACTGTCAGTAAAACTGGTCCTTCTAATAATGATGAAAAGAG
TGAGATGAGTTTTCCAGGAAGAACAGAAAGTCGATCATCTGGTTTAAAGCTTGCAGCATGTTCATTCTC
TGCAGATGTGTCACCTATGGAGCGAAGCAGTAATCAAGAGGGCAGTTTGGCAGAGGAGATAAACATT
CAAATGACAGATCAAGTGGCTGAAACTTGCAGTTCTGCTAGTTGGGAGAATGCTACTGTGGACACAGA
TATCATCCAGGGAACTCAAGACCAAGCAAAGCATCGTTTTTATAGGCCCCCTACACCTGGACTAAGAAG
AGTGAGACAAAAGAAATCTGTGATTGGCAGTGTGACCTTAGTATCTGAAACTGAGGTTCAAGAGAAAA
TGATTGGACAGTTTTCATATAGTGAAGAAAGGGAAAGTGGGGAAAACAAGTCTGAGTATCACATGTTT
ACACATTCTTGCAGTAAAATGTCATCAGTATCTAACAAACCAGTACTTGTTCAGATCAATAACAATGATC
AAATGGAGGAGTCATCATTAGAAAGAAAAAAGGAAAACAGTCTGCTTAAGTCAAGTGCAATAAGTGC
TGGTGTTATAGAAATTACAAGTCAGAAGATGTTAGAGATGTCACATAATAATGGTTTGCCATCAACTAT
ATCAAATAACTCAATTGTGGAGGAAGATGTAGTTGATTGTGTGGTATTGGACAACAAAACTGGTATCA
AGAACTTCAAAACTTATGGTAACACCAATGATAGGTTCAGTCCTATTTCAGCAGATGCAACCCATTTTTC
AAGTAATAACTCTGGAACTGACAAAAATATTTCTGAGGCTCCAGCTTCAGAAGCATCCTCTACTGTCAC
TGCAAGAATTGACAGACTAATTAATGAATTTGCTCAGTGTGGTTTAACAAAACTTCCAAAAAATGAAAA
GAAGATTTTGTCATCTGTTGCCAGCAAAAAGAAGAAAAAATCTCGACAGCAAGCAATAAATTCCAGGT
ATCAAGATGGACAGCTTGCAACCAAAGGAATTCTTAATAAGAATGAGAGAATAAACACAAAAGGTAG
AATTACAAAGGAAATGATAGTGCAAGATTCAGATAGTCCCCTTAAAGGAGGGATACTTTGTGAGGAAG
ACCTCCAGAAAAGTGATACTGTAATTGAATCAAATACTTTTTGTTCCAAAAGTAATCTCAATTCCACGAT
TTCCAAGAATTTCCATAGAAATAAATTAAATACTACTCAAAATTCCAAGGTTCAAGGACTTTTAACCAAA
AGAAAATCTAGATCACTAAATAAAATAAGCTTAGGAGCACCTAAAAAAAGAGAAATCGGTCAAAGAG
ATAAAGTGTTTCCTCACAATGAATCTAAATATTGCAAAAGTACTTTTGAAAACAAAAGTTTATTTCATGT
ATTTAACATCCTTGAGCAAAAACCCAAAGATTTTTATGCACCGCAATCTCAAGCAGAAGTGGCATCTGG
GTATTTGAGAGGAATGGCAAAGAAGAGTTTAGTTTCAAAAGTTACTGATTCACACATAACTTTAAAAA
GCCAGAAAAAACGTAAAGGGGATAAAGTGAAAGCAAGTGCTATTTTAAGTAAACAACATGCTACAACC
AGGGCAAATTCTTTAGCTTCTTTGAAAAAACCTGATTTTCCTGAGGCTATTGCTCATCATTCAATTCAAA
ATTATATACAGAGTTGGTTGCAGAACATAAATCCATATCCAACTTTAAAGCCTATAAAATCAGCTCCAG
TATGTAGAAATGAAACGAGTGTGGTAAATTGTAGCAATAATAGTTTTTCAGGGAATGATCCCCATACA
AATTCTGGAAAAATAAGTAATTTTGTTATGGAAAGTAATAAGCACATAACTAAAATTGCCGGTTTGACA
GGAGATAATCTATGTAAAGAGGGAGATAAGTCTTTTATTGCCAATGACACTGGTGAAGAAGATCTCCA
TGAGACACAGGTTGGATCTCTGAATGATGCTTATTTGGTTCCCCTGCATGAACACTGTACTTTGTCACA
GTCAGCTATTAATGATCATAATACTAAAAGTCATATAGCTGCTGAAAAATCAGGACCAGAGAAAAAA
CTTGTTTACCAGGAAATAAACCTAGCTAGAAAAAGGCAAAGTGTAGAGGCTGCCATTCAAGTAGATCC
TATAGAAGAGGAAACTCCAAAAGACCTCTTACCAGTCCTGATGCTTCACCAATTGCAAGCTTCAGTTCC
TGGTATTCACAAGACTCAGAATGGAGTTGTTCAAATGCCAGGTTCACTTGCAGGTGTTCCCTTTCATTCT
GCAATATGTAATTCATCCACTAATCTCCTTCTAGCTTGGCTCTTGGTGCTAAACCTAAAGGGAAGTATGA
ATAGCTTCTGTCAAGTTGATGCTCACAAGGCTACCAACAAATCTTCAGAAACACTTGCATTGTTGGAGA
TTCTAAAGCACATAGCTATCACAGAGGAAGCTGATGACTTGAAAGCTGCTGTTGCCAATTTAGTGGAG
TCAACTACAAGCCACTTTGGACTCAGTGAGAAAGAACAAGACATGGTTCCAATAGATCTTTCTGCAAAT
TGTTCCACGGTCAACATTCAGAGTGTTCCTAAGTGCAGTGAAAATGAAAGAACACAAGGAATCTCCTCT
TTGGATGGAGGTTGCTCTGCCAGTGAGGCATGTGCCCCTGAAGTCTGTGTTTTGGAAGTGACTTGCTCT
CCATGTGAGATGTGCACTGTAAATAAGGCTTATTCTCCAAAAGAGACATGTAACCCCAGTGACACTTTT
TTTCCTAGTGATGGTTATGGTGTGGATCAGACTTCTATGAATAAGGCTTGTTTCCTAGGAGAGGTCTGT
TCACTTACTGATACTGTGTTTTCTGATAAGGCTTGTGCTCAAAAGGAGAACCATACCTATGAGGGAGCT
TGCCCAATTGATGAGACCTACGTTCCTGTCAATGTCTGCAATACCATTGACTTTTTAAACTCCAAAGAAA
ACACATATACTGATAACTTGGATTCAACTGAAGAGTTAGAAAGAGGTGATGACATTCAGAAAGATCTA
AATATTTTGACAGACCCTGAATATAAAAATGGATTTAATACATTGGTGTCACATCAAAATGTCAGTAAT
TTAAGCTCCTGTGGCCTTTGCCTAAGTGAAAAAGAAGCAGAACTTGATAAGAAACATAGTTCTCTAGAT
GATTTTGAAAATTGTTCACTAAGGAAGTTTCAGGATGAAAATGCATATACTTCCTTTGATATGGAAGAA
CCACGGACTTCTGAAGAACCAGGCTCAATAACCAACAGCATGACATCAAGTGAAAGAAACATTTCAGA
ATTGGAATCTTTTGAAGAATTAGAAAACCATGACACTGATATCTTTAATACAGTGGTAAATGGAGGAG
AGCAAGCCACTGAAGAATTAATCCAAGAAGAGGTAGAGGCTAGTAAAACTTTAGAATTGATAGACATC
TCTAGTAAGAATATTATGGAAGAAAAAAGAATGAACGGTATAATTTATGAAATAATCAGTAAGAGGCT
GGCAACACCACCATCTTTAGATTTTTGCTATGATTCTAAGCAAAATAGTGAAAAGGAGACCAATGAAG
GAGAAACTAAGATGGTAAAAATGATGGTGAAAACTATGGAAACTGGAAGTTATTCAGAGTCCTCTCCT
GATTTAAAAAAATGCATCAAAAGTCCAGTGACTTCTGATTGGTCAGACTATCGGCCTGACAGTGACAGT
GAGCAGCCATATAAAACATCCAGTGATGATCCCAATGACAGTGGCGAACTTACCCAAGAGAAAGAATA
TAACATAGGATTTGTTAAAAGGGCAATAGAAAAACTGTACGGTAAAGCAGATATTATCAAACCATCTTT
TTTTCCTGGGTCTACCCGCAAATCTCAGGTTTGTCCTTATAATTCTGTGGAATTTCAGTGTTCCAGGAAA
GCAAGTCTTTATGATTCTGAAGGGCAGTCATTTGGCTCTTCTGAACAGGTATCTAGTAGTTCATCTATGT
TGCAGGAATTCCAGGAGGAAAGACAAGATAAGTGTGATGTTAGTGCTGTGAGGGACAATTATTGTAG
GGGTGACATTGTAGAACCTGGTACAAAACAAAATGATGATAGCAGAATCCTCACAGACATAGAGGAA
GGAGTACTGATTGACAAAGGCAAATGGCTTCTGAAAGAAAATCATTTGCTAAGGATGTCATCTGAAAA
TCCTGGCATGTGTGGCAATGCAGACACCACATCAGTGGACACCCTACTTGATAATAACAGCAGTGAGG
TACCATATTCACATTTTGGTAATTTGGCCCCAGGCCCAACGATGGATGAACTCTCCTCTTCAGAACTCGA
GGAACTGACTCAACCCCTTGAACTAAAATGCAATTACTTTAACATGCCTCATGGTAGTGACTCAGAACC
TTTTCATGAGGACTTGCTGGATGTTCGCAATGAAACCTGTGCCAAGGAAAGAATAGCAAATCATCATAC
AGAGGAGAAGGGTAGTCATCAGTCAGAAAGAGTATGCACATCTGTCACTCATTCCTTTATTTCTGCTGG
TAACAAAGTCTACCCTGTCTCTGATGATGCTATTAAAAACCAACCATTGCCTGGCAGTAATATGATTCAT
GGTACACTTCAGGAAGCTGACTCTTTGGATAAACTGTATGCTCTTTGTGGTCAACATTGCCCAATACTA
ACTGTTATTATCCAACCCATGAATGAGGAAGACCGAGGATTTGCATATCGCAAAGAATCTGATATTGAA
AATTTCTTGGGTTTTTATTTATGGATGAAAATACACCCATATTTACTTCAGACAGACAAAAATGTGTTCA
GGGAAGAGAACAATAAAGCAAGTATGAGACAAAATCTTATTGATAATGCCATTGGTGATATATTTGAT
CAGTTTTATTTCAGTAACACATTTGACTTGATGGGTAAAAGAAGAAAACAAAAAAGAATTAACTTCTTG
GGGTTAGAGGAAGAAGGTAATTTAAAGAAATTTCAACCAGATTTGAAGGAAAGGTTTTGTATGAATTT
CTTGCACACATCATTGTTAGTTGTGGGTAATGTGGATTCAAATACACAAGACCTCAGCGGTCAGACAAA
TGAAATCTTTAAAGCAGTCGATGAGAATAACAACTTATTAAATAACAGATTCCAGGGCTCAAGAACAA
ATCTCAACCAAGTAGTAAGAGAAAATATCAACTGTCATTACTTCTTTGAAATGCTTGGTCAAGCTTGCCT
CTTAGATATTTGCCAAGTTGAGACCTCCTTAAATATTAGCAACAGAAATATTTTAGAACTTTGTATGTTT
GAGGGTGAAAATCTTTTCATTTGGGAAGAGGAAGACATATTAAATTTAACTGATCTTGAAAGCAGTAG
AGAACAAGAAGATTTATAA
Amino acid sequence SEQ ID No. 859
MSDTPSTGFSIIHPTSSEGQVPPPRHLSLTHPVVAKRISFYKSGDPQFGGVRVVVNPRSFKSFDALLDNL
SRKVPLPFGVRNISTPRGRHSITRLEELEDGESYLCSHGRKVQPVDLDKARRRPRPWLSSRAISAHSPPH
PVAVAAPGMPRPPRSLVVFRNGDPKTRRAVLLSRRVTQSFEAFLQHLTEVMQRPVVKLYATDGRRVPSLQ
AVILSSGAVVAAGREPFKPGNYDIQKYLLPARLPGISQRVYPKGNAKSESRKISTHMSSSSRSQIYSVSS
EKTHNNDCYLDYSFVPEKYLALEKNDSQNLPIYPSEDDIEKSIIFNQDGTMTVEMKVRFRIKEEETIKWT
TTVSKTGPSNNDEKSEMSFPGRTESRSSGLKLAACSFSADVSPMERSSNQEGSLAEEINIQMTDQVAETC
SSASWENATVDTDIIQGTQDQAKHRFYRPPTPGLRRVRQKKSVIGSVTLVSETEVQEKMIGQFSYSEERE
SGENKSEYHMFTHSCSKMSSVSNKPVLVQINNNDQMEESSLERKKENSLLKSSAISAGVIEITSQKMLEM
SHNNGLPSTISNNSIVEEDVVDCVVLDNKTGIKNFKTYGNTNDRFSPISADATHFSSNNSGTDKNISEAP
ASEASSTVTARIDRLINEFAQCGLTKLPKNEKKILSSVASKKKKKSRQQAINSRYQDGQLATKGILNKNE
RINTKGRITKEMIVQDSDSPLKGGILCEEDLQKSDTVIESNTFCSKSNLNSTISKNFHRNKLNTTQNSKV
QGLLTKRKSRSLNKISLGAPKKREIGQRDKVFPHNESKYCKSTFENKSLFHVFNILEQKPKDFYAPQSQA
EVASGYLRGMAKKSLVSKVTDSHITLKSQKKRKGDKVKASAILSKQHATTRANSLASLKKPDFPEAIAHH
SIQNYIQSWLQNINPYPTLKPIKSAPVCRNETSVVNCSNNSFSGNDPHTNSGKISNFVMESNKHITKIAG
LTGDNLCKEGDKSFIANDTGEEDLHETQVGSLNDAYLVPLHEHCTLSQSAINDHNTKSHIAAEKSGPEKK
LVYQEINLARKRQSVEAAIQVDPIEEETPKDLLPVLMLHQLQASVPGIHKTQNGVVQMPGSLAGVPFHSA
ICNSSTNLLLAWLLVLNLKGSMNSFCQVDAHKATNKSSETLALLEILKHIAITEEADDLKAAVANLVEST
TSHFGLSEKEQDMVPIDLSANCSTVNIQSVPKCSENERTQGISSLDGGCSASEACAPEVCVLEVTCSPCE
MCTVNKAYSPKETCNPSDTFFPSDGYGVDQTSMNKACFLGEVCSLTDTVFSDKACAQKENHTYEGACPID
ETYVPVNVCNTIDFLNSKENTYTDNLDSTEELERGDDIQKDLNILTDPEYKNGFNTLVSHQNVSNLSSCG
LCLSEKEAELDKKHSSLDDFENCSLRKFQDENAYTSFDMEEPRTSEEPGSITNSMTSSERNISELESFEE
LENHDTDIFNTVVNGGEQATEELIQEEVEASKTLELIDISSKNIMEEKRMNGIIYEIISKRLATPPSLDF
CYDSKONSEKETNEGETKMVKMMVKTMETGSYSESSPDLKKCIKSPVTSDWSDYRPDSDSEQPYKTSSDD
PNDSGELTQEKEYNIGFVKRAIEKLYGKADIIKPSFFPGSTRKSQVCPYNSVEFQCSRKASLYDSEGQSF
GSSEQVSSSSSMLQEFQEERQDKCDVSAVRDNYCRGDIVEPGTKQNDDSRILTDIEEGVLIDKGKWLLKE
NHLLRMSSENPGMCGNADTTSVDTLLDNNSSEVPYSHFGNLAPGPTMDELSSSELEELTQPLELKCNYFN
MPHGSDSEPFHEDLLDVRNETCAKERIANHHTEEKGSHQSERVCTSVTHSFISAGNKVYPVSDDAIKNQP
LPGSNMIHGTLQEADSLDKLYALCGQHCPILTVIIQPMNEEDRGFAYRKESDIENFLGFYLWMKIHPYLL
QTDKNVFREENNKASMRQNLIDNAIGDIFDQFYFSNTFDLMGKRRKQKRINFLGLEEEGNLKKFQPDLKE
RFCMNFLHTSLLVVGNVDSNTQDLSGQTNEIFKAVDENNNLLNNRFQGSRTNLNQVVRENINCHYFFEML
GQACLLDICQVETSLNISNRNILELCMFEGENLFIWEEEDILNLTDLESSREQEDL
CRX, cone-rod homeobox (Ensembl: ENSG00000105392)
Nucleotide sequence SEQ ID No. 860
ATGATGGCGTATATGAACCCGGGGCCCCACTATTCTGTCAACGCCTTGGCCCTAAGTGGCCCCAGTGTG
G
ATCTGATGCACCAGGCTGTGCCCTACCCAAGCGCCCCCAGGAAGCAGCGGCGGGAGCGCACCACCTTC
AC
CCGGAGCCAACTGGAGGAGCTGGAGGCACTGTTTGCCAAGACCCAGTACCCAGACGTCTATGCCCGTG
AG
GAGGTGGCTCTGAAGATCAATCTGCCTGAGTCCAGGGTTCAGGTTTGGTTCAAGAACCGGAGGGCTAA
AT
GCAGGCAGCAGCGACAGCAGCAGAAACAGCAGCAGCAGCCCCCAGGGGGCCAGGCCAAGGCCCGGC
CTGC
CAAGAGGAAGGCGGGCACGTCCCCAAGACCCTCCACAGATGTGTGTCCAGACCCTCTGGGCATCTCAG
AT
TCCTACAGTCCCCCTCTGCCCGGCCCCTCAGGCTCCCCAACCACGGCAGTGGCCACTGTGTCCATCTGG
A
GCCCAGCCTCAGAGTCCCCTTTGCCTGAGGCGCAGCGGGCTGGGCTGGTGGCCTCAGGGCCGTCTCTG
AC
CTCCGCCCCCTATGCCATGACCTACGCCCCGGCCTCCGCTTTCTGCTCTTCCCCCTCCGCCTATGGGTCT
CCGAGCTCCTATTTCAGCGGCCTAGACCCCTACCTTTCTCCCATGGTGCCCCAGCTAGGGGGCCCGGCT
C
TTAGCCCCCTCTCTGGCCCCTCCGTGGGACCTTCCCTGGCCCAGTCCCCCACCTCCCTATCAGGCCAGAG
CTATGGCGCCTACAGCCCCGTGGATAGCTTGGAATTCAAGGACCCCACGGGCACCTGGAAATTCACCT
AC
AATCCCATGGACCCTCTGGACTACAAGGATCAGAGTGCCTGGAAGTTTCAGATCTTGTAG
Amino acid sequence SEQ ID No. 861
MMAYMNPGPHYSVNALALSGPSVDLMHQAVPYPSAPRKQRRERTTFTRSQLEELEALFAKTQYPDVYAR
E
EVALKINLPESRVQVWFKNRRAKCRQQRQQQKQQQQPPGGQAKARPAKRKAGTSPRPSTDVCPDPLGIS
D
SYSPPLPGPSGSPTTAVATVSIWSPASESPLPEAQRAGLVASGPSLTSAPYAMTYAPASAFCSSPSAYGS
PSSYFSGLDPYLSPMVPQLGGPALSPLSGPSVGPSLAQSPTSLSGQSYGAYSPVDSLEFKDPTGTWKFTY
NPMDPLDYKDQSAWKFQIL
GUCA1B, guanylate cyclase activator 1B (ENSG00000112599)
Nucleotide sequence SEQ ID No. 862
ATGGGGCAGGAGTTTAGCTGGGAGGAGGCGGAGGCAGCTGGCGAGATAGATGTGGCGGAGCTCCAG
GAGT
GGTACAAGAAGTTTGTGATGGAGTGCCCCAGCGGCACACTCTTTATGCATGAGTTTAAGCGCTTCTTCA
A
GGTCACAGACGATGAGGAGGCCTCCCAGTATGTAGAGGGCATGTTCCGAGCCTTCGACAAGAATGGG
GAC
AACACCATCGACTTCCTGGAGTACGTGGCAGCTCTGAATCTCGTGCTGAGGGGCACCCTGGAGCACAA
GC
TGAAGTGGACATTCAAGATCTATGATAAGGATGGCAATGGCTGCATCGACCGCCTGGAGCTACTCAAC
AT
TGTGGAGGGAATTTACCAGCTGAAGAAAGCCTGCCGGCGAGAGCTACAAACTGAGCAAGGCCAGCTG
CTC
ACACCCGAGGAGGTCGTGGACAGGATCTTCCTCCTGGTGGATGAGAATGGAGATGGCCAGCTGTCTCT
GA
ACGAGTTTGTTGAAGGTGCCCGTCGGGACAAGTGGGTGATGAAGATGCTGCAGATGGACATGAATCC
CAG
CAGCTGGCTCGCTCAGCAGAGACGGAAAAGTGCCATGTTCTGA
Aminoacid sequence SEQ ID No. 863
MGQEFSWEEAEAAGEIDVAELQEWYKKFVMECPSGTLFMHEFKRFFKVTDDEEASQYVEGMFRAFDKN
GD
NTIDFLEYVAALNLVLRGTLEHKLKWTFKIYDKDGNGCIDRLELLNIVEGIYQLKKACRRELQTEQGQLL
TPEEVVDRIFLLVDENGDGQLSLNEFVEGARRDKWVMKMLQMDMNPSSWLAQQRRKSAMF
RDH12, retinol dehydrogenase 12 (ENSG00000139988)
Nucleotide sequence SEQ ID No. 864
ATGCTGGTCACCTTGGGACTGCTCACCTCCTTCTTCTCGTTCCTGTATATGGTAGCTCCATCCATCAGGA
AGTTCTTTGCTGGTGGAGTGTGTAGAACAAATGTGCAGCTTCCTGGCAAGGTAGTGGTGATCACTGGC
GC
CAACACGGGCATTGGCAAGGAGACGGCCAGAGAGCTCGCTAGCCGAGGAGCCCGAGTCTATATTGCC
TGC
AGAGATGTACTGAAGGGGGAGTCTGCTGCCAGTGAAATCCGAGTGGATACAAAGAACTCCCAGGTGC
TGG
TGCGGAAATTGGACCTATCCGACACCAAATCTATCCGAGCCTTTGCTGAGGGCTTTCTGGCAGAGGAA
AA
GCAGCTCCATATTCTGATCAACAATGCGGGAGTAATGATGTGTCCATATTCCAAGACAGCTGATGGCTT
T
GAAACCCACCTGGGAGTCAACCACCTGGGCCACTTCCTCCTCACCTACCTGCTCCTGGAGCGGCTAAAG
G
TGTCTGCCCCTGCACGGGTGGTTAATGTGTCCTCGGTGGCTCACCACATTGGCAAGATTCCCTTCCACG
A
CCTCCAGAGCGAGAAGCGCTACAGCAGGGGTTTTGCCTATTGCCACAGCAAGCTGGCCAATGTGCTTT
TT
ACTCGTGAGCTGGCCAAGAGGCTCCAAGGCACCGGGGTCACCACCTACGCAGTGCACCCAGGCGTCG
TCC
GCTCTGAGCTGGTCCGGCACTCCTCCCTGCTCTGCCTGCTCTGGCGGCTCTTCTCCCCCTTTGTCAAGAC
GGCACGGGAGGGGGCGCAGACCAGCCTGCACTGCGCCCTGGCTGAGGGCCTGGAGCCCCTGAGTGG
CAAG
TACTTCAGTGACTGCAAGAGGACCTGGGTGTCTCCAAGGGCCCGAAATAACAAAACAGCTGAGCGCCT
AT
GGAATGTCAGCTGTGAGCTTCTAGGAATCCGGTGGGAGTAG
Aminoacid sequence SEQ ID No. 865
MLVTLGLLTSFFSFLYMVAPSIRKFFAGGVCRTNVQLPGKVVVITGANTGIGKETARELASRGARVYIAC
RDVLKGESAASEIRVDTKNSQVLVRKLDLSDTKSIRAFAEGFLAEEKQLHILINNAGVMMCPYSKTADGF
ETHLGVNHLGHFLLTYLLLERLKVSAPARVVNVSSVAHHIGKIPFHDLQSEKRYSRGFAYCHSKLANVLF
TRELAKRLQGTGVTTYAVHPGVVRSELVRHSSLLCLLWRLFSPFVKTAREGAQTSLHCALAEGLEPLSGK
YFSDCKRTWVSPRARNNKTAERLWNVSCELLGIRWE
N2RE3, nuclear receptor subfamily 2 group E member 3 (ENSG00000278570)
Nucleotide sequence SEQ ID No. 866
ATGGAGACCAGACCAACAGCTCTGATGAGCTCCACAGTGGCTGCAGCTGCGCCTGCAGCTGGGGCTG
CCTCCAGGAAGGAGTCTCCAGGCAGATGGGGCCTGGGGGAGGATCCCACAGGCGTGAGCCCCTCGCT
CCAGTGCCGCGTGTGCGGAGACAGCAGCAGCGGGAAGCACTATGGCATCTATGCCTGCAACGGCTGC
AGCGGCTTCTTCAAGAGGAGCGTACGGCGGAGGCTCATCTACAGGTGCCAGGTGGGGGCAGGGATGT
GCCCCGTGGACAAGGCCCACCGCAACCAGTGCCAGGCCTGCCGGCTGAAGAAGTGCCTGCAGGCGGG
GATGAACCAGGACGCCGTGCAGAACGAGCGCCAGCCGCGAAGCACAGCCCAGGTCCACCTGGACAGC
ATGGAGTCCAACACTGAGTCCCGGCCGGAGTCCCTGGTGGCTCCCCCGGCCCCGGCAGGGCGCAGCC
CACGGGGCCCCACACCCATGTCTGCAGCCAGAGCCCTGGGCCACCACTTCATGGCCAGCCTTATAACA
GCTGAAACCTGTGCTAAGCTGGAGCCAGAGGATGCTGATGAGAATATTGATGTCACCAGCAATGACCC
TGAGTTCCCCTCCTCTCCATACTCCTCTTCCTCCCCCTGCGGCCTGGACAGCATCCATGAGACCTCGGCT
CGCCTACTCTTCATGGCCGTCAAGTGGGCCAAGAACCTGCCTGTGTTCTCCAGCCTGCCCTTCCGGGAT
CAGGTGATCCTGCTGGAAGAGGCGTGGAGTGAACTCTTTCTCCTCGGGGCCATCCAGTGGTCTCTGCC
TCTGGACAGCTGTCCTCTGCTGGCACCGCCCGAGGCCTCTGCTGCCGGTGGTGCCCAGGGCCGGCTCA
CGCTGGCCAGCATGGAGACGCGTGTCCTGCAGGAAACTATCTCTCGGTTCCGGGCATTGGCGGTGGAC
CCCACGGAGTTTGCCTGCATGAAGGCCTTGGTCCTCTTCAAGCCAGAGACGCGGGGCCTGAAGGATCC
TGAGCACGTAGAGGCCTTGCAGGACCAGTCCCAAGTGATGCTGAGCCAGCACAGCAAGGCCCACCAC
CCCAGCCAGCCCGTGAGGTGA
Aminoacid sequence SEQ ID No. 867
METRPTALMSSTVAAAAPAAGAASRKESPGRWGLGEDPTGVSPSLQCRVCGDSSSGKHYGIYACNGCSGF
FKRSVRRRLIYRCQVGAGMCPVDKAHRNQCQACRLKKCLQAGMNQDAVQNERQPRSTAQVHLDSMES
NTE
SRPESLVAPPAPAGRSPRGPTPMSAARALGHHFMASLITAETCAKLEPEDADENIDVTSNDPEFPSSPYS
SSSPCGLDSIHETSARLLFMAVKWAKNLPVFSSLPFRDQVILLEEAWSELFLLGAIQWSLPLDSCPLLAP
PEASAAGGAQGRLTLASMETRVLQETISRFRALAVDPTEFACMKALVLFKPETRGLKDPEHVEALQDQSQ
VMLSQHSKAHHPSQPVR
NRL, neural retina leucine zipper (ENSG00000129535)
Nucleotide sequence SEQ ID No. 868
ATGGCCCTGCCCCCCAGCCCCCTGGCCATGGAATATGTCAATGACTTTGACTTGATGAAGTTTGAGGTA
A
AGCGGGAACCCTCTGAGGGCCGACCTGGCCCCCCTACAGCCTCACTGGGCTCCACACCTTACAGCTCA
GT
GCCTCCTTCACCCACCTTCAGTGAACCAGGCATGGTGGGGGCAACCGAGGGCACCCGGCCAGGCCTG
GAG
GAGCTGTACTGGCTGGCTACCCTGCAGCAGCAGCTGGGGGCTGGGGAGGCATTGGGGCTGAGTCCTG
AAG
AGGCCATGGAGCTGCTGCAGGGTCAGGGCCCAGTCCCTGTTGATGGGCCCCATGGCTACTACCCAGG
GAG
CCCAGAGGAGACAGGAGCCCAGCACGTCCAGCTGGCAGAGCGGTTTTCCGACGCGGCGCTGGTCTCG
ATG
TCTGTGCGGGAGCTAAACCGGCAGCTGCGGGGCTGCGGGCGCGACGAGGCGCTGCGGCTGAAGCAG
AGGC
GCCGCACGCTGAAGAACCGCGGCTACGCGCAGGCCTGTCGCTCCAAGCGGCTGCAGCAGCGGCGCGG
GCT
GGAGGCCGAGCGCGCCCGCCTGGCCGCCCAGCTGGACGCGCTGCGGGCCGAGGTGGCCCGCCTGGC
CCGG
GAGCGCGATCTCTACAAGGCTCGCTGTGACCGGCTAACCTCGAGCGGCCCCGGGTCCGGGGACCCCTC
CC
ACCTCTTCCTCTGA
Aminoacid sequence SEQ ID No. 869
MALPPSPLAMEYVNDFDLMKFEVKREPSEGRPGPPTASLGSTPYSSVPPSPTFSEPGMVGATEGTRPGLE
ELYWLATLQQQLGAGEALGLSPEEAMELLQGQGPVPVDGPHGYYPGSPEETGAQHVQLAERFSDAALVS
M
SVRELNRQLRGCGRDEALRLKQRRRTLKNRGYAQACRSKRLQQRRGLEAERARLAAQLDALRAEVARLAR
ERDLYKARCDRLTSSGPGSGDPSHLFL
ROM1, retinal outer segment membrane protein 1 (ENSG00000149489)
Nucleotide sequence SEQ ID No. 870
ATGGCGCCGGTGTTGCCCCTGGTGCTGCCCCTGCAGCCCCGCATCCGCCTGGCACAAGGGCTCTGGCT
CC
TCTCCTGGCTGCTGGCGCTGGCTGGTGGCGTCATCCTCCTCTGTAGTGGGCACCTCCTGGTCCAGCTAA
G
GCACCTTGGCACCTTCCTGGCTCCCTCCTGTCAGTTCCCTGTCCTGCCCCAGGCTGCCCTGGCAGCGGG
C
GCGGTGGCTCTGGGCACAGGACTAGTGGGTGTAGGAGCCAGCCGGGCAAGTCTGAATGCAGCTCTAT
ACC
CTCCCTGGCGAGGGGTCCTGGGCCCGCTGCTGGTGGCTGGCACGGCTGGTGGGGGGGGGCTCCTGGT
CGT
CGGCCTCGGGCTAGCCCTGGCTTTGCCTGGGAGTCTGGATGAGGCGCTGGAGGAGGGCCTGGTGACT
GCC
TTGGCTCACTACAAGGACACAGAGGTGCCTGGGCACTGTCAGGCCAAAAGGCTGGTGGATGAGCTGC
AAC
TGAGGTACCACTGCTGCGGGCGCCACGGGTACAAGGATTGGTTTGGGGTCCAGTGGGTCAGCAGCCG
TTA
CCTGGATCCCGGTGACCGGGATGTGGCTGACCGGATCCAGAGCAATGTAGAAGGCCTATACCTGACTG
AT
GGGGTCCCTTTCTCCTGTTGCAACCCCCACTCACCCCGGCCTTGCCTGCAAAACCGTCTTTCAGACTCCT
ACGCCCACCCCCTGTTCGATCCCCGACAACCCAACCAAAACCTCTGGGCCCAAGGGTGCCATGAGGTG
CT
GCTGGAGCACTTGCAGGACTTGGCAGGCACACTGGGTAGCATGCTGGCTGTCACCTTCCTACTGCAGG
CT
CTGGTGCTCCTTGGCCTGCGGTACCTGCAAACAGCACTGGAGGGGCTTGGAGGGGTCATTGATGCGG
GAG
GAGAGACCCAGGGCTATCTCTTTCCCAGTGGGCTGAAAGATATGCTGAAAACAGCATGGCTACAGGG
AGG
GGTTGCCTGCAGGCCAGCACCTGAGGAGGCCCCACCAGGAGAAGCACCTCCCAAGGAGGATCTATCT
GAG
GCCTAG
Aminoacid sequence SEQ ID No. 871
MAPVLPLVLPLQPRIRLAQGLWLLSWLLALAGGVILLCSGHLLVQLRHLGTFLAPSCQFPVLPQAALAAG
AVALGTGLVGVGASRASLNAALYPPWRGVLGPLLVAGTAGGGGLLVVGLGLALALPGSLDEALEEGLVTA
LAHYKDTEVPGHCQAKRLVDELQLRYHCCGRHGYKDWFGVQWVSSRYLDPGDRDVADRIQSNVEGLYLT
D
GVPFSCCNPHSPRPCLQNRLSDSYAHPLFDPRQPNQNLWAQGCHEVLLEHLQDLAGTLGSMLAVTFLLQA
LVLLGLRYLQTALEGLGGVIDAGGETQGYLFPSGLKDMLKTAWLQGGVACRPAPEEAPPGEAPPKEDLSE
A
OTX2, orthodenticle homeobox 2 (ENSG00000165588)
Nucleotide sequence SEQ ID No. 872
ATGATGTCTTATCTTAAGCAACCGCCTTACGCAGTCAATGGGCTGAGTCTGACCACTTCGGGTATGGAC
T
TGCTGCACCCCTCCGTGGGCTACCCGGGGCCCTGGGCTTCTTGTCCCGCAGCCACCCCCCGGAAACAG
CG
CCGGGAGAGGACGACGTTCACTCGGGCGCAGCTAGATGTGCTGGAAGCACTGTTTGCCAAGACCCGG
TAC
CCAGACATCTTCATGCGAGAGGAGGTGGCACTGAAAATCAACTTGCCCGAGTCGAGGGTGCAGGTAT
GGT
TTAAGAATCGAAGAGCTAAGTGCCGCCAACAACAGCAACAACAGCAGAATGGAGGTCAAAACAAAGT
GAG
ACCTGCCAAAAAGAAGACATCTCCAGCTCGGGAAGTGAGTTCAGAGAGTGGAACAAGTGGCCAATTC
ACT
CCCCCCTCTAGCACCTCAGTCCCGACCATTGCCAGCAGCAGTGCTCCTGTGTCTATCTGGAGCCCAGCTT
CCATCTCCCCACTGTCAGATCCCTTGTCCACCTCCTCTTCCTGCATGCAGAGGTCCTATCCCATGACCTA
TACTCAGGCTTCAGGTTATAGTCAAGGATATGCTGGCTCAACTTCCTACTTTGGGGGCATGGACTGTGG
A
TCATATTTGACCCCTATGCATCACCAGCTTCCCGGACCAGGGGCCACACTCAGTCCCATGGGTACCAAT
G
CAGTCACCAGCCATCTCAATCAGTCCCCAGCTTCTCTTTCCACCCAGGGATATGGAGCTTCAAGCTTGG
G
TTTTAACTCAACCACTGATTGCTTGGATTATAAGGACCAAACTGCCTCCTGGAAGCTTAACTTCAATGCT
GACTGCTTGGATTATAAAGATCAGACATCCTCGTGGAAATTCCAGGTTTTGTGA
Aminoacid sequence SEQ ID No. 873
MMSYLKQPPYAVNGLSLTTSGMDLLHPSVGYPGPWASCPAATPRKQRRERTTFTRAQLDVLEALFAKTRY
PDIFMREEVALKINLPESRVQVWFKNRRAKCRQQQQQQQNGGQNKVRPAKKKTSPAREVSSESGTSGQF
TPPSSTSVPTIASSSAPVSIWSPASISPLSDPLSTSSSCMQRSYPMTYTQASGYSQGYAGSTSYFGGMDCG
SYLTPMHHQLPGPGATLSPMGTNAVTSHLNQSPASLSTQGYGASSLGFNSTTDCLDYKDQTASWKLNFNA
DCLDYKDQTSSWKFQVL
GUCA1A, guanylate cyclase activator 1A (ENSG00000048545)
Nucleotide sequence SEQ ID No. 874
ATGGGCAACGTGATGGAGGGAAAGTCAGTGGAGGAGCTGAGCAGCACCGAGTGCCACCAGTGGTACAAGA
AGTTCATGACTGAGTGCCCCTCTGGCCAACTCACCCTCTATGAGTTCCGCCAGTTCTTCGGCCTCAAGAA
CCTGAGCCCGTCGGCCAGCCAGTACGTGGAACAGATGTTTGAGACTTTTGACTTCAACAAGGACGGCTAC
ATTGATTTCATGGAGTACGTGGCAGCGCTCAGCTTGGTCCTCAAGGGGAAGGTGGAACAGAAGCTCCGCT
GGTACTTCAAGCTCTATGATGTAGATGGCAACGGCTGCATTGACCGCGATGAGCTGCTCACCATCATCCA
GGCCATTCGCGCCATTAACCCCTGCAGCGATACCACCATGACTGCAGAGGAGTTCACCGATACAGTGTTC
TCCAAGATTGACGTCAACGGGGATGGGGAACTCTCCCTGGAAGAGTTTATAGAGGGCGTCCAGAAGGACC
AGATGCTCCTGGACACACTGACACGAAGCCTGGACCTTACCCGCATCGTGCGCAGGCTCCAGAATGGCGA
GCAAGACGAGGAGGGGGCTGACGAGGCCGCTGAGGCAGCCGGCTGA
Aminoacid sequence SEQ ID No. 875
MGNVMEGKSVEELSSTECHQWYKKFMTECPSGQLTLYEFRQFFGLKNLSPSASQYVEQMFETFDFNKDGY
IDFMEYVAALSLVLKGKVEQKLRWYFKLYDVDGNGCIDRDELLTIIQAIRAINPCSDTTMTAEEFTDTVF
SKIDVNGDGELSLEEFIEGVQKDQMLLDTLTRSLDLTRIVRRLONGEQDEEGADEAAEAAG
GUCY2D, guanylate cyclase 2D, retinal (ENSG00000132518)
Nucleotide sequence SEQ ID No. 876
ATGACCGCCTGCGCCCGCCGAGCGGGTGGGCTTCCGGACCCCGGGCTCTGCGGTCCCGCGTGGTGGGCTC
CGTCCCTGCCCCGCCTCCCCCGGGCCCTGCCCCGGCTCCCGCTCCTGCTGCTCCTGCTTCTGCTGCAGCC
CCCCGCCCTCTCCGCCGTGTTCACGGTGGGGGTCCTGGGCCCCTGGGCTTGCGACCCCATCTTCTCTCGG
GCTCGCCCGGACCTGGCCGCCCGCCTGGCCGCCGCCCGCCTGAACCGCGACCCCGGCCTGGCAGGCGGTC
CCCGCTTCGAGGTAGCGCTGCTGCCCGAGCCTTGCCGGACGCCGGGCTCGCTGGGGGCCGTGTCCTCCGC
GCTGGCCCGCGTGTCGGGCCTCGTGGGTCCGGTGAACCCTGCGGCCTGCCGGCCAGCCGAGCTGCTCGCC
GAAGAAGCCGGGATCGCGCTGGTGCCCTGGGGCTGCCCCTGGACGCAGGCGGAGGGCACCACGGCCCCTG
CCGTGACCCCCGCCGCGGATGCCCTCTACGCCCTGCTTCGCGCATTCGGCTGGGCGCGCGTGGCCCTGGT
CACCGCCCCCCAGGACCTGTGGGTGGAGGCGGGACGCTCACTGTCCACGGCACTCAGGGCCCGGGGCCTG
CCTGTCGCCTCCGTGACTTCCATGGAGCCCTTGGACCTGTCTGGAGCCCGGGAGGCCCTGAGGAAGGTTC
GGGACGGGCCCAGGGTCACAGCAGTGATCATGGTGATGCACTCGGTGCTGCTGGGTGGCGAGGAGCAGCG
CTACCTCCTGGAGGCCGCAGAGGAGCTGGGCCTGACCGATGGCTCCCTGGTCTTCCTGCCCTTCGACACG
ATCCACTACGCCTTGTCCCCAGGCCCGGAGGCCTTGGCCGCACTCGCCAACAGCTCCCAGCTTCGCAGGG
CCCACGATGCCGTGCTCACCCTCACGCGCCACTGTCCCTCTGAAGGCAGCGTGCTGGACAGCCTGCGCAG
GGCTCAAGAGCGCCGCGAGCTGCCCTCTGACCTCAATCTGCAGCAGGTCTCCCCACTCTTTGGCACCATC
TATGACGCGGTCTTCTTGCTGGCAAGGGGCGTGGCAGAAGCGCGGGCTGCCGCAGGTGGCAGATGGGTGT
CCGGAGCAGCTGTGGCCCGCCACATCCGGGATGCGCAGGTCCCTGGCTTCTGCGGGGACCTAGGAGGAGA
CGAGGAGCCCCCATTCGTGCTGCTAGACACGGACGCGGCGGGAGACCGGCTTTTTGCCACATACATGCTG
GATCCTGCCCGGGGCTCCTTCCTCTCCGCCGGTACCCGGATGCACTTCCCGCGTGGGGGATCAGCACCCG
GACCTGACCCCTCGTGCTGGTTCGATCCAAACAACATCTGCGGTGGAGGACTGGAGCCGGGCCTCGTCTT
TCTTGGCTTCCTCCTGGTGGTTGGGATGGGGCTGGCTGGGGCCTTCCTGGCCCATTATGTGAGGCACCGG
CTACTTCACATGCAAATGGTCTCCGGCCCCAACAAGATCATCCTGACCGTGGACGACATCACCTTTCTCC
ACCCACATGGGGGCACCTCTCGAAAGGTGGCCCAGGGGAGTCGATCAAGTCTGGGTGCCCGCAGCATGTC
AGACATTCGCAGCGGCCCCAGCCAACACTTGGACAGCCCCAACATTGGTGTCTATGAGGGAGACAGGGTT
TGGCTGAAGAAATTCCCAGGGGATCAGCACATAGCTATCCGCCCAGCAACCAAGACGGCCTTCTCCAAGC
TCCAGGAGCTCCGGCATGAGAACGTGGCCCTCTACCTGGGGCTTTTCCTGGCTCGGGGAGCAGAAGGCCC
TGCGGCCCTCTGGGAGGGCAACCTGGCTGTGGTCTCAGAGCACTGCACGCGGGGCTCTCTTCAGGACCTC
CTCGCTCAGAGAGAAATAAAGCTGGACTGGATGTTCAAGTCCTCCCTCCTGCTGGACCTTATCAAGGGAA
TAAGGTATCTGCACCATCGAGGCGTGGCTCATGGGCGGCTGAAGTCACGGAACTGCATAGTGGATGGCAG
ATTCGTACTCAAGATCACTGACCACGGCCACGGGAGACTGCTGGAAGCACAGAAGGTGCTACCGGAGCCT
CCCAGAGCGGAGGACCAGCTGTGGACAGCCCCGGAGCTGCTTAGGGACCCAGCCCTGGAGCGCCGGGGAA
CGCTGGCCGGCGACGTCTTTAGCTTGGCCATCATCATGCAAGAAGTAGTGTGCCGCAGTGCCCCTTATGC
CATGCTGGAGCTCACTCCCGAGGAAGTGGTGCAGAGGGTGCGGAGCCCCCCTCCACTGTGTCGGCCCTTG
GTGTCCATGGACCAGGCACCTGTCGAGTGTATCCTCCTGATGAAGCAGTGCTGGGCAGAGCAGCCGGAAC
TTCGGCCCTCCATGGACCACACCTTCGACCTGTTCAAGAACATCAACAAGGGCCGGAAGACGAACATCAT
TGACTCGATGCTTCGGATGCTGGAGCAGTACTCTAGTAACCTGGAGGATCTGATCCGGGAGCGCACGGAG
GAGCTGGAGCTGGAAAAGCAGAAGACAGACCGGCTGCTTACACAGATGCTGCCTCCGTCTGTGGCTGAGG
CCTTGAAGACGGGGACACCAGTGGAGCCCGAGTACTTTGAGCAAGTGACACTGTACTTTAGTGACATTGT
GGGCTTCACCACCATCTCTGCCATGAGTGAGCCCATTGAGGTTGTGGACCTGCTCAACGATCTCTACACA
CTCTTTGATGCCATCATTGGTTCCCACGATGTCTACAAGGTGGAGACAATAGGGGACGCCTATATGGTGG
CCTCGGGGCTGCCCCAGCGGAATGGGCAGCGACACGCGGCAGAGATCGCCAACATGTCACTGGACATCCT
CAGTGCCGTGGGCACTTTCCGCATGCGCCATATGCCTGAGGTTCCCGTGCGCATCCGCATAGGCCTGCAC
TCGGGTCCATGCGTGGCAGGCGTGGTGGGCCTCACCATGCCGCGGTACTGCCTGTTTGGGGACACGGTCA
ACACCGCCTCGCGCATGGAGTCCACCGGGCTGCCTTACCGCATCCACGTGAACTTGAGCACTGTGGGGAT
TCTCCGTGCTCTGGACTCGGGCTACCAGGTGGAGCTGCGAGGCCGCACGGAGCTGAAGGGCAAGGGCGCC
GAGGACACTTTCTGGCTAGTGGGCAGACGCGGCTTCAACAAGCCCATCCCCAAACCGCCTGACCTGCAAC
CGGGGTCCAGCAACCACGGCATCAGCCTGCAGGAGATCCCACCCGAGCGGCGACGGAAGCTGGAGAAGGC
GCGGCCGGGCCAGTTCTCTTGA
Aminoacid sequence SEQ ID No. 877
MTACARRAGGLPDPGLCGPAWWAPSLPRLPRALPRLPLLLLLLLLOPPALSAVFTVGVLGPWACDPIFSR
ARPDLAARLAAARLNRDPGLAGGPRFEVALLPEPCRTPGSLGAVSSALARVSGLVGPVNPAACRPAELLA
EEAGIALVPWGCPWTQAEGTTAPAVTPAADALYALLRAFGWARVALVTAPQDLWVEAGRSLSTALRARGL
PVASVTSMEPLDLSGAREALRKVRDGPRVTAVIMVMHSVLLGGEEQRYLLEAAEELGLTDGSLVFLPFDT
IHYALSPGPEALAALANSSQLRRAHDAVLTLTRHCPSEGSVLDSLRRAQERRELPSDLNLQQVSPLFGTI
YDAVFLLARGVAEARAAAGGRWVSGAAVARHIRDAQVPGFCGDLGGDEEPPFVLLDTDAAGDRLFATYML
DPARGSFLSAGTRMHFPRGGSAPGPDPSCWFDPNNICGGGLEPGLVFLGFLLVVGMGLAGAFLAHYVRHR
LLHMQMVSGPNKIILTVDDITFLHPHGGTSRKVAQGSRSSLGARSMSDIRSGPSQHLDSPNIGVYEGDRV
WLKKFPGDQHIAIRPATKTAFSKLQELRHENVALYLGLFLARGAEGPAALWEGNLAVVSEHCTRGSLQDL
LAQREIKLDWMFKSSLLLDLIKGIRYLHHRGVAHGRLKSRNCIVDGRFVLKITDHGHGRLLEAQKVLPEP
PRAEDQLWTAPELLRDPALERRGTLAGDVFSLAIIMQEVVCRSAPYAMLELTPEEVVQRVRSPPPLCRPL
VSMDQAPVECILLMKQCWAEQPELRPSMDHTFDLFKNINKGRKTNIIDSMLRMLEQYSSNLEDLIRERTE
ELELEKQKTDRLLTQMLPPSVAEALKTGTPVEPEYFEQVTLYFSDIVGFTTISAMSEPIEVVDLLNDLYT
LFDAIIGSHDVYKVETIGDAYMVASGLPQRNGQRHAAEIANMSLDILSAVGTFRMRHMPEVPVRIRIGLH
SGPCVAGVVGLTMPRYCLFGDTVNTASRMESTGLPYRIHVNLSTVGILRALDSGYQVELRGRTELKGKGA
EDTFWLVGRRGFNKPIPKPPDLQPGSSNHGISLQEIPPERRRKLEKARPGQFS
>pAAV2.1hGNAT1_hKFL15
SEQ ID No. 878
agcgcccaatacgcaaaccgcctctccccgcgcgttggccgattcattaatgcagctggcacgacaggtttcccgactggaaagcggg
cagtgagcgcaacgcaattaatgtgagttagctcactcattaggcaccccaggctttacactttatgcttccggctcgtatgttgtgtgga
attgtgagcggataacaatttcacacaggaaacagctatgaccatgattacgccagatttaattaaggctgcgcgctcgctcgctcact
gaggccgcccgggcaaagcccgggcgtcgggcgacctttggtcgcccggcctcagtgagcgagcgagcgcgcagagagggagtggc
caactccatcactaggggttccttgtagttaatgattaacccgccatgctacttatctacgtagccatgctctaggaagatcggaattcgc
ccttaagctagctccctgcaggtcataaaatcccagtccagagtcaccagcccttcttaaccacttcctactgtgtgaccctttcagccttt
acttcctcatcagtaaaatgaggctgatgatatgggcatccatactccagggccagtgtgagcttacaacaagataaggagtggtgctg
agcctggtgccgggcaggcagcaggcatgtttctcccaattatgccctctcactgccagccccacctccattgtcctcacccccagggct
caaggttctgccttcccctttctcagccctgaccctactgaacatgtctccccactcccaggcagtgccagggcctctcctggagggttgc
ggggacagaaggacagccggagtgcagagtcagcggttgagggattggggctatgccagcTAatCCgaagggttgggggggctga
gctggattcacctgtccttgtctctgattggctcttggacacccctagcccccaaatcccactaagcagccccaccagggattgcacagg
tccgtagagagccagTTGATTGCAGGTCCTCCTGGGGCCAGAAGGGTGCCTGGGAGGCCAGGTTCTGGGG
ATCCCCTCCATCCAGAAGAACCACCTGCTCACTCTGTCCCTTCGCCTGCTGCTGGGACCGCGGCCGCAT
GgaggtccacactaatcaagaccccctggatgccgaggtgcacaccaaccaggaccctctggacCATATGGTGGACCACTTA
CTTCCAGTGGACGAGAACTTCTCGTCGCCAAAATGCCCAGTTGGGTATCTGGGTGATAGGCTGGTTGG
CCGGCGGGCATATCACATGCTGCCCTCACCCGTCTCTGAAGATGACAGCGATGCCTCCAGCCCCTGCTC
CTGTTCCAGTCCCGACTCTCAAGCCCTCTGCTCCTGCTATGGTGGAGGCCTGGGCACCGAGAGCCAGG
ACAGCATCTTGGACTTCCTATTGTCCCAGGCCACGCTGGGCAGTGGCGGGGGCAGCGGCAGTAGCATT
GGGGCCAGCAGTGGCCCCGTGGCCTGGGGGCCCTGGCGAAGGGCAGCGGCCCCTGTGAAGGGGGAG
CATTTCTGCTTGCCCGAGTTTCCTTTGGGTGATCCTGATGACGTCCCACGGCCCTTCCAGCCTACCCTGG
AGGAGATTGAAGAGTTTCTGGAGGAGAACATGGAGCCTGGAGTCAAGGAGGTCCCTGAGGGCAACA
GCAAGGACTTGGATGCCTGCAGCCAGCTCTCAGCTGGGCCACACAAGAGCCACCTCCATCCTGGGTCC
AGCGGGAGAGAGCGCTGTTCCCCTCCACCAGGTGGTGCCAGTGCAGGAGGTGCCCAGGGCCCAGGTG
GGGGCCCCACGCCTGATGGCCCCATCCCAGTGTTGCTGCAGATCCAGCCCGTGCCTGTGAAGCAGGAA
TCGGGCACAGGGCCTGCCTCCCCTGGGCAAGCCCCAGAGAATGTCAAGGTTGCCCAGCTCCTGGTCAA
CATCCAGGGGCAGACCTTCGCACTCGTGCCCCAGGTGGTACCCTCCTCCAACTTGAACCTGCCCTCCAA
GTTTGTGCGCATTGCCCCTGTGCCCATTGCCGCCAAGCCTGTTGGATCGGGACCCCTGGGGCCTGGCCC
TGCCGGTCTCCTCATGGGCCAGAAGTTCCCCAAGAACCCAGCCGCAGAACTCATCAAAATGCACAAAT
GTACTTTCCCTGGCTGCAGCAAGATGTACACCAAAAGCAGCCACCTCAAGGCCCACCTGCGCCGGCAC
ACGGGTGAGAAGCCCTTCGCCTGCACCTGGCCAGGCTGCGGCTGGAGGTTCTCGCGCTCTGACGAGCT
GTCGCGGCACAGGCGCTCGCACTCAGGTGTGAAGCCGTACCAGTGTCCTGTGTGCGAGAAGAAGTTC
GCGCGGAGCGACCACCTCTCCAAGCACATCAAGGTGCACCGCTTCCCGCGGAGCAGCCGCTCCGTGCG
CTCCGTGAACTCTAGATACCCGTACGACGTTCCAGACTATGCATCTTGATAGAAgcaagcttggatccaatcaa
cctctggattacaaaatttgtgaaagattgactggtattcttaactatgttgctccttttacgctatgtggatacgctgctttaatgcctttgt
atcatgctattgcttcccgtatggctttcattttctcctccttgtataaatcctggttgctgtctctttatgaggagttgtggcccgttgtcagg
caacgtggcgtggtgtgcactgtgtttgctgacgcaacccccactggttggggcattgccaccacctgtcagctcctttccgggactttcg
ctttccccctccctattgccacggcggaactcatcgccgcctgccttgcccgctgctggacaggggctcggctgttgggcactgacaattc
cgtggtgttgtcggggaagctgacgtcctttccatggctgctcgcctgtgttgccacctggattctgcgcgggacgtccttctgctacgtcc
cttcggccctcaatccagcggaccttccttcccgcggcctgctgccggctctgcggcctcttccgcgtcttcgagatctgcctcgactgtgc
cttctagttgccagccatctgttgtttgcccctcccccgtgccttccttgaccctggaaggtgccactcccactgtcctttcctaataaaatg
aggaaattgcatcgcattgtctgagtaggtgtcattctattctggggggtggggtggggcaggacagcaagggggaggattgggaaga
caatagcaggcatgctggggactcgagttaagggcgaattcccgattaggatcttcctagagcatggctacgtagataagtagcatgg
cgggttaatcattaactacaaggaacccctagtgatggagttggccactccctctctgcgcgctcgctcgctcactgaggccgggcgacc
aaaggtcgcccgacgcccgggctttgcccgggcggcctcagtgagcgagcgagcgcgcagccttaattaacctaattcactggccgtc
gttttacaacgtcgtgactgggaaaaccctggcgttacccaacttaatcgccttgcagcacatccccctttcgccagctggcgtaatagc
gaagaggcccgcaccgatcgcccttcccaacagttgcgcagcctgaatggcgaatgggacgcgccctgtagcggcgcattaagcgcg
gcgggtgtggtggttacgcgcagcgtgaccgctacacttgccagcgccctagcgcccgctcctttcgctttcttcccttcctttctcgccac
gttcgccggctttccccgtcaagctctaaatcgggggctccctttagggttccgatttagtgctttacggcacctcgaccccaaaaaactt
gattagggtgatggttcacgtagtgggccatcgccccgatagacggtttttcgccctttgacgctggagttcacgttcctcaatagtggac
tcttgttccaaactggaacaacactcaaccctatctcggtctattcttttgatttataagggatttttccgatttcggcctattggttaaaaa
atgagctgatttaacaaaaatttaacgcgaattttaacaaaatattaacgtttataatttcaggtggcatctttcggggaaatgtgcgcg
gaacccctatttgtttatttttctaaatacattcaaatatgtatccgctcatgagacaataaccctgataaatgcttcaataatattgaaaa
aggaagagtatgagtattcaacatttccgtgtcgcccttattcccttttttgcggcattttgccttcctgtttttgctcacccagaaacgctg
gtgaaagtaaaagatgctgaagatcagttgggtgcacgagtgggttacatcgaactggatctcaatagtggtaagatccttgagagttt
tcgccccgaagaacgttttccaatgatgagcacttttaaagttctgctatgtggcgcggtattatcccgtattgacgccgggcaagagca
actcggtcgccgcatacactattctcagaatgacttggttgagtactcaccagtcacagaaaagcatcttacggatggcatgacagtaa
gagaattatgcagtgctgccataaccatgagtgataacactgcggccaacttacttctgacaacgatcggaggaccgaaggagctaac
cgcttttttgcacaacatgggggatcatgtaactcgccttgatcgttgggaaccggagctgaatgaagccataccaaacgacgagcgtg
acaccacgatgcctgtagtaatggtaacaacgttgcgcaaactattaactggcgaactacttactctagcttcccggcaacaattaata
gactggatggaggcggataaagttgcaggaccacttctgcgctcggcccttccggctggctggtttattgctgataaatctggagccggt
gagcgtgggtctcgcggtatcattgcagcactggggccagatggtaagccctcccgtatcgtagttatctacacgacggggagtcaggc
aactatggatgaacgaaatagacagatcgctgagataggtgcctcactgattaagcattggtaactgtcagaccaagtttactcatata
tactttagattgatttaaaacttcatttttaatttaaaaggatctaggtgaagatcctttttgataatctcatgaccaaaatcccttaacgtg
agttttcgttccactgagcgtcagaccccgtagaaaagatcaaaggatcttcttgagatcctttttttctgcgcgtaatctgctgcttgcaa
acaaaaaaaccaccgctaccagcggtggtttgtttgccggatcaagagctaccaactctttttccgaaggtaactggcttcagcagagc
gcagataccaaatactgtccttctagtgtagccgtagttaggccaccacttcaagaactctgtagcaccgcctacatacctcgctctgct
aatcctgttaccagtggctgctgccagtggcgataagtcgtgtcttaccgggttggactcaagacgatagttaccggataaggcgcagc
ggtcgggctgaacggggggttcgtgcacacagcccagcttggagcgaacgacctacaccgaactgagatacctacagcgtgagctat
gagaaagcgccacgcttcccgaagggagaaaggcggacaggtatccggtaagcggcagggtcggaacaggagagcgcacgaggga
gcttccagggggaaacgcctggtatctttatagtcctgtcgggtttcgccacctctgacttgagcgtcgatttttgtgatgctcgtcagggg
ggcggagcctatggaaaaacgccagcaacgcggcctttttacggttcctggccttttgctgcggttttgctcacatgttctttcctgcgtta
tcccctgattctgtggataaccgtattaccgcctttgagtgagctgataccgctcgccgcagccgaacgaccgagcgcagcgagtcagt
gagcgaggaagcggaag
>pAAV2.1-hGNAT1-hRHO
SEQ ID No. 879
agcgcccaatacgcaaaccgcctctccccgcgcgttggccgattcattaatgcagctggcacgacaggtttcccga
ctgg
aaagcgggcagtgagcgcaacgcaattaatgtgagttagctcactcattaggcaccccaggctttacactttatgc
ttcc
ggctcgtatgttgtgtggaattgtgagcggataacaatttcacacaggaaacagctatgaccatgattacgccaga
ttta
attaaggctgcgcgctcgctcgctcactgaggccgcccgggcaaagcccgggcgtcgggcgacctttggtcgcccg
gcct
cagtgagcgagcgagcgcgcagagagggagtggccaactccatcactaggggttccttgtagttaatgattaaccc
gcca
tgctacttatctacgtagccatgctctaggaagatcggaattcgcccttaagctagctccctgcaggtcataaaat
ccca
gtccagagtcaccagcccttcttaaccacttcctactgtgtgaccctttcagcctttacttcctcatcagtaaaat
gagg
ctgatgatatgggcatccatactccagggccagtgtgagcttacaacaagataaggagtggtgctgagcctggtgc
cggg
caggcagcaggcatgtttctcccaattatgccctctcactgccagccccacctccattgtcctcacccccagggct
caag
gttctgccttcccctttctcagccctgaccctactgaacatgtctccccactcccaggcagtgccagggcctctcc
tgga
gggttgcggggacagaaggacagccggagtgcagagtcagcggttgagggattggggctatgccagcTAatCCgaa
gggt
tgggggggctgagctggattcacctgtccttgtctctgattggctcttggacacccctagcccccaaatcccacta
agca
gccccaccagggattgcacaggtccgtagagagccagTTGATTGCAGGTCCTCCTGGGGCCAGAAGGGTGCCTGGG
AGGC
CAGGTTCTGGGGATCCCCTCCATCCAGAAGAACCACCTGCTCACTCTGTCCCTTCGCCTGCTGCTGGGACCGCGGC
CGCA
TGAATGGCACAGAAGGCCCTAACTTCTACGTGCCCTTCTCCAATGCGACGGGTGTGGTACGCAGCCCCTTCGAGTA
CCCA
CAGTACTACCTGGCTGAGCCATGGCAGTTCTCCATGCTGGCCGCCTACATGTTTCTGCTGATCGTGCTGGGCTTCC
CCAT
CAACTTCCTCACGCTCTACGTCACCGTCCAGCACAAGAAGCTGCGCACGCCTCTCAACTACATCCTGCTCAACCTA
GCCG
TGGCTGACCTCTTCATGGTCCTAGGTGGCTTCACCAGCACCCTCTACACCTCTCTGCATGGATACTTCGTCTTCGG
GCCC
ACAGGATGCAATTTGGAGGGCTTCTTTGCCACCCTGGGCGGTGAAATTGCCCTGTGGTCCTTGGTGGTCCTGGCCA
TCGA
GCGGTACGTGGTGGTGTGTAAGCCCATGAGCAACTTCCGCTTCGGGGAGAACCATGCCATCATGGGCGTTGCCTTC
ACCT
GGGTCATGGCGCTGGCCTGCGCCGCACCCCCACTCGCCGGCTGGTCCAGGTACATCCCCGAGGGCCTGCAGTGCTC
GTGT
GGAATCGACTACTACACGCTCAAGCCGGAGGTCAACAACGAGTCTTTTGTCATCTACATGTTCGTGGTCCACTTCA
CCAT
CCCCATGATTATCATCTTTTTCTGCTATGGGCAGCTCGTCTTCACCGTCAAGGAGGCCGCTGCCCAGCAGCAGGAG
TCAG
CCACCACACAGAAGGCAGAGAAGGAGGTCACCCGCATGGTCATCATCATGGTCATCGCTTTCCTGATCTGCTGGGT
GCCC
TACGCCAGCGTGGCATTCTACATCTTCACCCACCAGGGCTCCAACTTCGGTCCCATCTTCATGACCATCCCAGCGT
TCTT
TGCCAAGAGCGCCGCCATCTACAACCCTGTCATCTATATCATGATGAACAAGCAGTTCCGGAACTGCATGCTCACC
ACCA
TCTGCTGCGGCAAGAACCCACTGGGTGACGATGAGGCCTCTGCTACCGTGTCCAAGACGGAGACGAGCCAGGTGGC
CCCG
GCCTAAAagcttggatccaatcaacctctggattacaaaatttgtgaaagattgactggtattcttaactatgttg
ctcc
ttttacgctatgtggatacgctgctttaatgcctttgtatcatgctattgcttcccgtatggctttcattttctcc
tcct
tgtataaatcctggttgctgtctctttatgaggagttgtggcccgttgtcaggcaacgtggcgtggtgtgcactgt
gttt
gctgacgcaacccccactggttggggcattgccaccacctgtcagctcctttccgggactttcgctttccccctcc
ctat
tgccacggcggaactcatcgccgcctgccttgcccgctgctggacaggggctcggctgttgggcactgacaattcc
gtgg
tgttgtcggggaagctgacgtcctttccatggctgctcgcctgtgttgccacctggattctgcgcgggacgtcctt
ctgc
tacgtcccttcggccctcaatccagcggaccttccttcccgcggcctgctgccggctctgcggcctcttccgcgtc
ttcg
agatctgcctcgactgtgccttctagttgccagccatctgttgtttgcccctcccccgtgccttccttgaccctgg
aagg
tgccactcccactgtcctttcctaataaaatgaggaaattgcatcgcattgtctgagtaggtgtcattctattctg
gggg
gtggggtggggcaggacagcaagggggaggattgggaagacaatagcaggcatgctggggactcgagttaagggcg
aatt
cccgattaggatcttcctagagcatggctacgtagataagtagcatggcgggttaatcattaactacaaggaaccc
ctag
tgatggagttggccactccctctctgcgcgctcgctcgctcactgaggccgggcgaccaaaggtcgcccgacgccc
gggc
tttgcccgggcggcctcagtgagcgagcgagcgcgcagccttaattaacctaattcactggccgtcgttttacaac
gtcg
tgactgggaaaaccctggcgttacccaacttaatcgccttgcagcacatccccctttcgccagctggcgtaatagc
gaag
aggcccgcaccgatcgcccttcccaacagttgcgcagcctgaatggcgaatgggacgcgccctgtagcggcgcatt
aagc
gcggcgggtgtggtggttacgcgcagcgtgaccgctacacttgccagcgccctagcgcccgctcctttcgctttct
tccc
ttcctttctcgccacgttcgccggctttccccgtcaagctctaaatcgggggctccctttagggttccgatttagt
gctt
tacggcacctcgaccccaaaaaacttgattagggtgatggttcacgtagtgggccatcgccccgatagacggtttt
tcgc
cctttgacgctggagttcacgttcctcaatagtggactcttgttccaaactggaacaacactcaaccctatctcgg
tcta
ttcttttgatttataagggatttttccgatttcggcctattggttaaaaaatgagctgatttaacaaaaatttaac
gcga
attttaacaaaatattaacgtttataatttcaggtggcatctttcggggaaatgtgcgcggaacccctatttgttt
attt
ttctaaatacattcaaatatgtatccgctcatgagacaataaccctgataaatgcttcaataatattgaaaaagga
agag
tatgagtattcaacatttccgtgtcgcccttattcccttttttgcggcattttgccttcctgtttttgctcaccca
gaaa
cgctggtgaaagtaaaagatgctgaagatcagttgggtgcacgagtgggttacatcgaactggatctcaatagtgg
taag
atccttgagagttttcgccccgaagaacgttttccaatgatgagcacttttaaagttctgctatgtggcgcggtat
tatc
ccgtattgacgccgggcaagagcaactcggtcgccgcatacactattctcagaatgacttggttgagtactcacca
gtca
cagaaaagcatcttacggatggcatgacagtaagagaattatgcagtgctgccataaccatgagtgataacactgc
ggcc
aacttacttctgacaacgatcggaggaccgaaggagctaaccgcttttttgcacaacatgggggatcatgtaactc
gcct
tgatcgttgggaaccggagctgaatgaagccataccaaacgacgagcgtgacaccacgatgcctgtagtaatggta
acaa
cgttgcgcaaactattaactggcgaactacttactctagcttcccggcaacaattaatagactggatggaggcgga
taaa
gttgcaggaccacttctgcgctcggcccttccggctggctggtttattgctgataaatctggagccggtgagcgtg
ggtc
tcgcggtatcattgcagcactggggccagatggtaagccctcccgtatcgtagttatctacacgacggggagtcag
gcaa
ctatggatgaacgaaatagacagatcgctgagataggtgcctcactgattaagcattggtaactgtcagaccaagt
ttac
tcatatatactttagattgatttaaaacttcatttttaatttaaaaggatctaggtgaagatcctttttgataatc
tcat
gaccaaaatcccttaacgtgagttttcgttccactgagcgtcagaccccgtagaaaagatcaaaggatcttcttga
gatc
ctttttttctgcgcgtaatctgctgcttgcaaacaaaaaaaccaccgctaccagcggtggtttgtttgccggatca
agag
ctaccaactctttttccgaaggtaactggcttcagcagagcgcagataccaaatactgtccttctagtgtagccgt
agtt
aggccaccacttcaagaactctgtagcaccgcctacatacctcgctctgctaatcctgttaccagtggctgctgcc
agtg
gcgataagtcgtgtcttaccgggttggactcaagacgatagttaccggataaggcgcagcggtcgggctgaacggg
gggt
tcgtgcacacagcccagcttggagcgaacgacctacaccgaactgagatacctacagcgtgagctatgagaaagcg
ccac
gcttcccgaagggagaaaggcggacaggtatccggtaagcggcagggtcggaacaggagagcgcacgagggagctt
ccag
ggggaaacgcctggtatctttatagtcctgtcgggtttcgccacctctgacttgagcgtcgatttttgtgatgctc
gtca
ggggggcggagcctatggaaaaacgccagcaacgcggcctttttacggttcctggccttttgctgcggttttgctc
acat
gttctttcctgcgttatcccctgattctgtggataaccgtattaccgcctttgagtgagctgataccgctcgccgc
agcc
gaacgaccgagcgcagcgagtcagtgagcgaggaagcggaag
>pAAV2.1-hGNAT1-hKLF15-hGNAT1-Rho
SEQ ID No. 880
agcgcccaatacgcaaaccgcctctccccgcgcgttggccgattcattaatgcagctggcacgacaggtttcccga
ctggaaagcgggcagtgagcgcaacgcaattaatgtgagttagctcactcattaggcaccccaggctttacacttt
atgcttccggctcgtatgttgtgtggaattgtgagcggataacaatttcacacaggaaacagctatgaccatgatt
acgccagatttaattaaggctgcgcgctcgctcgctcactgaggccgcccgggcaaagcccgggcgtcgggcgacc
tttggtcgcccggcctcagtgagcgagcgagcgcgcagagagggagtggccaactccatcactaggggttccttgt
agttaatgattaacccgccatgctacttatctacgtagccatgctctaggaagatcggaattcgcccttaaGCTAG
CTcctcctagtgtcaccttggcccctcttagaagccaattaggccctcagtttctgcagcggggattaatatgatt
atgaaatctcccagatgctgattcagccaggagcttaggagggggaggtcactttataagggtctgggggggtcag
aacccagagtcatccagctggagccctgagtggctgagctcaggccttcgcagcattcttgggtgggagcagccac
gggtcagccacaagggccacagccCAATTGATGgaggtccacactaatcaagaccccctggatgccgaggtgcaca
ccaaccaggaccctctggacCATATGGTGGACCACTTACTTCCAGTGGACGAGAACTTCTCGTCGCCAAAATGCCC
AGTTGGGTATCTGGGTGATAGGCTGGTTGGCCGGCGGGCATATCACATGCTGCCCTCACCCGTCTCTGAAGATGAC
AGCGATGCCTCCAGCCCCTGCTCCTGTTCCAGTCCCGACTCTCAAGCCCTCTGCTCCTGCTATGGTGGAGGCCTGG
GCACCGAGAGCCAGGACAGCATCTTGGACTTCCTATTGTCCCAGGCCACGCTGGGCAGTGGCGGGGGCAGCGGCAG
TAGCATTGGGGCCAGCAGTGGCCCCGTGGCCTGGGGGCCCTGGCGAAGGGCAGCGGCCCCTGTGAAGGGGGAGCAT
TTCTGCTTGCCCGAGTTTCCTTTGGGTGATCCTGATGACGTCCCACGGCCCTTCCAGCCTACCCTGGAGGAGATTG
AAGAGTTTCTGGAGGAGAACATGGAGCCTGGAGTCAAGGAGGTCCCTGAGGGCAACAGCAAGGACTTGGATGCCTG
CAGCCAGCTCTCAGCTGGGCCACACAAGAGCCACCTCCATCCTGGGTCCAGCGGGAGAGAGCGCTGTTCCCCTCCA
CCAGGTGGTGCCAGTGCAGGAGGTGCCCAGGGCCCAGGTGGGGGCCCCACGCCTGATGGCCCCATCCCAGTGTTGC
TGCAGATCCAGCCCGTGCCTGTGAAGCAGGAATCGGGCACAGGGCCTGCCTCCCCTGGGCAAGCCCCAGAGAATGT
CAAGGTTGCCCAGCTCCTGGTCAACATCCAGGGGCAGACCTTCGCACTCGTGCCCCAGGTGGTACCCTCCTCCAAC
TTGAACCTGCCCTCCAAGTTTGTGCGCATTGCCCCTGTGCCCATTGCCGCCAAGCCTGTTGGATCGGGACCCCTGG
GGCCTGGCCCTGCCGGTCTCCTCATGGGCCAGAAGTTCCCCAAGAACCCAGCCGCAGAACTCATCAAAATGCACAA
ATGTACTTTCCCTGGCTGCAGCAAGATGTACACCAAAAGCAGCCACCTCAAGGCCCACCTGCGCCGGCACACGGGT
GAGAAGCCCTTCGCCTGCACCTGGCCAGGCTGCGGCTGGAGGTTCTCGCGCTCTGACGAGCTGTCGCGGCACAGGC
GCTCGCACTCAGGTGTGAAGCCGTACCAGTGTCCTGTGTGCGAGAAGAAGTTCGCGCGGAGCGACCACCTCTCCAA
GCACATCAAGGTGCACCGCTTCCCGCGGAGCAGCCGCTCCGTGCGCTCCGTGAACTctagatacccgtacgacgtt
ccagactatgcatcttgaCATATGGcctcgactgtgccttctagttgccagccatctgttgtttgcccctcccccg
tgccttccttgaccctggaaggtgccactcccactgtcctttcctaataaaatgaggaaattgcatcgcattgtct
gagtaggtgtcattctattctggggggtggggtggggcaggacagcaagggggaggattgggaagacaatagcagg
catgctggggaACTAGTtgtagttaatgattaacccgccatgctacttatctacgtagccatgctctaggaagatc
ggaattcgcccttaaGTTACGCTAGCtccctgcaggtcataaaatcccagtccagagtcaccagcccttcttaacc
acttcctactgtgtgaccctttcagcctttacttcctcatcagtaaaatgaggctgatgatatgggcatccatact
ccagggccagtgtgagcttacaacaagataaggagtggtgctgagcctggtgccgggcaggcagcaggcatgtttc
tcccaattatgccctctcactgccagccccacctccattgtcctcacccccagggctcaaggttctgccttcccct
ttctcagccctgaccctactgaacatgtctccccactcccaggcagtgccagggcctctcctggagggttgcgggg
acagaaggacagccggagtgcagagtcagcggttgagggattggggctatgccagcTAatCCgaagggttgggggg
gctgagctggattcacctgtccttgtctctgattggctcttggacacccctagcccccaaatcccactaagcagcc
ccaccagggattgcacaggtccgtagagagccagTTGATTGCAGGTCCTCCTGGGGCCAGAAGGGTGCCTGGGAGG
CCAGGTTCTGGGGATCCCCTCCATCCAGAAGAACCACCTGCTCACTCTGTCCCTTCGCCTGCTGCTGGGACCGCGG
CCGCATGAATGGCACAGAAGGCCCTAACTTCTACGTGCCCTTCTCCAATGCGACGGGTGTGGTACGCAGCCCCTTC
GAGTACCCACAGTACTACCTGGCTGAGCCATGGCAGTTCTCCATGCTGGCCGCCTACATGTTTCTGCTGATCGTGC
TGGGCTTCCCCATCAACTTCCTCACGCTCTACGTCACCGTCCAGCACAAGAAGCTGCGCACGCCTCTCAACTACAT
CCTGCTCAACCTAGCCGTGGCTGACCTCTTCATGGTCCTAGGTGGCTTCACCAGCACCCTCTACACCTCTCTGCAT
GGATACTTCGTCTTCGGGCCCACAGGATGCAATTTGGAGGGCTTCTTTGCCACCCTGGGCGGTGAAATTGCCCTGT
GGTCCTTGGTGGTCCTGGCCATCGAGCGGTACGTGGTGGTGTGTAAGCCCATGAGCAACTTCCGCTTCGGGGAGAA
CCATGCCATCATGGGCGTTGCCTTCACCTGGGTCATGGCGCTGGCCTGCGCCGCACCCCCACTCGCCGGCTGGTCC
AGGTACATCCCCGAGGGCCTGCAGTGCTCGTGTGGAATCGACTACTACACGCTCAAGCCGGAGGTCAACAACGAGT
CTTTTGTCATCTACATGTTCGTGGTCCACTTCACCATCCCCATGATTATCATCTTTTTCTGCTATGGGCAGCTCGT
CTTCACCGTCAAGGAGGCCGCTGCCCAGCAGCAGGAGTCAGCCACCACACAGAAGGCAGAGAAGGAGGTCACCCGC
ATGGTCATCATCATGGTCATCGCTTTCCTGATCTGCTGGGTGCCCTACGCCAGCGTGGCATTCTACATCTTCACCC
ACCAGGGCTCCAACTTCGGTCCCATCTTCATGACCATCCCAGCGTTCTTTGCCAAGAGCGCCGCCATCTACAACCC
TGTCATCTATATCATGATGAACAAGCAGTTCCGGAACTGCATGCTCACCACCATCTGCTGCGGCAAGAACCCACTG
GGTGACGATGAGGCCTCTGCTACCGTGTCCAAGACGGAGACGAGCCAGGTGGCCCCGGCCTAAAagcttggatcca
atcaacctctggattacaaaatttgtgaaagattgactggtattcttaactatgttgctccttttacgctatgtgg
atacgctgctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgtttgctgacg
caacccccactggttggggcattgccaccacctgtcagctcctttccgggactttcgctttccccctccctattgc
cacggcggaactcatcgccgcctgccttgcccgctgctggacaggggctcggctgttgggcactgacaattccgtg
gtgttgtcggggaagctgacgtcctttccatggctgctcgcctgtgttgccacctggattctgcgcgggacgtcct
tctgctacgtcccttcggccctcaatccagcggaccttccttcccgcggcctgctgccggctctgcggcctcttcc
gcgtcttcgagatctgcctcgactgtgccttctagttgccagccatctgttgtttgcccctcccccgtgccttcct
tgaccctggaaggtgccactcccactgtcctttcctaataaaatgaggaaattgcatcgcattgtctgagtaggtg
tcattctattctggggggtggggtggggcaggacagcaagggggaggattgggaagacaatagcaggcatgctggg
gactcgagttaagggcgaattcccgattaggatcttcctagagcatggctacgtagataagtagcatggcgggtta
atcattaactacaaggaacccctagtgatggagttggccactccctctctgcgcgctcgctcgctcactgaggccg
ggcgaccaaaggtcgcccgacgcccgggctttgcccgggcggcctcagtgagcgagcgagcgcgcagccttaatta
acctaattcactggccgtcgttttacaacgtcgtgactgggaaaaccctggcgttacccaacttaatcgccttgca
gcacatccccctttcgccagctggcgtaatagcgaagaggcccgcaccgatcgcccttcccaacagttgcgcagcc
tgaatggcgaatgggacgcgccctgtagcggcgcattaagcgcggcgggtgtggtggttacgcgcagcgtgaccgc
tacacttgccagcgccctagcgcccgctcctttcgctttcttcccttcctttctcgccacgttcgccggctttccc
cgtcaagctctaaatcgggggctccctttagggttccgatttagtgctttacggcacctcgaccccaaaaaacttg
attagggtgatggttcacgtagtgggccatcgccccgatagacggtttttcgccctttgacgctggagttcacgtt
cctcaatagtggactcttgttccaaactggaacaacactcaaccctatctcggtctattcttttgatttataaggg
atttttccgatttcggcctattggttaaaaaatgagctgatttaacaaaaatttaacgcgaattttaacaaaatat
taacgtttataatttcaggtggcatctttcggggaaatgtgcgcggaacccctatttgtttatttttctaaataca
ttcaaatatgtatccgctcatgagacaataaccctgataaatgcttcaataatattgaaaaaggaagagtatgagt
attcaacatttccgtgtcgcccttattcccttttttgcggcattttgccttcctgtttttgctcacccagaaacgc
tggtgaaagtaaaagatgctgaagatcagttgggtgcacgagtgggttacatcgaactggatctcaatagtggtaa
gatccttgagagttttcgccccgaagaacgttttccaatgatgagcacttttaaagttctgctatgtggcgcggta
ttatcccgtattgacgccgggcaagagcaactcggtcgccgcatacactattctcagaatgacttggttgagtact
caccagtcacagaaaagcatcttacggatggcatgacagtaagagaattatgcagtgctgccataaccatgagtga
taacactgcggccaacttacttctgacaacgatcggaggaccgaaggagctaaccgcttttttgcacaacatgggg
gatcatgtaactcgccttgatcgttgggaaccggagctgaatgaagccataccaaacgacgagcgtgacaccacga
tgcctgtagtaatggtaacaacgttgcgcaaactattaactggcgaactacttactctagcttcccggcaacaatt
aatagactggatggaggcggataaagttgcaggaccacttctgcgctcggcccttccggctggctggtttattgct
gataaatctggagccggtgagcgtgggtctcgcggtatcattgcagcactggggccagatggtaagccctcccgta
tcgtagttatctacacgacggggagtcaggcaactatggatgaacgaaatagacagatcgctgagataggtgcctc
actgattaagcattggtaactgtcagaccaagtttactcatatatactttagattgatttaaaacttcatttttaa
tttaaaaggatctaggtgaagatcctttttgataatctcatgaccaaaatcccttaacgtgagttttcgttccact
gagcgtcagaccccgtagaaaagatcaaaggatcttcttgagatcctttttttctgcgcgtaatctgctgcttgca
aacaaaaaaaccaccgctaccagcggtggtttgtttgccggatcaagagctaccaactctttttccgaaggtaact
ggcttcagcagagcgcagataccaaatactgtccttctagtgtagccgtagttaggccaccacttcaagaactctg
tagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagccc
agcttggagcgaacgacctacaccgaactgagatacctacagcgtgagctatgagaaagcgccacgcttcccgaag
ggagaaaggcggacaggtatccggtaagcggcagggtcggaacaggagagcgcacgagggagcttccagggggaaa
cgcctggtatctttatagtcctgtcgggtttcgccacctctgacttgagcgtcgatttttgtgatgctcgtcaggg
gggcggagcctatggaaaaacgccagcaacgcggcctttttacggttcctggccttttgctgcggttttgctcaca
tgttctttcctgcgttatcccctgattctgtggataaccgtattaccgcctttgagtgagctgataccgctcgccg
cagccgaacgaccgagcgcagcgagtcagtgagcgaggaagcggaag
>pAAV2.1-hGNAT1-hKLF8-hGNAT1-Rho
SEQ ID No. 881
agcgcccaatacgcaaaccgcctctccccgcgcgttggccgattcattaatgcagctggcacgacaggtttcccga
ctggaaagcgggcagtgagcgcaacgcaattaatgtgagttagctcactcattaggcaccccaggctttacacttt
atgcttccggctcgtatgttgtgtggaattgtgagcggataacaatttcacacaggaaacagctatgaccatgatt
acgccagatttaattaaggctgcgcgctcgctcgctcactgaggccgcccgggcaaagcccgggcgtcgggcgacc
tttggtcgcccggcctcagtgagcgagcgagcgcgcagagagggagtggccaactccatcactaggggttccttgt
agttaatgattaacccgccatgctacttatctacgtagccatgctctaggaagatcggaattcgcccttaaGCTAG
CTcctcctagtgtcaccttggcccctcttagaagccaattaggccctcagtttctgcagcggggattaatatgatt
atgaaatctcccagatgctgattcagccaggagcttaggagggggaggtcactttataagggtctgggggggtcag
aacccagagtcatccagctggagccctgagtggctgagctcaggccttcgcagcattcttgggtgggagcagccac
gggtcagccacaagggccacagccCAATTGATGgaggtccacactaatcaagaccccctggatgccgaggtgcaca
ccaaccaggaccctctggacCATATGGTCGATATGGATAAACTCATAAACAACTTGGAGGTCCAACTTAATTCAGA
AGGTGGCTCAATGCAGGTATTCAAGCAGGTCACTGCTTCTGTTCGGAACAGAGATCCCCCTGAGATAGAATACAGA
AGTAATATGACTTCTCCAACACTCCTGGATGCCAACCCCATGGAGAACCCAGCACTGTTTAATGACATCAAGATTG
AGCCCCCAGAAGAACTTTTGGCTAGTGATTTCAGCCTGCCCCAAGTGGAACCAGTTGACCTCTCCTTTCACAAGCC
CAAGGCTCCTCTCCAGCCTGCTAGCATGCTACAAGCTCCAATACGTCCCCCCAAGCCACAGTCTTCTCCCCAGACC
CTTGTGGTGTCCACGTCAACATCTGACATGAGCACTTCAGCAAACATTCCTACTGTTCTGACCCCAGGCTCTGTCC
TGACCTCCTCTCAGAGCACTGGTAGCCAGCAGATCTTACATGTCATTCACACTATCCCCTCAGTCAGTCTGCCAAA
TAAGATGGGTGGCCTGAAGACCATCCCAGTGGTAGTGCAGTCTCTGCCCATGGTGTATACTACTTTGCCTGCAGAT
GGGGGCCCTGCAGCCATTACAGTCCCACTCATTGGAGGAGATGGTAAAAATGCTGGATCAGTGAAAGTTGACCCCA
CCTCCATGTCTCCACTGGAAATTCCAAGTGACAGTGAGGAGAGTACAATTGAGAGTGGATCCTCAGCCTTGCAGAG
TCTGCAGGGACTACAGCAAGAGAGAGAAGCCTTATAAACTctagatacccgtacgacgttccagactatgcatctt
gaCATATGGcctcgactgtgccttctagttgccagccatctgttgtttgcccctcccccgtgccttccttgaccct
ggaaggtgccactcccactgtcctttcctaataaaatgaggaaattgcatcgcattgtctgagtaggtgtcattct
attctggggggtggggtggggcaggacagcaagggggaggattgggaagacaatagcaggcatgctggggaACTAG
TtgtagttaatgattaacccgccatgctacttatctacgtagccatgctctaggaagatcggaattcgcccttaaG
TTACGCTAGCtccctgcaggtcataaaatcccagtccagagtcaccagcccttcttaaccacttcctactgtgtga
ccctttcagcctttacttcctcatcagtaaaatgaggctgatgatatgggcatccatactccagggccagtgtgag
cttacaacaagataaggagtggtgctgagcctggtgccgggcaggcagcaggcatgtttctcccaattatgccctc
tcactgccagccccacctccattgtcctcacccccagggctcaaggttctgccttcccctttctcagccctgaccc
tactgaacatgtctccccactcccaggcagtgccagggcctctcctggagggttgcggggacagaaggacagccgg
agtgcagagtcagcggttgagggattggggctatgccagcTAatCCgaagggttgggggggctgagctggattcac
ctgtccttgtctctgattggctcttggacacccctagcccccaaatcccactaagcagccccaccagggattgcac
aggtccgtagagagccagTTGATTGCAGGTCCTCCTGGGGCCAGAAGGGTGCCTGGGAGGCCAGGTTCTGGGGATC
CCCTCCATCCAGAAGAACCACCTGCTCACTCTGTCCCTTCGCCTGCTGCTGGGACCGCGGCCGCATGAATGGCACA
GAAGGCCCTAACTTCTACGTGCCCTTCTCCAATGCGACGGGTGTGGTACGCAGCCCCTTCGAGTACCCACAGTACT
ACCTGGCTGAGCCATGGCAGTTCTCCATGCTGGCCGCCTACATGTTTCTGCTGATCGTGCTGGGCTTCCCCATCAA
CTTCCTCACGCTCTACGTCACCGTCCAGCACAAGAAGCTGCGCACGCCTCTCAACTACATCCTGCTCAACCTAGCC
GTGGCTGACCTCTTCATGGTCCTAGGTGGCTTCACCAGCACCCTCTACACCTCTCTGCATGGATACTTCGTCTTCG
GGCCCACAGGATGCAATTTGGAGGGCTTCTTTGCCACCCTGGGCGGTGAAATTGCCCTGTGGTCCTTGGTGGTCCT
GGCCATCGAGCGGTACGTGGTGGTGTGTAAGCCCATGAGCAACTTCCGCTTCGGGGAGAACCATGCCATCATGGGC
GTTGCCTTCACCTGGGTCATGGCGCTGGCCTGCGCCGCACCCCCACTCGCCGGCTGGTCCAGGTACATCCCCGAGG
GCCTGCAGTGCTCGTGTGGAATCGACTACTACACGCTCAAGCCGGAGGTCAACAACGAGTCTTTTGTCATCTACAT
GTTCGTGGTCCACTTCACCATCCCCATGATTATCATCTTTTTCTGCTATGGGCAGCTCGTCTTCACCGTCAAGGAG
GCCGCTGCCCAGCAGCAGGAGTCAGCCACCACACAGAAGGCAGAGAAGGAGGTCACCCGCATGGTCATCATCATGG
TCATCGCTTTCCTGATCTGCTGGGTGCCCTACGCCAGCGTGGCATTCTACATCTTCACCCACCAGGGCTCCAACTT
CGGTCCCATCTTCATGACCATCCCAGCGTTCTTTGCCAAGAGCGCCGCCATCTACAACCCTGTCATCTATATCATG
ATGAACAAGCAGTTCCGGAACTGCATGCTCACCACCATCTGCTGCGGCAAGAACCCACTGGGTGACGATGAGGCCT
CTGCTACCGTGTCCAAGACGGAGACGAGCCAGGTGGCCCCGGCCTAAAagcttggatccaatcaacctctggatta
caaaatttgtgaaagattgactggtattcttaactatgttgctccttttacgctatgtggatacgctgctttaatg
cctttgtatcatgctattgcttcccgtatggctttcattttctcctccttgtataaatcctggttgctgtctcttt
atgaggagttgtggcccgttgtcaggcaacgtggcgtggtgtgcactgtgtttgctgacgcaacccccactggttg
gggcattgccaccacctgtcagctcctttccgggactttcgctttccccctccctattgccacggcggaactcatc
gccgcctgccttgcccgctgctggacaggggctcggctgttgggcactgacaattccgtggtgttgtcggggaagc
tgacgtcctttccatggctgctcgcctgtgttgccacctggattctgcgcgggacgtccttctgctacgtcccttc
ggccctcaatccagcggaccttccttcccgcggcctgctgccggctctgcggcctcttccgcgtcttcgagatctg
cctcgactgtgccttctagttgccagccatctgttgtttgcccctcccccgtgccttccttgaccctggaaggtgc
cactcccactgtcctttcctaataaaatgaggaaattgcatcgcattgtctgagtaggtgtcattctattctgggg
ggtggggtggggcaggacagcaagggggaggattgggaagacaatagcaggcatgctggggactcgagttaagggc
gaattcccgattaggatcttcctagagcatggctacgtagataagtagcatggcgggttaatcattaactacaagg
aacccctagtgatggagttggccactccctctctgcgcgctcgctcgctcactgaggccgggcgaccaaaggtcgc
ccgacgcccgggctttgcccgggcggcctcagtgagcgagcgagcgcgcagccttaattaacctaattcactggcc
gtcgttttacaacgtcgtgactgggaaaaccctggcgttacccaacttaatcgccttgcagcacatccccctttcg
ccagctggcgtaatagcgaagaggcccgcaccgatcgcccttcccaacagttgcgcagcctgaatggcgaatggga
cgcgccctgtagcggcgcattaagcgcggcgggtgtggtggttacgcgcagcgtgaccgctacacttgccagcgcc
ctagcgcccgctcctttcgctttcttcccttcctttctcgccacgttcgccggctttccccgtcaagctctaaatc
gggggctccctttagggttccgatttagtgctttacggcacctcgaccccaaaaaacttgattagggtgatggttc
acgtagtgggccatcgccccgatagacggtttttcgccctttgacgctggagttcacgttcctcaatagtggactc
ttgttccaaactggaacaacactcaaccctatctcggtctattcttttgatttataagggatttttccgatttcgg
cctattggttaaaaaatgagctgatttaacaaaaatttaacgcgaattttaacaaaatattaacgtttataatttc
aggtggcatctttcggggaaatgtgcgcggaacccctatttgtttatttttctaaatacattcaaatatgtatccg
ctcatgagacaataaccctgataaatgcttcaataatattgaaaaaggaagagtatgagtattcaacatttccgtg
tcgcccttattcccttttttgcggcattttgccttcctgtttttgctcacccagaaacgctggtgaaagtaaaaga
tgctgaagatcagttgggtgcacgagtgggttacatcgaactggatctcaatagtggtaagatccttgagagtttt
cgccccgaagaacgttttccaatgatgagcacttttaaagttctgctatgtggcgcggtattatcccgtattgacg
ccgggcaagagcaactcggtcgccgcatacactattctcagaatgacttggttgagtactcaccagtcacagaaaa
gcatcttacggatggcatgacagtaagagaattatgcagtgctgccataaccatgagtgataacactgcggccaac
ttacttctgacaacgatcggaggaccgaaggagctaaccgcttttttgcacaacatgggggatcatgtaactcgcc
ttgatcgttgggaaccggagctgaatgaagccataccaaacgacgagcgtgacaccacgatgcctgtagtaatggt
aacaacgttgcgcaaactattaactggcgaactacttactctagcttcccggcaacaattaatagactggatggag
gcggataaagttgcaggaccacttctgcgctcggcccttccggctggctggtttattgctgataaatctggagccg
gtgagcgtgggtctcgcggtatcattgcagcactggggccagatggtaagccctcccgtatcgtagttatctacac
gacggggagtcaggcaactatggatgaacgaaatagacagatcgctgagataggtgcctcactgattaagcattgg
taactgtcagaccaagtttactcatatatactttagattgatttaaaacttcatttttaatttaaaaggatctagg
tgaagatcctttttgataatctcatgaccaaaatcccttaacgtgagttttcgttccactgagcgtcagaccccgt
agaaaagatcaaaggatcttcttgagatcctttttttctgcgcgtaatctgctgcttgcaaacaaaaaaaccaccg
ctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacata
cctcgctctgctaatcctgttaccagtggctgctgccagtggcgataagtcgtgtcttaccgggttggactcaaga
cgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacag
gtatccggtaagcggcagggtcggaacaggagagcgcacgagggagcttccagggggaaacgcctggtatctttat
agtcctgtcgggtttcgccacctctgacttgagcgtcgatttttgtgatgctcgtcaggggggCggagcctatgga
aaaacgccagcaacgcggcctttttacggttcctggccttttgctgcggttttgctcacatgttctttcctgcgtt
atcccctgattctgtggataaccgtattaccgcctttgagtgagctgataccgctcgccgcagccgaacgaccgag
cgcagcgagtcagtgagcgaggaagcggaag

### Definitions

### Embodiments [See Claim Set]

The present invention provides a nucleic acid construct comprising:
a) a nucleotide sequence encoding a first promoter;
b) a nucleotide sequence encoding a transcription factor
   wherein the nucleotide sequence of a) is operably linked to and drives the expression of the nucleotide sequence of b) in rod cells or cone cells of the retina where the protein encoded by the nucleotide sequence of b) is not physiologically expressed and
   wherein the protein encoded by said nucleotide sequence of b) recognizes at least a nucleotide sequence belonging to a gene which mutated form is responsible for a retinal dystrophy thereby silencing the expression of said gene.

Preferably the gene which mutated form is responsible for the retinal dystrophy is selected from RHO, PRPH2, CRX, RP1, GUCA1B, RDH12, N2RE3, NRL, ROM1, OTX2, GUCA1A, GUCY2D.

Preferably the transcription factor is selected from:
any one transcription factors described in Table 2 when the gene is RHO,
any one transcription factors described in Table 4 when the gene is CRX,
any one transcription factors described in Table 5 when the gene is GUCA1B,
any one transcription factors described in Table 6 when the gene is PRP2,
any one transcription factors described in Table 7 when the gene is RDH12,
any one transcription factors described in Table 8 when the gene is RP1
any one transcription factors described in Table 9 when the gene is GUCA1A
any one transcription factors described in Table 10 when the gene is GUCY2D
any one transcription factors described in Table 11 when the gene is N2RE3
any one transcription factors described in Table 12 when the gene is NRL
any one transcription factors described in Table 13 when the gene is OTX2
any one transcription factors described in Table 14 when the gene is ROM1,
preferably the transcription factor is selected from hKLF15, hKLF8, hZNF780A, hHMX1, MZF-1, hZN14, hZNF333, hZNF709, hZNF35.

Preferably the nucleic acid construct further comprises a nucleotide sequence coding for a wild-type form of a mutated coding sequence, wherein said mutated coding sequence is responsible for the retinal dystrophy, preferably said wild-type form of a mutated coding sequence is selected from the group consisting of RHO, PRPH2, CRX, RP1, GUCA1B, RDH12, N2RE3, NRL, ROM1, OTX2, GUCA1A, GUCY2D or the nucleic acid construct according to any one of claims 1 to 3 in combination with a second nucleic acid construct comprising a nucleotide sequence coding for a wild-type form of a mutated coding sequence, wherein said mutated coding sequence is responsible for the retinal dystrophy, preferably said wild-type form of a mutated coding sequence is selected from the group consisting of RHO, PRPH2, CRX, RP1, GUCA1B, RDH12, N2RE3, NRL, ROM1, OTX2, GUCA1A, GUCY2D.

In other words, the nucleotide sequence coding for a wild-type form of a mutated coding sequence may be part of the same construct as the Transcription factor or may be used in combination, as a separate independent construct.

Preferably said nucleotide sequence coding for a wild-type form of a mutated coding sequence is under the control of a nucleotide sequence of a second promoter.

Preferably the first and/or second promoter is GNAT1 or a promoter of a gene is selected from RHO, PRPH2, CRX, RP1, GUCA1B, RDH12, N2RE3, NRL, ROM1, OTX2, GUCA1A, GUCY2D.

Preferably the nucleotide sequence of the construct comprises any one of SEQ ID No. 837 to SEQ ID No. 881.

Preferably the retinal dystrophy is selected from retinitis pigmentosa, Leber's congenital amaurosis, cone dystrophy or cone-rod dystrophy.

The present invention also provides an expression vector that comprises the nucleic acid construct according to the invention, the expression vector may also comprise a second nucleic acid construct comprising a nucleotide sequence coding for a wild-type form of a mutated coding sequence, wherein said mutated coding sequence is responsible for the retinal dystrophy.

Preferably the vector is selected from the group consisting of: adenoviral vector, lentiviral vector, retroviral vector, Adeno associated vector (AAV) or naked plasmid DNA vector.

The present invention also provides a host cell comprising the nucleic acid construct, or an expression vector of the invention.

The present invention also provides viral particle that comprises a nucleic acid construct according to the invention or an expression vector according to the invention.

Preferably the viral particle comprises capsid proteins of an AAV.

More preferably the viral particle comprises capsid proteins of an AAV of a serotype selected from one or more of the groups consisting of AAV1, AAV2, AAV3, AAV4, AAV5, AAV6, AAV7, AAV8 AAV9 and AAV 10, preferably from the AAV2 or AAV8 serotype.

The present invention also provides pharmaceutical composition that comprises a nucleic acid construct or an expression vector or a host cell or a viral particle as defined above and a pharmaceutically acceptable carrier.

The present invention also provides a kit comprising a nucleic acid construct, an expression vector, a host cell or a viral particle or a pharmaceutical composition as defined above in one or more containers, optionally further comprising instructions or packaging materials that describe how to administer the nucleic acid construct, vector, host cell, viral particle or pharmaceutical composition to a patient.

The present invention also provides a nucleic acid construct, an expression vector, a host cell or a viral particle as defined above, for use as a medicament, preferably for use in the treatment of retinal dystrophy, preferably the retinal dystrophy is selected from retinitis pigmentosa, Leber's congenital amaurosis, cone dystrophy or cone-rod dystrophy.

The present invention also provides a nucleic acid construct, or an expression vector as defined above for the production of viral particles.

### DETAILED DESCRIPTION

### Diseases and Disease Genes of the Invention:

Rod-cone dystrophies, also known as retinitis pigmentosa (RP), are a clinically and genetically heterogeneous group of progressive inherited retinal disorders, which often starts with night blindness and leads to visual field constriction and secondary macular involvement.

In many cases, it may eventually result in loss of central vision and complete blindness [Wright et al., 2010]. RP occurs in one of 4,000 births and affects more than 1 million individuals worldwide. The mode of inheritance can be X-linked (xl), autosomal dominant (ad), or autosomal recessive (ar). In addition, many patients represent isolated cases, due to the absence of family history of RP. To date, mutations in 23 different genes are associated with adRP (http://www.sph.uth.tmc.edu/Retnet/) and the majority of prevalence studies reveal rhodopsin (RHO; MIM #180380) being the most frequently mutated gene in adRP [Audo et al. 2010b; Sullivan et al. 2006]. In addition, PRPF31 (MIM #606419), PRPH2 (MIM #179605), and RP1 (MIM #603937) were proposed to represent major genes underlying this form of RP [Audo et al., 2010a; Sullivan et al., 2006].

### Rhodopsin (RHO),

RHO mutations may be dominant for either of two reasons (Wilson and Wensel 2003; Mendes et al. 2005). Rhodopsin forms dimeric complexes in the disc membrane (Fotiadis et al. 2003), and mutant proteins might interfere with the function of normal rhodopsin or its assembly in the membrane, thereby exerting dominant negative effects.

Alternatively, gain-of-function mutations could cause rhodopsin to be intrinsically damaging to the rod cell. It may be possible to treat dominant negative mutations by increasing the level of the normal protein (supplementation). For mutations that cause rhodopsin to be injurious, however, suppressing the expression of the mutant proteins may also be required.

Still preferred disease genes are: CRX, Peripherin 2 (PRPH2), Retinitis pigmentosa 1 protein (RP1), Nuclear receptor subfamily 2 group E3 (N2RE3), Neural retina leucine zipper http://www.ncbi.nlm.nih.gov/gene/4901 (NRL)

### Retinal Outer Segment Membrane Protein 1 (ROM1)

This gene is a member of a photoreceptor-specific gene family and encodes an integral membrane protein found in the photoreceptor disk rim of the eye Mutations therein are responsible for rod dystrophies: OTX2, GUCA1B, RDH12; Mutations in the following genes are responsible for cone dystrophies: GUCA1A, guanylate cyclase activator 1A, GUCY2D, guanylate cyclase 2D, retinal.

### Promoters of the Invention:

Promoters of the invention are rod specific promoters including hGNAT1 promoter of SEQ ID NO. 12, and rod specific promoters of SEQ ID from 13 to 23, also disclosed in WO2017137493, included herein by reference.

Further promoters of the invention are cone-specific promoters, for instance red opsin gene regulatory region described in LI Q et al., Vision Research 48 (2008) 332-338, incorporated herein by reference: a 1 kb fragment of the upstream sequence of human red opsin gene containing a 1.6 kb BamHI-StuI fragment, extending from −3.1 to −4.6 kb joined to a proximal promoter of 495 bp of the human red pigment gene.

### Transcription Factors of the Invention:

Suitable transcription factors of the present invention are endogenous transcription factors which recognize the proximal regulatory region, preferably within the core promoter element, of a disease gene of the invention as defined herein and are not expressed in rod-photoreceptor cells.

Said regulatory region is defined as a DNA sequence within the proximal promoter region upstream or downstream of the transcription start site (TSS) (−250 from TSS and +150 from the TSS, total 400 bp). The TF may target DNA sequences which are either on the plus or minus strands of the said regulatory region.

The proximal promoter targeted sequence may include:
open chromatin sequences as assessed by the presence of transcription factors and co-factors such as p300 and the deposition of histone marks such as monomethylation of histone H3 lysine 4 (H3K4) and acetylation of H3K27, including H3K4me3, H3K4me2, H3K4me1, and H3K27AC, thus, almost exclusively in regions of low nucleosome occupancy, including
1—ATAC mapped sequences
2—MNase mapped sequences
3—DNasel mapped sequences
4—MNase mapped sequences

The proximal promoter targeted sequence may further include:
1—TATA box (also known as the Goldberg-Hogness box) eukaryotes sequence and TATA box proximal sequences.
2—CAAT box (also CAT box): typically located about 75-80 bases upstream of the transcription initiation site and about 150 bases upstream of the TATA box, and CAAT box proximal sequences.
3—E-box (enhancer box) typically an element present in the proximal core promoter regulatory region and E box proximal sequences.
4—GT box or GC box present in the proximal core promoter regulatory region and both GT box or GC box proximal sequences.
5—Phylogenetic conserved regulatory sequences.

The transcription factors of the invention are as indicated in Tables 2, 3, 4, 5, 6, 7 with their respective sequences.

Preferred transcription factors are as follows.

hKLF15

KLF15 belongs to the Kruppel-like factor (KLF) gene family (16), which possess a zinc-finger structure (KRAB-ZNF TFs) and recognize the core motif CACCC present in the hRHOcis (16).

KLF15 has a wide matrix sequence highly overlapping the ZF6-cis sequence (Table 2) and is expressed throughout the retina but not in photoreceptors (17) and thus can be excluded from having a regulatory function in these cells. In addition, although KLF15 exerts a wide range of regulatory functions in different organs and in system homeostasis (18-20), the mouse knock-out does not exhibit prominent phenotypes (21)

### Zinc Finger Protein 780A (O75290)

Binds the hPRP2 promoter not expressed in the retina

### MZF-1, Myeloid Zinc Finger 1 (P28698)

### Pituitary Homeobox 1 (P78337)

Bind hCRX promoter, not expressed in the retina

### HMX1 (Q9NP08)

Binds hRP1 promoter, not expressed in the retina

### Zinc Finger Protein 300 (Q96RE9-3)

Binds GUCA1B promoter, not expressed in the retina

### Zinc Finger Protein 333 (Q96JL9)

### Zinc Finger Protein 709 (Q8N972)

Bind RDH12 promoter, not expressed in the retina

### Zinc Finger Protein 35 (ZNF35)

Binds GUCA1A promoter, not expressed in the retina

### EXAMPLES

In order to identify transcription factors suitable for ectopic expression in rod cells in order to silence the Rhodopsin gene, the inventors searched initially for endogenous TFs with a DNA-binding preference for the ZF6-cis sequence motif ((−88 to −58 from the transcription start site, TSS), a 20 bp DNA sequence motif in the RHO promoter as defined in (12, 13) but that are not expressed in rod photoreceptors (the RHO-expressing cells). To retrieve TFs the inventors used Transfac analysis (15), which provides data on eukaryotic TF consensus binding sequences (based on Positional Weight Matrices, PWM), using as bait a 32 bp DNA sequence centred on the ZF6-cis sequence of the human RHO promoter (−88 to −58 from the RHO TSS, here named hRHO-cis). Among the set of retrieved TFs (FIG. 1A) KLF-15 belongs to the Kruppel-like factor (KLF) gene family (16), which possess a zinc-finger structure (KRAB-ZNF TFs) and recognize the “GT-box” and the core motif CACCC present in the hRHOcis (16). KLF15 has a wide matrix sequence highly overlapping the ZF6-cis sequence (Table 2) and is expressed throughout the retina but not in photoreceptors (17) and thus can be excluded from having a regulatory function in these cells. In addition, although KLF15 exerts a wide range of regulatory functions in different organs and in system homeostasis (18-20), the mouse knock-out does not exhibit prominent phenotypes (21).

TABLE 2
Transcription factor Position Weight Matrix (PWM, Transfac) recognizing the
RHODOPSIN proximal promoter.

| | | Position | Core | Matrix | |
|---|---|---|---|---|---|
| Matrix | Factor name | (strand) | score | score | Sequence |
| V$ZFP281_05 | ZFP281 | (+) | 1.000 | 0.983 | tgaacaCCCCCaatctc |
| | secondary motif | | | | SEQ ID No. 43 |
| V$TIEG1_Q6 | TIEG1 | (−) | 1.000 | 0.998 | GaacaCCCCC |
| | | | | | SEQ ID No. 44 |
| V$LRF_Q3 | LRF | (−) | 0.992 | 0.962 | gaacaCCCCCa |
| | | | | | SEQ ID No. 45 |
| V$KLF15_Q2 | KLF15 | (−) | 0.996 | 0.956 | gaacACCCCcaatc |
| | | | | | SEQ ID No. 46 |
| V$KLF8_Q5_01 | KLF8 | (−) | 1.000 | 0.970 | gaaCACCCcc |
| | | | | | SEQ ID No. 47 |
| V$ZIC3_01 | Zic3 | (−) | 1.000 | 0.961 | gaaCACCCc |
| | | | | | SEQ ID No. 48 |
| V$TBX5_02 | Tbx5 | (−) | 1.000 | 0.967 | gaACACCccc |
| | | | | | SEQ ID No. 49 |
| V$LRF_Q2 | LRF | (−) | 1.000 | 0.969 | aacaCCCCC |
| | | | | | SEQ ID No. 50 |
| V$KLF8_Q5 | KLF8 | (−) | 1.000 | 0.971 | aCACCCccaa |
| | | | | | SEQ ID No. 51 |
| V$KLF_Q3 | KLF | (−) | 0.994 | 0.955 | acaCCCCC |
| | | | | | SEQ ID No. 52 |
| V$ZXDB_01 | ZXDB | (−) | 1.000 | 0.955 | cACCCCc |
| | | | | | SEQ ID No. 53 |
| V$ZXDL_02 | ZXDL | (−) | 0.993 | 0.958 | cACCCCc |
| | | | | | SEQ ID No. 54 |
| V$CHCH_01 | Churchill | (−) | 0.983 | 0.983 | cACCCC |
| | | | | | SEQ ID No. 55 |
| V$CPBP_Q6 | CPBP | (+) | 0.997 | 0.994 | CACCCcc |
| | | | | | SEQ ID No. 56 |
| V$CPBP_Q6 | CPBP | (+) | 0.966 | 0.959 | ACCCCca |
| | | | | | SEQ ID No. 57 |
| VYCHCH_01 | Churchill | (−) | 0.986 | 0.975 | aCCCCC |
| | | | | | SEQ ID No. 58 |
| V$YB1_Q4 | YB-1 | (+) | 1.000 | 0.978 | acccCCAATct |
| | | | | | SEQ ID No. 59 |
| VYCHCH_01 | Churchill | (−) | 0.986 | 0.986 | cCCCCA |
| | | | | | SEQ ID No. 60 |
| V$CPBP_Q6 | CPBP | (+) | 1.000 | 0.996 | CCCCCaa |
| | | | | | SEQ ID No. 61 |
| V$MOVOB_01 | MOVO-B | (−) | 1.000 | 0.962 | CCCCCaa |
| | | | | | SEQ ID No. 62 |
| V$GEN_INI2_B | GEN_INI | (+) | 0.995 | 0.976 | cccCAATC |
| | | | | | SEQ ID No. 63 |
| V$GEN_INI3_B | GEN_INI | (+) | 0.993 | 0.979 | cccCAATC |
| | | | | | SEQ ID No. 64 |
| V$GEN_INI_B | GEN_INI | (+) | 0.995 | 0.973 | cccCAATC |
| | | | | | SEQ ID No. 65 |
| V$GFI1B_Q6 | Gfi1b | (+) | 0.995 | 0.972 | cccAATCTcc |
| | | | | | SEQ ID No. 66 |
| V$GATA6_01 | GATA-6 | (−) | 0.975 | 0.973 | ccCAATCtcc |
| | | | | | SEQ ID No. 67 |
| V$GATA3_01 | GATA-3 | (−) | 0.977 | 0.977 | ccCAATCtc |
| | | | | | SEQ ID No. 68 |
| VYGATA2_01 | GATA-2 | (−) | 0.979 | 0.976 | ccCAATCtcc |
| | | | | | SEQ ID No. 69 |
| V$GATA1_01 | GATA-1 | (−) | 0.997 | 0.994 | ccCAATCtcc |
| | | | | | SEQ ID No. 70 |
| V$HOXA7_01 | HOXA7 | (+) | 1.000 | 1.000 | cCAATCt |
| | | | | | SEQ ID No. 71 |
| V$IK2_01 | Ik-2 | (−) | 1.000 | 0.953 | aatcTCCCAgat |
| | | | | | SEQ ID No. 72 |
| VYRELA_03 | RelA-p65 | (−) | 1.000 | 0.952 | aatctCCCAGa |
| | | | | | SEQ ID No. 73 |
| V$IK_Q5 | Ikaros | (−) | 1.000 | 0.965 | atCTCCCaga |
| | | | | | SEQ ID No. 74 |
| V$NGN2_Q3 | Ngn-2 | (+) | 1.000 | 0.953 | atctccCAGATgctga |
| | | | | | SEQ ID No. 75 |
| V$IK_Q5_01 | Ikaros | (−) | 1.000 | 0.995 | tcTCCCA |
| | | | | | SEQ ID No. 76 |
| V$CPBP_Q6 | CPBP | (+) | 0.995 | 0.995 | CTCCCag |
| | | | | | SEQ ID No. 77 |
| V$CHCH_01 | Churchill | (−) | 0.984 | 0.984 | cTCCCA |
| | | | | | SEQ ID No. 78 |
| V$E2A_Q6_01 | E2A | (−) | 0.990 | 0.962 | ctcccAGATGctg |
| | | | | | SEQ ID No. 79 |
| V$HEN2_Q2 | HEN2 | (−) | 1.000 | 0.997 | tcccAGATG |
| | | | | | SEQ ID No. 80 |
| V$E2A_Q6 | E2A | (−) | 0.976 | 0.962 | cccAGATG |
| | | | | | SEQ ID No. 81 |
| V$GATA6_01 | GATA-6 | (+) | 0.955 | 0.953 | ccaGATGCtg |
| | | | | | SEQ ID No. 82 |
| V$GATA2_01 | GATA-2 | (+) | 0.982 | 0.978 | ccaGATGCtg |
| | | | | | SEQ ID No. 83 |
| V$GATA1_01 | GATA-1 | (+) | 0.993 | 0.990 | ccaGATGCtg |
| | | | | | SEQ ID No. 84 |
| V$TALLIKE_Q6 | Tal like | (−) | 0.995 | 0.955 | ccAGATGctgat |
| | | | | | SEQ ID No. 85 |
| V$HTF4_Q2 | HTF4 | (−) | 0.968 | 0.969 | ccAGATG |
| | | | | | SEQ ID No. 86 |
| V$NRL_01 | NRL | (+) | 1.000 | 0.970 | cagatGCTGAt |
| | | | | | SEQ ID No. 87 |
| V$NMYC_02 | NMYC | (−) | 1.000 | 1.000 | cAGATG |
| | | | | | SEQ ID No. 88 |
| V$E2A_Q6_02 | E2A | (+) | 0.990 | 0.988 | CAGATgct |
| | | | | | SEQ ID No. 89 |
| V$TAL1_Q6_01 | Tal-1 | (+) | 1.000 | 0.953 | CAGATgc |
| | | | | | SEQ ID No. 90 |
| V$NMYC_02 | NMYC | (+) | 0.954 | 0.965 | CAGATg |
| | | | | | SEQ ID No. 91 |
| V$MAFA_Q4 | MAFA | (−) | 1.000 | 0.998 | atGCTGA |
| | | | | | SEQ ID No. 92 |
| VYMAFK_Q3 | MafK | (−) | 1.000 | 0.955 | atGCTGAttca |
| | | | | | SEQ ID No. 93 |
| V$MAFB_Q4_01 | MAFB | (−) | 1.000 | 1.000 | tGCTGAt |
| | | | | | SEQ ID No. 94 |
| V$FRA2_Q4_01 | Fra-2 | (−) | 0.983 | 0.979 | tgcTGATTca |
| | | | | | SEQ ID No. 95 |

The inventors confirmed that Klf15 is not expressed in terminally differentiated rod photoreceptors using immunofluorescence analysis in mouse, porcine and human retina (FIG. 1B, FIG. 4). Antibody staining showed Klf15 expression in the ganglion cell layer (GCL) and inner nuclear layers (INL) but an apparent lack of expression in the outer nuclear layer (ONL) (FIG. 1B, FIG. 4). However, in the pig retina the inventors found expression of Klf15 also in cone photoreceptors (FIG. 4C). To further confirm that KLF15 is not expressed in rods, the inventors used a procedure to isolate a population of porcine rods for analysis. Specifically, porcine rods were labelled by subretinal injection of an AAV vector containing eGFP under the control of the rod-specific promoter element GNAT1 (AAV8-hGNAT1-eGFP(12)). Fifteen days post injection, eGFP-positive rods were dissociated and sorted by FACS and the inventors measured Klf15 mRNA levels by qReal Time PCR (qPCR), but no Klf15 expression could be observed (FIG. 1C). The inventors next evaluated the affinity of human KLF15 for the hRHO-cis. KLF15 showed high affinity for the hRHO-cis similar to that of the synthetic TF ZF6-DB (FIG. 1D). Furthermore, chromatin immunoprecipitation (ChIP) showed proper hRHO-cis genomic occupancy by KLF15 (FIG. 1E). These data suggest that KLF15 and the synthetic TF ZF6-DB show analogous binding properties despite protein structural differences (KLF15 has a KRAB effector domain at the N-terminus and 3 zinc-fingers at the C-terminus while ZF6-DB has 6 zinc-fingers without an effector domain).

The inventors used the wild-type porcine retina to investigate the ability of KLF15 to repress Rho expression. The hRHO-cis sequence is highly conserved between pigs and humans (FIG. 2A). Sub-retinal injection of a low dose of an AAV8 vector containing the human KLF15 (hKLF15) under the rod-specific GNAT1 promoter in adult pigs (2×1010 genome copies (gc) of AAV8-GNAT1-hKLF15 vector), showed that hKLF15, 15 days after delivery, resulted in 45% and the 38% repression of the Rho transcript and protein levels, respectively, in the transduced area (FIG. 2B,C). Consistently, morphological analysis showed the collapse of Rho-deprived outer segments (OS). Despite Rho depletion, the integrity of the outer nuclear layers (ONL) was maintained at this short time point (FIG. 2D,E), in agreement with what has been observed with the synthetic TF ZF6-DB (12, 13). To determine genome-wide transcriptional changes that might be caused by the ectopic expression of hKLF15 the inventors evaluated by RNA sequencing (RNA-Seq) retina 15 days after subretinal injection of an AAV8-CMV-hKLF15. The inventors found 156 differentially expressed genes (DEGs), of which 3 were rod-photoreceptor specific (Rho, Gnat1 and Crx, Table 3).

TABLE 3
List of differentially expressed genes (DEGs) in porcine retina
upon hKLF15 ectopic expression.

| | | Log₂ | |
|---|---|---|---|
| Ensembl | Gene | (Fold | |
| gene ID | Name | Change) | FDR |
| ENSSSCG00000011796 | CRYGS | 1.96 | 3.06E−08 |
| ENSSSCG00000015595 | ATF3 | 1.63 | 3.06E−08 |
| ENSSSCG00000022111 | | 1.47 | 3.04E−06 |
| ENSSSCG00000028038 | | 1.75 | 3.04E−06 |
| ENSSSCG00000000492 | LYZ | 1.67 | 6.80E−06 |
| ENSSSCG00000006472 | CRABP2 | 1.59 | 6.80E−06 |
| ENSSSCG00000006979 | MSR1 | 1.6 | 1.86E−05 |
| ENSSSCG00000013612 | ACP5 | 1.6 | 3.59E−05 |
| ENSSSCG00000027130 | TNFRSF12A | 1.45 | 3.59E−05 |
| ENSSSCG00000000660 | A2M | 1.36 | 6.48E−05 |
| ENSSSCG00000005638 | LCN2 | 1.51 | 9.51E−05 |
| ENSSSCG00000007208 | TRIB3 | 1.45 | 0.000172977 |
| ENSSSCG00000005267 | ANXA1 | 1.21 | 0.000206727 |
| ENSSSCG00000004195 | ARG1 | 1.07 | 0.000526059 |
| ENSSSCG00000012880 | CPT1A | −0.9 | 0.000674971 |
| ENSSSCG00000030344 | CLDN19 | −1.38 | 0.000692567 |
| ENSSSCG00000011590 | RHO | 0.84 | 0.001711068 |
| ENSSSCG00000025390 | | 1.1 | 0.001718333 |
| ENSSSCG00000010210 | SLC16A9 | 1.27 | 0.0017615 |
| ENSSSCG00000010613 | ITPRIP | 1.14 | 0.0017615 |
| ENSSSCG00000003524 | C1QA | 1.11 | 0.002267724 |
| ENSSSCG00000025698 | SERPINE1 | 1.32 | 0.002267724 |
| ENSSSCG00000010647 | ADRB1 | 1.2 | 0.00284353 |
| ENSSSCG00000024059 | VAT1 | 0.59 | 0.003155809 |
| ENSSSCG00000009644 | ADAM28 | 1.17 | 0.00324015 |
| ENSSSCG00000013586 | LRRC8E | −0.96 | 0.005732384 |
| ENSSSCG00000017956 | CD68 | 1.17 | 0.005732384 |
| ENSSSCG00000009216 | SPP1 | 1.06 | 0.00620305 |
| ENSSSCG00000024246 | | −0.86 | 0.006214199 |
| ENSSSCG00000026526 | CATSPER4 | −1.2 | 0.006817098 |
| ENSSSCG00000022309 | GPR34 | 1.06 | 0.008883936 |
| ENSSSCG00000029371 | C5AR1 | 0.96 | 0.009231119 |
| ENSSSCG00000023033 | PARP3 | 0.66 | 0.012830289 |
| ENSSSCG00000000734 | | 1.16 | 0.012985203 |
| ENSSSCG00000010224 | EGR2 | 1.16 | 0.012985203 |
| ENSSSCG00000017920 | CXCL16 | 1.11 | 0.013156332 |
| ENSSSCG00000003369 | ICMT | 0.66 | 0.015022376 |
| ENSSSCG00000008786 | | 1.14 | 0.015022376 |
| ENSSSCG00000010705 | GMFG | 1.15 | 0.015022376 |
| ENSSSCG00000011322 | CCR1 | 1.11 | 0.015022376 |
| ENSSSCG00000027550 | PLCD1 | 0.72 | 0.015881265 |
| ENSSSCG00000016286 | PRSS56 | 1.12 | 0.016454742 |
| ENSSSCG00000003135 | KCNJ14 | −0.7 | 0.017845183 |
| ENSSSCG00000017343 | GFAP | 1 | 0.017845183 |
| ENSSSCG00000000368 | MMP19 | 1.02 | 0.018011387 |
| ENSSSCG00000012881 | CPT1A | −0.89 | 0.018011387 |
| ENSSSCG00000024609 | GNAT1 | −0.6 | 0.018011387 |
| ENSSSCG00000012364 | VSIG4 | 1.11 | 0.018520249 |
| ENSSSCG00000003170 | SLC17A7 | −0.61 | 0.018637969 |
| ENSSSCG00000010277 | SLC29A3 | 0.76 | 0.020219438 |
| ENSSSCG00000024495 | SELPLG | 0.96 | 0.020219438 |
| ENSSSCG00000006620 | TUFT1 | 0.59 | 0.022946535 |
| ENSSSCG00000009437 | KBTBD7 | −0.44 | 0.024024029 |
| ENSSSCG00000006243 | PENK | −1 | 0.027072384 |
| ENSSSCG00000006634 | TNFAIP8L2 | 1.08 | 0.029542591 |
| ENSSSCG00000010732 | FAM53B | −0.65 | 0.030276515 |
| ENSSSCG00000007115 | THBD | 0.95 | 0.031539432 |
| ENSSSCG00000010684 | RGS10 | 0.96 | 0.031539432 |
| ENSSSCG00000000136 | CSF2RB | 1.08 | 0.032019368 |
| ENSSSCG00000001025 | DSP | −1.05 | 0.032019368 |
| ENSSSCG00000002720 | CLEC18A | 0.78 | 0.032019368 |
| ENSSSCG00000008239 | CAPG | 0.9 | 0.032019368 |
| ENSSSCG00000025899 | | −0.83 | 0.032019368 |
| ENSSSCG00000011397 | SLC38A3 | −0.63 | 0.032019964 |
| ENSSSCG00000030638 | CH242-16815.2 | 1.03 | 0.032019964 |
| ENSSSCG00000006610 | S100A11 | 1.04 | 0.033960107 |
| ENSSSCG00000000648 | CLEC7A | 1.06 | 0.037120112 |
| ENSSSCG00000003124 | CRX | −0.48 | 0.037120112 |
| ENSSSCG00000005465 | SUSD1 | 0.9 | 0.037120112 |
| ENSSSCG00000013909 | CRLF1 | −1.01 | 0.040720279 |
| ENSSSCG00000003601 | HCRTR1 | −0.84 | 0.040858391 |
| ENSSSCG00000011713 | P2Y12R | 0.9 | 0.040858391 |
| ENSSSCG00000011808 | SST | −1.05 | 0.040858391 |
| ENSSSCG00000012018 | CHODL | 0.88 | 0.040858391 |
| ENSSSCG00000005229 | VLDLR | −0.49 | 0.042531039 |
| ENSSSCG00000000195 | PRPH | 0.99 | 0.043790204 |
| ENSSSCG00000004024 | | 0.91 | 0.043790204 |
| ENSSSCG00000004578 | ANXA2 | 0.79 | 0.043790204 |
| ENSSSCG00000005339 | | 0.89 | 0.043790204 |
| ENSSSCG00000004789 | THBS1 | 0.98 | 0.04498596 |
| ENSSSCG00000023374 | SRGN | 0.99 | 0.04498596 |
| ENSSSCG00000014920 | FZD4 | −0.62 | 0.045862814 |
| ENSSSCG00000002297 | RDH12 | −0.56 | 0.047239854 |
| ENSSSCG00000015405 | CD36 | 1.03 | 0.047239854 |
| ENSSSCG00000006183 | SBSPON | 0.72 | 0.04831452 |
| ENSSSCG00000028363 | TMPRSS9 | 1.02 | 0.05122971 |
| ENSSSCG00000014921 | PRSS23 | 0.79 | 0.051623438 |
| ENSSSCG00000002883 | DMKN | 0.89 | 0.052970778 |
| ENSSSCG00000015336 | SLC25A13 | −0.37 | 0.052970778 |
| ENSSSCG00000030485 | ELFN1 | −0.7 | 0.053472411 |
| ENSSSCG00000003465 | FBLIM1 | 0.47 | 0.055856973 |
| ENSSSCG00000003526 | C1QB | 0.88 | 0.055856973 |
| ENSSSCG00000009002 | TLR2 | 0.96 | 0.055856973 |
| ENSSSCG00000011470 | ABHD6 | −0.43 | 0.055856973 |
| ENSSSCG00000026302 | MKI67 | 1.01 | 0.055856973 |
| ENSSSCG00000026592 | TLR6 | 0.94 | 0.055856973 |
| ENSSSCG00000028579 | | 0.91 | 0.055856973 |
| ENSSSCG00000012941 | SLC29A2 | −0.86 | 0.058398102 |
| ENSSSCG00000026710 | CARHSP1 | 0.62 | 0.058398102 |
| ENSSSCG00000015353 | SCIN | 0.97 | 0.058409179 |
| ENSSSCG00000002533 | WDR20 | −0.42 | 0.059119358 |
| ENSSSCG00000015320 | CALCR | 0.89 | 0.060084418 |
| ENSSSCG00000021084 | S100A6 | 1 | 0.060084418 |
| ENSSSCG00000024816 | CEBPB | 0.96 | 0.060084418 |
| ENSSSCG00000005591 | GPR144 | −0.99 | 0.060095154 |
| ENSSSCG00000001930 | PKM | −0.45 | 0.062094944 |
| ENSSSCG00000006862 | VCAM1 | 0.83 | 0.062094944 |
| ENSSSCG00000010581 | PSD | −0.53 | 0.062094944 |
| ENSSSCG00000023557 | CCRL2 | 0.88 | 0.062094944 |
| ENSSSCG00000008937 | AMBN | 0.98 | 0.065516749 |
| ENSSSCG00000003275 | | 0.94 | 0.066152027 |
| ENSSSCG00000007625 | ARPC1B | 0.87 | 0.066152027 |
| ENSSSCG00000004291 | NT5E | −0.62 | 0.066476444 |
| ENSSSCG00000006379 | CD48 | 0.98 | 0.068197428 |
| ENSSSCG00000026184 | MPP4 | 0.48 | 0.068290441 |
| ENSSSCG00000017439 | KRT32 | 0.94 | 0.070614513 |
| ENSSSCG00000021557 | | 0.88 | 0.070614513 |
| ENSSSCG00000027046 | USP9Y | 0.89 | 0.070614513 |
| ENSSSCG00000022236 | FOLR1 | 0.85 | 0.071210433 |
| ENSSSCG00000005503 | TLR4 | 0.95 | 0.077548044 |
| ENSSSCG00000008123 | ARID5A | 0.84 | 0.077548044 |
| ENSSSCG00000024725 | C2orf71 | −0.65 | 0.077548044 |
| ENSSSCG00000025741 | SNX20 | 0.97 | 0.077548044 |
| ENSSSCG00000000223 | | 0.88 | 0.08061489 |
| ENSSSCG00000000958 | DYRK2 | −0.62 | 0.08061489 |
| ENSSSCG00000001469 | SLA-DMB | 0.77 | 0.08061489 |
| ENSSSCG00000015258 | GLB1L2 | 0.75 | 0.08061489 |
| ENSSSCG00000026084 | MFSD7 | 0.71 | 0.08061489 |
| ENSSSCG00000028103 | | −0.66 | 0.08061489 |
| ENSSSCG00000028711 | CASP1 | 0.84 | 0.08061489 |
| ENSSSCG00000010603 | NEURL1 | −0.55 | 0.081874872 |
| ENSSSCG00000030921 | APOA1 | 0.79 | 0.081874872 |
| ENSSSCG00000004554 | | 0.89 | 0.083812341 |
| ENSSSCG00000005587 | NEK6 | 0.85 | 0.084781746 |
| ENSSSCG00000003651 | RHBDL2 | 0.8 | 0.08506656 |
| ENSSSCG00000029852 | WNT5A | −0.81 | 0.08506656 |
| ENSSSCG00000017473 | TOP2A | 0.91 | 0.088275952 |
| ENSSSCG00000016851 | OSMR | 0.71 | 0.089873226 |
| ENSSSCG00000009445 | PCDH8 | 0.64 | 0.092169462 |
| ENSSSCG00000008624 | | −0.55 | 0.092945279 |
| ENSSSCG00000008664 | FAM84A | 0.82 | 0.092945279 |
| ENSSSCG00000017087 | GM2A | 0.74 | 0.092945279 |
| ENSSSCG00000003525 | C1QC | 0.86 | 0.093207956 |
| ENSSSCG00000015706 | LYPD1 | 0.44 | 0.093207956 |
| ENSSSCG00000022056 | GPR37L1 | 0.62 | 0.093207956 |
| ENSSSCG00000026583 | TLR1 | 0.85 | 0.093207956 |
| ENSSSCG00000027196 | GIMAP6 | 0.86 | 0.093207956 |
| ENSSSCG00000010347 | LRIT2 | −0.7 | 0.093618926 |
| ENSSSCG00000007240 | | 0.94 | 0.094933926 |
| ENSSSCG00000015395 | | −0.52 | 0.096757612 |
| ENSSSCG00000007831 | CACNG3 | −0.51 | 0.097636479 |
| ENSSSCG00000011398 | SEMA3F | −0.61 | 0.097636479 |
| ENSSSCG00000015113 | ABCG4 | −0.52 | 0.097636479 |
| ENSSSCG00000010683 | GRK5 | −0.5 | 0.098219952 |
| ENSSSCG00000001887 | SCAMP5 | −0.43 | 0.099643731 |
| ENSSSCG00000015907 | GALNT3 | 0.73 | 0.099928158 |

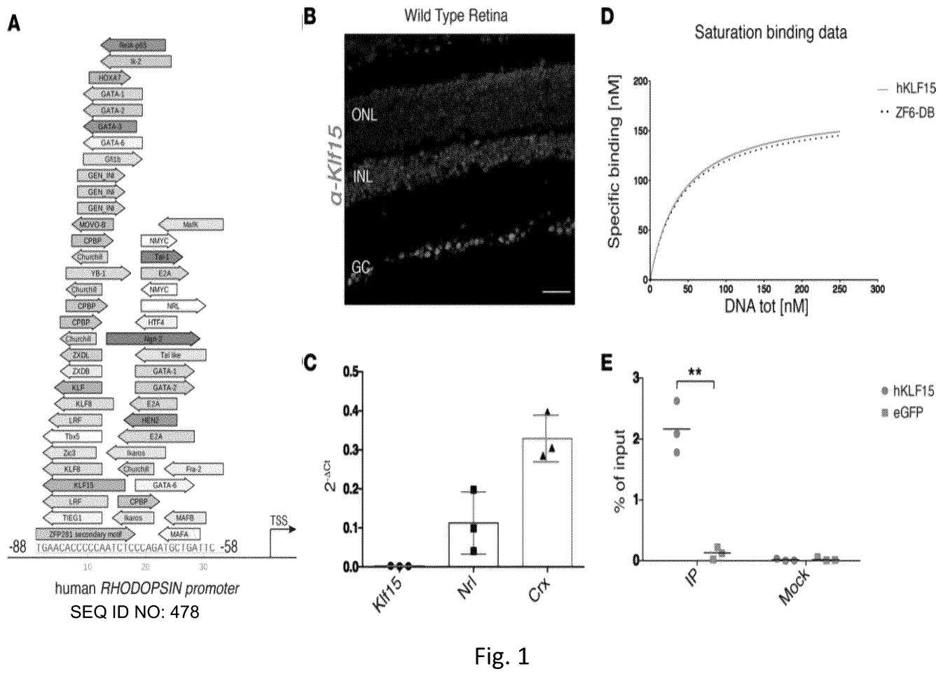

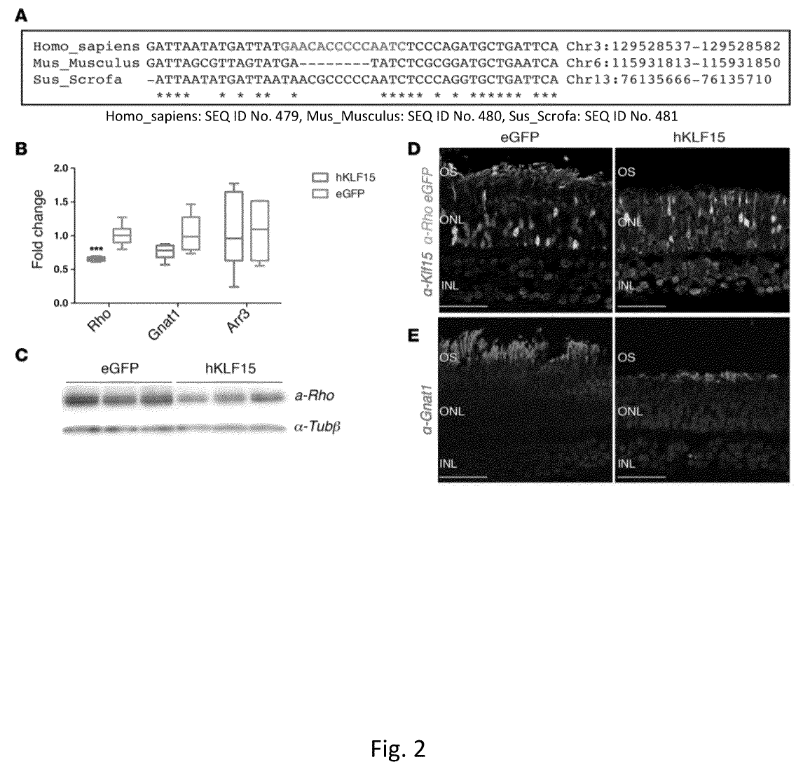

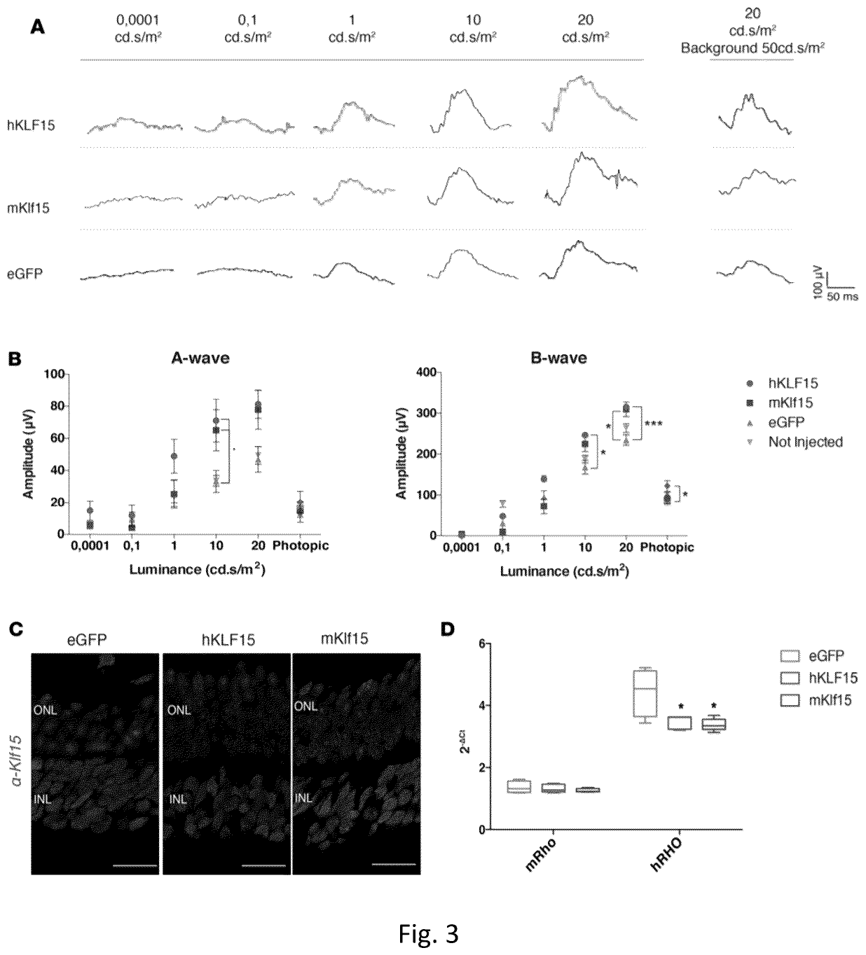

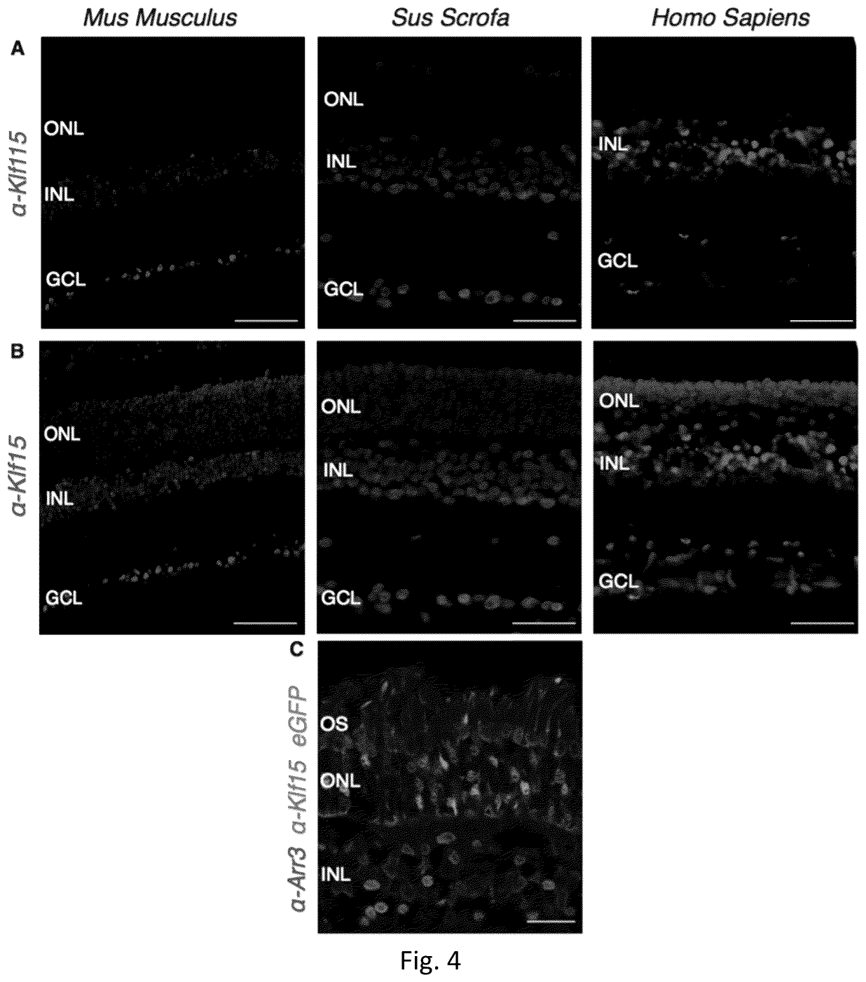

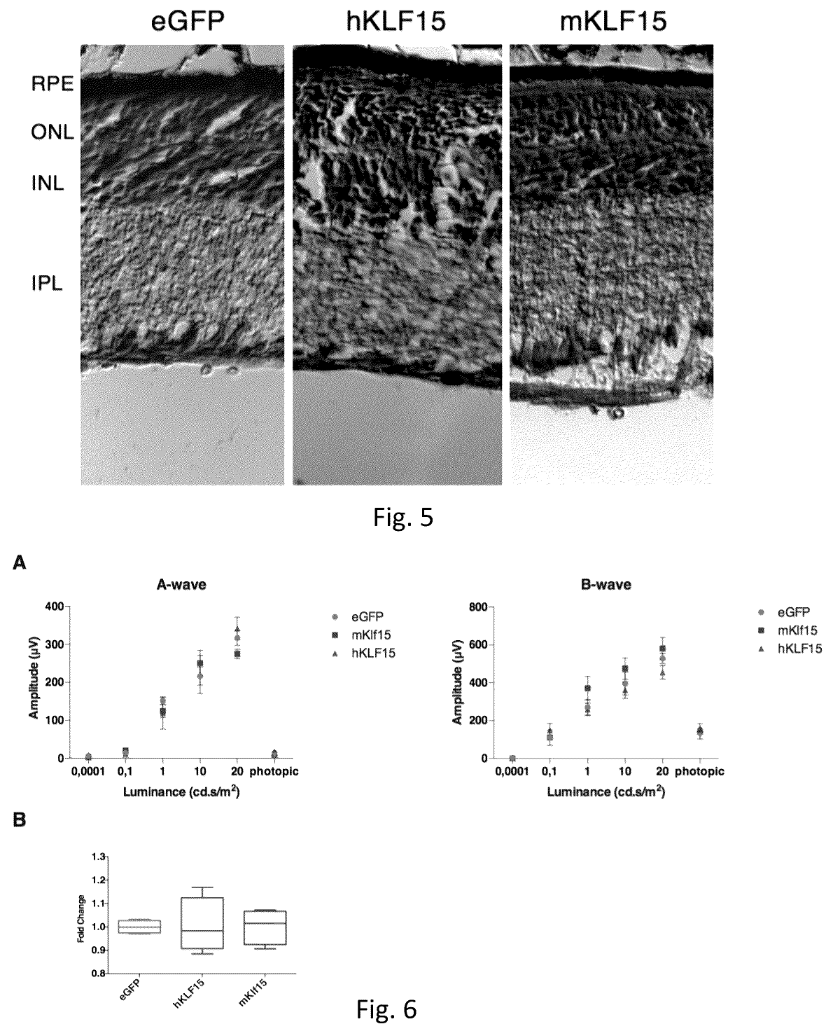

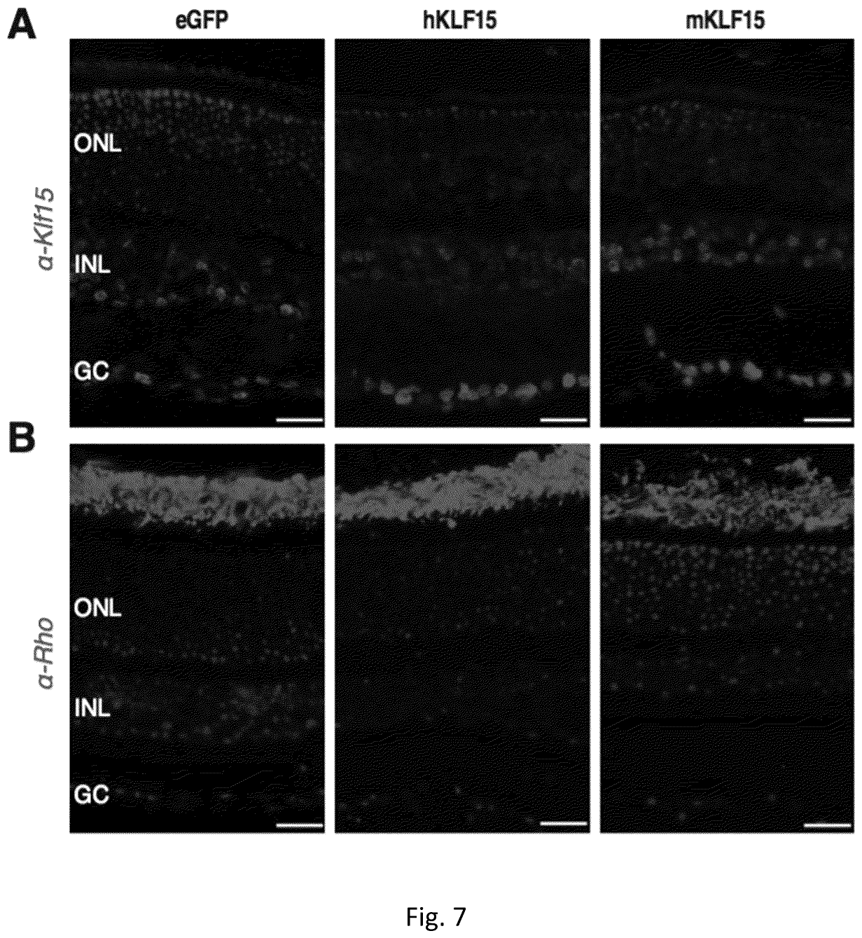

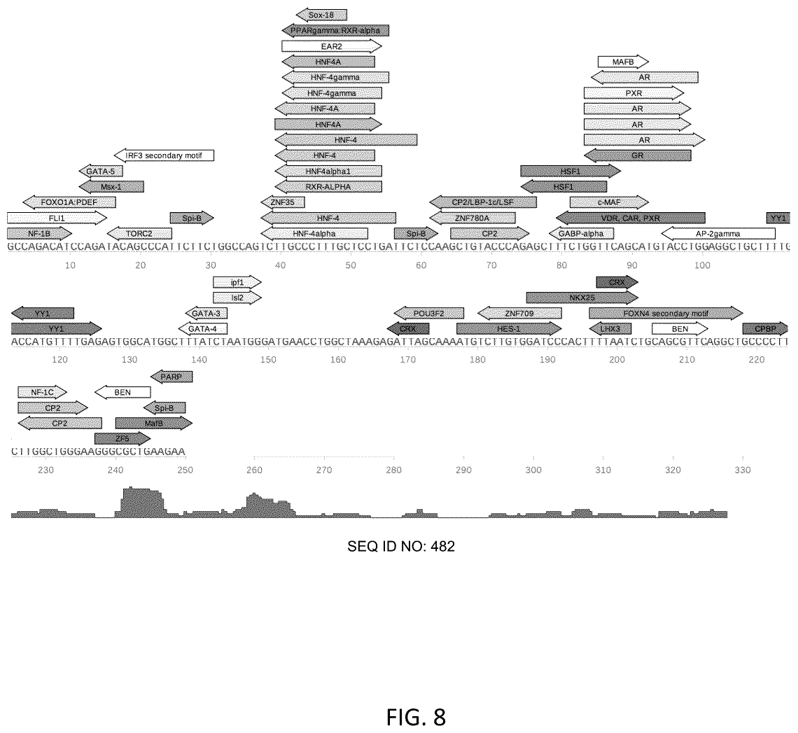

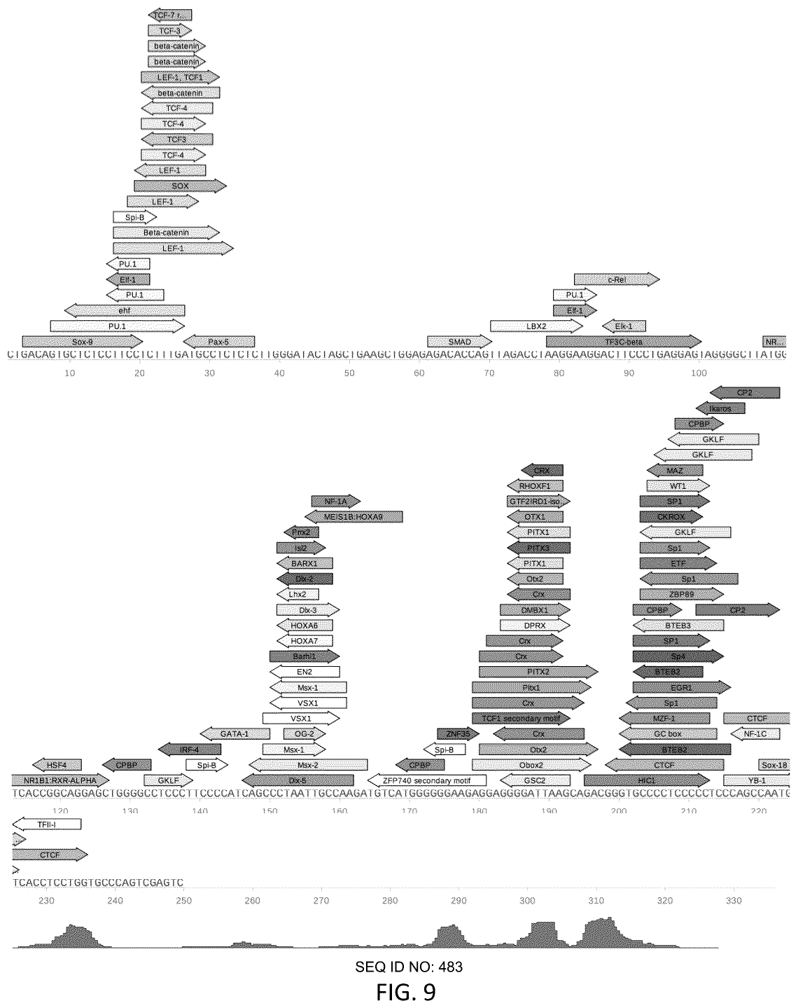

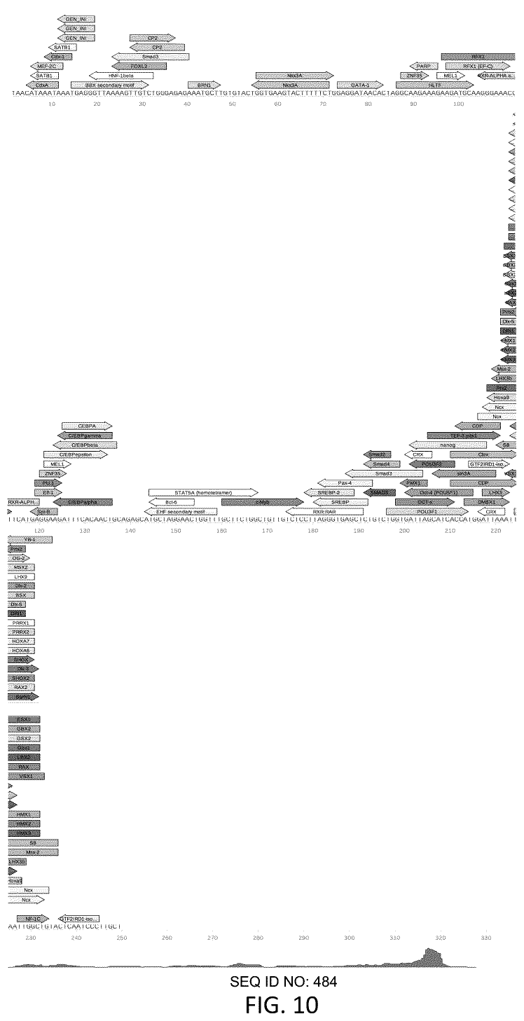

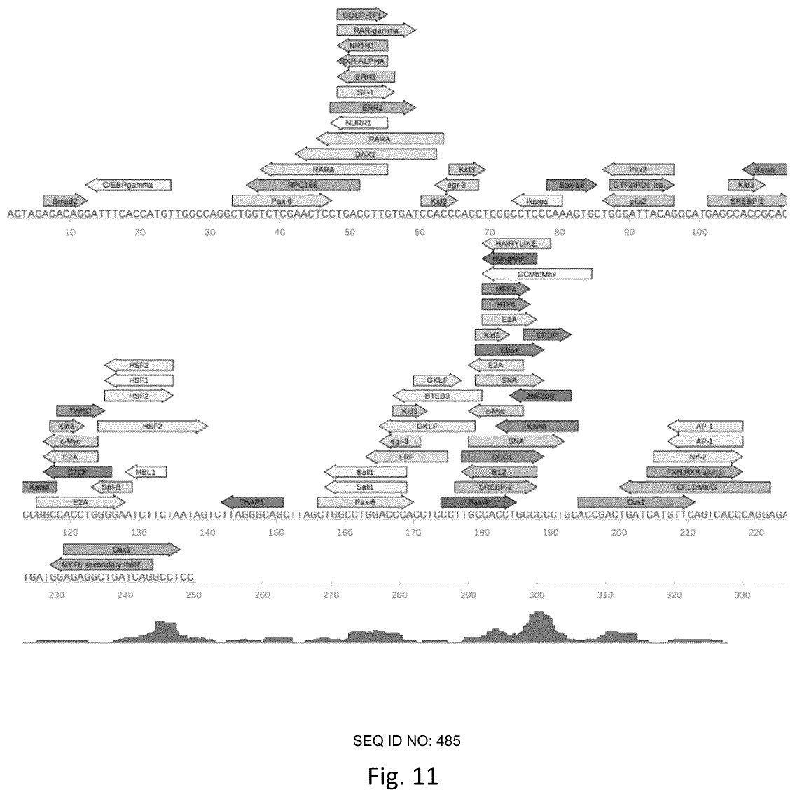

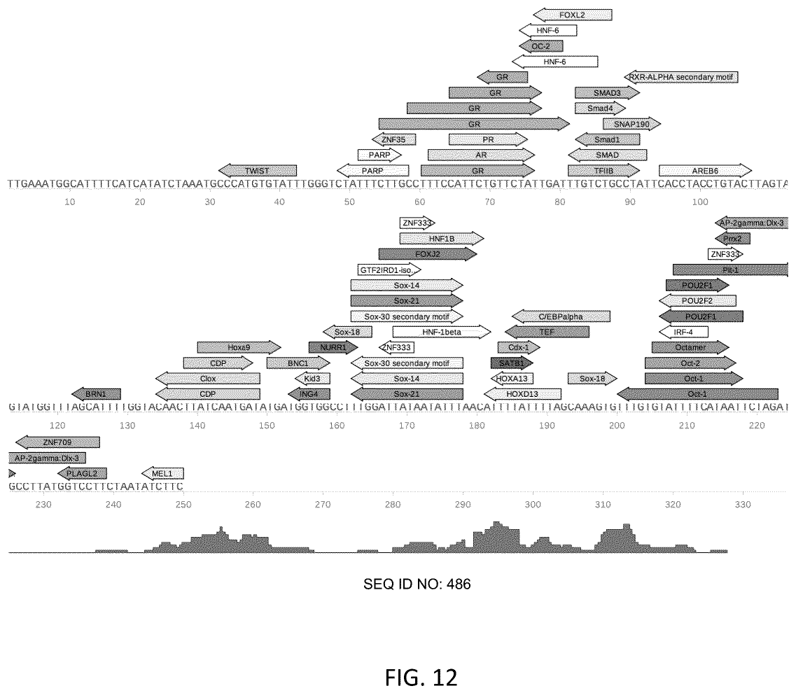

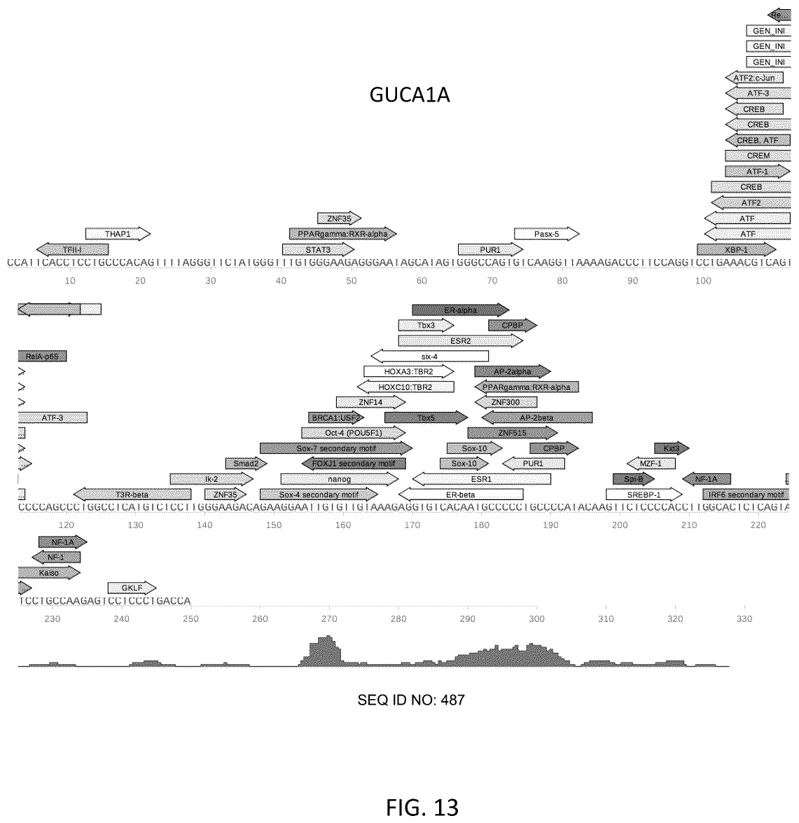

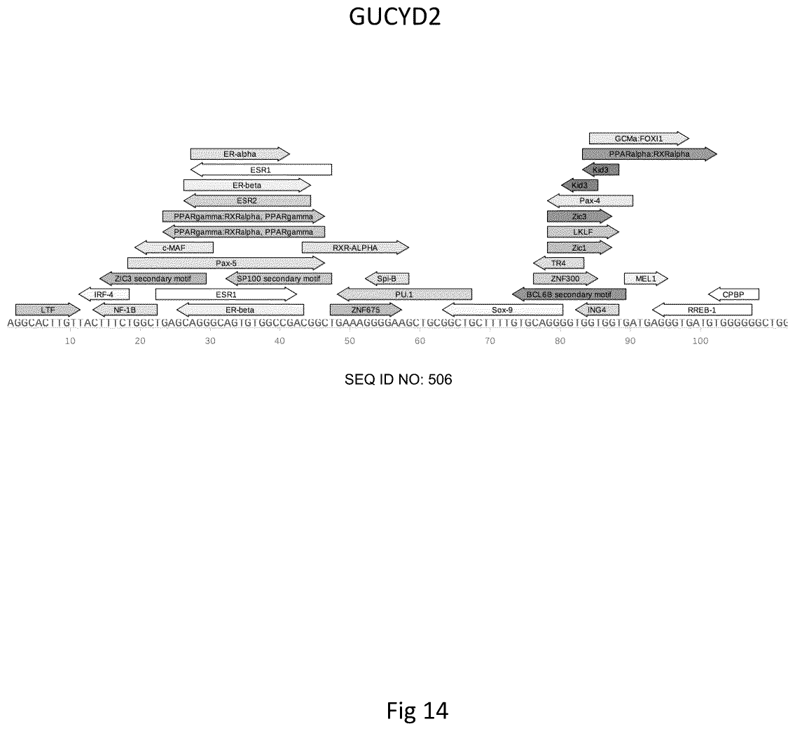

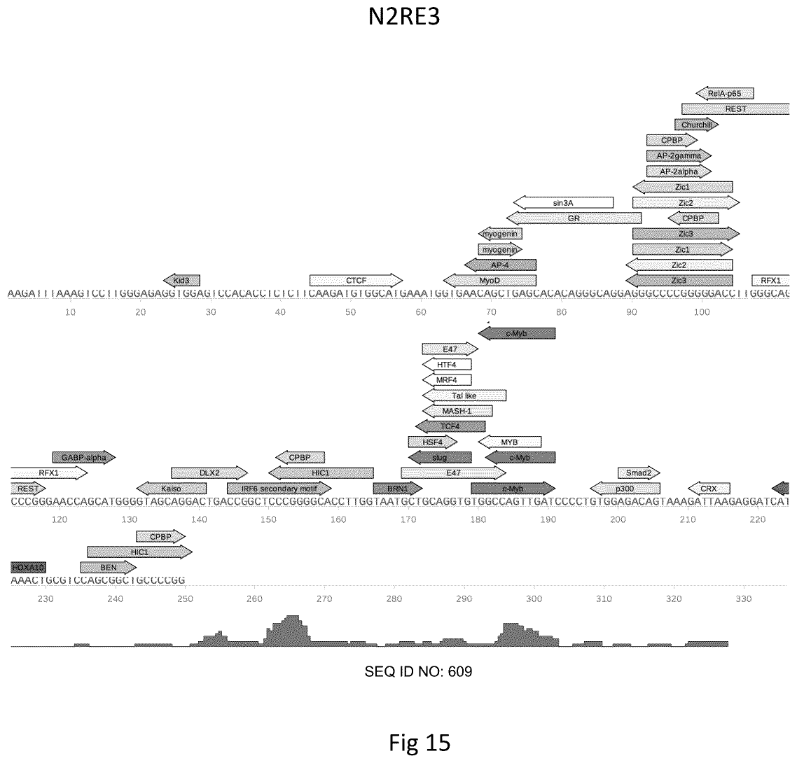

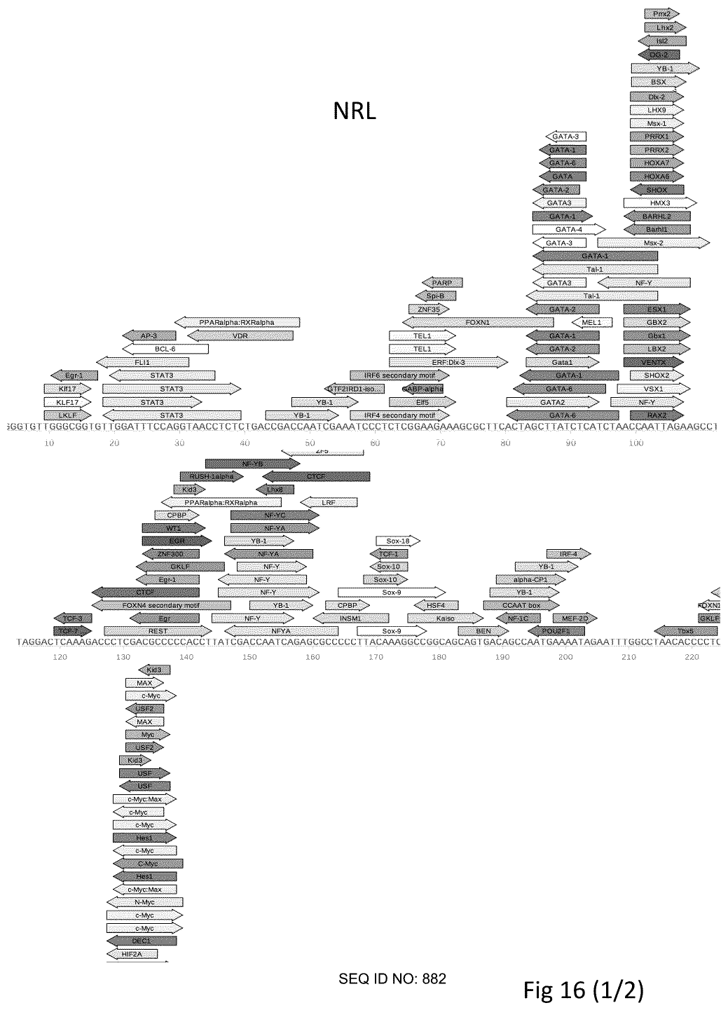

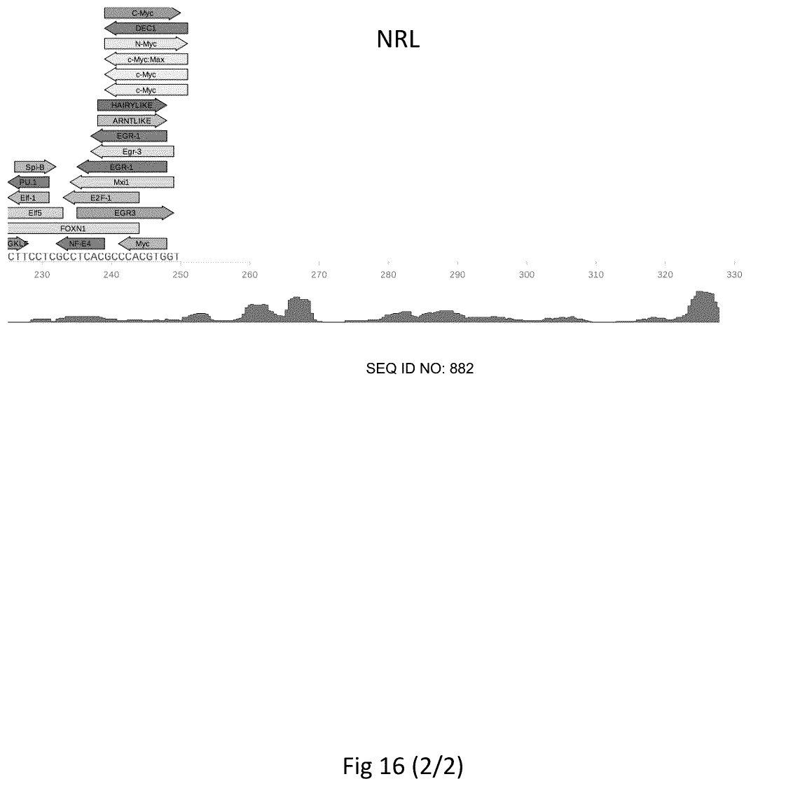

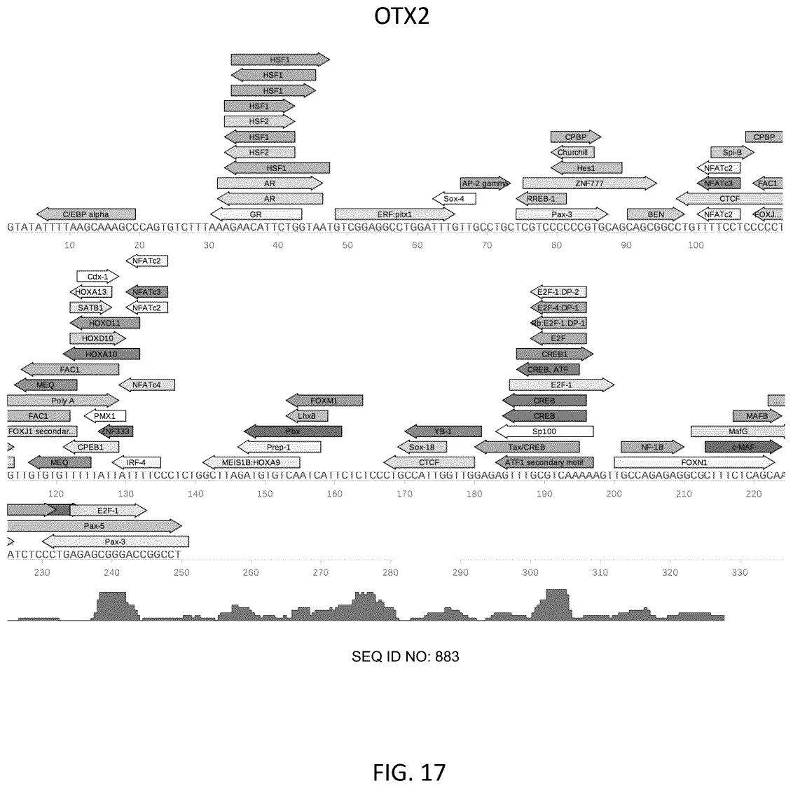

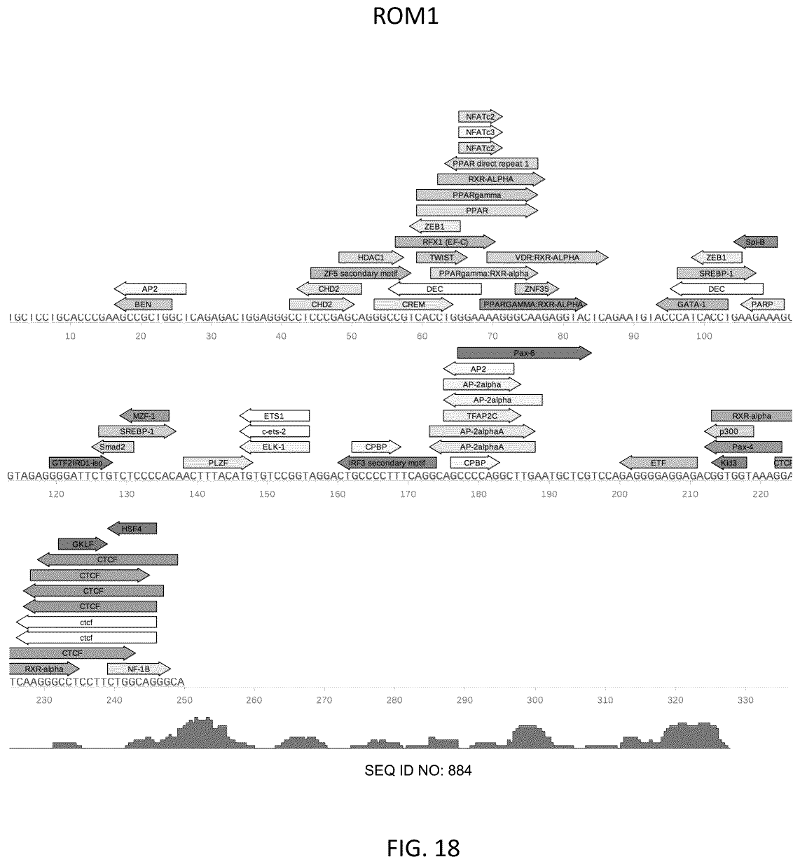

To test whether RHO repression mediated by the ectopic expression of hKLF15 could produce a therapeutic effect, the inventors delivered AAV8-GNAT1-hKLF15 into the transgenic RHO-P347S mouse model of adRP (23). This adRP mouse model harbors the P347S human RHO mutant allele, including the hRHO-cis motif, and the endogenous murine Rho alleles (23). Interestingly, despite extensive promoter conservation with humans, the murine Rho promoter diverges in the hRHO-cis sequence motif (FIG. 2A). The inventors took advantage experimentally of this sequence motif difference to determine the specificity of hKLF15 for the human hRHO-cis RHO regulatory sequence. The inventors expected that the selective binding and repression of the human RHO transgenic promoter by KLF15 would result in preservation of retinal function due to the silencing of the P347S RHO-mutation. Subretinal delivery of AAV8-GNAT1-hKLF15 in P14 P347S mice resulted in significant repression of the human RHO mutant transgene transcript but left unchanged expression from the endogenous murine Rho alleles (FIG. 3D). The selective silencing of the P347S RHO mutation resulted in the preservation of retinal structure and function, evaluated by electroretinography (ERG) and histological analysis 30 days after delivery (FIG. 3A-C, FIG. 5). Similar human-specific P347S mutant RHO repression was observed in P14 P347S mice injected with an AAV containing the murine Klf15 orthologous gene, which shows complete conservation of the C-terminus zinc-finger DNA-binding domain and partial conservation of the N-terminus (FIG. 3). Notably, these findings support the notion that the recognition of hRHO-cis by KLF15 is independent of the specific Rho chromosomal location (the P347S adRP mouse model harbors the mutant RHO in non-specific loci), that local sequence features may contribute to the observed effect (24), and that the human and murine KLF15 genes based on their conservation operate similarly on the hRHO-cis sequence. To evaluate tolerability and potential toxicity of ectopic expression of Klf15 in rods, the inventors subretinally injected adult wild-type mice with the human or the murine Klf15 gene (AAV8-GNAT1-hKLF15; AAV8-GNAT1-mKlf15, respectively). Eighty days after delivery, the retina of treated animals showed no changes in Rho transcript levels (qPCR) and no detrimental effects on retinal ERG electrophysiological responses or histological appearance (FIGS. 6,7).

In this study the inventors have shown that the cell-specific factors, in which a TF ectopically expressed operates, restrict its activity. In particular, ectopic expression of KLF15, which is involved in a wide variety of organ functions, in terminally differentiated rod photoreceptors silenced RHO expression with limited off-targeting effects. The results show that the cell-specific context may limit TF activities that control wide and coherent genetic programs, which, for instance, determine developmental and somatic photoreceptor identity transitions in the mammalian retina (1, 25, 26). KLF15 belongs to the largest TF group (KRAB-ZNF TFs) in the mammalian genome with an estimated repertoire of around 400 KRAB-ZNF TFs. In addition, KRAB-ZNF TFs shows highly differential tissue patterns of expression (27, 28). Thus, in principle, this TF somatic ectopic gene transfer approach could be extended to other gene targets by combining TF preferences with cell-specific expression and genome accessibility maps (10, 14). Of note, gene expression profiles in diverse tissues of the human body and across individuals are being increasingly identified (29).

Ectopic expression of KLF15 resulted in efficient Rho silencing similar to that shown by synthetic TFs (12, 13). Silencing of the severe RHO-P347S gain-of-function mutation in the adRP mouse model translated into structural and functional protection of the retina from degeneration. Coupling Rho transcriptional silencing with replacement, as others and the inventors described (30) and the safety and efficacy of AAV retinal gene transfer (31), supports further development of this strategy for the treatment of adRP. In summary, the inventors provided a proof-of-concept of a novel mode to efficiently and specifically silence a gene by ectopic expression of a TF in a novel cell-specific context.

### Example 2

The inventors obtained similar results as per FIGS. 8-14 where transfac analysis is applied to the identification of transcription factors binding the regulatory sequence of the following promoters, defined as a genomic DNA sequence spanning 250 bps from the transcription start site:

TABLE 4
Transcription factor Position Weight Matrix (PWM, Transfac) recognizing the
Human CRX promoter (−250 bp from TSS)

| | | Position | Core | Matrix | |
|---|---|---|---|---|---|
| Matrix | Factor name | (strand) | score | score | Sequence |
| V$SOX9_09 | Sox-9 | 3 (+) | 0.975 | 0.949 | gACAGTgctctccttcc |
| | | | | | SEQ ID No. 96 |
| V$PU1_Q4 | PU.1 | 7 (+) | 1.000 | 0.954 | gtgctctccTTCCTcttt |
| | | | | | g |
| | | | | | SEQ ID No. 97 |
| V$EHF_10 | ehf | 9 (−) | 1.000 | 0.987 | gctctccTTCCTctttg |
| | | | | | SEQ ID No. 98 |
| V$PU1_Q6 | PU.1 | 15 (−) | 1.000 | 1.000 | CTTCCtct |
| | | | | | SEQ ID No. 99 |
| V$ELF1_Q5 | Elf-1 | 15 (−) | 1.000 | 1.000 | cTTCCT |
| | | | | | SEQ ID No. 100 |
| V$SPI1_Q5 | PU.1 | 15 (−) | 1.000 | 1.000 | cTTCCT |
| | | | | | SEQ ID No. 100 |
| V$LEF1_04 | LEF-1 | 16 (+) | 1.000 | 0.934 | ttcctcTTTGAtgcctc |
| | | | | | SEQ ID No. 101 |
| V$BETACATENIN_ | Beta-catenin | 16 (+) | 1.000 | 0.986 | ttcctCTTTGatgcc |
| Q6 | | | | | SEQ ID No. 102 |
| V$SPIB_Q3 | Spi-B | 16 (+) | 1.000 | 1.000 | TTCCTc |
| | | | | | SEQ ID No. 103 |
| V$LEF1_07 | LEF-1 | 18 (+) | 1.000 | 0.996 | cctcTTTGAt |
| | | | | | SEQ ID No. 104 |
| V$SOX_Q6 | SOX | 19 (+) | 1.000 | 0.869 | ctCTTTGatgcct |
| | | | | | SEQ ID No. 105 |
| V$LEF1_Q2_01 | LEF-1 | 19 (−) | 1.000 | 0.997 | ctCTTTGatg |
| | | | | | SEQ ID No. 106 |
| V$TCF4_01 | TCF-4 | 20 (+) | 1.000 | 1.000 | tcTTTGAtg |
| | | | | | SEQ ID No. 107 |
| V$TCF3_Q6_01 | TCF3 | 20 (−) | 1.000 | 0.981 | tCTTTGatgc |
| | | | | | SEQ ID No. 108 |
| V$TCF7L2_06 | TCF-4 | 20 (+) | 1.000 | 0.998 | tCTTTGatg |
| | | | | | SEQ ID No. 107 |
| V$TCF4_Q5_02 | TCF-4 | 20 (−) | 1.000 | 1.000 | tCTTTGatgc |
| | | | | | SEQ ID No. 108 |
| V$BETACATENIN_ | beta-catenin | 20 (−) | 1.000 | 0.994 | tCTTTGatgcc |
| Q3 | | | | | SEQ ID No. 109 |
| V$LEF1TCF1_Q4 | LEF-1, TCF1 | 20 (+) | 1.000 | 0.981 | tCTTTGatgcc |
| | | | | | SEQ ID No. 109 |
| V$BETACATENIN_ | beta-catenin | 21 (+) | 1.000 | 1.000 | CTTTGatg |
| Q6_01 | | | | | SEQ ID No. 110 |
| V$BETACATENIN_ | beta-catenin | 21 (+) | 1.000 | 1.000 | CTTTGatg |
| Q3_01 | | | | | SEQ ID No. 110 |
| V$TCF3_Q6 | TCF-3 | 21 (+) | 1.000 | 1.000 | CTTTGa |
| | | | | | SEQ ID No. 111 |
| V$LEF1_Q2 | TCF-7 related | 21 (−) | 1.000 | 1.000 | CTTTGa |
| | | | | | SEQ ID No. 111 |
| V$PAX5_Q6 | Pax-5 | 26 (−) | 0.998 | 0.994 | atGCCTCtct |
| | | | | | SEQ ID No. 112 |
| V$SMAD_Q6 | SMAD | 61 (+) | 1.000 | 0.997 | AGACAccag |
| | | | | | SEQ ID No. 113 |
| V$LBX2_03 | LBX2 | 70 (+) | 0.656 | 0.931 | ttagacctAAGGA |
| | | | | | SEQ ID No. 114 |
| V$GTF3C2_01 | TF3C-beta | 78 (+) | 0.787 | 0.781 | aaggaaggacttcccT |
| | | | | | GAGGag |
| | | | | | SEQ ID No. 115 |
| V$ELF1_Q5 | Elf-1 | 79 (+) | 1.000 | 1.000 | AGGAAg |
| | | | | | SEQ ID No. 116 |
| V$SPI1_Q5 | PU.1 | 79 (+) | 1.000 | 1.000 | AGGAAg |
| | | | | | SEQ ID No. 116 |
| V$CREL_Q6 | c-Rel | 82 (+) | 0.927 | 0.916 | aaggacTTCCCt |
| | | | | | SEQ ID No. 117 |
| VELK1_Q6 | Elk-1 | 86 (−) | 1.000 | 1.000 | aCTTCC |
| | | | | | SEQ ID No. 118 |
| V$NR1B1RXRA_ | NR1B1: RXR- | 109 (+) | 1.000 | 0.847 | atGGTCAccggcagg |
| 01 | ALPHA | | | | agc |
| | | | | | SEQ ID No. 119 |
| V$HSF4_Q3 | HSF4 | 116 (−) | 1.000 | 1.000 | ccGGCAG |
| | | | | | SEQ ID No. 120 |
| V$CPBP_Q6 | CPBP | 126 (−) | 1.000 | 1.000 | ctGGGGC |
| | | | | | SEQ ID No. 121 |
| V$GKLF_Q4 | GKLF | 132 (+) | 1.000 | 1.000 | CCTCCct |
| | | | | | SEQ ID No. 122 |
| V$IRF4_07 | IRF-4 | 134 (−) | 1.000 | 0.970 | tccCTTCCc |
| | | | | | SEQ ID No. 123 |
| V$SPIB_Q3 | Spi-B | 138 (+) | 1.000 | 1.000 | TTCCCc |
| | | | | | SEQ ID No. 124 |
| V$GATA1_01 | GATA-1 | 140 (−) | 1.000 | 0.996 | ccCCATCagc |
| | | | | | SEQ ID No. 125 |
| V$DLX5_01 | DIx-5 | 146 (−) | 0.971 | 0.959 | cagccctAATTGccaa |
| | | | | | SEQ ID No. 126 |
| V$MSX2_01 | Msx-2 | 147 (−) | 1.000 | 0.883 | agcccTAATTgccaag |
| | | | | | a |
| | | | | | SEQ ID No. 127 |
| V$MSX1_01 | Msx-1 | 149 (+) | 1.000 | 0.969 | cccTAATTg |
| | | | | | SEQ ID No. 128 |
| V$V$X1_03 | V$X1 | 149 (+) | 1.000 | 0.926 | cccTAATTgcc |
| | | | | | SEQ ID No. 129 |
| V$V$X1_03 | V$X1 | 150 (−) | 0.992 | 0.946 | cctAATTGcca |
| | | | | | SEQ ID No. 130 |
| V$MSX1_Q5_01 | Msx-1 | 150 (−) | 1.000 | 0.977 | ccTAATTgcca |
| | | | | | SEQ ID No. 130 |
| VEN2_03 | EN2 | 150 (−) | 1.000 | 0.994 | ccTAATTgcc |
| | | | | | SEQ ID No. 131 |
| V$BARHL1_04 | Barhl1 | 150 (+) | 1.000 | 0.998 | ccTAATTgcc |
| | | | | | SEQ ID No. 131 |
| V$HOXA7_08 | HOXA7 | 151 (−) | 1.000 | 0.997 | cTAATTgc |
| | | | | | SEQ ID No. 132 |
| V$HOXA6_03 | HOXA6 | 151 (−) | 1.000 | 0.996 | cTAATTgc |
| | | | | | SEQ ID No. 132 |
| V$DLX3_Q6 | DIx-3 | 151 (+) | 1.000 | 0.999 | cTAATTgcc |
| | | | | | SEQ ID No. 133 |
| V$LHX2_Q4 | Lhx2 | 151 (−) | 1.000 | 1.000 | cTAATT |
| | | | | | SEQ ID No. 134 |
| V$DLX2_03 | DIx-2 | 151 (−) | 1.000 | 0.999 | cTAATTgc |
| | | | | | SEQ ID No. 132 |
| V$BARX1_04 | BARX1 | 151 (−) | 1.000 | 1.000 | cTAATTgc |
| | | | | | SEQ ID No. 132 |
| V$ISL2_02 | Is12 | 151 (+) | 1.000 | 0.984 | CTAATtg |
| | | | | | SEQ ID No. 135 |
| V$OG2_01 | OG-2 | 152 (+) | 1.000 | 1.000 | TAATTg |
| | | | | | SEQ ID No. 136 |
| V$PRRX2_03 | Prrx2 | 152 (−) | 1.000 | 1.000 | TAATT |
| | | | | | SEQ ID No. 137 |
| V$MEIS1BHOXA9_ | MEIS1B: HOXA9 | 155 (−) | 1.000 | 0.844 | ttgccaagaTGTCA |
| 02 | | | | | SEQ ID No. 138 |
| V$NF1A_Q6_01 | NF-1A | 156 (+) | 1.000 | 1.000 | tGCCAAg |
| | | | | | SEQ ID No. 139 |
| V$ZFP740_04 | ZFP740 | 164 (−) | 1.000 | 0.915 | tgtcatgGGGGGaag |
| | secondary motif | | | | ag |
| | | | | | SEQ ID No. 140 |
| V$CPBP_Q6 | CPBP | 168 (−) | 1.000 | 1.000 | atGGGGG |
| | | | | | SEQ ID No. 141 |
| V$SPIB_Q3 | Spi-B | 172 (−) | 1.000 | 1.000 | gGGGAA |
| | | | | | SEQ ID No. 142 |
| V$ZNF35_04 | ZNF35 | 174 (+) | 1.000 | 1.000 | gGAAGA |
| | | | | | SEQ ID No. 143 |
| V$OBOX2_02 | Obox2 | 179 (+) | 1.000 | 0.942 | aggagggGATTAagc |
| | | | | | ag |
| | | | | | SEQ ID No. 144 |
| V$TCF1_07 | TCF1 secondary | 179 (+) | 1.000 | 0.981 | aggagggGATTAag |
| | motif | | | | SEQ ID No. 145 |
| V$CRX_02 | Crx | 179 (+) | 1.000 | 0.954 | aggagggGATTAagc |
| | | | | | a |
| | | | | | SEQ ID No. 146 |
| V$PITX1_01 | Pitx1 | 179 (+) | 1.000 | 0.973 | aggaggGGATTaagc |
| | | | | | ag |
| | | | | | SEQ ID No. 144 |
| V$OTX2_01 | Otx2 | 180 (+) | 1.000 | 0.970 | ggagggGATTAagca |
| | | | | | ga |
| | | | | | SEQ ID No. 147 |
| V$PITX2_01 | PITX2 | 180 (+) | 1.000 | 0.965 | ggagggGATTAagca |
| | | | | | ga |
| | | | | | SEQ ID No. 147 |
| V$CRX_06 | Crx | 180 (+) | 1.000 | 0.980 | ggagggGATTAa |
| | | | | | SEQ ID No. 148 |
| V$CRX_05 | Crx | 181 (+) | 1.000 | 0.963 | gagggGATTAa |
| | | | | | SEQ ID No. 149 |
| V$CRX_Q4 | Crx | 182 (−) | 1.000 | 0.981 | agggGATTAagca |
| | | | | | SEQ ID No. 150 |
| V$GSC2_01 | GSC2 | 183 (−) | 1.000 | 0.997 | gggGATTAag |
| | | | | | SEQ ID No. 151 |
| V$DPRX_01 | DPRX | 183 (+) | 1.000 | 0.995 | gggGATTAag |
| | | | | | SEQ ID No. 151 |
| V$DMBX1_02 | DMBX1 | 183 (+) | 1.000 | 0.999 | gggGATTAag |
| | | | | | SEQ ID No. 151 |
| V$CRX_Q4_02 | Crx | 184 (−) | 1.000 | 0.996 | ggGATTAag |
| | | | | | SEQ ID No. 152 |
| V$OTX2_Q3_01 | Otx2 | 184 (−) | 1.000 | 1.000 | ggGATTAa |
| | | | | | SEQ ID No. 153 |
| V$PITX1_04 | PITX1 | 184 (−) | 1.000 | 1.000 | ggGATTAa |
| | | | | | SEQ ID No. 153 |
| V$PITX3_03 | PITX3 | 184 (−) | 1.000 | 1.000 | ggGATTAag |
| | | | | | SEQ ID No. 152 |
| V$PITX1_03 | PITX1 | 184 (−) | 1.000 | 1.000 | ggGATTAag |
| | | | | | SEQ ID No. 152 |
| V$OTX1_04 | OTX1 | 184 (−) | 1.000 | 0.996 | ggGATTAa |
| | | | | | SEQ ID No. 153 |
| V$GTF2IRD1_01 | GTF2IRD1- | 184 (+) | 1.000 | 0.990 | ggGATTAag |
| | isoform2 | | | | SEQ ID No. 152 |
| V$RHOXF1_02 | RHOXF1 | 184 (−) | 1.000 | 1.000 | gGGATTaa |
| | | | | | SEQ ID No. 153 |
| V$CRX_Q4_01 | CRX | 186 (−) | 1.000 | 1.000 | GATTAa |
| | | | | | SEQ ID No. 154 |
| V$HIC1_03 | HIC1 | 195 (+) | 1.000 | 0.958 | gacgggTGCCCctccc |
| | | | | | cc |
| | | | | | SEQ ID No. 155 |
| V$CTCF_16 | CTCF | 198 (−) | 1.000 | 0.903 | gggtgcccctCCCCCtc |
| | | | | | SEQ ID No. 156 |
| V$BTEB2_Q3 | BTEB2 | 200 (−) | 1.000 | 0.980 | gtgcccctcCCCCTcc |
| | | | | | SEQ ID No. 157 |
| V$GC_01 | GC box | 200 (−) | 0.956 | 0.952 | gtgccCCTCCccct |
| | | | | | SEQ ID No. 158 |
| V$MZF1_02 | MZF-1 | 200 (−) | 1.000 | 0.965 | gtgCCCCTccccc |
| | | | | | SEQ ID No. 159 |
| V$SP1_Q4_01 | Sp1 | 201 (−) | 0.964 | 0.970 | tgccCCTCCccct |
| | | | | | SEQ ID No. 160 |
| V$EGR1_16 | EGR1 | 202 (+) | 1.000 | 0.966 | gcccctCCCCCtcc |
| | | | | | SEQ ID No. 161 |
| V$BTEB2_Q3_01 | BTEB2 | 202 (−) | 0.995 | 0.997 | gccccTCCCC |
| | | | | | SEQ ID No. 162 |
| V$SP4_Q3 | Sp4 | 202 (+) | 0.991 | 0.985 | gcccCTCCCcctc |
| | | | | | SEQ ID No. 163 |
| V$SP1_09 | SP1 | 202 (+) | 0.985 | 0.989 | gccCCTCCccc |
| | | | | | SEQ ID No. 164 |
| V$BTEB3_Q5 | BTEB3 | 202 (−) | 1.000 | 0.967 | gccCCTCCccctc |
| | | | | | SEQ ID No. 163 |
| V$CPBP_Q6 | CPBP | 202 (+) | 1.000 | 1.000 | GCCCCtc |
| | | | | | SEQ ID No. 165 |
| V$ZBP89_Q4_01 | ZBP89 | 203 (+) | 1.000 | 1.000 | cccctCCCCCtc |
| | | | | | SEQ ID No. 166 |
| V$SP1_08 | Sp1 | 203 (−) | 1.000 | 0.977 | cccctCCCCCtccc |
| | | | | | SEQ ID No. 167 |
| V$ETF_Q6_01 | ETF | 203 (+) | 0.965 | 0.976 | ccCCTCCccct |
| | | | | | SEQ ID No. 168 |
| V$SP1_Q2_01 | Sp1 | 203 (+) | 0.972 | 0.979 | ccCCTCCccc |
| | | | | | SEQ ID No. 169 |
| V$GKLF_Q3_01 | GKLF | 203 (−) | 0.989 | 0.992 | cCCCTCcccctcc |
| | | | | | SEQ ID No. 170 |
| V$CKROX_Q2 | CKROX | 203 (+) | 1.000 | 1.000 | cCCCTCccc |
| | | | | | SEQ ID No. 171 |
| V$SP1_03 | SP1 | 203 (+) | 0.997 | 0.998 | CCCCTccccc |
| | | | | | SEQ ID No. 169 |
| V$WT1_Q6 | WT1 | 204 (+) | 1.000 | 1.000 | cCCTCCccc |
| | | | | | SEQ ID No. 172 |
| V$MAZ_Q6 | MAZ | 204 (−) | 1.000 | 1.000 | ccCTCCCc |
| | | | | | SEQ ID No. 173 |
| V$GKLF_Q3 | GKLF | 205 (−) | 0.986 | 0.977 | cctccCCCTCccag |
| | | | | | SEQ ID No. 174 |
| V$GKLF_Q3_01 | GKLF | 207 (−) | 0.992 | 0.992 | tCCCCCtcccagc |
| | | | | | SEQ ID No. 175 |
| V$CPBP_Q6 | CPBP | 208 (+) | 1.000 | 1.000 | CCCCCtc |
| | | | | | SEQ ID No. 176 |
| V$CP2_Q4 | CP2 | 211 (+) | 1.000 | 0.997 | cctCCCAGccaa |
| | | | | | SEQ ID No. 177 |
| VIK_Q5_01 | Ikaros | 211 (−) | 1.000 | 1.000 | ccTCCCA |
| | | | | | SEQ ID No. 178 |
| V$CP2_Q6 | CP2 | 213 (−) | 1.000 | 1.000 | tcCCAGCcaa |
| | | | | | SEQ ID No. 179 |
| V$YB1_Q4 | YB-1 | 215 (+) | 1.000 | 0.992 | ccagCCAATgt |
| | | | | | SEQ ID No. 180 |
| V$CTCF_05 | CTCF | 215 (+) | 0.957 | 0.864 | ccagccaatgtCACCT |
| | | | | | cctgg |
| | | | | | SEQ ID No. 181 |
| V$NF1C_Q6 | NF-1C | 216 (−) | 1.000 | 1.000 | caGCCAA |
| | | | | | SEQ ID No. 182 |
| V$SOX18_Q5 | Sox-18 | 220 (+) | 1.000 | 1.000 | CAATGtc |
| | | | | | SEQ ID No. 183 |
| V$TFIII_Q6_01 | TFII-I | 225 (−) | 0.984 | 0.989 | tcacCTCCTg |
| | | | | | SEQ ID No. 184 |

TABLE 5
Transcription factor Position Weight Matrix (PWM, Transfac) recognizing the Human
GUCA1B promoter (−250 bp from TSS)

| | | Position | Core | Matrix | |
|---|---|---|---|---|---|
| Matrix | Factor name | (strand) | score | score | Sequence |
| V$SMAD2_Q6 | Smad2 | 6 (+) | 1.000 | 1.000 | AGACAg |
| | | | | | SEQ ID No. 185 |
| V$CEBPG_Q6 | C/EBPgamma | 12 (−) | 1.000 | 0.973 | gATTTCaccatg |
| 01 | | | | | SEQ ID No. 186 |
| V$PAX6_Q2 | Pax-6 | 33 (+) | 0.801 | 0.805 | ctggtcTCGAActc |
| | | | | | SEQ ID No. 187 |
| V$RPC155_01 | RPC155 | 35 (−) | 1.000 | 1.000 | ggtcTCGAActcctga |
| | | | | | SEQ ID No. 188 |
| V$RARA_08 | RARA | 37 (−) | 1.000 | 0.771 | tctcgaactccTGACCt |
| | | | | | t |
| | | | | | SEQ ID No. 189 |
| V$DAX1_01 | DAX1 | 42 (−) | 1.000 | 0.991 | aactcctGACCTtgtga |
| | | | | | tcc |
| | | | | | SEQ ID No. 190 |
| VYRARA_08 | RARA | 45 (−) | 0.781 | 0.776 | tcctgaccttgTGATCc |
| | | | | | a |
| | | | | | SEQ ID No. 191 |
| V$NR4A2_01 | NURR1 | 47 (−) | 1.000 | 0.967 | cTGACCtt |
| | | | | | SEQ ID No. 192 |
| V$ESRRA_10 | ERR1 | 47 (+) | 1.000 | 0.981 | ctGACCTtgtga |
| | | | | | SEQ ID No. 193 |
| V$SF1_Q6 | SF-1 | 48 (+) | 1.000 | 1.000 | tgaCCTTG |
| | | | | | SEQ ID No. 194 |
| V$ERR3_Q2 | ERR3 | 48 (−) | 1.000 | 1.000 | tgACCTTg |
| | | | | | SEQ ID No. 194 |
| V$RXRA_12 | RXR-ALPHA | 48 (−) | 1.000 | 1.000 | TGACCtt |
| | | | | | SEQ ID No. 195 |
| V$NR1B1_01 | NR1B1 | 48 (−) | 1.000 | 1.000 | TGACCtt |
| | | | | | SEQ ID No. 195 |
| V$RARG_Q3 | RAR-gamma | 48 (+) | 1.000 | 0.988 | TGACCttgtga |
| | | | | | SEQ ID No. 196 |
| V$COUPTF1_ | COUP-TF1 | 48 (+) | 1.000 | 1.000 | TGACCtt |
| Q6_01 | | | | | SEQ ID No. 195 |
| V$KID3_01 | Kid3 | 60 (+) | 1.000 | 1.000 | CCACC |
| | | | | | SEQ ID No. 197 |
| V$EGR3_Q3 | egr-3 | 62 (−) | 1.000 | 1.000 | aCCCAC |
| | | | | | SEQ ID No. 198 |
| V$KID3_01 | Kid3 | 64 (+) | 1.000 | 1.000 | CCACC |
| | | | | | SEQ ID No. 197 |
| V$IK_Q5_01 | Ikaros | 73 (−) | 1.000 | 1.000 | ccTCCCA |
| | | | | | SEQ ID No. 199 |
| V$SOX18_Q5 | Sox-18 | 78 (+) | 1.000 | 1.000 | CAAAGtg |
| | | | | | SEQ ID No. 200 |
| V$PITX2_Q6 | pitx2 | 86 (−) | 1.000 | 1.000 | tgGGATTaca |
| | | | | | SEQ ID No. 201 |
| V$PITX2_Q4 | Pitx2 | 86 (−) | 1.000 | 1.000 | tgGGATTaca |
| | | | | | SEQ ID No. 201 |
| V$GTF2IRD1_ | GTF2IRD1- | 87 (+) | 1.000 | 0.983 | ggGATTAca |
| 01 | isoform2 | | | | SEQ ID No. 202 |
| V$SREBP2_Q6 | SREBP-2 | 101 (+) | 1.000 | 0.991 | gaGCCACcgcac |
| | | | | | SEQ ID No. 203 |
| V$KID3_01 | Kid3 | 104 (+) | 1.000 | 1.000 | CCACC |
| | | | | | SEQ ID No. 197 |
| V$KAISO_Q2 | Kaiso | 106 (−) | 0.888 | 0.922 | aCCGCAcccggc |
| | | | | | SEQ ID No. 204 |
| V$E2A_Q6_01 | E2A | 115 (+) | 1.000 | 0.991 | ggcCACCTgggga |
| | | | | | SEQ ID No. 205 |
| V$CTCF_06 | CTCF | 116 (−) | 0.800 | 0.877 | gccacCTGGG |
| | | | | | SEQ ID No. 206 |
| V$E2A_Q6_02 | E2A | 116 (−) | 1.000 | 1.000 | gccACCTG |
| | | | | | SEQ ID No. 207 |
| V$CMYC_Q6_ | c-Myc | 116 (−) | 0.946 | 0.960 | gcCACCTg |
| 01 | | | | | SEQ ID No. 207 |
| V$KID3_01 | Kid3 | 117 (+) | 1.000 | 1.000 | CCACC |
| | | | | | SEQ ID No. 197 |
| V$TWIST_Q6 | TWIST | 118 (+) | 1.000 | 1.000 | CACCTgg |
| | | | | | SEQ ID No. 208 |
| V$SPIB_Q3 | Spi-B | 123 (−) | 1.000 | 1.000 | gGGGAA |
| | | | | | SEQ ID No. 209 |
| V$HSF2_Q6 | HSF2 | 124 (+) | 1.000 | 0.945 | gggaatcTTCTAatag |
| | | | | | SEQ ID No. 210 |
| V$HSF2_01 | HSF2 | 125 (+) | 0.999 | 0.995 | GGAATcttct |
| | | | | | SEQ ID No. 211 |
| V$HSF1_01 | HSF1 | 125 (−) | 0.998 | 0.982 | ggaatCTTCT |
| | | | | | SEQ ID No. 211 |
| V$HSF2_01 | HSF2 | 125 (−) | 0.998 | 0.987 | ggaatCTTCT |
| | | | | | SEQ ID No. 211 |
| V$PRDM16_05 | MEL1 | 128 (−) | 1.000 | 1.000 | aTCTTC |
| | | | | | SEQ ID No. 212 |
| V$THAP1_03 | THAP1 | 142 (−) | 1.000 | 0.994 | ttaGGGCAg |
| | | | | | SEQ ID No. 213 |
| V$PAX6_Q2 | Pax-6 | 156 (+) | 0.768 | 0.806 | ctggccTGGACcca |
| | | | | | SEQ ID No. 214 |
| V$SALL1_02 | Sall1 | 157 (−) | 1.000 | 0.940 | tggcctGGACCc |
| | | | | | SEQ ID No. 215 |
| V$SALL1_04 | Sall1 | 157 (−) | 1.000 | 0.939 | tggcctGGACCc |
| | | | | | SEQ ID No. 215 |
| V$LRF_Q6 | LRF | 163 (−) | 1.000 | 0.987 | ggaCCCACctcc |
| | | | | | SEQ ID No. 216 |
| V$EGR3_Q3 | egr-3 | 165 (−) | 1.000 | 1.000 | aCCCAC |
| | | | | | SEQ ID No. 198 |
| V$GKLF_Q3 | GKLF | 165 (−) | 0.990 | 0.975 | acccaCCTCCcttg |
| | | | | | SEQ ID No. 217 |
| V$KID3_01 | Kid3 | 167 (+) | 1.000 | 1.000 | CCACC |
| | | | | | SEQ ID No. 197 |
| V$BTEB3_Q5 | BTEB3 | 167 (−) | 1.000 | 0.965 | ccaCCTCCcttgc |
| | | | | | SEQ ID No. 218 |
| V$GKLF_Q4 | GKLF | 170 (+) | 1.000 | 1.000 | CCTCCct |
| | | | | | SEQ ID No. 122 |
| V$PAX4_Q2 | Pax-4 | 174 (+) | 1.000 | 0.904 | ccttgCCACCt |
| | | | | | SEQ ID No. 219 |
| V$SREBP2_Q6 | SREBP-2 | 176 (+) | 1.000 | 0.991 | ttGCCACctgcc |
| | | | | | SEQ ID No. 220 |
| V$E12_Q6 | E12 | 177 (−) | 1.000 | 0.992 | tgccACCTGcc |
| | | | | | SEQ ID No. 221 |
| V$DEC1_Q3 | DEC1 | 177 (+) | 0.966 | 0.943 | tgccACCTGccc |
| | | | | | SEQ ID No. 222 |
| V$SNA_Q4 | SNA | 178 (+) | 1.000 | 0.992 | gcCACCTgccccct |
| | | | | | SEQ ID No. 223 |
| V$CMYC_Q6_ | c-Myc | 178 (−) | 0.946 | 0.960 | gcCACCTg |
| 01 | | | | | SEQ ID No. 207 |
| V$E2A_Q6_02 | E2A | 178 (−) | 1.000 | 1.000 | gccACCTG |
| | | | | | SEQ ID No. 207 |
| V$SNA_Q6 | SNA | 179 (+) | 1.000 | 1.000 | cCACCTgccc |
| | | | | | SEQ ID No. 224 |
| V$EBOX_Q6_ | Ebox | 179 (+) | 0.998 | 0.996 | cCACCTgccc |
| 01 | | | | | SEQ ID No. 224 |
| V$KID3_01 | Kid3 | 179 (+) | 1.000 | 1.000 | CCACC |
| | | | | | SEQ ID No. 197 |
| V$E2A_Q6 | E2A | 180 (+) | 1.000 | 1.000 | CACCTgcc |
| | | | | | SEQ ID No. 225 |
| V$HTF4_Q2 | HTF4 | 180 (+) | 1.000 | 1.000 | CACCTgc |
| | | | | | SEQ ID No. 226 |
| V$MRF4_Q3 | MRF4 | 180 (+) | 1.000 | 1.000 | CACCTgc |
| | | | | | SEQ ID No. 226 |
| V$GCM2MAX_ | GCMb:Max | 180 (−) | 0.909 | 0.889 | CACCTgccccctgcac |
| 01 | | | | | SEQ ID No. 227 |
| V$MYOGENIN_ | myogenin | 180 (−) | 1.000 | 1.000 | cACCTGcc |
| Q6 | | | | | SEQ ID No. 225 |
| VHAIRYLIKE_ | HAIRYLIKE | 180 (−) | 0.993 | 0.993 | cACCTGcccc |
| Q3 | | | | | SEQ ID No. 228 |
| V$KAISO_Q2 | Kaiso | 182 (−) | 1.000 | 0.988 | cCTGCCccctgc |
| | | | | | SEQ ID No. 229 |
| V$ZNF300_04 | ZNF300 | 184 (−) | 1.000 | 0.991 | tgcCCCCTg |
| | | | | | SEQ ID No. 230 |
| V$CPBP_Q6 | CPBP | 186 (+) | 1.000 | 1.000 | CCCCCtg |
| | | | | | SEQ ID No. 231 |
| V$CUX1_03 | Cux1 | 194 (+) | 1.000 | 0.953 | accgacTGATCatgttc |
| | | | | | SEQ ID No. 232 |
| V$TCF11MAFG_ | TCF11: MafG | 200 (−) | 0.963 | 0.834 | tgatcatgttcaGTCAC |
| 01 | | | | | ccagg |
| | | | | | SEQ ID No. 233 |
| V$FXR_Q3 | FXR: RXR-alpha | 204 (+) | 0.897 | 0.876 | catgttcagTCACC |
| | | | | | SEQ ID No. 234 |
| V$NRF2_Q4 | Nrf-2 | 205 (+) | 1.000 | 0.904 | atgttcAGTCAcc |
| | | | | | SEQ ID No. 235 |
| V$AP1_Q4 | AP-1 | 207 (−) | 1.000 | 0.983 | gttcAGTCAcc |
| | | | | | SEQ ID No. 236 |
| V$AP1FJ_Q2 | AP-1 | 207 (−) | 1.000 | 0.986 | gttcaGTCACc |
| | | | | | SEQ ID No. 236 |
| V$MYF6_04 | MYF6 secondary | 229 (−) | 1.000 | 0.895 | ggagaGGCTGatcag |
| | motif | | | | SEQ ID No. 237 |
| V$CUX1_03 | Cux1 | 231 (+) | 1.000 | 0.920 | agaggcTGATCaggcc |
| | | | | | t |
| | | | | | SEQ ID No. 238 |

_

TABLE 6
Transcription factor Position Weight Matrix (PWM, Transfac) recognizing the Human
PRPH2 promoter (−250 bp from TSS)

| | | Position | Core | Matrix | |
|---|---|---|---|---|---|
| Matrix | Factor name | (strand) | score | score | Sequence |
| V$NF1B_Q6_ | NF-1B | 1 (+) | 1.000 | 1.000 | GCCAGacat |
| 01 | | | | | SEQ ID No. 239 |
| V$FLI1_04 | FLI1 | 1 (+) | 0.816 | 0.762 | gccagacATCCAga |
| | | | | | SEQ ID No. 240 |
| V$FOXO1SPDEF_ | FOXO1A: PDEF | 3 (−) | 1.000 | 0.934 | cagaCATCCagat |
| 01 | | | | | SEQ ID No. 241 |
| V$MSX1_01 | Msx-1 | 11 (−) | 0.928 | 0.950 | cAGATAcag |
| | | | | | SEQ ID No. 242 |
| V$GATA5_Q4 | GATA-5 | 11 (−) | 1.000 | 1.000 | cAGATA |
| | | | | | SEQ ID No. 243 |
| V$TORC2_Q3 | TORC2 | 15 (−) | 1.000 | 1.000 | tacaGCCCA |
| | | | | | SEQ ID No. 244 |
| V$IRF3_06 | IRF3 secondary | 16 (−) | 0.981 | 0.915 | acagcCCATTcttc |
| | motif | | | | SEQ ID No. 245 |
| V$SPIB_Q3 | Spi-B | 24 (+) | 1.000 | 1.000 | TTCTTc |
| | | | | | SEQ ID No. 246 |
| V$HNF4A_Q6_ | HNF-4alpha | 37 (−) | 1.000 | 0.976 | tcttgccCTTTGctc |
| 01 | | | | | SEQ ID No. 247 |
| V$HNF4_01 | HNF-4 | 37 (−) | 1.000 | 0.966 | tcttgccCTTTGctcct |
| | | | | | ga |
| | | | | | SEQ ID No. 248 |
| V$ZNF35_04 | ZNF35 | 37 (−) | 1.000 | 1.000 | TCTTGc |
| | | | | | SEQ ID No. 249 |
| V$RXRA_13 | RXR-ALPHA | 39 (−) | 1.000 | 0.816 | ttgcCCTTTgctcct |
| | | | | | SEQ ID No. 250 |
| V$HNF4_01_B | HNF4alpha1 | 39 (−) | 1.000 | 0.931 | ttgccCTTTGctcct |
| | | | | | SEQ ID No. 250 |
| V$HNF4_Q6_ | HNF-4 | 39 (−) | 1.000 | 0.970 | ttgccCTTTGctcc |
| 01 | | | | | SEQ ID No. 251 |
| V$HNF4_Q6_ | HNF-4 | 39 (−) | 1.000 | 0.976 | ttgccCTTTGctcctga |
| 04 | | | | | ttc |
| | | | | | SEQ ID No. 252 |
| V$HNF4A_11 | HNF4A | 39 (+) | 1.000 | 0.951 | ttgccCTTTGctcct |
| | | | | | SEQ ID No. 250 |
| V$HNF4A_Q3 | HNF-4A | 39 (−) | 1.000 | 0.973 | ttgccCTTTGctcc |
| | | | | | SEQ ID No. 251 |
| V$HNF4G_05 | HNF-4gamma | 40 (−) | 1.000 | 0.972 | tgccCTTTGctcct |
| | | | | | SEQ ID No. 252 |
| V$HNF4G_04 | HNF-4gamma | 40 (−) | 1.000 | 0.963 | tgccCTTTGctcctg |
| | | | | | SEQ ID No. 253 |
| V$HNF4A_03 | HNF4A | 40 (−) | 1.000 | 0.968 | tgccCTTTGctcc |
| | | | | | SEQ ID No. 254 |
| V$EAR2_Q2 | EAR2 | 40 (+) | 1.000 | 0.910 | tgccCTTTGctcct |
| | | | | | SEQ ID No. 252 |
| V$PPARGRXRA_ | PPARgamma: RXR- | 40 (−) | 1.000 | 0.896 | tgcCCTTTgctcctg |
| 01 | alpha | | | | SEQ ID No. 253 |
| V$SOX18_Q5 | Sox-18 | 42 (−) | 1.000 | 1.000 | ccCTTTG |
| | | | | | SEQ ID No. 255 |
| V$SPIB_Q3 | Spi-B | 56 (+) | 1.000 | 1.000 | TTCTCc |
| | | | | | SEQ ID No. 256 |
| V$ZNF780A_ | ZNF780A | 61 (−) | 1.000 | 1.000 | caagctGTACCc |
| 01 | | | | | SEQ ID No. 257 |
| V$CP2_02 | CP2/LBP-1c/LSF | 61 (−) | 0.957 | 0.954 | caagctgtacCCAGA |
| | | | | | SEQ ID No. 258 |
| V$CP2_01 | CP2 | 64 (+) | 1.000 | 0.937 | gctgtaCCCAG |
| | | | | | SEQ ID No. 259 |
| V$HSF1_Q5 | HSF1 | 74 (−) | 1.000 | 0.962 | gagctTTCTGgt |
| | | | | | SEQ ID No. 260 |
| V$HSF1_Q6_ | HSF1 | 74 (+) | 1.000 | 0.970 | gagctTTCTGgttc |
| 01 | | | | | SEQ ID No. 261 |
| V$GABPA_08 | GABP-alpha | 78 (−) | 0.865 | 0.880 | tttcTGGTT |
| | | | | | SEQ ID No. 262 |
| V$DR3_Q4 | VDR, CAR, PXR | 79 (−) | 1.000 | 0.851 | ttctgGTTCAgcatgta |
| | | | | | cctg |
| | | | | | SEQ ID No. 263 |
| V$CMAF_Q5 | c-MAF | 81 (+) | 1.000 | 0.968 | ctggtTCAGCa |
| | | | | | SEQ ID No. 264 |
| V$NR3C1_10 | GR | 83 (−) | 0.996 | 0.863 | ggttcagcaTGTACc |
| | | | | | SEQ ID No. 265 |
| V$AR_14 | AR | 83 (+) | 0.963 | 0.904 | ggttcagcaTGTACct |
| | | | | | g |
| | | | | | SEQ ID No. 266 |
| V$AR_04 | AR | 83 (+) | 0.994 | 0.908 | ggttcagcaTGTACc |
| | | | | | SEQ ID No. 265 |
| V$AR_01 | AR | 83 (+) | 0.977 | 0.895 | ggttcagcaTGTACc |
| | | | | | SEQ ID No. 265 |
| V$NR112_01 | PXR | 83 (+) | 1.000 | 0.882 | gGTTCAgcatgtac |
| | | | | | SEQ ID No. 267 |
| VAR_10 | AR | 84 (−) | 0.981 | 0.951 | gttcagcaTGTACct |
| | | | | | SEQ ID No. 268 |
| V$MAFB_Q4_ | MAFB | 85 (+) | 1.000 | 1.000 | tTCAGCa |
| 01 | | | | | SEQ ID No. 269 |
| V$AP2GAMMA_ | AP-2gamma | 94 (−) | 0.944 | 0.960 | tACCTGgaggctgctt |
| Q4 | | | | | SEQ ID No. 270 |
| V$YY1_01 | YY1 | 109 (+) | 1.000 | 0.983 | tttgACCATgttttgag |
| | | | | | SEQ ID No. 271 |
| V$YY1_08 | YY1 | 113 (−) | 1.000 | 0.959 | accaTGTTT |
| | | | | | SEQ ID No. 272 |
| V$GATA4_Q5_ | GATA-4 | 137 (−) | 1.000 | 1.000 | ttTATCT |
| 01 | | | | | SEQ ID No. 273 |
| V$GATA3_Q4 | GATA-3 | 138 (−) | 1.000 | 1.000 | tTATCT |
| | | | | | SEQ ID No. 274 |
| V$ISL2_02 | Isl2 | 142 (+) | 1.000 | 1.000 | CTAATgg |
| | | | | | SEQ ID No. 275 |
| V$IPF1_Q5 | ipf1 | 142 (+) | 1.000 | 1.000 | cTAATGg |
| | | | | | SEQ ID No. 275 |
| V$CRX_Q4_01 | CRX | 167 (−) | 1.000 | 1.000 | GATTAg |
| | | | | | SEQ ID No. 276 |
| V$POU3F2_02 | POU3F2 | 168 (−) | 0.783 | 0.879 | attagCAAAA |
| | | | | | SEQ ID No. 277 |
| V$HES1_Q2 | HES-1 | 177 (+) | 0.989 | 0.964 | atgtcTTGTGgatcc |
| | | | | | SEQ ID No. 278 |
| V$ZNF709_02 | ZNF709 | 180 (−) | 0.985 | 0.980 | tCTTGTggatcc |
| | | | | | SEQ ID No. 279 |
| V$NKX25_03 | NKX25 | 187 (+) | 1.000 | 0.859 | gatccCACTTttaatc |
| | | | | | SEQ ID No. 280 |
| V$LHX3_Q3 | LHX3 | 196 (−) | 1.000 | 1.000 | tTTAAT |
| | | | | | SEQ ID No. 281 |
| V$FOXN4_04 | FOXN4 secondary | 196 (+) | 0.837 | 0.752 | tttaatctgcAGCGTtc |
| | motif | | | | aggct |
| | | | | | SEQ ID No. 282 |
| V$CRX_Q4_01 | CRX | 197 (+) | 1.000 | 1.000 | tTAATC |
| | | | | | SEQ ID No. 283 |
| V$BEN_01 | BEN | 205 (+) | 1.000 | 0.947 | CAGCGttc |
| | | | | | SEQ ID No. 284 |
| V$CPBP_Q6 | CPBP | 218 (+) | 1.000 | 1.000 | GCCCCtt |
| | | | | | SEQ ID No. 285 |
| V$CP2_Q4 | CP2 | 226 (−) | 1.000 | 0.997 | ttggCTGGGaag |
| | | | | | SEQ ID No. 286 |
| V$CP2_Q6 | CP2 | 226 (+) | 1.000 | 1.000 | ttgGCTGGga |
| | | | | | SEQ ID No. 287 |
| V$NF1C_Q6 | NF-1C | 226 (+) | 1.000 | 1.000 | TTGGCtg |
| | | | | | SEQ ID No. 288 |
| V$ZF5_01 | ZF5 | 237 (+) | 1.000 | 0.926 | GGGCGctg |
| | | | | | SEQ ID No. 289 |
| V$BEN_01 | BEN | 237 (−) | 1.000 | 0.952 | gggCGCTG |
| | | | | | SEQ ID No. 289 |
| V$MAFB_Q4 | MafB | 240 (+) | 1.000 | 0.987 | CGCTGAagaaa |
| | | | | | SEQ ID No. 290 |
| V$SPIB_Q3 | Spi-B | 244 (−) | 1.000 | 1.000 | gAAGAA |
| | | | | | SEQ ID No. 291 |
| V$PARP_Q4 | PARP | 245 (−) | 1.000 | 1.000 | aAGAAA |
| | | | | | SEQ ID No. 292 |

TABLE 7
Transcription factor Position Weight Matrix (PWM, Transfac) recognizing the Human
RDH12 promoter (−250 bp from TSS)

| | | Position | Core | Matrix | |
|---|---|---|---|---|---|
| Matrix | Factor name | (strand) | score | score | Sequence |
| V$TWIST_Q6_ | TWIST | 31 (−) | 0.998 | 0.996 | ccCATGTgtat |
| 01 | | | | | SEQ ID No. 293 |
| V$PARP_Q3 | PARP | 48 (−) | 1.000 | 0.989 | ctaTTTCTtg |
| | | | | | SEQ ID No. 294 |
| V$PARP_Q4 | PARP | 51 (+) | 1.000 | 1.000 | TTTCTt |
| | | | | | SEQ ID No. 295 |
| V$ZNF35_04 | ZNF35 | 53 (−) | 1.000 | 1.000 | TCTTGc |
| | | | | | SEQ ID No. 249 |
| V$GR_01 | GR | 54 (+) | 1.000 | 0.949 | cttgcctttccattcTGTT |
| | | | | | Ctattgat |
| | | | | | SEQ ID No. 296 |
| V$GR_Q6 | GR | 58 (+) | 1.000 | 0.975 | cctttccattcTGTTCtat |
| | | | | | SEQ ID No. 297 |
| V$GRE_C | GR | 60 (+) | 1.000 | 0.846 | tttccattctGTTCTa |
| | | | | | SEQ ID No. 298 |
| V$AR_Q6_01 | AR | 61 (+) | 1.000 | 0.983 | ttccattcTGTTCta |
| | | | | | SEQ ID No. 299 |
| V$PR_Q6 | PR | 64 (+) | 1.000 | 1.000 | cattcTGTTCt |
| | | | | | SEQ ID No. 300 |
| V$GR_Q6_02 | GR | 64 (+) | 1.000 | 0.999 | cattcTGTTCtat |
| | | | | | SEQ ID No. 301 |
| V$GR_Q4 | GR | 68 (−) | 1.000 | 1.000 | cTGTTCt |
| | | | | | SEQ ID No. 302 |
| V$HNF6_Q6 | HNF-6 | 73 (−) | 1.000 | 0.987 | ctATTGAtttgt |
| | | | | | SEQ ID No. 303 |
| V$OC2_Q3 | OC-2 | 74 (−) | 1.000 | 1.000 | tATTGA |
| | | | | | SEQ ID No. 304 |
| V$HNF6_Q4 | HNF-6 | 74 (−) | 1.000 | 1.000 | tATTGAtt |
| | | | | | SEQ ID No. 305 |
| V$FOXL2_Q5 | FOXL2 | 76 (−) | 0.971 | 0.967 | ttgATTTGtct |
| | | | | | SEQ ID No. 306 |
| V$TFIIB_Q6_ | TFIIB | 81 (+) | 0.990 | 0.987 | tTGTCTgcct |
| 02 | | | | | SEQ ID No. 307 |
| V$SMAD_Q6_ | SMAD | 81 (−) | 1.000 | 0.994 | ttGTCTGccta |
| 01 | | | | | SEQ ID No. 308 |
| V$SMAD1_Q6 | Smad1 | 82 (−) | 1.000 | 0.998 | tgTCTGCct |
| | | | | | SEQ ID No. 309 |
| V$SMAD4_Q6_ | Smad4 | 82 (+) | 1.000 | 1.000 | tGTCTGc |
| 01 | | | | | SEQ ID No. 310 |
| V$SMAD3_Q6 | SMAD3 | 82 (+) | 1.000 | 0.983 | tGTCTGcct |
| | | | | | SEQ ID No. 311 |
| V$SNAP190_ | SNAP190 | 86 (+) | 0.948 | 0.955 | tgcCTATT |
| 02 | | | | | SEQ ID No. 312 |
| V$RXRA_04 | RXR-ALPHA | 89 (−) | 0.997 | 0.911 | ctattcACCTAcctgt |
| | secondary motif | | | | SEQ ID No. 313 |
| V$AREB6_01 | AREB6 | 94 (+) | 1.000 | 0.966 | cacctACCTGtac |
| | | | | | SEQ ID No. 314 |
| V$BRN1_Q6 | BRN1 | 122 (−) | 1.000 | 1.000 | aGCATTt |
| | | | | | SEQ ID No. 315 |
| V$CDP_02 | CDP | 134 (−) | 0.930 | 0.915 | caacttaTCAATgat |
| | | | | | SEQ ID No. 316 |
| V$CLOX_01 | Clox | 134 (−) | 0.941 | 0.898 | caacttaTCAATgat |
| | | | | | SEQ ID No. 317 |
| V$CUX1_07 | CDP | 138 (+) | 0.970 | 0.942 | ttaTCAATga |
| | | | | | SEQ ID No. 318 |
| V$HOXA9_Q5 | Hoxa9 | 140 (+) | 1.000 | 0.911 | atcAATGAtatg |
| | | | | | SEQ ID No. 319 |
| V$BNC1_01 | BNC1 | 150 (+) | 1.000 | 1.000 | TGATGgtgg |
| | | | | | SEQ ID No. 320 |
| V$ING4_01 | ING4 | 153 (−) | 1.000 | 1.000 | TGGTGg |
| | | | | | SEQ ID No. 321 |
| V$KID3_01 | Kid3 | 154 (−) | 1.000 | 1.000 | GGTGG |
| | | | | | SEQ ID No. 322 |
| V$NURR1_Q3 | NURR1 | 156 (+) | 1.000 | 1.000 | tgGCCTT |
| | | | | | SEQ ID No. 323 |
| V$SOX18_Q5 | Sox-18 | 158 (−) | 1.000 | 1.000 | gcCTTTG |
| | | | | | SEQ ID No. 324 |
| V$SOX21_03 | Sox-21 | 162 (−) | 1.000 | 0.972 | ttggattATAATattt |
| | | | | | SEQ ID No. 325 |
| V$SOX14_03 | Sox-14 | 162 (−) | 1.000 | 0.962 | ttggattATAATattt |
| | | | | | SEQ ID No. 325 |
| V$SOX40_04 | Sox-30 secondary | 162 (−) | 1.000 | 0.971 | ttggatTATAAtattt |
| | motif | | | | SEQ ID No. 325 |
| V$SOX40_04 | Sox-30 secondary | 162 (+) | 1.000 | 0.975 | ttggaTTATAatattt |
| | motif | | | | SEQ ID No. 325 |
| V$SOX21_03 | Sox-21 | 162 (+) | 1.000 | 0.987 | ttggATTATaatattt |
| | | | | | SEQ ID No. 325 |
| V$SOX14_03 | Sox-14 | 162 (+) | 1.000 | 0.969 | ttggATTATaatattt |
| | | | | | SEQ ID No. 325 |
| V$GTF2IRD1_ | GTF2IRD1-isoform2 | 163 (+) | 1.000 | 0.968 | tgGATTAta |
| 01 | | | | | SEQ ID No. 326 |
| V$ZNF333_ | ZNF333 | 166 (−) | 1.000 | 1.000 | ATTAT |
| 01 | | | | | SEQ ID No. 327 |
| V$FOXJ2_02 | FOXJ2 | 166 (+) | 1.000 | 0.939 | attATAATatttaa |
| | | | | | SEQ ID No. 328 |
| V$HNF1B_Q6_ | HNF-1beta | 168 (+) | 1.000 | 0.915 | tataatatTTAACa |
| 01 | | | | | SEQ ID No. 329 |
| V$HNF1B_04 | HNF1B | 169 (+) | 1.000 | 0.950 | ataatatTTAAC |
| | | | | | SEQ ID No. 330 |
| V$ZNF333_ | ZNF333 | 169 (+) | 1.000 | 1.000 | ATAAT |
| 01 | | | | | SEQ ID No. 331 |
| V$HOXD13_ | HOXD13 | 181 (−) | 1.000 | 0.979 | atTTTATttta |
| Q6 | | | | | SEQ ID No. 332 |
| V$HOXA13_ | HOXA13 | 182 (−) | 1.000 | 1.000 | tTTTAT |
| 01 | | | | | SEQ ID No. 333 |
| V$SATB1_Q5_ | SATB1 | 182 (+) | 1.000 | 1.000 | tTTTAT |
| 01 | | | | | |
| V$CDX1_Q5 | Cdx-1 | 183 (+) | 1.000 | 1.000 | TTTATt |
| | | | | | SEQ ID No. 334 |
| V$TEF_Q6 | TEF | 184 (−) | 0.985 | 0.914 | TTATTttagcaa |
| | | | | | SEQ ID No. 335 |
| V$CEBPA_01 | C/EBPalpha | 185 (−) | 0.977 | 0.978 | tattttaGCAAAgt |
| | | | | | SEQ ID No. 336 |
| V$SOX18_Q5 | Sox-18 | 193 (+) | 1.000 | 1.000 | CAAAGtg |
| | | | | | SEQ ID No. 337 |
| V$OCT1_04 | Oct-1 | 200 (−) | 0.952 | 0.955 | tttgtgtattTTCATaatt |
| | | | | | ctag |
| | | | | | SEQ ID No. 338 |
| V$OCT1_05 | Oct-1 | 204 (+) | 0.934 | 0.911 | tgtaTTTTCataat |
| | | | | | SEQ ID No. 339 |
| V$POU2F2_ | Oct-2 | 204 (+) | 0.879 | 0.913 | tgtattTTCATaa |
| 08 | | | | | SEQ ID No. 340 |
| V$OCT_Q6 | Octamer | 205 (+) | 0.957 | 0.960 | gtattTTCATa |
| | | | | | SEQ ID No. 341 |
| V$IRF4_Q6 | IRF-4 | 206 (−) | 1.000 | 1.000 | taTTTTC |
| | | | | | SEQ ID No. 342 |
| V$POU2F1_ | POU2F1 | 206 (−) | 0.884 | 0.935 | tattTTCATaat |
| 02 | | | | | SEQ ID No. 343 |
| V$POU2F2_ | POU2F2 | 206 (−) | 0.890 | 0.935 | tattTTCATaa |
| 02 | | | | | SEQ ID No. 344 |
| V$POU2F1_ | POU2F1 | 207 (+) | 0.973 | 0.981 | aTTTTCata |
| Q4 | | | | | SEQ ID No. 345 |
| V$PIT1_Q6 | Pit-1 | 208 (+) | 1.000 | 0.902 | ttTTCATaattctagatg |
| | | | | | SEQ ID No. 346 |
| VYZNF333_ | ZNF333 | 213 (+) | 1.000 | 1.000 | ATAAT |
| 01 | | | | | SEQ ID No. 331 |
| V$PRRX2_03 | Prrx2 | 214 (−) | 1.000 | 1.000 | TAATT |
| | | | | | SEQ ID No. 347 |
| V$TFAP2CDLX3_ | AP-2gamma: DIx-3 | 214 (−) | 1.000 | 0.934 | TAATTctagatgccttat |
| 03 | | | | | ggtc |
| | | | | | SEQ ID No. 348 |
| V$ZNF709_ | ZNF709 | 226 (−) | 1.000 | 0.967 | cCTTATggtcct |
| 02 | | | | | SEQ ID No. 349 |
| V$PLAGL2_03 | PLAGL2 | 232 (−) | 1.000 | 1.000 | GGTCCtt |
| | | | | | SEQ ID No. 350 |
| V$PRDM16_ | MEL1 | 244 (−) | 1.000 | 1.000 | aTCTTC |
| 05 | | | | | SEQ ID No. 351 |

TABLE 8
Transcription factor Position Weight Matrix (PWM, Transfac) recognizing the Human
RP1 promoter (−250 bp from TSS)

| | | Position | Core | Matrix | |
|---|---|---|---|---|---|
| Matrix | Factor name | (strand) | score | score | Sequence |
| V$CDXA_01 | CdxA | 4 (−) | 1.000 | 1.000 | caTAAAT |
| | | | | | SEQ ID No. 352 |
| V$SATB1_Q5_ | SATB1 | 5 (−) | 1.000 | 1.000 | ATAAAt |
| 01 | | | | | SEQ ID No. 353 |
| V$MEF2C_Q4 | MEF-2C | 5 (−) | 1.000 | 1.000 | atAAATA |
| | | | | | SEQ ID No. 354 |
| V$CDX1_Q5 | Cdx-1 | 8 (−) | 1.000 | 1.000 | aATAAA |
| | | | | | SEQ ID No. 355 |
| V$SATB1_Q5_ | SATB1 | 9 (−) | 1.000 | 1.000 | ATAAAt |
| 01 | | | | | SEQ ID No. 356 |
| V$GEN_INI_B | GEN_INI | 11 (−) | 0.996 | 0.997 | AAATGagg |
| | | | | | SEQ ID No. 357 |
| V$GEN_INI3_ | GEN_INI | 11 (−) | 0.997 | 0.997 | AAATGagg |
| B | | | | | SEQ ID No. 357 |
| V$GEN_INI2_ | GEN_INI | 11 (−) | 1.000 | 1.000 | AAATGagg |
| B | | | | | SEQ ID No. 357 |
| V$BBX_04 | BBX secondary | 14 (+) | 1.000 | 0.922 | tgaggGTTAAaagttg |
| | motif | | | | t |
| | | | | | SEQ ID No. 358 |
| V$HNF1B_Q6_ | HNF-1beta | 18 (−) | 1.000 | 0.912 | gGTTAAaagttgtc |
| 01 | | | | | SEQ ID No. 358 |
| V$FOXL2_Q2 | FOXL2 | 23 (−) | 0.983 | 0.952 | AAAGTtgtctgg |
| | | | | | SEQ ID No. 359 |
| V$SMAD3_03 | Smad3 | 23 (−) | 1.000 | 0.989 | aaagttGTCTGggaga |
| | | | | | g |
| | | | | | SEQ ID No. 360 |
| V$CP2_Q4 | CP2 | 27 (−) | 1.000 | 0.988 | ttgtCTGGGaga |
| | | | | | SEQ ID No. 361 |
| V$CP2_Q6 | CP2 | 27 (+) | 1.000 | 1.000 | ttgTCTGGga |
| | | | | | SEQ ID No. 362 |
| V$BRN1_Q6 | BRN1 | 40 (+) | 1.000 | 1.000 | aAATGCt |
| | | | | | SEQ ID No. 363 |
| V$NKX3A_02 | Nkx3A | 54 (−) | 1.000 | 0.934 | tggtgaaGTACTttttc |
| | | | | | SEQ ID No. 364 |
| V$NKX3A_02 | Nkx3A | 55 (+) | 1.000 | 0.904 | ggtgaAGTACtttttct |
| | | | | | SEQ ID No. 365 |
| V$GATA1_05 | GATA-1 | 73 (+) | 1.000 | 0.967 | gagGATAAca |
| | | | | | SEQ ID No. 366 |
| V$HLTF_Q4 | HLTF | 86 (+) | 0.966 | 0.892 | ggcaAGAAAgaagat |
| | | | | | gc |
| | | | | | SEQ ID No. 367 |
| V$ZNF35_04 | ZNF35 | 87 (+) | 1.000 | 1.000 | gCAAGA |
| | | | | | SEQ ID No. 368 |
| V$PARP_Q4 | PARP | 89 (−) | 1.000 | 1.000 | aAGAAA |
| | | | | | SEQ ID No. 292 |
| V$PRDM16_ | MEL1 | 95 (+) | 1.000 | 1.000 | GAAGAt |
| 05 | | | | | SEQ ID No. 369 |
| V$RFX1_02 | RFX1 | 96 (+) | 0.982 | 0.931 | aagatgcaaggGAAA |
| | | | | | Cct |
| | | | | | SEQ ID No. 370 |
| V$EFC_Q6 | RFX1 (EF-C) | 97 (+) | 0.820 | 0.864 | aGATGCaagggaaa |
| | | | | | SEQ ID No. 371 |
| V$RXRA_04 | RXR-ALPHA | 104 (−) | 1.000 | 0.902 | agggaaACCTTcatga |
| | secondary motif | | | | SEQ ID No. 372 |
| V$SPIB_Q3 | Spi-B | 118 (−) | 1.000 | 1.000 | gAGGAA |
| | | | | | SEQ ID No. 373 |
| V$ELF1_Q5 | Elf-1 | 119 (+) | 1.000 | 1.000 | AGGAAg |
| | | | | | SEQ ID No. 116 |
| V$SPI1_Q5 | PU.1 | 119 (+) | 1.000 | 1.000 | AGGAAg |
| | | | | | SEQ ID No. 116 |
| V$ZNF35_04 | ZNF35 | 120 (+) | 1.000 | 1.000 | gGAAGA |
| | | | | | SEQ ID No. 143 |
| V$PRDM16_ | MEL1 | 121 (+) | 1.000 | 1.000 | GAAGAt |
| 05 | | | | | SEQ ID No. 369 |
| V$CEBPE_Q6 | C/EBPepsilon | 121 (+) | 1.000 | 0.971 | gaagATTTCacaac |
| | | | | | SEQ ID No. 374 |
| V$CEBPA_05 | C/EBPalpha | 123 (−) | 0.902 | 0.932 | agaTTTCAcaact |
| | | | | | SEQ ID No. 375 |
| V$CEBPB_01 | C/EBPbeta | 123 (−) | 0.996 | 0.980 | agATTTCacaactg |
| | | | | | SEQ ID No. 376 |
| V$CEBPG_Q6_ | C/EBPgamma | 124 (−) | 1.000 | 0.983 | gATTTCacaact |
| 01 | | | | | SEQ ID No. 377 |
| V$CEBPA_03 | CEBPA | 125 (+) | 0.930 | 0.945 | aTTTCAcaact |
| | | | | | SEQ ID No. 378 |
| V$EHF_07 | EHF secondary | 143 (−) | 0.968 | 0.946 | atgctAGGAActggtt |
| | motif | | | | SEQ ID No. 379 |
| V$BCL6_Q3_ | Bcl-6 | 144 (−) | 0.984 | 0.966 | tgctAGGAAc |
| 01 | | | | | SEQ ID No. 380 |
| V$STAT5A_ | STAT5A | 144 (+) | 0.991 | 0.834 | tgctaGGAACtggtttg |
| 02 | (homotetramer) | | | | cttctgg |
| | | | | | SEQ ID No. 381 |
| V$CMYB_01 | c-Myb | 160 (+) | 0.988 | 0.937 | gcttctggctGTTGTct |
| | | | | | c |
| | | | | | SEQ ID No. 382 |
| V$RXRRAR_ | RXR: RAR | 174 (−) | 0.931 | 0.847 | tctccttagggTGAGCt |
| 01 | | | | | SEQ ID No. 383 |
| V$SREBP2_Q6_ | SREBP-2 | 178 (−) | 1.000 | 0.989 | cttagGGTGAg |
| 01 | | | | | SEQ ID No. 384 |
| V$SREBP_Q3 | SREBP | 180 (−) | 1.000 | 0.982 | taGGGTGagctc |
| | | | | | SEQ ID No. 385 |
| V$PAX4_03 | Pax-4 | 181 (−) | 1.000 | 0.956 | aGGGTGagctct |
| | | | | | SEQ ID No. 386 |
| V$SMAD3_03 | Smad3 | 187 (−) | 1.000 | 0.990 | agctctGTCTGgtgatt |
| | | | | | SEQ ID No. 387 |
| V$SMAD3_Q6_ | SMAD3 | 191 (−) | 1.000 | 1.000 | ctGTCTG |
| 02 | | | | | SEQ ID No. 388 |
| V$SMAD4_ | Smad4 | 191 (−) | 1.000 | 1.000 | ctGTCTGg |
| Q4 | | | | | SEQ ID No. 389 |
| V$SMAD2_ | Smad2 | 191 (−) | 1.000 | 1.000 | cTGTCT |
| Q6 | | | | | SEQ ID No. 390 |
| V$POU3F1_ | POU3F1 | 196 (+) | 0.939 | 0.898 | tggtgattAGCATcacc |
| Q6 | | | | | a |
| | | | | | SEQ ID No. 391 |
| V$OCT_C | OCT-x | 198 (+) | 0.884 | 0.882 | gtgaTTAGCatca |
| | | | | | SEQ ID No. 392 |
| V$PMX1_Q6 | PMX1 | 199 (−) | 1.000 | 1.000 | tGATTA |
| | | | | | SEQ ID No. 393 |
| V$OCT4_02 | Oct-4 (POU5F1) | 200 (−) | 0.723 | 0.875 | gattagcatcACCAT |
| | | | | | SEQ ID No. 394 |
| V$CRX_Q4_ | CRX | 200 (−) | 1.000 | 1.000 | GATTAg |
| 01 | | | | | SEQ ID No. 395 |
| V$POU3F2_ | POU3F2 | 201 (−) | 0.783 | 0.879 | attagCATCA |
| 02 | | | | | SEQ ID No. 396 |
| V$NANOG_ | nanog | 201 (−) | 0.661 | 0.848 | attagcatcACCATgg |
| 10 | | | | | a |
| | | | | | SEQ ID No. 397 |
| V$TEAD4PITX1_ | TEF-3: pitx1 | 205 (+) | 0.862 | 0.925 | gCATCAccatggatta |
| 01 | | | | | SEQ ID No. 398 |
| V$SIN3A_01 | sin3A | 206 (−) | 0.775 | 0.769 | cATCACcatggatt |
| | | | | | SEQ ID No. 399 |
| V$CDP_02 | CDP | 210 (+) | 0.907 | 0.908 | accATGGAttaaatt |
| | | | | | SEQ ID No. 400 |
| V$CLOX_01 | Clox | 210 (+) | 0.909 | 0.904 | accATGGAttaaatt |
| | | | | | SEQ ID No. 400 |
| V$CUX1_07 | CDP | 211 (−) | 0.928 | 0.931 | ccATGGAtta |
| | | | | | SEQ ID No. 401 |
| V$DMBX1_02 | DMBX1 | 213 (+) | 1.000 | 0.996 | atgGATTAaa |
| | | | | | SEQ ID No. 402 |
| V$GTF2IRD1_ | GTF2IRD1-isoform2 | 214 (+) | 1.000 | 0.959 | tgGATTAaa |
| 01 | | | | | SEQ ID No. 403 |
| V$CRX_Q4_ | CRX | 216 (−) | 1.000 | 1.000 | GATTAa |
| 01 | | | | | SEQ ID No. 404 |
| V$NCX_02 | Ncx | 216 (+) | 1.000 | 0.895 | gattaaATTAAttggct |
| | | | | | SEQ ID No. 405 |
| V$LHX3_Q3 | LHX3 | 217 (+) | 1.000 | 1.000 | ATTAAa |
| | | | | | SEQ ID No. 406 |
| V$NCX_02 | Ncx | 217 (−) | 1.000 | 0.892 | attaaaTTAATtggctg |
| | | | | | SEQ ID No. 407 |
| V$HOXA9_ | Hoxa9 | 218 (−) | 1.000 | 0.978 | ttaAATTAat |
| Q5_01 | | | | | SEQ ID No. 408 |
| V$PRX2_Q2 | Prx2 | 218 (+) | 0.990 | 0.992 | TTAAAttaa |
| | | | | | SEQ ID No. 409 |
| V$LHX3b_01 | LHX3b | 219 (−) | 1.000 | 0.977 | taaATTAAtt |
| | | | | | SEQ ID No. 410 |
| V$MSX2_01 | Msx-2 | 219 (−) | 1.000 | 0.916 | taaatTAATTggctgta |
| | | | | | SEQ ID No. 411 |
| V$S8_01 | S8 | 220 (−) | 1.000 | 0.995 | aaatTAATTggctgta |
| | | | | | SEQ ID No. 412 |
| V$HMX3_03 | HMX3 | 221 (−) | 1.000 | 0.990 | aattAATTGgc |
| | | | | | SEQ ID No. 413 |
| V$HMX2_02 | HMX2 | 221 (−) | 1.000 | 0.981 | aattAATTGgc |
| | | | | | SEQ ID No. 413 |
| V$HMX1_05 | HMX1 | 221 (−) | 1.000 | 0.974 | aattAATTGgc |
| | | | | | SEQ ID No. 413 |
| V$DRI1_01 | DRI1 | 221 (+) | 1.000 | 1.000 | aATTAA |
| | | | | | SEQ ID No. 414 |
| V$DLX5_Q3 | DIx-5 | 221 (+) | 1.000 | 1.000 | AATTAa |
| | | | | | SEQ ID No. 414 |
| V$PRRX2_03 | Prrx2 | 221 (+) | 1.000 | 1.000 | AATTA |
| | | | | | SEQ ID No. 415 |
| V$V$X1_03 | V$X1 | 222 (−) | 0.992 | 0.989 | attAATTGgct |
| | | | | | SEQ ID No. 416 |
| V$RAX_03 | RAX | 222 (−) | 1.000 | 1.000 | atTAATTggc |
| | | | | | SEQ ID No. 417 |
| V$LBX2_04 | LBX2 | 222 (−) | 1.000 | 0.998 | atTAATTggc |
| | | | | | SEQ ID No. 417 |
| V$GBX1_04 | Gbx1 | 222 (−) | 1.000 | 0.999 | atTAATTggc |
| | | | | | SEQ ID No. 417 |
| V$GSX2_02 | GSX2 | 222 (−) | 1.000 | 0.988 | atTAATTggc |
| | | | | | SEQ ID No. 417 |
| V$GBX2_04 | GBX2 | 222 (−) | 1.000 | 0.998 | atTAATTggc |
| | | | | | SEQ ID No. 417 |
| V$ESX1_03 | ESX1 | 222 (−) | 1.000 | 0.997 | atTAATTggc |
| | | | | | SEQ ID No. 417 |
| V$BARHL1_ | Barhl1 | 222 (+) | 1.000 | 0.997 | atTAATTggc |
| 04 | | | | | SEQ ID No. 417 |
| V$RAX2_01 | RAX2 | 223 (−) | 1.000 | 1.000 | tTAATTgg |
| | | | | | SEQ ID No. 418 |
| V$SHOX2_03 | SHOX2 | 223 (−) | 1.000 | 1.000 | tTAATTgg |
| | | | | | SEQ ID No. 418 |
| V$DLX3_Q6 | DIx-3 | 223 (+) | 1.000 | 0.999 | tTAATTggc |
| | | | | | SEQ ID No. 419 |
| V$SHOX_02 | SHOX | 223 (+) | 1.000 | 1.000 | tTAATTgg |
| | | | | | SEQ ID No. 418 |
| V$HOXA6_03 | HOXA6 | 223 (−) | 1.000 | 0.991 | tTAATTgg |
| | | | | | SEQ ID No. 418 |
| V$HOXA7_08 | HOXA7 | 223 (−) | 1.000 | 0.997 | tTAATTgg |
| | | | | | SEQ ID No. 418 |
| V$PRRX2_06 | PRRX2 | 223 (−) | 1.000 | 1.000 | tTAATTgg |
| | | | | | SEQ ID No. 418 |
| V$PRRX1_02 | PRRX1 | 223 (−) | 1.000 | 1.000 | tTAATTgg |
| | | | | | SEQ ID No. 418 |
| V$DRI1_01 | DRI1 | 223 (−) | 1.000 | 1.000 | TTAATt |
| | | | | | SEQ ID No. 420 |
| V$DLX5_Q3 | DIx-5 | 223 (−) | 1.000 | 1.000 | tTAATT |
| | | | | | SEQ ID No. 420 |
| V$BSX_03 | BSX | 223 (−) | 1.000 | 1.000 | tTAATTgg |
| | | | | | SEQ ID No. 418 |
| V$DLX2_03 | DIx-2 | 223 (−) | 1.000 | 1.000 | tTAATTgg |
| | | | | | SEQ ID No. 418 |
| V$LHX9_03 | LHX9 | 223 (−) | 1.000 | 1.000 | tTAATTgg |
| | | | | | SEQ ID No. 418 |
| V$MSX2_04 | MSX2 | 223 (−) | 1.000 | 1.000 | tTAATTgg |
| | | | | | SEQ ID No. 418 |
| V$OG2_01 | OG-2 | 224 (+) | 1.000 | 1.000 | TAATTg |
| | | | | | SEQ ID No. 421 |
| V$PRRX2_03 | Prrx2 | 224 (−) | 1.000 | 1.000 | TAATT |
| | | | | | SEQ ID No. 422 |
| V$YB1_Q4 | YB-1 | 224 (−) | 1.000 | 0.994 | taATTGGctgt |
| | | | | | SEQ ID No. 423 |
| V$NF1C_Q6 | NF-1C | 227 (+) | 1.000 | 1.000 | TTGGCtg |
| | | | | | SEQ ID No. 288 |
| V$GTF2IRD1_ | GTF2IRD1-isoform2 | 236 (−) | 0.973 | 0.966 | ctCAATCcc |
| 01 | | | | | SEQ ID No. 424 |

TABLE 9
Transcription factor Position Weight Matrix (PWM, Transfac) recognizing the
hGUCA1A promoter (−250 bp from TSS)

| | | Position | Core | Matrix | |
|---|---|---|---|---|---|
| Matrix | Factor name | (strand) | score | score | Sequence |
| V$TFIII_Q6_01 | TFII-I | 5 (−) | 0.984 | 0.989 | tcacCTCCTg |
| | | | | | SEQ ID No. 425 |
| V$THAP1_03 | THAP1 | 12 (+) | 1.000 | 0.995 | cTGCCCaca |
| | | | | | SEQ ID No. 426 |
| V$STAT3_Q4 | STAT3 | 40 (+) | 1.000 | 0.974 | ttgtGGGAAg |
| | | | | | SEQ ID No. 427 |
| V$PPARGRXRA_ | PPARgamma: | 41 (+) | 0.961 | 0.853 | tgtgggaAGAGGga |
| 01 | RXR-alpha | | | | a |
| | | | | | SEQ ID No. 428 |
| V$ZNF35_04 | ZNF35 | 45 (+) | 1.000 | 1.000 | gGAAGA |
| | | | | | SEQ ID No. 143 |
| V$PUR1_Q4 | PUR1 | 65 (+) | 1.000 | 1.000 | ggGCCAGtg |
| | | | | | SEQ ID No. 429 |
| V$PAX5_07 | Pasx-5 | 73 (+) | 0.871 | 0.889 | GTCAAggtt |
| | | | | | SEQ ID No. 430 |
| V$XBP1_02 | XBP-1 | 99 (+) | 1.000 | 0.870 | cctgaaACGTC |
| | | | | | SEQ ID No. 431 |
| V$ATF_01 | ATF | 100 (−) | 1.000 | 0.955 | ctgaaaCGTCAgtc |
| | | | | | SEQ ID No. 432 |
| V$ATF_B | ATF | 100 (−) | 1.000 | 0.947 | ctgaaaCGTCAg |
| | | | | | SEQ ID No. 433 |
| V$CREBP1_Q2 | ATF2 | 101 (−) | 1.000 | 0.899 | tgaaaCGTCAgt |
| | | | | | SEQ ID No. 434 |
| V$CREB_Q2_01 | CREB | 101 (+) | 1.000 | 0.950 | tgaaaCGTCAgtcc |
| | | | | | SEQ ID No. 435 |
| V$ATF1_Q6_01 | ATF-1 | 103 (+) | 1.000 | 0.972 | aaaCGTCAg |
| | | | | | SEQ ID No. 436 |
| V$CREM_Q6 | CREM | 103 (+) | 1.000 | 0.960 | aaaCGTCAgtc |
| | | | | | SEQ ID No. 437 |
| V$CREBATF_Q6 | CREB, ATF | 103 (−) | 1.000 | 0.981 | aaaCGTCAg |
| | | | | | SEQ ID No. 438 |
| V$CREB_Q4_01 | CREB | 103 (−) | 1.000 | 0.954 | aaaCGTCAgtc |
| | | | | | SEQ ID No. 439 |
| V$CREB_01 | CREB | 103 (−) | 1.000 | 0.946 | aaaCGTCA |
| | | | | | SEQ ID No. 440 |
| V$ATF3_02 | ATF-3 | 103 (−) | 1.000 | 0.832 | aaACGTCagtcccca |
| | | | | | gccct |
| | | | | | SEQ ID No. 441 |
| V$CREBP1CJUN_ | ATF2: c-Jun | 103 (−) | 1.000 | 0.891 | aaACGTCa |
| 01 | | | | | SEQ ID No. 440 |
| V$GEN_INI2_B | GEN_INI | 106 (+) | 0.998 | 0.992 | cgtCAGTC |
| | | | | | SEQ ID No. 442 |
| V$GEN_INI3_B | GEN_INI | 106 (+) | 0.996 | 0.989 | cgtCAGTC |
| | | | | | SEQ ID No. 442 |
| V$GEN_INI_B | GEN_INI | 106 (+) | 0.999 | 0.991 | cgtCAGTC |
| | | | | | SEQ ID No. 442 |
| V$RELA_03 | RelA-p65 | 109 (−) | 1.000 | 0.986 | cagtcCCCAGc |
| | | | | | SEQ ID No. 443 |
| V$T3RBETA_ | T3R-beta | 121 (−) | 0.966 | 0.961 | cTGGCCtcatgtctcc |
| Q6_01 | | | | | t |
| | | | | | SEQ ID No. 444 |
| V$IK2_01 | Ik-2 | 135 (+) | 1.000 | 0.995 | cctTGGGAagac |
| | | | | | SEQ ID No. 445 |
| V$ZNF35_04 | ZNF35 | 140 (+) | 1.000 | 1.000 | gGAAGA |
| | | | | | SEQ ID No. 143 |
| V$SMAD2_Q6 | Smad2 | 143 (+) | 1.000 | 1.000 | AGACAg |
| | | | | | SEQ ID No. 185 |
| V$SOX5_07 | Sox-4 secondary | 148 (+) | 1.000 | 0.975 | gaaggaATTGTgttg |
| | motif | | | | ta |
| | | | | | SEQ ID No. 446 |
| V$SOX7_04 | Sox-7 secondary | 148 (+) | 1.000 | 0.867 | gaaggaATTGTgttg |
| | motif | | | | taaagag |
| | | | | | SEQ ID No. 447 |
| V$NANOG_10 | nanog | 151 (+) | 0.982 | 0.858 | ggaATTGTgttgtaa |
| | | | | | ag |
| | | | | | SEQ ID No. 448 |
| V$FOXJ1_04 | FOXJ1 | 154 (−) | 1.000 | 0.925 | attgtGTTGTaaaga |
| | secondary motif | | | | SEQ ID No. 449 |
| V$OCT4_02 | Oct-4 (POU5F1) | 154 (+) | 1.000 | 0.905 | ATTGTgttgtaaaga |
| | | | | | SEQ ID No. 450 |
| V$BRCA_01 | BRCA1: USF2 | 155 (+) | 1.000 | 0.999 | ttgTGTTG |
| | | | | | SEQ ID No. 451 |
| V$ZNF14_02 | ZNF14 | 159 (+) | 1.000 | 1.000 | gttGTAAAga |
| | | | | | SEQ ID No. 452 |
| V$HOXC10EOMES_ | HOXC10: TBR2 | 162 (−) | 1.000 | 0.916 | gtaaagAGGTGtca |
| 01 | | | | | SEQ ID No. 453 |
| V$HOXA3EOMES_ | HOXA3: TBR2 | 163 (+) | 0.843 | 0.915 | TAAAGaggtgtca |
| 01 | | | | | SEQ ID No. 454 |
| V$SIX4_01 | six-4 | 164 (−) | 1.000 | 0.963 | aaagaGGTGTcaca |
| | | | | | atg |
| | | | | | SEQ ID No. 455 |
| V$TBX5_01 | Tbx5 | 166 (+) | 1.000 | 0.998 | agaGGTGTcaca |
| | | | | | SEQ ID No. 456 |
| V$ESR2_03 | ER-beta | 168 (−) | 0.829 | 0.833 | aggTGTCAcaatgcc |
| | | | | | ccc |
| | | | | | SEQ ID No. 457 |
| V$ESR2_01 | ESR2 | 168 (+) | 0.903 | 0.880 | aggTGTCAcaatgcc |
| | | | | | ccc |
| | | | | | SEQ ID No. 458 |
| V$TBX3_04 | Tbx3 | 168 (+) | 1.000 | 1.000 | aGGTGTca |
| | | | | | SEQ ID No. 459 |
| V$ESR1_01 | ESR1 | 170 (−) | 0.880 | 0.828 | gTGTCAcaatgcccc |
| | | | | | ctgcc |
| | | | | | SEQ ID No. 460 |
| V$ESR1_04 | ER-alpha | 170 (+) | 0.941 | 0.924 | gTGTCAcaatgccc |
| | | | | | SEQ ID No. 461 |
| V$SOX10_Q6_ | Sox-10 | 174 (+) | 1.000 | 1.000 | cACAATg |
| 01 | | | | | SEQ ID No. 462 |
| V$SOX10_Q3 | Sox-10 | 175 (+) | 1.000 | 0.999 | ACAATgcc |
| | | | | | SEQ ID No. 463 |
| V$ZNF515_Q6 | ZNF515 | 178 (+) | 1.000 | 0.960 | atgCCCCCtgccc |
| 01 | | | | | SEQ ID No. 464 |
| V$ZNF300_04 | ZNF300 | 179 (−) | 1.000 | 0.991 | tgcCCCCTg |
| | | | | | SEQ ID No. 465 |
| V$PPARGRXRA_ | PPARgamma: | 179 (−) | 0.798 | 0.859 | tgcCCCCTgccccat |
| 01 | RXR-alpha | | | | SEQ ID No. 466 |
| V$AP2ALPHA_ | AP-2alpha | 179 (+) | 1.000 | 0.992 | tGCCCCctgcc |
| Q6 | | | | | SEQ ID No. 467 |
| V$AP2BETA_ | AP-2beta | 180 (−) | 1.000 | 0.975 | gCCCCCtgccccata |
| Q3 | | | | | c |
| | | | | | SEQ ID No. 468 |
| V$CPBP_Q6 | CPBP | 181 (+) | 1.000 | 1.000 | CCCCCtg |
| | | | | | SEQ ID No. 231 |
| V$PUR1_Q4 | PUR1 | 183 (−) | 1.000 | 0.999 | ccCTGCCcc |
| | | | | | SEQ ID No. 469 |
| V$CPBP_Q6 | CPBP | 187 (+) | 1.000 | 1.000 | GCCCCat |
| | | | | | SEQ ID No. 470 |
| V$SREBP1_02 | SREBP-1 | 198 (+) | 0.800 | 0.852 | gtTCTCCccac |
| | | | | | SEQ ID No. 471 |
| V$SPIB_Q3 | Spi-B | 199 (+) | 1.000 | 1.000 | TTCTCc |
| | | | | | SEQ ID No. 256 |
| V$MZF1_Q5 | MZF-1 | 201 (−) | 1.000 | 1.000 | cTCCCCa |
| | | | | | SEQ ID No. 472 |
| V$KID3_01 | Kid3 | 205 (+) | 1.000 | 1.000 | CCACC |
| | | | | | SEQ ID No. 197 |
| V$NFIA_Q6_01 | NF-1A | 209 (−) | 1.000 | 1.000 | cTTGGCa |
| | | | | | SEQ ID No. 473 |
| V$IRF6_04 | IRF6 secondary | 212 (+) | 0.904 | 0.915 | ggcacTCTCAgtatc |
| | motif | | | | SEQ ID No. 474 |
| V$KAISO_01 | Kaiso | 224 (+) | 1.000 | 0.994 | atCCTGCcaa |
| | | | | | SEQ ID No. 475 |
| V$NF1_Q6_02 | NF-1 | 227 (−) | 1.000 | 1.000 | ctGCCAA |
| | | | | | SEQ ID No. 476 |
| V$NF1A_Q6_01 | NF-1A | 228 (+) | 1.000 | 1.000 | tGCCAAg |
| | | | | | SEQ ID No. 477 |
| V$GKLF_Q4 | GKLF | 238 (+) | 1.000 | 1.000 | CCTCCct |
| | | | | | SEQ ID No. 122 |

TABLE 10
Transcription factor Position Weight Matrix (PWM, Transfac) recognizing the
hGUCYD2 promoter (−250 bp from TSS)

| | | Position | Core | Matrix | |
|---|---|---|---|---|---|
| Matrix | Factor name | (strand) | score | score | Sequence |
| V$LTF_Q6 | LTF | 2 (+) | 1.000 | 0.984 | gGCACTtgt |
| | | | | | SEQ ID No. 488 |
| V$IRF4_Q6 | IRF-4 | 11 (−) | 1.000 | 1.000 | taCTTTC |
| | | | | | SEQ ID No. 489 |
| V$NF1B_ | NF-1B | 13 (−) | 1.000 | 0.997 | ctttCTGGC |
| Q6_01 | | | | | SEQ ID No. 490 |
| V$ZIC3_05 | ZIC3 secondary motif | 14 (−) | 0.890 | 0.869 | tttctGGCTGagcag |
| | | | | | SEQ ID No. 491 |
| V$PAX5_01 | Pax-5 | 18 (+) | 0.988 | 0.884 | tggctgagCAGGGcagt |
| | | | | | gtggccgacgg |
| | | | | | SEQ ID No. 492 |
| V$CMAF_ | c-MAF | 19 (−) | 1.000 | 0.943 | gGCTGAgcagg |
| Q5 | | | | | SEQ ID No. 493 |
| V$ESR1_01 | ESR1 | 22 (+) | 0.927 | 0.868 | tgagcagggcagtgTGG |
| | | | | | CCg |
| | | | | | SEQ ID No. 494 |
| V$PPARG_ | PPARgamma: RXRalpha, | 23 (−) | 0.832 | 0.690 | gagcagggcagtgTGGC |
| 02 | PPARgamma | | | | Cgacgg |
| | | | | | SEQ ID No. 495 |
| V$PPARG_ | PPARgamma: RXRalpha, | 23 (+) | 0.853 | 0.712 | gagcaGGGCAgtgtgg |
| 02 | PPARgamma | | | | ccgacgg |
| | | | | | SEQ ID No. 496 |
| V$ESR2_03 | ER-beta | 25 (−) | 0.951 | 0.830 | gcaGGGCAgtgtggcc |
| | | | | | ga |
| | | | | | SEQ ID No. 497 |
| V$ESR2_01 | ESR2 | 26 (−) | 0.876 | 0.873 | cagggcagtgTGGCCg |
| | | | | | ac |
| | | | | | SEQ ID No. 498 |
| V$ESR2_03 | ER-beta | 26 (+) | 0.934 | 0.887 | cagggcagtgTGGCCg |
| | | | | | ac |
| | | | | | SEQ ID No. 499 |
| V$ESR1_01 | ESR1 | 27 (−) | 0.947 | 0.876 | aGGGCAgtgtggccga |
| | | | | | cggc |
| | | | | | SEQ ID No. 500 |
| V$ESR1_04 | ER-alpha | 27 (+) | 0.841 | 0.873 | aGGGCAgtgtggcc |
| | | | | | SEQ ID No. 501 |
| V$SP100_ | SP100 secondary motif | 32 (−) | 1.000 | 0.931 | agtgtggcCGACGgc |
| 04 | | | | | SEQ ID No. 502 |
| V$RXRA_ | RXR-ALPHA | 43 (+) | 1.000 | 0.816 | cggctgAAAGGggaa |
| 13 | | | | | SEQ ID No. 503 |
| V$ZNF675_ | ZNF675 | 47 (+) | 1.000 | 1.000 | tgaAAGGGga |
| 01 | | | | | SEQ ID No. 504 |
| V$PU1_Q4 | PU.1 | 48 (−) | 0.962 | 0.929 | gaaagGGGAAgctgcg |
| | | | | | gct |
| | | | | | SEQ ID No. 505 |
| V$SPIB_Q3 | Spi-B | 52 (−) | 1.000 | 1.000 | gGGGAA |
| | | | | | SEQ ID No. 209 |
| V$SOX9_ | Sox-9 | 63 (−) | 1.000 | 0.971 | ggctgctTTTGTgcagg |
| Q5 | | | | | SEQ ID No. 507 |
| V$BCL6B_ | BCL6B secondary | 73 (−) | 0.967 | 0.964 | gtgcagGGGTGgtggt |
| 04 | motif | | | | SEQ ID No. 508 |
| V$ZNF300_ | ZNF300 | 76 (+) | 1.000 | 0.984 | cAGGGGtgg |
| 04 | | | | | SEQ ID No. 509 |
| V$NR2C2_ | TR4 | 76 (−) | 1.000 | 0.871 | caGGGGT |
| 04 | | | | | SEQ ID No. 510 |
| V$ZIC1_01 | Zic1 | 78 (+) | 1.000 | 0.900 | ggGGTGGtg |
| | | | | | SEQ ID No. 511 |
| V$LKLF_ | LKLF | 78 (+) | 1.000 | 1.000 | gGGGTGgtgg |
| Q3 | | | | | SEQ ID No. 512 |
| V$ZIC3_01 | Zic3 | 78 (+) | 1.000 | 0.936 | gGGGTGgtg |
| | | | | | SEQ ID No. 511 |
| V$PAX4_03 | Pax-4 | 78 (−) | 1.000 | 0.991 | gGGGTGgtggtg |
| | | | | | SEQ ID No. 513 |
| V$KID3_01 | Kid3 | 80 (−) | 1.000 | 1.000 | GGTGG |
| | | | | | SEQ ID No. 322 |
| V$ING4_01 | ING4 | 82 (−) | 1.000 | 1.000 | TGGTGg |
| | | | | | SEQ ID No. 514 |
| V$KID3_01 | Kid3 | 83 (−) | 1.000 | 1.000 | GGTGG |
| | | | | | SEQ ID No. 322 |
| V$PPARA_ | PPARalpha: RXRalpha | 83 (+) | 0.945 | 0.932 | ggtGGTGAtgagggtga |
| 02 | | | | | tg |
| | | | | | SEQ ID No. 515 |
| V$GCM1FOXI1_ | GCMa: FOXI1 | 84 (+) | 0.957 | 0.927 | gtggtgatGAGGGt |
| 01 | | | | | SEQ ID No. 516 |
| V$PRDM16_ | MEL1 | 89 (+) | 1.000 | 1.000 | GATGAg |
| 04 | | | | | SEQ ID No. 517 |
| V$RREB1_ | RREB-1 | 93 (−) | 0.901 | 0.889 | agggtgatGTGGGg |
| 01 | | | | | SEQ ID No. 518 |
| V$CPBP_ | CPBP | 101 (−) | 1.000 | 1.000 | gtGGGGG |
| Q6 | | | | | SEQ ID No. 519 |
| V$MZF1_02 | MZF-1 | 117 (+) | 1.000 | 0.959 | catggAGGGGaaa |
| | | | | | SEQ ID No. 520 |
| V$SPIB_Q3 | Spi-B | 123 (−) | 1.000 | 1.000 | gGGGAA |
| | | | | | SEQ ID No. 209 |
| V$IRF3_06 | IRF3 secondary motif | 123 (+) | 1.000 | 0.910 | ggggAAAGGatctg |
| | | | | | SEQ ID No. 521 |
| V$SMAD4_ | SMAD4 | 132 (−) | 1.000 | 0.894 | atcTGGCTgactacc |
| Q6 | | | | | SEQ ID No. 522 |
| V$GTF3C2_ | TF3C-beta | 133 (+) | 0.830 | 0.753 | tctggctgactacctGGA |
| 01 | | | | | AGcc |
| | | | | | SEQ ID No. 523 |
| V$MAFB_ | MafB | 137 (+) | 1.000 | 1.000 | GCTGAc |
| 01 | | | | | SEQ ID No. 524 |
| V$DRRS_ | DRRS | 137 (−) | 1.000 | 1.000 | gctGACTAcc |
| 02 | | | | | SEQ ID No. 525 |
| V$STAT3_ | STAT3 | 141 (+) | 0.974 | 0.929 | actaccTGGAAgccag |
| 03 | | | | | SEQ ID No. 526 |
| V$REST_16 | REST | 146 (+) | 1.000 | 0.828 | ctggaagccagGACAG |
| | | | | | atccc |
| | | | | | SEQ ID No. 527 |
| V$REST_01 | REST | 148 (−) | 1.000 | 0.835 | ggaagccaGGACAgat |
| | | | | | cccacc |
| | | | | | SEQ ID No. 528 |
| V$GR_Q6 | GR | 153 (−) | 0.989 | 0.958 | ccaGGACAgatcccacc |
| | | | | | cc |
| | | | | | SEQ ID No. 529 |
| V$PAX4_03 | Pax-4 | 160 (+) | 1.000 | 0.986 | agatccCACCCc |
| | | | | | SEQ ID No. 530 |
| V$BCL6B_ | BCL6B secondary | 161 (+) | 0.967 | 0.969 | gatccCACCCcagaaa |
| 04 | motif | | | | SEQ ID No. 531 |
| V$SALL2_ | SALL2 | 164 (−) | 1.000 | 1.000 | ccCACCC |
| 01 | | | | | SEQ ID No. 532 |
| V$KID3_01 | Kid3 | 165 (+) | 1.000 | 1.000 | CCACC |
| | | | | | SEQ ID No. 197 |
| V$ZNF300_ | ZNF300 | 165 (−) | 1.000 | 0.984 | ccaCCCCAg |
| 04 | | | | | SEQ ID No. 533 |
| V$SREBP1_ | SREBP-1 | 166 (+) | 1.000 | 1.000 | CACCCca |
| Q6 | | | | | SEQ ID No. 534 |
| V$SOX9_ | Sox-9 | 167 (+) | 0.937 | 0.940 | accccAGAAAggcgca |
| Q5 | | | | | g |
| | | | | | SEQ ID No. 535 |
| V$NR2C2_ | TR4 | 167 (+) | 1.000 | 0.871 | ACCCCag |
| 04 | | | | | SEQ ID No. 536 |
| V$IRF3_06 | IRF3 secondary motif | 170 (+) | 1.000 | 0.963 | ccagAAAGGcgcag |
| | | | | | SEQ ID No. 537 |
| V$ZIC3_05 | ZIC3 secondary motif | 176 (+) | 0.863 | 0.892 | aggcgCAGTAggggc |
| | | | | | SEQ ID No. 538 |
| V$PRDM16_ | MEL1 | 192 (−) | 1.000 | 1.000 | cTCATC |
| 04 | | | | | SEQ ID No. 539 |
| V$ZF5_B | ZF5 | 204 (−) | 0.888 | 0.900 | taGCCCGcccctc |
| | | | | | SEQ ID No. 540 |
| V$BCL6B_ | BCL6B secondary | 204 (+) | 1.000 | 0.987 | tagccCGCCCctccct |
| 04 | motif | | | | SEQ ID No. 541 |
| V$SP1_01 | Sp1 | 205 (−) | 1.000 | 0.967 | agcCCGCCcc |
| | | | | | SEQ ID No. 542 |
| V$ETF_Q6_ | ETF | 206 (+) | 1.000 | 0.930 | gcCCGCCcctc |
| 01 | | | | | SEQ ID No. 543 |
| V$MAZ_Q6_ | MAZ | 207 (−) | 1.000 | 0.957 | cccgccCCTCCcta |
| 01 | | | | | SEQ ID No. 544 |
| V$GKLF_Q3 | GKLF | 208 (−) | 0.990 | 0.990 | ccgccCCTCCctac |
| | | | | | SEQ ID No. 545 |
| V$CPBP_Q6 | CPBP | 210 (+) | 1.000 | 1.000 | GCCCCtc |
| | | | | | SEQ ID No. 165 |
| V$GKLF_Q4 | GKLF | 213 (+) | 1.000 | 1.000 | CCTCCct |
| | | | | | SEQ ID No. 122 |
| V$PAX7_04 | PAX-7 | 214 (+) | 1.000 | 0.824 | ctccctacCTAAT |
| | | | | | SEQ ID No. 546 |
| V$OG2_02 | OG-2 | 217 (−) | 1.000 | 0.958 | cctacctAATTAaggac |
| | | | | | SEQ ID No. 547 |
| V$MSX2_01 | Msx-2 | 217 (+) | 1.000 | 0.903 | cctacctAATTAaggac |
| | | | | | SEQ ID No. 547 |
| V$DLX5_01 | Dlx-5 | 217 (−) | 1.000 | 0.949 | cctacctAATTAagga |
| | | | | | SEQ ID No. 548 |
| V$PAX4_05 | Pax-4 | 217 (−) | 1.000 | 0.946 | cctacctAATTAaggac |
| | | | | | SEQ ID No. 547 |
| V$CART1_ | CART1 | 217 (−) | 1.000 | 0.940 | cctacctAATTAaggac |
| 02 | | | | | SEQ ID No. 547 |
| V$POU6F1_ | POU6F1 | 217 (−) | 0.949 | 0.955 | cctaccTAATTaaggac |
| 03 | | | | | SEQ ID No. 547 |
| V$POU6F1_ | POU6F1 | 217 (−) | 0.915 | 0.899 | cctaccTAATTaaggac |
| 02 | | | | | SEQ ID No. 547 |
| V$CART1_ | CART1 | 218 (+) | 1.000 | 0.946 | ctaccTAATTaaggacc |
| 02 | | | | | SEQ ID No. 549 |
| V$PAX4_05 | Pax-4 | 218 (+) | 1.000 | 0.946 | ctaccTAATTaaggacc |
| | | | | | SEQ ID No. 549 |
| V$OG2_02 | OG-2 | 218 (+) | 1.000 | 0.966 | ctaccTAATTaaggacc |
| | | | | | SEQ ID No. 549 |
| V$DLX5_01 | Dlx-5 | 219 (+) | 1.000 | 0.948 | taccTAATTaaggacc |
| | | | | | SEQ ID No. 550 |
| V$DLX3_Q3 | Dlx-3 | 219 (+) | 1.000 | 0.993 | tacctAATTAag |
| | | | | | SEQ ID No. 551 |
| V$LHX2_Q6 | Lhx2 | 220 (−) | 1.000 | 0.998 | accTAATTaag |
| | | | | | SEQ ID No. 552 |
| V$V$X1_03 | V$X1 | 220 (+) | 1.000 | 0.960 | accTAATTaag |
| | | | | | SEQ ID No. 552 |
| V$IPF1_03 | ipf1 | 220 (+) | 1.000 | 0.972 | accTAATTaa |
| | | | | | SEQ ID No. 553 |
| V$GSX2_02 | GSX2 | 221 (+) | 1.000 | 0.997 | cctAATTAag |
| | | | | | SEQ ID No. 554 |
| V$HOXA2_ | HOXA2 | 221 (+) | 1.000 | 0.996 | cctAATTAag |
| 03 | | | | | SEQ ID No. 554 |
| V$HOXB2_ | HOXB2 | 221 (+) | 1.000 | 0.999 | cctAATTAag |
| 01 | | | | | SEQ ID No. 554 |
| V$HOXB5_ | HOXB5 | 221 (+) | 1.000 | 0.999 | cctAATTAag |
| 03 | | | | | SEQ ID No. 554 |
| V$HOXD3_ | Hoxd3 | 221 (+) | 1.000 | 0.998 | cctAATTAag |
| 03 | | | | | SEQ ID No. 554 |
| V$LHX2_02 | LHX2 | 221 (+) | 1.000 | 0.998 | cctAATTAag |
| | | | | | SEQ ID No. 554 |
| V$MEOX2_ | MEOX2 | 221 (+) | 1.000 | 0.987 | cctAATTAag |
| 01 | | | | | SEQ ID No. 554 |
| V$MIXL1_01 | MIXL1 | 221 (+) | 1.000 | 0.999 | cctAATTAag |
| | | | | | SEQ ID No. 554 |
| V$EMX1_04 | EMX1 | 221 (−) | 1.000 | 0.996 | cctAATTAag |
| | | | | | SEQ ID No. 554 |
| V$DLX1_05 | D1x1 | 221 (+) | 1.000 | 0.999 | cctAATTAag |
| | | | | | SEQ ID No. 554 |
| V$GSX1_01 | GSX1 | 221 (+) | 1.000 | 1.000 | cctAATTAag |
| | | | | | SEQ ID No. 554 |
| V$EVX2_03 | EVX2 | 221 (+) | 1.000 | 0.995 | cctAATTAag |
| | | | | | SEQ ID No. 554 |
| V$EVX1_03 | EVX1 | 221 (+) | 1.000 | 0.993 | cctAATTAag |
| | | | | | SEQ ID No. 554 |
| V$EMX1_01 | EMX1 | 221 (−) | 1.000 | 0.997 | ccTAATTaag |
| | | | | | SEQ ID No. 554 |
| V$GSX1_01 | GSX1 | 221 (−) | 1.000 | 0.991 | ccTAATTaag |
| | | | | | SEQ ID No. 554 |
| V$GSX2_02 | GSX2 | 221 (−) | 1.000 | 0.986 | ccTAATTaag |
| | | | | | SEQ ID No. 554 |
| V$HOXB5_ | HOXB5 | 221 (−) | 1.000 | 0.997 | ccTAATTaag |
| 03 | | | | | SEQ ID No. 554 |
| V$HOXD3_ | Hoxd3 | 221 (−) | 1.000 | 0.997 | ccTAATTaag |
| 03 | | | | | SEQ ID No. 554 |
| V$MIXL1_01 | MIXL1 | 221 (−) | 1.000 | 0.999 | ccTAATTaag |
| | | | | | SEQ ID No. 554 |
| V$EMX1_04 | EMX1 | 221 (+) | 1.000 | 0.996 | CcTAATTaag |
| | | | | | SEQ ID No. 554 |
| V$ALX3_03 | ALX3 | 221 (+) | 1.000 | 0.995 | cctAATTAag |
| | | | | | SEQ ID No. 554 |
| V$DLX1_03 | Dlx-1 | 221 (+) | 1.000 | 0.999 | cctAATTAag |
| | | | | | SEQ ID No. 554 |
| V$EMX1_01 | EMX1 | 221 (+) | 1.000 | 0.996 | cctAATTAag |
| | | | | | SEQ ID No. 554 |
| V$SHOX_01 | SHOX | 222 (+) | 1.000 | 1.000 | ctAATTAa |
| | | | | | SEQ ID No. 555 |
| V$SHOX2_ | Shox2 | 222 (+) | 1.000 | 1.000 | ctAATTAa |
| 04 | | | | | SEQ ID No. 555 |
| V$UNCX_03 | UNCX | 222 (+) | 1.000 | 1.000 | ctAATTAa |
| | | | | | SEQ ID No. 555 |
| V$UNCX_05 | Uncx | 222 (+) | 1.000 | 1.000 | ctAATTAa |
| | | | | | SEQ ID No. 555 |
| V$VAX1_03 | VAX1 | 222 (+) | 1.000 | 0.998 | ctAATTAa |
| | | | | | SEQ ID No. 555 |
| V$VAX2_03 | VAX2 | 222 (+) | 1.000 | 0.998 | ctAATTAa |
| | | | | | SEQ ID No. 555 |
| V$LBX1_02 | LBX1 | 222 (−) | 1.000 | 1.000 | ctAATTAa |
| | | | | | SEQ ID No. 555 |
| V$SHOX_02 | SHOX | 222 (−) | 1.000 | 1.000 | ctAATTAa |
| | | | | | SEQ ID No. 555 |
| V$SATB1_ | SATB1 | 222 (+) | 0.970 | 0.928 | ctaatTAAGGacccta |
| Q3 | | | | | SEQ ID No. 556 |
| V$RAX2_01 | RAX2 | 222 (+) | 1.000 | 0.999 | ctAATTAa |
| | | | | | SEQ ID No. 555 |
| V$PRRX1_ | PRRX1 | 222 (+) | 1.000 | 1.000 | ctAATTAa |
| 02 | | | | | SEQ ID No. 555 |
| V$UNCX_03 | UNCX | 222 (−) | 1.000 | 0.998 | cTAATTaa |
| | | | | | SEQ ID No. 555 |
| V$LHX2_Q4 | Lhx2 | 222 (−) | 1.000 | 1.000 | cTAATT |
| | | | | | SEQ ID No. 557 |
| V$IPF1_01 | ipf1 | 222 (−) | 1.000 | 0.983 | ctAATTAagg |
| | | | | | SEQ ID No. 558 |
| V$ISX_04 | ISX | 222 (+) | 1.000 | 1.000 | ctAATTAa |
| | | | | | SEQ ID No. 555 |
| V$LMX1A_ | LMX1A | 222 (+) | 1.000 | 0.996 | ctAATTAa |
| 02 | | | | | SEQ ID No. 555 |
| V$LMX1B_ | LMX1B | 222 (+) | 1.000 | 1.000 | ctAATTAa |
| 03 | | | | | SEQ ID No. 555 |
| V$LHX4_03 | Lhx4 | 222 (+) | 1.000 | 0.998 | ctAATTAa |
| | | | | | SEQ ID No. 555 |
| V$NKX61_ | NKX6-1 | 222 (+) | 1.000 | 1.000 | ctAATTAa |
| 07 | | | | | SEQ ID No. 555 |
| V$NKX62_ | NKX6-2 | 222 (+) | 1.000 | 1.000 | ctAATTAa |
| 01 | | | | | SEQ ID No. 555 |
| V$PMX1_Q6 | PMX1 | 223 (−) | 1.000 | 1.000 | tAATTA |
| | | | | | SEQ ID No. 559 |
| V$MSX2 Q6 | Msx-2 | 223 (+) | 1.000 | 1.000 | TAATTaa |
| | | | | | SEQ ID No. 560 |
| V$PMX1_Q6 | PMX1 | 223 (+) | 1.000 | 1.000 | TAATTa |
| | | | | | SEQ ID No. 559 |
| V$PRRX2_ | Prrx2 | 223 (−) | 1.000 | 1.000 | TAATT |
| 03 | | | | | SEQ ID No. 561 |
| V$PRRX2_ | Prrx2 | 224 (+) | 1.000 | 1.000 | AATTA |
| 03 | | | | | SEQ ID No. 415 |
| V$DLX5_Q3 | Dlx-5 | 224 (+) | 1.000 | 1.000 | AATTAa |
| | | | | | SEQ ID No. 414 |
| V$DRI1_01 | DRI1 | 224 (+) | 1.000 | 1.000 | aATTAA |
| | | | | | SEQ ID No. 414 |
| V$PLAGL2_ | PLAGL2 | 228 (+) | 1.000 | 1.000 | aaGGACC |
| 03 | | | | | SEQ ID No. 562 |
| V$POU6F1_ | POU6F1 | 231 (+) | 0.889 | 0.872 | gaccctAATCAgcttgg |
| 02 | | | | | SEQ ID No. 563 |
| V$PITX1_Q6 | PITX1 | 231 (+) | 1.000 | 0.940 | gacccTAATCa |
| | | | | | SEQ ID No. 564 |
| V$LHX8_01 | Lhx8 | 231 (+) | 0.861 | 0.886 | gacccTAATCagcttgg |
| | | | | | SEQ ID No. 565 |
| V$IPF1_02 | ipf1 | 233 (+) | 1.000 | 0.923 | ccCTAATcag |
| | | | | | SEQ ID No. 566 |
| V$CRX_Q4_ | CRX | 235 (+) | 1.000 | 1.000 | cTAATC |
| 01 | | | | | SEQ ID No. 567 |
| V$PMX1_Q6 | PMX1 | 236 (+) | 1.000 | 1.000 | TAATCa |
| | | | | | SEQ ID No. 568 |

TABLE 11
Transcription factor Position Weight Matrix (PWM, Transfac) recognizing the NR2E3
promoter (−250 bp from TSS)

| | | Position | Core | Matrix | |
|---|---|---|---|---|---|
| Matrix | Factor name | (strand) | score | score | Sequence |
| V$KID3_01 | Kid3 | 23 (−) | 1.000 | 1.000 | GGTGG |
| | | | | | SEQ ID No. 322 |
| V$CTCF_08 | CTCF | 44 (+) | 1.000 | 0.883 | caagatgTGGCAt |
| | | | | | SEQ ID No. 569 |
| V$MYOD1_02 | MyoD | 63 (−) | 1.000 | 0.985 | gtgaacAGCTGag |
| | | | | | SEQ ID No. 570 |
| V$AP4_Q5 | AP-4 | 66 (−) | 1.000 | 0.998 | aacAGCTGag |
| | | | | | SEQ ID No. 571 |
| V$MYOGENIN_ | myogenin | 68 (+) | 1.000 | 1.000 | CAGCTg |
| Q6_01 | | | | | SEQ ID No. 572 |
| V$MYOGENIN_ | myogenin | 68 (−) | 1.000 | 1.000 | cAGCTG |
| Q6_01 | | | | | SEQ ID No. 572 |
| V$GR_Q6 | GR | 72 (−) | 0.986 | 0.961 | tgaGCACAcagggca |
| | | | | | ggag |
| | | | | | SEQ ID No. 573 |
| V$SIN3A_01 | sin3A | 73 (−) | 1.000 | 0.768 | gAGCACacagggca |
| | | | | | SEQ ID No. 574 |
| V$ZIC3_04 | Zic3 | 89 (−) | 1.000 | 0.850 | agggccCCGGGgga |
| | | | | | c |
| | | | | | SEQ ID No. 575 |
| V$ZIC2_04 | Zic2 | 89 (−) | 1.000 | 0.862 | agggccccGGGGGa |
| | | | | | c |
| | | | | | SEQ ID No. 575 |
| V$ZIC1_04 | Zic1 | 90 (+) | 0.866 | 0.814 | gggccccgGGGGAc |
| | | | | | SEQ ID No. 576 |
| V$ZIC3_04 | Zic3 | 90 (+) | 1.000 | 0.849 | gggcCCCGGgggac |
| | | | | | c |
| | | | | | SEQ ID No. 577 |
| V$ZIC2_04 | Zic2 | 90 (+) | 0.852 | 0.861 | ggGCCCCgggggac |
| | | | | | c |
| | | | | | SEQ ID No. 577 |
| V$ZIC1_04 | Zic1 | 90 (−) | 0.668 | 0.816 | gGGCCCcgggggac |
| | | | | | SEQ ID No. 576 |
| V$AP2ALPHA_01 | AP-2alpha | 92 (+) | 1.000 | 1.000 | GCCCCgggg |
| | | | | | SEQ ID No. 578 |
| V$AP2GAMMA_ | AP-2gamma | 92 (+) | 0.998 | 0.998 | GCCCCgggg |
| 01 | | | | | SEQ ID No. 578 |
| V$CPBP_Q6 | CPBP | 92 (+) | 1.000 | 1.000 | GCCCCgg |
| | | | | | SEQ ID No. 579 |
| V$CPBP_Q6 | CPBP | 95 (−) | 1.000 | 1.000 | ccGGGGG |
| | | | | | SEQ ID No. 580 |
| V$CHCH_01 | Churchill | 96 (+) | 1.000 | 1.000 | CGGGGg |
| | | | | | SEQ ID No. 581 |
| V$REST_16 | REST | 97 (+) | 0.895 | 0.798 | gggggaccttgGGCA |
| | | | | | Gcccgg |
| | | | | | SEQ ID No. 582 |
| V$RELA_05 | RelA-p65 | 99 (−) | 1.000 | 0.923 | GGGACctt |
| | | | | | SEQ ID No. 583 |
| V$RFX1_01 | RFX1 | 107 (+) | 0.982 | 0.949 | gggcagcccgGGAA |
| | | | | | Cca |
| | | | | | SEQ ID No. 584 |
| V$GABPA_08 | GABP-alpha | 119 (+) | 0.865 | 0.856 | AACCAgcat |
| | | | | | SEQ ID No. 585 |
| V$KAISO_01 | Kaiso | 131 (−) | 1.000 | 0.991 | gtaGCAGGac |
| | | | | | SEQ ID No. 586 |
| V$DLX2_Q6 | DLX2 | 136 (+) | 0.975 | 0.933 | aggACTGAccg |
| | | | | | SEQ ID No. 587 |
| V$IRF6_04 | IRF6 | 144 (+) | 0.966 | 0.913 | ccggcTCCCGgggca |
| | secondary | | | | SEQ ID No. 588 |
| | motif | | | | |
| V$HIC1_02 | HIC1 | 150 (−) | 1.000 | 0.978 | cccgGGGCAccttgg |
| | | | | | SEQ ID No. 589 |
| V$CPBP_Q6 | CPBP | 151 (−) | 1.000 | 1.000 | ccGGGGC |
| | | | | | SEQ ID No. 590 |
| V$BRN1_Q6 | BRN1 | 165 (+) | 1.000 | 1.000 | tAATGCt |
| | | | | | SEQ ID No. 591 |
| V$E47_01 | E47 | 169 (+) | 1.000 | 0.986 | gctgCAGGTgtggcc |
| | | | | | SEQ ID No. 592 |
| V$SLUG_Q6_02 | slug | 170 (−) | 1.000 | 1.000 | ctgcAGGTG |
| | | | | | SEQ ID No. 593 |
| V$HSF4_Q3 | HSF4 | 170 (+) | 1.000 | 1.000 | CTGCAgg |
| | | | | | SEQ ID No. 594 |
| V$TCF4_04 | TCF4 | 171 (−) | 1.000 | 1.000 | tgcAGGTGtg |
| | | | | | SEQ ID No. 595 |
| V$MASH1_Q6_02 | MASH-1 | 172 (−) | 1.000 | 1.000 | gcAGGTGtgg |
| | | | | | SEQ ID No. 596 |
| V$TALLIKE_Q6 | Tal like | 172 (−) | 1.000 | 0.985 | gcAGGTGtggcc |
| | | | | | SEQ ID No. 597 |
| V$MRF4_Q3 | MRF4 | 172 (−) | 1.000 | 1.000 | gcAGGTG |
| | | | | | SEQ ID No. 598 |
| V$HTF4_Q2 | HTF4 | 172 (−) | 1.000 | 1.000 | gcAGGTG |
| | | | | | SEQ ID No. 598 |
| V$E47_05 | E47 | 172 (+) | 1.000 | 1.000 | gCAGGTgt |
| | | | | | SEQ ID No. 599 |
| V$MYB_Q4 | c-Myb | 179 (+) | 1.000 | 0.997 | tggcCAGTTgat |
| | | | | | SEQ ID No. 600 |
| V$MYB_Q5_01 | MYB | 180 (−) | 1.000 | 1.000 | ggcCAGTTg |
| | | | | | SEQ ID No. 601 |
| V$CMYB_Q5 | c-Myb | 180 (−) | 1.000 | 0.995 | ggcCAGTTgat |
| | | | | | SEQ ID No. 602 |
| V$MYB_Q6 | c-Myb | 181 (−) | 1.000 | 0.998 | gcCAGTTgat |
| | | | | | SEQ ID No. 603 |
| V$P300_Q5 | p300 | 196 (−) | 1.000 | 1.000 | gtggaGACAG |
| | | | | | SEQ ID No. 604 |
| V$SMAD2_Q6 | Smad2 | 200 (+) | 1.000 | 1.000 | AGACAg |
| | | | | | SEQ ID No. 185 |
| V$CRX_Q4_01 | CRX | 210 (−) | 1.000 | 1.000 | GATTAa |
| | | | | | SEQ ID No. 605 |
| V$HOXA10_Q5 | HOXA10 | 222 (−) | 1.000 | 0.997 | CATAAAct |
| | | | | | SEQ ID No. 606 |
| V$BEN_01 | BEN | 235 (+) | 1.000 | 0.957 | CAGCGgct |
| | | | | | SEQ ID No. 607 |
| V$HIC1_02 | HIC1 | 236 (+) | 1.000 | 0.980 | agcggcTGCCCcggg |
| | | | | | SEQ ID No. 608 |
| V$CPBP_Q6 | CPBP | 243 (+) | 1.000 | 1.000 | GCCCCgg |
| | | | | | SEQ ID No. 579 |

TABLE 12
Transcription factor Position Weight Matrix (PWM, Transfac) recognizing the NRL
promoter (−250 bp from TSS)

| | | Position | Core | Matrix | |
|---|---|---|---|---|---|
| Matrix | Factor name | (strand) | score | score | Sequence |
| V$LKLF_02 | LKLF | 9 (+) | 1.000 | 1.000 | tGGGCGg |
| | | | | | SEQ ID No. 610 |
| V$KLF17_01 | KLF17 | 9 (+) | 1.000 | 1.000 | tgGGCGG |
| | | | | | SEQ ID No. 610 |
| V$KLF17_02 | Klf17 | 9 (+) | 1.000 | 1.000 | tgGGCGG |
| | | | | | SEQ ID No. 610 |
| V$EGR1_13 | Egr-1 | 10 (−) | 1.000 | 1.000 | ggGCGGT |
| | | | | | SEQ ID No. 611 |
| V$FLI1_04 | FLI1 | 17 (−) | 0.816 | 0.749 | gtTGGATttccagg |
| | | | | | SEQ ID No. 612 |
| V$STAT3_01 | STAT3 | 18 (−) | 0.775 | 0.821 | ttggatttccaGGTAAcctct |
| | | | | | SEQ ID No. 613 |
| V$STAT3_12 | STAT3 | 18 (+) | 1.000 | 0.919 | ttggaTTTCCaggta |
| | | | | | SEQ ID No. 614 |
| V$STAT3_01 | STAT3 | 18 (+) | 1.000 | 0.809 | ttggaTTTCCaggtaacctct |
| | | | | | SEQ ID No. 613 |
| V$STAT3_03 | STAT3 | 19 (−) | 0.974 | 0.923 | tggatTTCCAggtaac |
| | | | | | SEQ ID No. 615 |
| V$BCL6_04 | BCL-6 | 21 (−) | 1.000 | 0.917 | gattTCCAGgtaa |
| | | | | | SEQ ID No. 616 |
| V$AP3_Q6_01 | AP-3 | 21 (−) | 1.000 | 1.000 | gaTTTCCa |
| | | | | | SEQ ID No. 617 |
| V$PPARA_02 | PPARalpha: | 29 (−) | 1.000 | 0.896 | ggtaacctctcTGACCgac |
| | RXRalpha | | | | SEQ ID No. 618 |
| V$VDR_03 | VDR | 31 (−) | 1.000 | 0.901 | taacctctcTGACCga |
| | | | | | SEQ ID No. 619 |
| V$YB1_Q4 | YB-1 | 43 (+) | 1.000 | 0.984 | ccgaCCAATcg |
| | | | | | SEQ ID No. 620 |
| V$YB1_Q3 | YB-1 | 47 (+) | 1.000 | 0.981 | CCAATcgaaa |
| | | | | | SEQ ID No. 621 |
| V$GTF2IRD1_ | GTF2IRD1- | 52 (−) | 0.934 | 0.941 | cgAAATCcc |
| 01 | isoform2 | | | | SEQ ID No. 622 |
| V$IRF4_04 | IRF4 secondary | 56 (+) | 1.000 | 0.940 | atcccTCTCGgaaga |
| | motif | | | | SEQ ID No. 623 |
| V$IRF6_04 | IRF6 secondary | 56 (+) | 1.000 | 0.940 | atcccTCTCGgaaga |
| | motif | | | | SEQ ID No. 623 |
| V$ELF5_03 | Elf5 | 62 (+) | 1.000 | 0.980 | ctcGGAAGaa |
| | | | | | SEQ ID No. 624 |
| V$ERFDLX3_ | ERF: DIx-3 | 62 (+) | 1.000 | 0.921 | ctCGGAAgaaagcgcttc |
| 01 | | | | | SEQ ID No. 625 |
| V$TEL1_02 | TEL1 | 62 (+) | 1.000 | 0.988 | ctCGGAAgaa |
| | | | | | SEQ ID No. 624 |
| V$TEL1_01 | TEL1 | 62 (+) | 1.000 | 0.988 | ctCGGAAgaa |
| | | | | | SEQ ID No. 624 |
| V$GABPA_Q4 | GABP-alpha | 64 (−) | 1.000 | 1.000 | cGGAAG |
| | | | | | SEQ ID No. 626 |
| V$FOXN1_01 | FOXN1 | 64 (−) | 0.832 | 0.756 | cggaagaaagcGCTTCact |
| | | | | | agct |
| | | | | | SEQ ID No. 627 |
| V$ZNF35_04 | ZNF35 | 65 (+) | 1.000 | 1.000 | gGAAGA |
| | | | | | SEQ ID No. 143 |
| V$SPIB_Q3 | Spi-B | 66 (−) | 1.000 | 1.000 | gAAGAA |
| | | | | | SEQ ID No. 291 |
| V$PARP_Q4 | PARP | 67 (−) | 1.000 | 1.000 | aAGAAA |
| | | | | | SEQ ID No. 292 |
| V$GATA6_04 | GATA-6 | 80 (−) | 1.000 | 0.976 | actagcTTATCtcatct |
| | | | | | SEQ ID No. 628 |
| V$GATA2_09 | GATA2 | 80 (+) | 1.000 | 0.966 | actagcTTATCtca |
| | | | | | SEQ ID No. 629 |
| V$GATA6_08 | GATA-6 | 81 (−) | 1.000 | 0.958 | ctagcTTATCtcat |
| | | | | | SEQ ID No. 630 |
| V$GATA1_Q6 | GATA-1 | 82 (−) | 1.000 | 0.994 | tagcTTATCtcatct |
| | | | | | SEQ ID No. 631 |
| V$GATA1_11 | Gata1 | 83 (+) | 1.000 | 0.971 | agcTTATCtca |
| | | | | | SEQ ID No. 632 |
| V$GATA2_10 | GATA-2 | 83 (−) | 1.000 | 0.989 | agcTTATCtca |
| | | | | | SEQ ID No. 633 |
| V$GATA1_14 | GATA-1 | 83 (−) | 1.000 | 0.988 | agcTTATCtca |
| | | | | | SEQ ID No. 632 |
| V$GATA2_11 | GATA-2 | 83 (−) | 1.000 | 0.987 | agcTTATCtca |
| | | | | | SEQ ID No. 632 |
| V$TAL1_05 | Tal-1 | 83 (−) | 1.000 | 0.981 | agcTTATCtcatctaaccaa |
| | | | | | SEQ ID No. 634 |
| V$GATA3_10 | GATA3 | 84 (−) | 1.000 | 0.978 | gctTATCT |
| | | | | | SEQ ID No. 635 |
| V$TAL1_04 | Tal-1 | 84 (−) | 1.000 | 0.979 | gcTTATCtcatctaaccaa |
| | | | | | SEQ ID No. 636 |
| V$GATA1_13 | GATA-1 | 84 (−) | 1.000 | 0.982 | gcTTATCtcatctaaccaa |
| | | | | | SEQ ID No. 636 |
| V$GATA3_11 | GATA-3 | 84 (−) | 1.000 | 0.994 | gcTTATCt |
| | | | | | SEQ ID No. 637 |
| V$GATA4_03 | GATA-4 | 84 (+) | 1.000 | 0.981 | gcTTATCtcat |
| | | | | | SEQ ID No. 638 |
| V$GATA1_10 | GATA-1 | 84 (+) | 1.000 | 0.984 | gcTTATCtc |
| | | | | | SEQ ID No. 639 |
| V$GATA3_07 | GATA3 | 84 (−) | 1.000 | 0.998 | gcTTATCt |
| | | | | | SEQ ID No. 640 |
| V$GATA2_Q5 | GATA-2 | 84 (−) | 1.000 | 1.000 | gcTTATC |
| | | | | | SEQ ID No. 641 |
| V$GATA_Q6 | GATA | 85 (−) | 1.000 | 1.000 | cTTATCt |
| | | | | | SEQ ID No. 642 |
| V$GATA6_Q5 | GATA-6 | 85 (−) | 1.000 | 1.000 | cTTATCt |
| | | | | | SEQ ID No. 642 |
| V$GATA1_12 | GATA-1 | 85 (−) | 1.000 | 1.000 | cTTATCt |
| | | | | | SEQ ID No. 642 |
| V$GATA3_Q4 | GATA-3 | 86 (−) | 1.000 | 1.000 | tTATCT |
| | | | | | SEQ ID No. 274 |
| V$PRDM16_ | MEL1 | 90 (−) | 1.000 | 1.000 | cTCATC |
| 04 | | | | | SEQ ID No. 643 |
| V$NFY_C | NF-Y | 94 (−) | 0.800 | 0.874 | tctaaccAATTAga |
| | | | | | SEQ ID No. 644 |
| V$MSX2_01 | Msx-2 | 94 (+) | 1.000 | 0.955 | tctaaccAATTAgaagc |
| | | | | | SEQ ID No. 645 |
| V$NFY_Q6 | NF-Y | 96 (+) | 1.000 | 0.983 | taaCCAATtag |
| | | | | | SEQ ID No. 646 |
| V$V$X1_03 | V$X1 | 97 (+) | 0.992 | 0.953 | aacCAATTaga |
| | | | | | SEQ ID No. 647 |
| V$VENTX_01 | VENTX | 98 (+) | 1.000 | 0.985 | accaATTAG |
| | | | | | SEQ ID No. 648 |
| V$LBX2_04 | LBX2 | 98 (+) | 1.000 | 1.000 | accAATTAga |
| | | | | | SEQ ID No. 649 |
| V$GBX1_04 | Gbx1 | 98 (+) | 1.000 | 0.999 | accAATTAga |
| | | | | | SEQ ID No. 649 |
| V$GBX2_04 | GBX2 | 98 (+) | 1.000 | 0.998 | accAATTAga |
| | | | | | SEQ ID No. 649 |
| V$ESX1_03 | ESX1 | 98 (+) | 1.000 | 0.997 | accAATTAga |
| | | | | | SEQ ID No. 649 |
| V$BARHL1_04 | Barhl1 | 98 (−) | 1.000 | 0.999 | accAATTAga |
| | | | | | SEQ ID No. 649 |
| V$BARHL2_04 | BARHL2 | 98 (−) | 1.000 | 0.999 | accAATTAga |
| | | | | | SEQ ID No. 649 |
| V$HMX3_03 | HMX3 | 98 (+) | 1.000 | 0.970 | acCAATTagaa |
| | | | | | SEQ ID No. 650 |
| V$RAX2_01 | RAX2 | 99 (+) | 1.000 | 1.000 | ccAATTAg |
| | | | | | SEQ ID No. 651 |
| V$SHOX2_03 | SHOX2 | 99 (+) | 1.000 | 1.000 | ccAATTAg |
| | | | | | SEQ ID No. 651 |
| V$SHOX_02 | SHOX | 99 (−) | 1.000 | 0.999 | ccAATTAg |
| | | | | | SEQ ID No. 651 |
| V$HOXA6_03 | HOXA6 | 99 (+) | 1.000 | 0.987 | ccAATTAg |
| | | | | | SEQ ID No. 651 |
| V$HOXA7_08 | HOXA7 | 99 (+) | 1.000 | 0.994 | ccAATTAg |
| | | | | | SEQ ID No. 651 |
| V$PRRX2_06 | PRRX2 | 99 (+) | 1.000 | 1.000 | ccAATTAg |
| | | | | | SEQ ID No. 651 |
| V$PRRX1_02 | PRRX1 | 99 (+) | 1.000 | 1.000 | ccAATTAg |
| | | | | | SEQ ID No. 651 |
| V$MSX1_05 | Msx-1 | 99 (+) | 1.000 | 1.000 | ccAATTAg |
| | | | | | SEQ ID No. 651 |
| VLHX9_03 | LHX9 | 99 (+) | 1.000 | 1.000 | ccAATTAg |
| | | | | | SEQ ID No. 651 |
| V$DLX2_03 | DIx-2 | 99 (+) | 1.000 | 1.000 | ccAATTAg |
| | | | | | SEQ ID No. 651 |
| V$BSX_03 | BSX | 99 (+) | 1.000 | 1.000 | ccAATTAg |
| | | | | | SEQ ID No. 651 |
| V$YB1_Q3 | YB-1 | 99 (+) | 1.000 | 0.985 | ccAATtagaa |
| | | | | | SEQ ID No. 652 |
| V$OG2_01 | OG-2 | 100 (−) | 1.000 | 1.000 | cAATTA |
| | | | | | SEQ ID No. 653 |
| V$ISL2_02 | Isl2 | 100 (−) | 1.000 | 0.984 | caATTAG |
| | | | | | SEQ ID No. 654 |
| V$LHX2_Q4 | Lhx2 | 101 (+) | 1.000 | 1.000 | AATTAg |
| | | | | | SEQ ID No. 655 |
| V$PRRX2_03 | Prrx2 | 101 (+) | 1.000 | 1.000 | AATTA |
| | | | | | SEQ ID No. 339 |
| V$LEF1_Q2 | TCF-7 related | 119 (+) | 1.000 | 1.000 | tCAAAG |
| | | | | | SEQ ID No. 656 |
| V$TCF3_Q6 | TCF-3 | 119 (−) | 1.000 | 1.000 | tCAAAG |
| | | | | | SEQ ID No. 656 |
| V$FOXN4_04 | FOXN4 secondary | 125 (−) | 0.839 | 0.765 | accctcgACGCCcccacctt |
| | motif | | | | at |
| | | | | | SEQ ID No. 657 |
| V$CTCF_16 | CTCF | 125 (−) | 1.000 | 0.909 | accctcgacgCCCCCac |
| | | | | | SEQ ID No. 658 |
| V$REST_13 | REST | 127 (+) | 1.000 | 0.753 | cctcgacGCCCCcacct |
| | | | | | SEQ ID No. 659 |
| V$EGR_Q6 | Egr | 131 (−) | 1.000 | 0.979 | gacgCCCCCac |
| | | | | | SEQ ID No. 660 |
| V$EGR1_Q3 | Egr-1 | 132 (−) | 1.000 | 0.971 | acgCCCCCac |
| | | | | | SEQ ID No. 661 |
| V$GKLF_Q3 | GKLF | 132 (−) | 0.977 | 0.976 | acgccCCCACctta |
| | | | | | SEQ ID No. 662 |
| V$ZNF300_04 | ZNF300 | 133 (−) | 1.000 | 0.983 | cgcCCCCAc |
| | | | | | SEQ ID No. 663 |
| V$EGR_Q3 | EGR | 133 (+) | 1.000 | 0.987 | cgCCCCCacct |
| | | | | | SEQ ID No. 664 |
| V$WT1_Q6_ | WT1 | 133 (+) | 1.000 | 0.996 | CGCCCccacc |
| 01 | | | | | SEQ ID No. 665 |
| V$CPBP_Q6 | CPBP | 135 (+) | 1.000 | 1.000 | CCCCCac |
| | | | | | SEQ ID No. 666 |
| V$PPARA_02 | PPARalpha: | 136 (−) | 0.865 | 0.841 | ccccaccttatCGACCaat |
| | RXRalpha | | | | SEQ ID No. 667 |
| V$KID3_01 | Kid3 | 138 (+) | 1.000 | 1.000 | CCACC |
| | | | | | SEQ ID No. 197 |
| V$RUSH1A_02 | RUSH-1alpha | 139 (+) | 1.000 | 0.999 | cacCTTATcg |
| | | | | | SEQ ID No. 668 |
| V$NFYB_01 | NF-YB | 143 (+) | 1.000 | 0.992 | ttatcgaCCAATcag |
| | | | | | SEQ ID No. 669 |
| V$NFY_Q6_01 | NF-Y | 144 (+) | 1.000 | 0.990 | tatcgaCCAATca |
| | | | | | SEQ ID No. 670 |
| V$NFY_01 | NF-Y | 145 (+) | 1.000 | 0.992 | atcgaCCAATcagagc |
| | | | | | SEQ ID No. 671 |
| V$NFY_C | NF-Y | 145 (−) | 1.000 | 0.921 | atcgaccAATCAga |
| | | | | | SEQ ID No. 672 |
| V$NFYA_03 | NFYA | 146 (−) | 1.000 | 0.973 | tcgaCCAATcagagcgcc |
| | | | | | SEQ ID No. 673 |
| V$NFYA_02 | NF-YA | 146 (−) | 1.000 | 0.984 | tcgaCCAATcagag |
| | | | | | SEQ ID No. 674 |
| V$YB1_Q4 | YB-1 | 146 (+) | 1.000 | 0.987 | tcgaCCAATca |
| | | | | | SEQ ID No. 675 |
| V$NFYA_Q5 | NF-YA | 147 (+) | 1.000 | 0.978 | cgaCCAATcagagc |
| | | | | | SEQ ID No. 676 |
| V$NFYC_Q5 | NF-YC | 147 (+) | 1.000 | 0.970 | cgaCCAATcagagc |
| | | | | | SEQ ID No. 677 |
| V$NFY_Q3 | NF-Y | 148 (+) | 1.000 | 0.988 | gaCCAATcaga |
| | | | | | SEQ ID No. 678 |
| V$YB1_Q3 | YB-1 | 150 (+) | 1.000 | 0.995 | CCAATcagag |
| | | | | | SEQ ID No. 679 |
| V$LHX8_06 | Lhx8 | 151 (−) | 1.000 | 1.000 | cAATCA |
| | | | | | SEQ ID No. 680 |
| V$CTCF_16 | CTCF | 152 (−) | 1.000 | 0.978 | aatcagagcgCCCCCtt |
| | | | | | SEQ ID No. 681 |
| V$ZF5_B | ZF5 | 155 (−) | 0.919 | 0.935 | caGAGCGccccct |
| | | | | | SEQ ID No. 682 |
| V$LRF_Q2 | LRF | 158 (−) | 1.000 | 0.992 | agcgCCCCC |
| | | | | | SEQ ID No. 683 |
| VINSM1_01 | INSM1 | 160 (−) | 1.000 | 0.979 | cgcCCCCTtaca |
| | | | | | SEQ ID No. 684 |
| V$CPBP_Q6 | CPBP | 162 (+) | 1.000 | 1.000 | CCCCCtt |
| | | | | | SEQ ID No. 685 |
| V$SOX9_Q5 | Sox-9 | 164 (+) | 1.000 | 0.979 | cccttACAAAggccggc |
| | | | | | SEQ ID No. 686 |
| V$SOX9_Q4 | Sox-9 | 167 (+) | 1.000 | 0.963 | ttaCAAAGgcc |
| | | | | | SEQ ID No. 687 |
| V$SOX10_Q6_ | Sox-10 | 168 (+) | 1.000 | 1.000 | tACAAAg |
| 01 | | | | | SEQ ID No. 688 |
| V$SOX10_01 | Sox-10 | 169 (−) | 1.000 | 1.000 | ACAAAg |
| | | | | | SEQ ID No. 689 |
| V$TCF1_Q5 | TCF-1 | 169 (−) | 1.000 | 1.000 | aCAAAG |
| | | | | | SEQ ID No. 689 |
| V$SOX18_Q5 | Sox-18 | 170 (+) | 1.000 | 1.000 | CAAAGgc |
| | | | | | SEQ ID No. 690 |
| V$KAISO_Q2 | Kaiso | 175 (+) | 0.986 | 0.947 | gccggcAGCAGt |
| | | | | | SEQ ID No. 691 |
| V$HSF4_Q3 | HSF4 | 176 (−) | 1.000 | 1.000 | ccGGCAG |
| | | | | | SEQ ID No. 692 |
| V$BEN_02 | BEN | 183 (+) | 0.769 | 0.850 | CAGTGaca |
| | | | | | SEQ ID No. 693 |
| V$CAAT_01 | CCAAT box | 187 (+) | 1.000 | 0.982 | gacagCCAATga |
| | | | | | SEQ ID No. 694 |
| V$YB1_Q4 | YB-1 | 188 (+) | 1.000 | 0.995 | acagCCAATga |
| | | | | | SEQ ID No. 695 |
| V$NF1C_Q6 | NF-1C | 189 (−) | 1.000 | 1.000 | caGCCAA |
| | | | | | SEQ ID No. 696 |
| V$ALPHACP1_ | alpha-CP1 | 189 (+) | 1.000 | 0.887 | cagCCAATgaa |
| 01 | | | | | SEQ ID No. 697 |
| V$YB1_Q3 | YB-1 | 192 (+) | 1.000 | 0.971 | CCAATgaaaa |
| | | | | | SEQ ID No. 698 |
| V$POU2F1_ | POU2F1 | 194 (−) | 0.973 | 0.978 | aatGAAAAt |
| Q4 | | | | | SEQ ID No. 699 |
| V$IRF4_Q6 | IRF-4 | 197 (+) | 1.000 | 1.000 | GAAAAta |
| | | | | | SEQ ID No. 700 |
| V$MEF2D_Q4 | MEF-2D | 198 (+) | 1.000 | 1.000 | aAAATAg |
| | | | | | SEQ ID No. 701 |
| V$TBX5_02 | Tbx5 | 214 (−) | 1.000 | 0.971 | taACACCcct |
| | | | | | SEQ ID No. 702 |
| V$GKLF_Q4 | GKLF | 221 (+) | 1.000 | 1.000 | CCTCCtt |
| | | | | | SEQ ID No. 703 |
| V$FOXN1_01 | FOXN1 | 221 (−) | 0.830 | 0.767 | cctccttcctcGCCTCacgcc |
| | | | | | ca |
| | | | | | SEQ ID No. 704 |
| V$ELF5_03 | Elf5 | 223 (−) | 1.000 | 0.981 | tcCTTCCtcg |
| | | | | | SEQ ID No. 705 |
| V$ELF1_Q5 | Elf-1 | 225 (−) | 1.000 | 1.000 | cTTCCT |
| | | | | | SEQ ID No. 706 |
| V$SPI1_Q5 | PU.1 | 225 (−) | 1.000 | 1.000 | cTTCCT |
| | | | | | SEQ ID No. 706 |
| V$SPIB_Q3 | Spi-B | 226 (+) | 1.000 | 1.000 | TTCCTc |
| | | | | | SEQ ID No. 707 |
| V$NFE4_Q5_ | NF-E4 | 232 (−) | 1.000 | 1.000 | gCCTCAc |
| 01 | | | | | SEQ ID No. 708 |
| V$E2F1_09 | E2F-1 | 233 (−) | 0.901 | 0.932 | cctCACGCcca |
| | | | | | SEQ ID No. 709 |
| V$MXI1_03 | Mxi1 | 234 (−) | 1.000 | 0.989 | ctcacgccCACGTgg |
| | | | | | SEQ ID No. 710 |
| V$EGR3_Q6 | EGR3 | 235 (+) | 1.000 | 0.966 | tcacgCCCACgtgg |
| | | | | | SEQ ID No. 711 |
| V$EGR1_18 | EGR-1 | 235 (−) | 1.000 | 0.955 | tcacgCCCACgtg |
| | | | | | SEQ ID No. 712 |
| V$EGR3_01 | Egr-3 | 237 (−) | 1.000 | 0.873 | acgcCCACGtgg |
| | | | | | SEQ ID No. 713 |
| V$EGR1_02 | EGR-1 | 237 (−) | 1.000 | 0.928 | acgCCCACgtg |
| | | | | | SEQ ID No. 714 |
| V$ARNTLIKE_ | ARNTLIKE | 238 (+) | 1.000 | 0.995 | cgccCACGTg |
| Q6 | | | | | SEQ ID No. 715 |
| V$HAIRYLIKE_ | HAIRYLIKE | 238 (+) | 1.000 | 0.991 | cgccCACGTg |
| Q3 | | | | | SEQ ID No. 715 |
| V$CMYC_02 | c-Myc | 239 (−) | 1.000 | 0.959 | gcccACGTGgtg |
| | | | | | SEQ ID No. 716 |
| V$CMYC_01 | c-Myc | 239 (−) | 1.000 | 0.922 | gcccACGTGgtg |
| | | | | | SEQ ID No. 716 |
| V$MYCMAX_ | c-Myc: Max | 239 (−) | 1.000 | 0.973 | gcccACGTGgtg |
| 02 | | | | | SEQ ID No. 716 |
| V$NMYC_01 | N-Myc | 239 (+) | 1.000 | 0.993 | gcccACGTGgtg |
| | | | | | SEQ ID No. 716 |
| V$DEC1_Q3 | DEC1 | 239 (−) | 1.000 | 0.942 | gccCACGTggtg |
| | | | | | SEQ ID No. 716 |
| V$CMYC_Q3 | C-Myc | 239 (+) | 1.000 | 0.978 | gccCACGTggt |
| | | | | | SEQ ID No. 717 |
| V$HIF2A_Q6 | HIF2A | 239 (−) | 1.000 | 1.000 | gccCACGT |
| | | | | | SEQ ID No. 718 |
| V$DEC1_Q2 | DEC1 | 239 (−) | 1.000 | 0.948 | gccCACGTggt |
| | | | | | SEQ ID No. 717 |
| V$CMYC_02 | c-Myc | 239 (+) | 1.000 | 0.948 | gccCACGTggtg |
| | | | | | SEQ ID No. 716 |
| V$CMYC_01 | c-Myc | 239 (+) | 1.000 | 0.897 | gccCACGTggtg |
| | | | | | SEQ ID No. 716 |
| V$NMYC_01 | N-Myc | 239 (−) | 1.000 | 0.993 | gccCACGTggtg |
| | | | | | SEQ ID No. 716 |
| V$MYCMAX_B | c-Myc: Max | 240 (−) | 1.000 | 0.949 | cccaCGTGGt |
| | | | | | SEQ ID No. 719 |
| V$HES1_02 | Hes1 | 240 (−) | 0.988 | 0.977 | cccACGTGgt |
| | | | | | SEQ ID No. 719 |
| V$CMYC_Q3 | C-Myc | 240 (−) | 1.000 | 0.979 | cccACGTGgtg |
| | | | | | SEQ ID No. 720 |
| V$MYC_02 | c-Myc | 240 (−) | 1.000 | 0.997 | cccACGTGgt |
| | | | | | SEQ ID No. 719 |
| V$HES1_02 | Hes1 | 240 (+) | 0.988 | 0.978 | ccCACGTggt |
| | | | | | SEQ ID No. 719 |
| V$MYC_02 | c-Myc | 240 (+) | 1.000 | 0.995 | ccCACGTggt |
| | | | | | SEQ ID No. 719 |
| V$CMYC_Q6_ | c-Myc | 240 (−) | 1.000 | 0.983 | ccCACGTg |
| 01 | | | | | SEQ ID No. 721 |
| V$MYCMAX_B | c-Myc: Max | 240 (+) | 1.000 | 0.948 | cCCACGtggt |
| | | | | | SEQ ID No. 719 |
| V$MYC_Q2 | Myc | 241 (−) | 1.000 | 1.000 | ccACGTG |
| | | | | | SEQ ID No. 722 |
| V$USF_C | USF | 241 (−) | 1.000 | 0.996 | ccACGTGg |
| | | | | | SEQ ID No. 723 |
| V$USF_C | USF | 241 (+) | 1.000 | 0.996 | cCACGTgg |
| | | | | | SEQ ID No. 723 |
| V$KID3_01 | Kid3 | 241 (+) | 1.000 | 1.000 | CCACG |
| | | | | | SEQ ID No. 724 |
| V$USF2_Q6 | USF2 | 242 (+) | 1.000 | 1.000 | CACGTg |
| | | | | | SEQ ID No. 725 |
| V$MYC_Q2 | Myc | 242 (+) | 1.000 | 1.000 | CACGTgg |
| | | | | | SEQ ID No. 726 |
| V$MAX_14 | MAX | 242 (−) | 1.000 | 1.000 | cACGTg |
| | | | | | SEQ ID No. 725 |
| V$USF2_Q6 | USF2 | 242 (−) | 1.000 | 1.000 | cACGTG |
| | | | | | SEQ ID No. 725 |
| V$CMYC_Q6_ | c-Myc | 242 (+) | 1.000 | 0.987 | cACGTGgt |
| 01 | | | | | SEQ ID No. 727 |
| V$MAX_14 | MAX | 242 (+) | 1.000 | 1.000 | cACGTG |
| | | | | | SEQ ID No. 725 |
| V$KID3_01 | Kid3 | 244 (−) | 1.000 | 1.000 | CGTGG |
| | | | | | SEQ ID No. 728 |

TABLE 14
Transcription factor Position Weight Matrix (PWM, Transfac) recognizing the ROM1
promoter (−250 bp from TSS)

| | | Position | Core | Matrix | |
|---|---|---|---|---|---|
| Matrix | Factor name | (strand) | score | score | Sequence |
| V$BEN_01 | BEN | 16 (−) | 1.000 | 0.957 | agcCGCTG |
| | | | | | SEQ ID No. 787 |
| V$AP2_Q4 | AP2 | 16 (−) | 0.986 | 0.988 | agccgCTGGC |
| | | | | | SEQ ID No. 788 |
| V$CHD2_01 | CHD2 | 41 (+) | 0.811 | 0.803 | ccTCCCGag |
| | | | | | SEQ ID No. 789 |
| V$CHD2_01 | CHD2 | 42 (−) | 0.811 | 0.789 | ctCCCGAgc |
| | | | | | SEQ ID No. 790 |
| V$ZFP161_04 | ZF5 secondary | 44 (+) | 0.829 | 0.855 | ccCGAGCagggccg |
| | motif | | | | SEQ ID No. 791 |
| V$HDAC1_Q3 | HDAC1 | 48 (+) | 1.000 | 0.959 | agCAGGGcc |
| | | | | | SEQ ID No. 792 |
| V$CREM_Q6 | CREM | 53 (+) | 1.000 | 0.962 | ggcCGTCAcct |
| | | | | | SEQ ID No. 793 |
| V$DEC_Q1 | DEC | 55 (−) | 0.973 | 0.936 | ccgtcACCTGgga |
| | | | | | SEQ ID No. 794 |
| V$EFC_Q6 | RFX1 (EF-C) | 56 (+) | 0.910 | 0.881 | cGTCACctgggaaa |
| | | | | | SEQ ID No. 795 |
| V$ZEB1_03 | ZEB1 | 58 (−) | 1.000 | 1.000 | tcACCTG |
| | | | | | SEQ ID No. 796 |
| V$TWIST_Q6 | TWIST | 59 (+) | 1.000 | 1.000 | CACCTgg |
| | | | | | SEQ ID No. 797 |
| V$PPARG_03 | PPAR | 59 (+) | 1.000 | 0.860 | cacctgggaAAAGGgca |
| | | | | | SEQ ID No. 798 |
| V$PPARG_08 | PPARgamma | 59 (+) | 1.000 | 0.887 | cacctgggaAAAGGgca |
| | | | | | SEQ ID No. 798 |
| V$PPARGRXRA_ | PPARgamma: | 61 (+) | 1.000 | 0.918 | cctgggaAAAGGgca |
| 01 | RXR-alpha | | | | SEQ ID No. 799 |
| V$RXRA_13 | RXR-ALPHA | 62 (+) | 1.000 | 0.823 | ctgggaAAAGGgcaa |
| | | | | | SEQ ID No. 800 |
| V$PPARDRI_ | PPAR direct | 63 (−) | 0.888 | 0.871 | tgggaaaAGGGCa |
| Q2 | repeat 1 | | | | SEQ ID No. 801 |
| V$NFAT1_Q6 | NFATc2 | 65 (+) | 1.000 | 1.000 | GGAAAa |
| | | | | | SEQ ID No. 802 |
| V$NFAT4_Q3 | NFATc3 | 65 (+) | 1.000 | 1.000 | GGAAAa |
| | | | | | SEQ ID No. 802 |
| V$NFAT1_Q4 | NFATc2 | 65 (+) | 1.000 | 1.000 | GGAAAa |
| | | | | | SEQ ID No. 802 |
| V$PPARGRXRA_ | PPARGAMMA: | 68 (+) | 0.943 | 0.903 | aaagggcaAGAGGta |
| 03 | XRR-ALPHA | | | | SEQ ID No. 803 |
| V$VDRRXRA_ | VDR: RXR- | 69 (+) | 0.942 | 0.895 | aaGGGCAagaggtactc |
| 01 | ALPHA | | | | SEQ ID No. 804 |
| V$ZNF35_04 | ZNF35 | 73 (+) | 1.000 | 1.000 | gCAAGA |
| | | | | | SEQ ID No. 805 |
| V$GATA1_01 | GATA-1 | 93 (−) | 1.000 | 0.997 | acCCATCacc |
| | | | | | SEQ ID No. 806 |
| V$DEC_Q1 | DEC | 95 (−) | 0.973 | 0.921 | ccatcACCTGaag |
| | | | | | SEQ ID No. 807 |
| V$SREBP1_01 | SREBP-1 | 96 (+) | 0.937 | 0.954 | catCACCTgaa |
| | | | | | SEQ ID No. 808 |
| V$ZEB1_03 | ZEB1 | 98 (−) | 1.000 | 1.000 | tcACCTG |
| | | | | | SEQ ID No. 809 |
| V$SPIB_Q3 | Spi-B | 104 (−) | 1.000 | 1.000 | gAAGAA |
| | | | | | SEQ ID No. 291 |
| V$PARP_Q4 | PARP | 105 (−) | 1.000 | 1.000 | aAGAAA |
| | | | | | SEQ ID No. 292 |
| V$GTF2IRD1_ | GTF2IRD1- | 119 (+) | 0.930 | 0.938 | ggGATTCtg |
| 01 | isoform2 | | | | SEQ ID No. 810 |
| V$SMAD2_Q6 | Smad2 | 125 (−) | 1.000 | 1.000 | cTGTCT |
| | | | | | SEQ ID No. 314 |
| V$SREBP1_02 | SREBP-1 | 126 (+) | 0.800 | 0.869 | tgTCTCCccac |
| | | | | | SEQ ID No. 811 |
| V$MZF1_Q5 | MZF-1 | 129 (−) | 1.000 | 1.000 | cTCCCCa |
| | | | | | SEQ ID No. 812 |
| V$PLZFB_Q3 | PLZF | 138 (+) | 0.981 | 0.977 | aCTTTAcatg |
| | | | | | SEQ ID No. 813 |
| V$ELK1_05 | ELK-1 | 146 (−) | 0.929 | 0.893 | tgtGTCCGgt |
| | | | | | SEQ ID No. 814 |
| V$CETS2_02 | c-ets-2 | 146 (−) | 1.000 | 0.907 | tgtgTCCGGt |
| | | | | | SEQ ID No. 814 |
| V$ETS1_02 | ETS1 | 146 (−) | 1.000 | 0.903 | tgtgTCCGGt |
| | | | | | SEQ ID No. 814 |
| V$IRF3_06 | IRF3 secondary | 160 (−) | 1.000 | 0.923 | ctgccCCTTTcagg |
| | motif | | | | SEQ ID No. 815 |
| V$CPBP_Q6 | CPBP | 162 (+) | 1.000 | 1.000 | GCCCCtt |
| | | | | | SEQ ID No. 816 |
| V$AP2ALPHA_ | AP-2alphaA | 173 (−) | 0.980 | 0.908 | gcagccccAGGCTtg |
| 03 | | | | | SEQ ID No. 817 |
| V$AP2ALPHA_ | AP-2alphaA | 173 (+) | 0.890 | 0.908 | gcAGCCCcaggcttg |
| 03 | | | | | SEQ ID No. 818 |
| V$TFAP2C_03 | TFAP2C | 175 (+) | 0.971 | 0.982 | aGCCCCaggct |
| | | | | | SEQ ID No. 819 |
| V$TFAP2A_09 | AP-2alpha | 175 (−) | 0.976 | 0.962 | agcccCAGGCttga |
| | | | | | SEQ ID No. 820 |
| V$AP2ALPHA_ | AP-2alpha | 175 (+) | 1.000 | 0.991 | agcccCAGGCt |
| Q4 | | | | | SEQ ID No. 821 |
| V$AP2_Q4 | AP2 | 175 (−) | 1.000 | 0.999 | agcccCAGGC |
| | | | | | SEQ ID No. 822 |
| V$CPBP_Q6 | CPBP | 176 (+) | 1.000 | 1.000 | GCCCCag |
| | | | | | SEQ ID No. 823 |
| V$PAX6_05_01 | Pax-6 | 177 (+) | 0.749 | 0.795 | ccccaGGCTTgaatgctc |
| | | | | | g |
| | | | | | SEQ ID No. 824 |
| V$ETF_Q6_01 | ETF | 200 (−) | 0.965 | 0.939 | gaggGGAGGag |
| | | | | | SEQ ID No. 825 |
| V$PAX4_Q2 | Pax-4 | 212 (−) | 1.000 | 0.940 | cGGTGGtaaag |
| | | | | | SEQ ID No. 826 |
| V$EP300_05 | p300 | 212 (−) | 0.793 | 0.870 | cgGTGGT |
| | | | | | SEQ ID No. 827 |
| V$KID3_01 | Kid3 | 213 (−) | 1.000 | 1.000 | GGTGG |
| | | | | | SEQ ID No. 322 |
| V$RXRA_15 | RXR-alpha | 213 (+) | 1.000 | 0.811 | ggtggtaaaggaTCAAG |
| | | | | | ggcct |
| | | | | | SEQ ID No. 828 |
| V$CTCF_05 | CTCF | 222 (+) | 0.880 | 0.846 | ggatcaagggcCTCCTtc |
| | | | | | tgg |
| | | | | | SEQ ID No. 829 |
| V$CTCF_17 | ctcf | 226 (−) | 0.941 | 0.897 | caagggcCTCCTtctggc |
| | | | | | ag |
| | | | | | SEQ ID No. 830 |
| V$CTCF_18 | ctcf | 226 (−) | 0.921 | 0.884 | caagggcCTCCTtctggc |
| | | | | | ag |
| | | | | | SEQ ID No. 830 |
| V$CTCF_03 | CTCF | 227 (−) | 0.945 | 0.908 | aagggcCTCCTtctggca |
| | | | | | g |
| | | | | | SEQ ID No. 831 |
| V$CTCF_01 | CTCF | 227 (−) | 0.917 | 0.915 | aagggCCTCCttctggca |
| | | | | | gg |
| | | | | | SEQ ID No. 832 |
| V$CTCF_04 | CTCF | 228 (+) | 0.837 | 0.810 | aGGGCCtccttctggca |
| | | | | | SEQ ID No. 833 |
| V$CTCF_02 | CTCF | 229 (−) | 0.930 | 0.921 | gggCCTCCttctggcagg |
| | | | | | gc |
| | | | | | SEQ ID No. 834 |
| V$GKLF_Q4 | GKLF | 232 (+) | 1.000 | 1.000 | CCTCCtt |
| | | | | | SEQ ID No. 703 |
| V$NF1B_Q6 | NF-1B | 239 (+) | 1.000 | 1.000 | CTGGCaggg |
| | | | | | SEQ ID No. 835 |
| V$HSF4_Q3 | HSF4 | 239 (−) | 1.000 | 1.000 | ctGGCAG |
| | | | | | SEQ ID No. 836 |

TABLE 13
Transcription factor Position Weight Matrix (PWM, Transfac) recognizing the OTX2
promoter (−250 bp from TSS)

| | | Position | Core | Matrix | |
|---|---|---|---|---|---|
| Matrix | Factor name | (strand) | score | score | Sequence |
| V$CEBP_Q2 | C/EBP alpha | 5 (−) | 0.984 | 0.976 | attttaaGCAAAgc |
| | | | | | SEQ ID No. 729 |
| V$GR_Q6_02 | GR | 30 (−) | 1.000 | 0.998 | aaaGAACAttctg |
| | | | | | SEQ ID No. 730 |
| V$AR_Q6_01 | AR | 31 (−) | 1.000 | 0.980 | aaGAACAttctggta |
| | | | | | SEQ ID No. 731 |
| V$AR_10 | AR | 31 (+) | 1.000 | 0.947 | aaGAACAttctggta |
| | | | | | SEQ ID No. 731 |
| V$HSF1_04 | HSF1 | 32 (−) | 0.752 | 0.881 | agaacattCTGGTaa |
| | | | | | SEQ ID No. 732 |
| V$HSF2_01 | HSF2 | 32 (−) | 1.000 | 0.996 | agaacATTCT |
| | | | | | SEQ ID No. 733 |
| V$HSF1_01 | HSF1 | 32 (−) | 1.000 | 0.994 | agaacATTCT |
| | | | | | SEQ ID No. 733 |
| V$HSF2_01 | HSF2 | 32 (+) | 0.997 | 0.994 | AGAACattct |
| | | | | | SEQ ID No. 733 |
| V$HSF1_01 | HSF1 | 32 (+) | 1.000 | 0.985 | AGAACattct |
| | | | | | SEQ ID No. 733 |
| V$HSF1_Q5_01 | HSF1 | 33 (+) | 1.000 | 0.997 | gaacaTTCTGgt |
| | | | | | SEQ ID No. 734 |
| V$HSF1_Q5 | HSF1 | 33 (−) | 1.000 | 0.987 | gaacaTTCTGgt |
| | | | | | SEQ ID No. 734 |
| V$HSF1_Q6_01 | HSF1 | 33 (+) | 1.000 | 0.991 | gaacaTTCTGgtaa |
| | | | | | SEQ ID No. 735 |
| V$ERFPITX1_01 | ERF: pitx1 | 48 (+) | 0.927 | 0.909 | gtcGGAGGcctggattt |
| | | | | | SEQ ID No. 736 |
| V$SOX4_Q5 | Sox-4 | 62 (−) | 1.000 | 1.000 | tTTGTT |
| | | | | | SEQ ID No. 737 |
| V$AP2GAMMA_ | AP-2 gamma | 66 (+) | 1.000 | 1.000 | ttGCCTG |
| Q5 | | | | | SEQ ID No. 738 |
| V$PAX3_01 | Pax-3 | 74 (+) | 1.000 | 0.783 | TCGTCcccccgtg |
| | | | | | SEQ ID No. 739 |
| V$RREB1_06 | RREB-1 | 74 (−) | 0.990 | 0.993 | tcGTCCC |
| | | | | | SEQ ID No. 740 |
| V$ZNF777_02 | ZNF777 | 75 (+) | 1.000 | 0.713 | cgtcccCCCGTgcagcagc |
| | | | | | SEQ ID No. 741 |
| V$HES1_02 | Hes1 | 79 (−) | 0.960 | 0.956 | cccCCGTGca |
| | | | | | SEQ ID No. 742 |
| V$CHCH_01 | Churchill | 79 (−) | 1.000 | 1.000 | cCCCCG |
| | | | | | SEQ ID No. 743 |
| V$CPBP_Q6 | CPBP | 79 (+) | 1.000 | 1.000 | CCCCCgt |
| | | | | | SEQ ID No. 744 |
| V$BEN_01 | BEN | 90 (+) | 1.000 | 0.996 | CAGCGgcc |
| | | | | | SEQ ID No. 745 |
| V$CTCF_16 | CTCF | 97 (−) | 1.000 | 0.913 | ctgttttcctCCCCCtg |
| | | | | | SEQ ID No. 746 |
| V$NFAT1_Q6 | NFATc2 | 100 (−) | 1.000 | 1.000 | tTTTCC |
| | | | | | SEQ ID No. 747 |
| V$NFAT4_Q3 | NFATc3 | 100 (−) | 1.000 | 1.000 | tTTTCC |
| | | | | | SEQ ID No. 747 |
| V$NFAT1_Q4 | NFATc2 | 100 (−) | 1.000 | 1.000 | tTTTCC |
| | | | | | SEQ ID No. 747 |
| V$SPIB_Q3 | Spi-B | 102 (+) | 1.000 | 1.000 | TTCCTc |
| | | | | | SEQ ID No. 748 |
| V$CPBP_Q6 | CPBP | 107 (+) | 1.000 | 1.000 | CCCCCtg |
| | | | | | SEQ ID No. 231 |
| V$FOXJ1_04 | FOXJ1 | 108 (−) | 1.000 | 0.934 | cccctGTTGTgtgtt |
| | secondary | | | | SEQ ID No. 749 |
| | motif | | | | |
| V$FAC1_01 | FAC1 | 108 (−) | 1.000 | 0.939 | cccctGTTGTgtgt |
| | | | | | SEQ ID No. 750 |
| V$LDSPOLYA_B | Poly A | 113 (+) | 1.000 | 0.948 | gttgTGTGTttttatt |
| | | | | | SEQ ID No. 751 |
| V$MEQ_01 | MEQ | 114 (−) | 1.000 | 0.995 | tTGTGTgtt |
| | | | | | SEQ ID No. 752 |
| V$FAC1_01 | FAC1 | 115 (−) | 0.978 | 0.940 | tgtgtGTTTTtatt |
| | | | | | SEQ ID No. 753 |
| V$MEQ_01 | MEQ | 116 (−) | 1.000 | 0.973 | gTGTGTttt |
| | | | | | SEQ ID No. 754 |
| V$CPEB1_01 | CPEB1 | 121 (−) | 1.000 | 1.000 | tTTTTAtt |
| | | | | | SEQ ID No. 755 |
| V$HOXA10_04 | HOXA10 | 121 (−) | 1.000 | 0.963 | ttTTTATtatt |
| | | | | | SEQ ID No. 756 |
| V$HOXD10_Q6 | HOXD10 | 122 (+) | 1.000 | 1.000 | tTTTATta |
| | | | | | SEQ ID No. 757 |
| V$HOXD11_04 | HOXD11 | 122 (−) | 1.000 | 0.984 | tTTTATtatt |
| | | | | | SEQ ID No. 758 |
| V$SATB1_Q5_01 | SATB1 | 122 (+) | 1.000 | 1.000 | tTTTAT |
| | | | | | SEQ ID No. 759 |
| V$HOXA13_01 | HOXA13 | 122 (−) | 1.000 | 1.000 | tTTTAT |
| | | | | | SEQ ID No. 759 |
| V$CDX1_Q5 | Cdx-1 | 123 (+) | 1.000 | 1.000 | TTTATt |
| | | | | | SEQ ID No. 334 |
| V$PMX1_Q6 | PMX1 | 124 (−) | 1.000 | 1.000 | tTATTA |
| | | | | | SEQ ID No. 760 |
| V$ZNF333_01 | ZNF333 | 126 (−) | 1.000 | 1.000 | ATTAT |
| | | | | | SEQ ID No. 327 |
| V$IRF4_Q6 | IRF-4 | 128 (−) | 1.000 | 1.000 | taTTTTC |
| | | | | | SEQ ID No. 761 |
| V$NFAT3_Q3_01 | NFATc4 | 129 (−) | 1.000 | 1.000 | atTTTCCc |
| | | | | | SEQ ID No. 762 |
| V$NFAT1_Q6 | NFATc2 | 130 (−) | 1.000 | 1.000 | tTTTCC |
| | | | | | SEQ ID No. 747 |
| V$NFAT4_Q3 | NFATc3 | 130 (−) | 1.000 | 1.000 | tTTTCC |
| | | | | | SEQ ID No. 747 |
| V$NFAT1_Q4 | NFATc2 | 130 (−) | 1.000 | 1.000 | tTTTCC |
| | | | | | SEQ ID No. 747 |
| V$MEIS1BHOXA9_ | MEIS1B: | 141 (−) | 1.000 | 0.842 | gcttagatgTGTCA |
| 02 | HOXA9 | | | | SEQ ID No. 763 |
| V$PREP1_Q3 | Prep-1 | 146 (−) | 1.000 | 0.968 | gatgTGTCAatc |
| | | | | | SEQ ID No. 764 |
| V$PBX1_05 | Pbx | 147 (−) | 1.000 | 0.967 | atgtgtCAATCatt |
| | | | | | SEQ ID No. 765 |
| V$LHX8_06 | Lhx8 | 153 (−) | 1.000 | 1.000 | cAATCA |
| | | | | | SEQ ID No. 766 |
| V$FOXM1_Q3 | FOXM1 | 153 (−) | 0.969 | 0.897 | caatCATTCtc |
| | | | | | SEQ ID No. 767 |
| V$CTCF_08 | CTCF | 167 (−) | 1.000 | 0.957 | cTGCCAttggttg |
| | | | | | SEQ ID No. 768 |
| V$SOX18_Q5 | Sox-18 | 169 (−) | 1.000 | 1.000 | gcCATTG |
| | | | | | SEQ ID No. 769 |
| V$YB1_Q4 | YB-1 | 170 (−) | 1.000 | 0.983 | ccATTGGttgg |
| | | | | | SEQ ID No. 770 |
| V$TAXCREB_02 | Tax/CREB | 180 (−) | 1.000 | 0.811 | gagagtttgCGTCAa |
| | | | | | SEQ ID No. 771 |
| V$ATF1_04 | ATF1 | 183 (−) | 1.000 | 0.861 | agtttgCGTCAaaa |
| | secondary | | | | SEQ ID No. 772 |
| | motif | | | | |
| V$SP100_03 | Sp100 | 183 (−) | 0.958 | 0.949 | agtTTGCGtcaaaa |
| | | | | | SEQ ID No. 773 |
| V$CREB_Q2 | CREB | 184 (−) | 1.000 | 0.929 | gtttgCGTCAaa |
| | | | | | SEQ ID No. 774 |
| V$CREB_Q4 | CREB | 184 (−) | 1.000 | 0.936 | gtttgCGTCAaa |
| | | | | | SEQ ID No. 774 |
| V$E2F_01 | E2F-1 | 185 (+) | 0.800 | 0.781 | tttgcgtCAAAAagt |
| | | | | | SEQ ID No. 775 |
| V$CREBATF_Q6 | CREB, ATF | 186 (−) | 1.000 | 0.958 | ttgCGTCAa |
| | | | | | SEQ ID No. 776 |
| V$CREB1_03 | CREB1 | 186 (+) | 1.000 | 0.920 | ttgCGTCAaaa |
| | | | | | SEQ ID No. 777 |
| V$E2F_02 | E2F | 188 (−) | 0.852 | 0.914 | gcgTCAAA |
| | | | | | SEQ ID No. 778 |
| V$E2F1DP1RB_ | Rb:E2F-1: | 188 (−) | 0.832 | 0.890 | gCGTCAaa |
| 01 | DP-1 | | | | SEQ ID No. 778 |
| V$E2F4DP1_01 | E2F-4: DP-1 | 188 (−) | 0.826 | 0.892 | gCGTCAaa |
| | | | | | SEQ ID No. 778 |
| V$E2F1DP2_01 | E2F-1: DP-2 | 188 (−) | 0.863 | 0.912 | gCGTCAaa |
| | | | | | SEQ ID No. 778 |
| V$FOXN1_01 | FOXN1 | 200 (+) | 0.830 | 0.753 | tgccagaGAGGCgctttctcag |
| | | | | | c |
| | | | | | SEQ ID No. 779 |
| V$NF1B_Q6_01 | NF-1B | 201 (+) | 1.000 | 0.997 | GCCAGagag |
| | | | | | SEQ ID No. 780 |
| V$MAFG_Q3 | MafG | 211 (+) | 1.000 | 0.830 | cgctttcTCAGCaaa |
| | | | | | SEQ ID No. 781 |
| V$CMAF_Q5 | c-MAF | 213 (+) | 1.000 | 0.992 | ctttcTCAGCa |
| | | | | | SEQ ID No. 782 |
| V$MAFB_Q4_01 | MAFB | 217 (+) | 1.000 | 1.000 | cTCAGCa |
| | | | | | SEQ ID No. 783 |
| V$PAX5_02 | Pax-5 | 222 (+) | 0.973 | 0.765 | caaatctccctgaGAGCGggac |
| | | | | | cggcct |
| | | | | | SEQ ID No. 784 |
| V$PAX3_B | Pax-3 | 230 (−) | 0.818 | 0.822 | cctgagagCGGGAccggcctc |
| | | | | | SEQ ID No. 785 |
| V$E2F1 09 | E2F-1 | 234 (+) | 1.000 | 0.916 | agaGCGGGacc |
| | | | | | SEQ ID No. 786 |

### Methods

### Prediction of TF Binding

The promoter sequence of RHO was analyzed using Transfac® with the “Vertebrate” database using high quality matrices and a “Core score” and “Matrix score” higher than 0.95. The sequence analyzed was Chr3:129528551-129528581 corresponding to −88 to −58 from the Transcriptional Start Site (TSS) of human Rhodopsin.

### Plasmid Construction

The human KLF15 CDS and the murine KLF15 CDS were synthetized by Eurofins MWG®. The fragments were cloned in pAAV2.1 under the control of the CMV or hGNAT1 promoter using NotI and HindIII restriction enzymes.

### AAV Vector Preparation

AAV vectors were produced by the TIGEM AAV Vector Core, by triple transfection of HEK293 cells followed by two rounds of CsCl2 purification. For each viral preparation, physical titers [genome copies (GC)/mL] were determined by averaging the titer achieved by dot-blot analysis and by PCR quantification using TaqMan (Applied Biosystems, Carlsbad, CA, USA) (12, 13).

### Animal Models

All procedures were performed in accordance with institutional guidelines for animal research and all of the animal studies were approved by the authors. P347S+/+ animals (23) and C57BL/6 were bred in the animal facility of the Biotechnology Centre of the Cardarelli Hospital (Naples, Italy) with C57BL/6 mice (Charles Rivers Laboratories, Calco, Italy), to obtain the P347S+/− mice.

### Vector Administration

### Mice

Intraperitoneal injection of ketamine and medetomidine were administered (100 mg/kg and 0.25 mg/kg respectively), then AAV vectors were delivered sub-retinally via a trans-scleral transchoroidal approach (12, 13).

### Pigs

Eleven-week-old Large White (LW) female piglets were used. Pigs were fasted overnight leaving water ad libitum. The anaesthetic and surgical procedures for pigs were previously described (12). Each viral vector was injected in a total volume of 100 μl, resulting in the formation of a subretinal bleb with a typical ‘dome-shaped’ retinal detachment, with a size corresponding to 5 optical discs (12, 13).

### Human Retina

In collaboration with the Eye Bank of Venice, the inventors collected retina samples from a donor in compliance with the tenets of the Declaration of Helsinki and after obtaining the informed consent from the donor's next of kin.

### Cloning and Protein Purification

DNA fragments encoding the sequence of the engineered transcription factors ZF6-DB and hKLF15, to be expressed as maltose-binding protein (MBP) fusion were generated by PCR using the plasmids pAAV2.1 CMV-hKLF15 and pAAV2.1 CMV-ZF6-DB as a DNA template. The following oligonucleotides were used as primers: primer 1, (GGAATTCCATATGGTGGACCACTTACTTCCAG, SEQ ID No. 1) and primer 2, (CGGGATCCTCAGTTCACGGAGCGCACGGAG, SEQ ID No. 2), for hKLF15 primer 3, (GGAATTCCATATGCTGGAACCTGGCGAAAAACCG, SEQ ID No. 3) and primer 4, (CGGGATCCCTATCTAGAAGTCTTTTTACCGGTATG, SEQ ID No. 4) for ZF6-DB. All PCR products were digested with the restriction enzymes Ndel and BamH1 and cloned into an Ndel BamH1-digested pMal C5G (New England Biolabs) bacterial expression vector. All the plasmids obtained were sequenced to confirm that there were no mutations in the coding sequences. The fusion proteins were expressed in the Escherichia coli BL21DE3 host strain. The transformed cells were grown in rich medium plus 0.2% glucose (according to the protocol from New England Biolabs) at 37° C. until the absorbance at 600 nm was 0.6-0.8, at which time the medium was supplemented with 200 μM ZnSO4, and protein expression was induced with 0.3 mM isopropyl 1-thio-β-D-galactopyranoside and was allowed to proceed for 2 h. The cells were then harvested, resuspended in 1×PBS (pH 7.4), 1 mM phenylmethylsulfonyl fluoride, 1 μM leupeptin, 1 μM aprotinin, and 10 μg/ml lysozyme, sonicated, and centrifuged for 30 min at 27,500 rpm. The supernatant was then loaded on an amylose resin (New England Biolabs) according to the manufacturer's protocol. To remove the MBP from the proteins, bound fusion proteins were cleaved in situ on the amylose resin with Factor Xa (1 unit/20 μg of MBP fusion protein) in FXa buffer (20 mM Tris, pH 8.0, 100 mM NaCl, 2 mM CaCl2) for 24-48 h at 4° C. and collected in the same buffer after centrifugation at 500 rpm for 5 min. The supernatant containing the protein without the MBP tag was then recovered.

### Gel Mobility Shift Analysis

The affinity binding constant of proteins for the hRHO proximal promoter sequence was measured by a gel mobility shift assay by performing a titration of the proteins with the oligonucleotides. The purified proteins were incubated for 15 min on ice with a hRHO 65 bp duplex oligonucleotide in the presence of 25 mM Hepes (pH 7.9), 50 mM KCl, 6.25 mM MgCl2, 1% Nonidet P-40, 5% glycerol. After incubation, the mixture was loaded on a 5% polyacrylamide gel (29:1 acrylamide/bisacrylamide ratio) and run in 0.5 TBE at 4° C. (200 V for 4 h). Protein concentration was determined by a modified version of the Bradford procedure. After electrophoresis, the gel was stained with the fluorescent dye SYBR® Green I Nucleic acid gel stain (Invitrogen) to visualize DNA. 2.5 μM of the hKLF15 protein was incubated with increasing concentrations (145, 150, 170, 175, 190, 195, 200, 220, 240, and 250 nM) of the duplex hRHO 65 bp oligonucleotide. In the case of ZF6-DB, 1.5 μM of the protein was incubated with increasing concentrations (145, 150, 170, 175, 195, 210, 220, 225, 240, and 250 nM) of the duplex hRho 65 bp. Scatchard analysis of the gel shift binding data was performed to obtain the Kd values (12). All numerical values were obtained by computer quantification of the image using a Typhoon FLA 9500 biomolecular imager (GE Healthcare Life Sciences).

gReal Time PCR

RNA from tissues were isolated using RNAeasy Mini Kit (Qiagen), according to the manufacturer's protocol. cDNA was amplified from 1 μg isolated RNA using QuantiTect Reverse Transcription Kit (Qiagen), as indicated in the manufacturer's instructions.

PCR using the cDNA as template was performed in a total volume of 20 μl, using 10 μl LightCycler 480 SYBR Green I Master Mix (Roche) and 400 nM primers under the following conditions: pre-Incubation, 50° C. for 5 min, cycling: 45 cycles of 95° C. for 10 s, 60° C. for 20 s and 72° C. for 20 s. Each sample was analyzed in duplicate in two independent experiments. Transcript levels of pig retinae were measured by real-time PCR using the LightCycler 480 (Roche) and the following primers: pRho_forward (ATCAACTTCCTCACGCTCTAC, SEQ ID No. 5) and pRho_reverse (ATGAAGAGGTCAGCCACTGCC, SEQ ID No. 6), pGnat1_forward (TGTGGAAGGACTCGGGTATC, SEQ ID No. 7) and pGnat1_reverse (GTCTTGACACGTGAGCGTA, SEQ ID No. 8), pArr3_forward (TGACAACTGCGAGAAACAGG, SEQ ID No. 9) and pArr3_reverse (CACAGGACACCATCAGGTTG, SEQ ID No. 10), pCrx_forward (GAGCTGGAGTCCTTGTTTGC, SEQ ID No. 11) and pCrx_reverse (CGTGGAGGATCTTGGAGAAG, SEQ ID No. 24), pNrl_forward (CAGAGCTGCTGCAGTGTCA, SEQ ID No. 25) and pNrl_reverse (GTTCAACTCGCGCACAGAC, SEQ ID No. 26), pKlf15_forward (GCAGGACAGCATCTTGGACT, SEQ ID No. 27) and pKlf15_reverse (ACAGGAGCTGGTGTTTTTCG, SEQ ID No. 28). All of the reactions were standardized against porcine Actp using the following primers: Act_Forward (ACGGCATCGTCACCAACTG, SEQ ID No. 29) and Act_reverse (CTGGGTCATCTTCTCACGG, SEQ ID No. 30). Transcript levels of mouse retinae were measured by real-time PCR using the LightCycler 480 (Roche) and the following primers: mRho_Forward (GACTCTGCCAGCTTTCTTTGCT, SEQ ID No. 31) and mRho_Reverse (GCGTCGTCATCTCCCAGTGGA, SEQ ID No. 32), hRho_Forward (CCATCCCAGCGTTCTTTGCC, SEQ ID No. 33) and hRho_Reverse (CCTCATCGTCACCCAGTGGG, SEQ ID No. 34), mGnat1_Forward (GACCGAGCCTCAGAATACCA, SEQ ID No. 35) and mGnat1_Reverse (GGAGAATTGAGTCTCGATAATACCA, SEQ ID No. 36); All of the reactions were standardized against porcine Acts using the following primers: mAct_Forward (CAAGATCATTGCTCCTCCTGA, SEQ ID No. 37) and mAct_reverse (CATGCTACTCCTGCTTGCTGA, SEQ ID No. 38), mGapdh_forward (GTCGGTGTGAACGGATTTG, SEQ ID No. 39) and mGapdh_reverse (CAATGAAGGGGTCGTTGATG, SEQ ID No. 40).

### Immunostaining

Frozen retinal sections were washed once with PBS and then fixed for 10 min in 4% PFA. Sections were blocked and permeabilized with 0.3% Triton X-100 and 5% donkey serum in TBS for 1 hour. The primary antibody mouse anti-KLF15 (1:200, abcam, ab185958) was diluted in a blocking solution and incubated overnight at room temperature. The secondary antibody (Alexa Fluor® 594, anti-rabbit 1:1000, Molecular Probes, Invitrogen, Carlsbad, CA) was incubated for 1 hour. Vectashield (Vector Lab Inc., Peterborough, UK) was used to visualize nuclei. Frozen retinal sections were permeabilized with 0.2% Triton X-100 and 1% NGS for 1 hour, rinsed in PBS, blocked in 10% normal goat serum (NGS), and then incubated overnight at 4° C. with rabbit human cone arrestin (hCAR) antibody, kindly provided by Dr. Cheryl M. Craft (Doheny Eye Institute, Los Angeles, CA) diluted 1:10,000 in 10% NGS. After three rinses with 0.1 M PBS, sections were incubated in goat anti-rabbit IgG conjugated with Texas red (Alexa Fluor® 594, anti-rabbit 1:1000, Molecular Probes, Invitrogen, Carlsbad, CA) for 1 hour followed by three rinses with PBS. Frozen retinal sections were permeabilized with 0.1% Triton X-100, rinsed in PBS, blocked in 10% normal goat serum (NGS), and then incubated overnight at 4° C. in a mouse anti-1D4 rhodopsin antibody diluted 1:500 in 10% NGS. After three rinses with 0.1 M PBS, sections were incubated in goat anti-mouse IgG conjugated with Texas red (Alexa Fluor® 594, anti-mouse 1:1000, Molecular Probes, Invitrogen, Carlsbad, CA) for 1 hour followed by three washes with PBS. Frozen retinal sections were permeabilized with 0.1% Triton X-100, rinsed in PBS, blocked in 10% normal goat serum (NGS), and then incubated overnight at 4° C. in a rabbit Gα T1-K20 (1:300, Santa Cruz Biotechnology) in blocking solution. After three rinses with 0.1 M PBS, sections were incubated in goat anti-mouse IgG conjugated with Texas red (Alexa Fluor® 594, anti-rabbit 1:500, Molecular Probes, Invitrogen, Carlsbad, CA) for 1 hour followed by three washes with PBS.

Mouse eyes were enucleated and fixed with 4% formaldehyde in 0.1 M sodium phosphate buffer, pH 7.4 for 16 h at 4° C. The tissues were then dehydrated through a graded sucrose series and embedded in OCT. Sections (12 μm thick) were cut. Hematoxylin and eosin (H&E) staining was performed. Sections were photographed using either a Zeiss 800 Confocal Microscope (Carl Zeiss, Oberkochen, Germany) or a Leica Fluorescence Microscope System (Leica Microsystems GmbH, Wetzlar, Germany).

### Western Blot Analyses

Western blot analysis was performed on harvested retina. Samples were lysed in hypotonic buffer (10 mM Tris-HCl [pH 7.5], 10 mM NaCl, 1.5 mM MgCl2, 1% CHAPS, 1 mM PMSF, and protease inhibitors) and 20 μg of these lysates were separated by 12% SDS-PAGE. After the blots were obtained, specific proteins were labeled with anti-1D4 antibody anti-Rhodopsin-1D4 (1:1000; Abcam, Cambridge, MA), and anti-β-tubulin (1:10,000; Sigma-Aldrich, Milan, Italy) antibodies.

### Chromatin Immunoprecipitation Experiments (ChIP)

For ChIP experiments, HEK293 cells were transfected by CaCl2) with pAAV2.1 CMV-hKLF15 or pAAV2.1 CMV-eGFP. The cells were harvested after 48 hours. ChIP was performed as follows: cells were homogenized mechanically and cross linked using 1% formaldehyde in PBS at room temperature for 10 minutes, then quenched by adding glycine at final concentration 125 mM and incubated at room temperature for 5 minutes. Cells were washed three times in cold PBS 1× and then lysed in cell lysis buffer (Pipes 5 mM pH 8.0, Igepal 0.5%, KCl 85 mM) for 15 min. Nuclei were lysed in nucleus lysis buffer (Tris HCl pH8.0 50 mM, EDTA 10 mM, SDS 0.8%) for 30 min. Chromatin was sheared using Covaris s220. The sheared chromatin was immunoprecipitated over night with anti-KLF15 (2G8) ChIP grade (Abcam, ab81604, Cambridge, MA). The immunoprecipitated chromatin was incubated 3 hours with magnetic protein A/G beads (Invitrogen, Carlsbad, CA). Beads were than washed with wash buffers and DNA eluted in elution buffer (Tris HCl pH8 50 mM, EDTA 1 mM, SDS 1%). Real time PCR was performed using primers on rhodopsin TSS, hRHOTSSFw (TGACCTCAGGCTTCCTCCTA, SEQ ID No. 41) and hRHOTSSRv (ATCAGCATCTGGGAGATTGG, SEQ ID No. 42).

### FACS Rods Sorting

Injected porcine retinas with AAV8-GNAT1-eGFP (dose 1×1012 gc) were disaggregated using Papain Dissociation System (Worthington biochemical corporation) following the manufacturer's protocol. Dissociated retinal cells were analysed using BD FACSAria Ill and sorted, dividing eGFP positive cells (rods) from the eGFP negative fraction.

### Electrophysiological Testing

The method used was described previously (12, 13). Briefly, mice were dark reared for three hours and anesthetized. Flash electroretinograms (ERGs) were evoked by 10-ms light flashes generated through a Ganzfeld stimulator (CSO, Costruzione Strumenti Oftalmici, Florence, Italy) and registered as previously described. ERGs and b-wave thresholds were assessed using the following protocol. Eyes were stimulated with light flashes increasing from −5.2 to +1.3 log cd*s/m2 (which correspond to 1×10-5.2 to 20.0 cd*s/m2) in scotopic conditions. The log unit interval between stimuli was 0.3 log from −5.4 to 0.0 log cd*s/m2, and 0.6 log from 0.0 to +1.3 log cd*s/m2. For ERG analysis in scotopic conditions the responses evoked by 11 stimuli (from −4 to +1.3 log cd*s/m2) with an interval of 0.6 log unit were considered. To minimize the noise, three ERG responses were averaged at each 0.6 log unit stimulus from −4 to 0.0 log cd*s/m2 while one ERG response was considered for higher (0.0-+1.3 log cd*s/m2) stimuli. The time interval between stimuli was 10 seconds from −5.4 to 0.7 log cd*s/m2, 30 see from 0.7 to +1 log cd*s/m2, or 120 seconds from +1 to +1.3 log cd*s/m2. a- and b-wave amplitudes recorded in scotopic conditions were plotted as a function of increasing light intensity (from −4 to +1.3 log cd*s/m2). The photopic ERG was recorded after the scotopic session by stimulating the eye with ten 10 ms flashes of 20.0 cd*s/m2 over a constant background illumination of 50 cd/m2.

### RNASeq Library Preparation, Sequencing and Alignment

The 16 libraries were prepared using the TruSeq RNA v2 Kit (Illumina, San Diego, CA) according to the manufacturer's protocol. Libraries were sequenced on the Illumina HiSeq 1000 platform and in 100-nt paired-end format to obtain approximately 30 million read pairs per sample as reported (12, 13).

### Differential Expression Analysis

The dataset was composed of 16 samples and 25,325 genes, divided in 3 experimental groups: 6 Controls, 4 KLF15-treated, 6 ZF6-DB-treated (12, 13).

### Data Management

All analyses, except for the reads quality filtering, alignment and expression estimates, were performed in the R statistical environment (v.3.2.0) (32). Plots were generated with ggplot2 R/Bioconductor package (v.1.0.1) (12, 13).

### Statistical Analyses

Data are presented as mean±Error bars indicate standard error mean (SEM). Statistical significance was computed using the Student's two-sided t-test and p-values<0.05 were considered significant. No statistical methods were used to estimate the sample size and no animals were excluded.

### Study Approval

### Animals

Animal experimentation: All procedures were performed in accordance with institutional guidelines for animal research and all of the animal studies were approved by the authors. The protocol was approved by the Italian Ministry for Health (IACUC protocols #114/2015-PR).

### Human Retina

The “Fondazione Banca degli Occhi del Veneto” (Eye Bank of Venice) provided retina samples from a donor in compliance with the tenets of the Declaration of Helsinki and after obtaining the informed consent from the donor's next of kin.

### REFERENCES

1. Swaroop A, Kim D, and Forrest D. Transcriptional regulation of photoreceptor development and homeostasis in the mammalian retina. Nat Rev Neurosci. 2010; 11(8):563-76.
2. Levo M, and Segal E. In pursuit of design principles of regulatory sequences. Nat Rev Genet. 2014; 15(7):453-68.
3. Rohs R, Jin X, West S M, Joshi R, Honig B, and Mann R S. Origins of specificity in protein-DNA recognition. Annu Rev Biochem. 2010; 79(233-69.
4. Seeman N C, Rosenberg J M, and Rich A. Sequence-specific recognition of double helical nucleic acids by proteins. Proc Natl Acad Sci USA. 1976; 73(3):804-8.
5. Klug A. The discovery of zinc fingers and their applications in gene regulation and genome manipulation. Annu Rev Biochem. 2010; 79(213-31.
6. Pavletich N P, and Pabo C O. Zinc finger-DNA recognition: crystal structure of a Zif268-DNA complex at 2.1 A. Science. 1991; 252(5007):809-17.
7. Weirauch M T, Cote A, Norel R, Annala M, Zhao Y, Riley T R, Saez-Rodriguez J, Cokelaer T, Vedenko A, Talukder S, et al. Evaluation of methods for modeling transcription factor sequence specificity. Nat Biotechnol. 2013; 31(2):126-34.
8. Jolma A, Yin Y, Nitta K R, Dave K, Popov A, Taipale M, Enge M, Kivioja T, Morgunova E, and Taipale J. DNA-dependent formation of transcription factor pairs alters their binding specificity. Nature. 2015; 527(7578):384-8.
9. Reiter F, WienerroitherS, and Stark A. Combinatorial function of transcription factors and cofactors. Curr Opin Genet Dev. 2017; 43(73-81.
10. Thurman R E, Rynes E, Humbert R, Vierstra J, Maurano M T, Haugen E, Sheffield N C, Stergachis A B, Wang H, Vernot B, et al. The accessible chromatin landscape of the human genome. Nature. 2012; 489(7414):75-82.
11. Hartong D T, Berson E L, and Dryja T P. Retinitis pigmentosa. Lancet. 2006; 368(9549):1795-809.
12. Botta S, Marrocco E, de Prisco N, Curion F, Renda M, Sofia M, Lupo M, Carissimo A, Bacci M L, Gesualdo C, et al. Rhodopsin targeted transcriptional silencing by DNA-binding. Elife. 2016; 5(e12242.
13. Mussolino C, Sanges D, Marrocco E, Bonetti C, Di Vicino U, Marigo V, Auricchio A, Meroni G, and Surace E M. Zinc-finger-based transcriptional repression of rhodopsin in a model of dominant retinitis pigmentosa. EMBO Mol Med. 2011; 3(3):118-28.
14. Mo A, Luo C, Davis F P, Mukamel E A, Henry G L, Nery J R, Urich M A, Picard S, Lister R, Eddy S R, et al. Epigenomic landscapes of retinal rods and cones. Elife. 2016; 5(e11613.
15. Wingender E, Dietze P, Karas H, and Knuppel R. TRANSFAC: a database on transcription factors and their DNA binding sites. Nucleic Acids Res. 1996; 24(1):238-41.
16. Pearson R, Fleetwood J, Eaton S, Crossley M, and Bao S. Kruppel-like transcription factors: a functional family. Int J Biochem Cell Biol. 2008; 40(10):1996-2001.
17. Otteson D C, Liu Y, Lai H, Wang C, Gray S, Jain M K, and Zack D J. Kruppel-like factor 15, a zinc-finger transcriptional regulator, represses the rhodopsin and interphotoreceptor retinoid-binding protein promoters. Invest Ophthalmol Vis Sci. 2004; 45(8):2522-30.
18. Gray S, Wang B, Orihuela Y, Hong E G, Fisch S, Haldar S, Cline G W, Kim J K, Peroni O D, Kahn B B, et al. Regulation of gluconeogenesis by Kruppel-like factor 15. Cell Metab. 2007; 5(4):305-12.
19. Jeyaraj D, Haldar S M, Wan X, McCauley M D, RippergerJA, Hu K, Lu Y, Eapen B L, Sharma N, Ficker E, et al. Circadian rhythms govern cardiac repolarization and arrhythmogenesis. Nature. 2012; 483(7387):96-9.
20. Lu Y, Zhang L, Liao X, Sangwung P, Prosdocimo D A, Zhou G, Votruba A R, Brian L, Han Y J, Gao H, et al. Kruppel-like factor 15 is critical for vascular inflammation. J Clin Invest. 2013; 123(10):4232-41.
21. Fisch S, Gray S, Heymans S, Haldar S M, Wang B, Pfister O, Cui L, Kumar A, Lin Z, Sen-Banerjee S, et al. Kruppel-like factor 15 is a regulator of cardiomyocyte hypertrophy. Proc Natl Acad Sci USA. 2007; 104(17):7074-9.
22. Sasse S K, Mailloux C M, Barczak A J, Wang Q, Altonsy M O, Jain M K, Haldar S M, and Gerber A N. The glucocorticoid receptor and KLF15 regulate gene expression dynamics and integrate signals through feed-forward circuitry. Mol Cell Biol. 2013; 33(11):2104-15.
23. Li T, Snyder W K, Olsson J E, and Dryja T P. Transgenic mice carrying the dominant rhodopsin mutation P347S: evidence for defective vectorial transport of rhodopsin to the outer segments. Proc Natl Acad Sci USA. 1996; 93(24):14176-81.
24. White M A, Myers C A, Corbo J C, and Cohen B A. Massively parallel in vivo enhancer assay reveals that highly local features determine the cis-regulatory function of ChIP-seq peaks. Proc Natl Acad Sci USA. 2013; 110(29):11952-7.
25. Montana C L, Lawrence K A, Williams N L, Tran N M, Peng G H, Chen S, and Corbo J C. Transcriptional regulation of neural retina leucine zipper (Nrl), a photoreceptor cell fate determinant. J Biol Chem. 2011; 286(42):36921-31.
26. Yu W, Mookherjee S, Chaitankar V, Hiriyanna S, Kim J W, Brooks M, Ataeijannati Y, Sun X, Dong L, Li T, et al. Nrl knockdown byAAV-delivered CRISPR/Cas9 prevents retinal degeneration in mice. Nat Commun. 2017; 8(14716.
27. Imbeault M, Helleboid P Y, and Trono D. KRAB zinc-finger proteins contribute to the evolution of gene regulatory networks. Nature. 2017; 543(7646):550-4.
28. Nowick K, Hamilton A T, Zhang H, and Stubbs L. Rapid sequence and expression divergence suggest selection for novel function in primate-specific KRAB-ZNF genes. Mol Biol Evol. 2010; 27(11):2606-17.
29. Consortium G T, Laboratory D A, Coordinating Center—Analysis Working G, Statistical Methods groups-Analysis Working G, Enhancing Gg, Fund NIHC, Nih/Nci, Nih/Nhgri, Nih/Nimh, Nih/Nida, et al. Genetic effects on gene expression across human tissues. Nature. 2017; 550(7675):204-13.
30. Auricchio A, Smith A J, and Ali R R. The Future Looks Brighter After 25 Years of Retinal Gene Therapy. Hum Gene Ther. 2017; 28(11):982-7.
31. Bennett J. Taking Stock of Retinal Gene Therapy: Looking Back and Moving Forward. Mol Ther. 2017; 25(5):1076-94.
32. Huber W, Carey V J, Gentleman R, Anders S, Carlson M, Carvalho B S, Bravo H C, Davis S, Gatto L, Girke T, et al. Orchestrating high-throughput genomic analysis with Bioconductor. Nat Methods. 2015; 12(2):115-21.
33. Solovei I, Kreysing M, Lanctot C, Kosem S, Peichl L, Cremer T, Guck J, and Joffe B. Nuclear architecture of rod photoreceptor cells adapts to vision in mammalian evolution. Cell. 2009; 137(2):356-68.

1. A nucleic acid construct comprising:
a) a nucleotide sequence encoding a first promoter;
b) a nucleotide sequence encoding a transcription factor
wherein the nucleotide sequence of a) is operably linked to and drives the expression of the nucleotide sequence of b) in rod cells or cone cells of the retina where the protein encoded by the nucleotide sequence of b) is not physiologically expressed and
wherein the protein encoded by said nucleotide sequence of b) recognizes a nucleotide sequence belonging to a gene which mutated form is responsible for a retinal dystrophy thereby silencing the expression of said gene.

2. The nucleic acid construct according claim 1 wherein the gene which mutated form is responsible for the retinal dystrophy is selected from RHO, PRPH2, CRX, RP1, GUCA1B, RDH12, N2RE3, NRL, ROM1, OTX2, GUCA1A, GUCY2D.

3. The nucleic acid construct according to claim 1 wherein the transcription factor is selected from:
any one transcription factors described in Table 2 when the gene is RHO,
any one transcription factors described in Table 4 when the gene is CRX,
any one transcription factors described in Table 5 when the gene is GUCA1B,
any one transcription factors described in Table 6 when the gene is PRP2,
any one transcription factors described in Table 7 when the gene is RDH12,
any one transcription factors described in Table 8 when the gene is RP1
any one transcription factors described in Table 9 when the gene is GUCA1A
any one transcription factors described in Table 10 when the gene is GUCY2D
any one transcription factors described in Table 11 when the gene is N2RE3
any one transcription factors described in Table 12 when the gene is NRL
any one transcription factors described in Table 13 when the gene is OTX2
any one transcription factors described in Table 14 when the gene is ROM1.

4. The nucleic acid construct according to claim 1, further comprising a nucleotide sequence coding for a wild-type form of a mutated coding sequence, wherein said mutated coding sequence is responsible for the retinal dystrophy.

5. The nucleic acid constructs according to claim 4, wherein said nucleotide sequence coding for a wild-type form of a mutated coding sequence is under the control of a nucleotide sequence of a second promoter.

6. The nucleic acid construct according to claim 1, wherein the first and/or second promoter is GNAT1, any one of promoter defined by SEQ ID No. 13 to 23, red opsin or a promoter of a gene is selected from RHO, PRPH2, CRX, RP1, GUCA1B, RDH12, N2RE3, 5RL, ROM1, OTX2, GUCA1A, GUCY2D.

7. The nucleic acid construct according to claim 1, wherein the nucleotide sequence of the construct comprises any one of SEQ TD No. 837 to SEQ ID No. 881.

8. The nucleic acid construct according to claim 1, wherein the retinal dystrophy is selected from retinitis pigmentosa, Leber's congenital amaurosis, cone dystrophy or cone-rod dystrophy.

9. An expression vector that comprises the nucleic acid construct according to claim 1 and a second nucleic acid construct comprising a nucleotide sequence coding for a wild-type form of a mutated coding sequence, wherein said mutated coding sequence is responsible for the retinal dystrophy.

10. The expression vector according to claim 9, wherein the vector is selected from the group consisting of: adenoviral vector, lentiviral vector, retroviral vector, Adeno associated vector (AAV) and naked plasmid DNA vector.

11. A host cell comprising the nucleic acid construct according to claim 1.

12. A viral particle that comprises a nucleic acid construct according to claim 1.

13. The viral particle according to claim 12, wherein the viral particle comprises capsid proteins of an AAV.

14. The viral particle according to claim 13, wherein the viral particle comprises capsid proteins of an AAV of a serotype selected from one or more of the group consisting of AAV1, AAV2, AAV3, AAV4, AAV5, AAV6, AAV7, AAV8 AAV9 and AAV 10.

15. A pharmaceutical composition that comprises a nucleic acid construct according to claim 1, and a pharmaceutically acceptable carrier.

16. A kit comprising a nucleic acid construct according to claim 1 in one or more containers, optionally further comprising instructions or packaging materials that describe how to administer the nucleic acid construct, vector, host cell, viral particle or pharmaceutical composition to a patient.

17. (canceled)

18. A method for the treatment of retinal dystrophy, comprising administering a nucleic acid construct of claim 1 to a patient in need thereof.

19. (canceled)


SEQUENCE LISTING (883 sequences)

SEQ ID NO: 1 - DNA, 32 bp, Artificial Sequence
  synthetic primer
ggaattccatatggtggaccacttacttccag

SEQ ID NO: 2 - DNA, 30 bp, Artificial Sequence
  synthetic primer
cgggatcctcagttcacggagcgcacggag

SEQ ID NO: 3 - DNA, 34 bp, Artificial Sequence
  synthetic primer
ggaattccatatgctggaacctggcgaaaaaccg

SEQ ID NO: 4 - DNA, 35 bp, Artificial Sequence
  synthetic primer
cgggatccctatctagaagtctttttaccggtatg

SEQ ID NO: 5 - DNA, 21 bp, Artificial Sequence
  synthetic primer
atcaacttcctcacgctctac

SEQ ID NO: 6 - DNA, 21 bp, Artificial Sequence
  synthetic primer
atgaagaggtcagccactgcc

SEQ ID NO: 7 - DNA, 20 bp, Artificial Sequence
  synthetic primer
tgtggaaggactcgggtatc

SEQ ID NO: 8 - DNA, 19 bp, Artificial Sequence
  synthetic primer
gtcttgacacgtgagcgta

SEQ ID NO: 9 - DNA, 20 bp, Artificial Sequence
  synthetic primer
tgacaactgcgagaaacagg

SEQ ID NO: 10 - DNA, 20 bp, Artificial Sequence
  synthetic primer
cacaggacaccatcaggttg

SEQ ID NO: 11 - DNA, 20 bp, Artificial Sequence
  synthetic primer
gagctggagtccttgtttgc

SEQ ID NO: 12 - DNA, 654 bp, Artificial Sequence
  synthetic primer
tccctgcaggtcataaaatcccagtccagagtcaccagcccttcttaaccacttcctactgtgtgaccctttcagcctttacttcctcatcagtaaaatgaggctgatgatatgggcatccatactccagggccagtgtgagcttacaacaagataaggagtggtgctgagcctggtgccgggcaggcagcaggcatgtttctcccaattatgccctctcactgccagccccacctccattgtcctcacccccagggctcaaggttctgccttcccctttctcagccctgaccctactgaacatgtctccccactcccaggcagtgccagggcctctcctggagggttgcggggacagaaggacagccggagtgcagagtcagcggttgagggattggggctatgccagctaatccgaagggttgggggggctgagctggattcacctgtccttgtctctgattggctcttggacacccctagcccccaaatcccactaagcagccccaccagggattgcacaggtccgtagagagccagttgattgcaggtcctcctggggccagaagggtgcctgggaggccaggttctggggatcccctccatccagaagaaccacctgctcactctgtcccttcgcctgctgctgggacc

SEQ ID NO: 13 - DNA, 254 bp, Artificial Sequence
  synthetic primer
tcctcctagtgtcaccttggcccctcttagaagccaattaggccctcagtttctgcagcggggattaatatgattatgaacacccccaatcgatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 14 - DNA, 259 bp, Artificial Sequence
  synthetic primer
tcctcctagtgtcaccttggcccctcttagaagccaattaggccctcagtttctgcagcggggattaatatgattatgaacacccccaatctcaactcgtaggattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 15 - DNA, 259 bp, Artificial Sequence
  synthetic primer
tcctcctagtgtcaccttggcccctcttagaagccaattaggccctcagtttctgcagcggggattaatatgattatgaacacccccacgagaaactctgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 16 - DNA, 259 bp, Artificial Sequence
  synthetic primer
tcctcctagtgtcaccttggcccctcttagaagccaattaggccctcagtttctgcagcggggattaatatgattagtccacaccccacgagaaactctgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 17 - DNA, 259 bp, Artificial Sequence
  synthetic primer
tcctcctagtgtcaccttggcccctcttagaagccaattaggccctcagtttctgcagcggggattaatatgattatgaacacatgatatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 18 - DNA, 254 bp, Artificial Sequence
  synthetic primer
tcctcctagtgtcaccttggcccctcttagaagccaattaggccctcagtttctgcagcggggattaatatgattatgaacacatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 19 - DNA, 259 bp, Artificial Sequence
  synthetic primer
tcctcctagtgtcaccttggcccctcttagaagccaattaggccctcagtttctgcagcggggattaatatgattagtccacaccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 20 - DNA, 259 bp, Artificial Sequence
  synthetic primer
tcctcctagtgtcaccttggcccctcttagaagccaattaggccctcagtttctgcagcggggattaatatgattacgaccgtatcggggttagggagtgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 21 - DNA, 254 bp, Artificial Sequence
  synthetic primer
tcctcctagtgtcaccttggcccctcttagaagccaattaggccctcagtttctgcagcggggattaatatgattatcccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 22 - DNA, 259 bp, Artificial Sequence
  synthetic primer
tcctcctagtgtcaccttggcccctcttagaagccaattaggccctcagtttctgcagcggggattaatatgattagagggattggtgctatgccagctgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 23 - DNA, 251 bp, Artificial Sequence
  synthetic primer
tcctcctagtgtcaccttggcccctcttagaagccaattaggccctcagtttctgcagcggggattaatatgattatgaaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 24 - DNA, 20 bp, Artificial Sequence
  synthetic primer
cgtggaggatcttggagaag

SEQ ID NO: 25 - DNA, 19 bp, Artificial Sequence
  synthetic primer
cagagctgctgcagtgtca

SEQ ID NO: 26 - DNA, 19 bp, Artificial Sequence
  synthetic primer
gttcaactcgcgcacagac

SEQ ID NO: 27 - DNA, 20 bp, Artificial Sequence
  synthetic primer
gcaggacagcatcttggact

SEQ ID NO: 28 - DNA, 20 bp, Artificial Sequence
  synthetic primer
acaggagctggtgtttttcg

SEQ ID NO: 29 - DNA, 19 bp, Artificial Sequence
  synthetic primer
acggcatcgtcaccaactg

SEQ ID NO: 30 - DNA, 19 bp, Artificial Sequence
  synthetic primer
ctgggtcatcttctcacgg

SEQ ID NO: 31 - DNA, 22 bp, Artificial Sequence
  synthetic primer
gactctgccagctttctttgct

SEQ ID NO: 32 - DNA, 21 bp, Artificial Sequence
  synthetic primer
gcgtcgtcatctcccagtgga

SEQ ID NO: 33 - DNA, 20 bp, Artificial Sequence
  synthetic primer
ccatcccagcgttctttgcc

SEQ ID NO: 34 - DNA, 20 bp, Artificial Sequence
  synthetic primer
cctcatcgtcacccagtggg

SEQ ID NO: 35 - DNA, 20 bp, Artificial Sequence
  synthetic primer
gaccgagcctcagaatacca

SEQ ID NO: 36 - DNA, 25 bp, Artificial Sequence
  synthetic primer
ggagaattgagtctcgataatacca

SEQ ID NO: 37 - DNA, 21 bp, Artificial Sequence
  synthetic primer
caagatcattgctcctcctga

SEQ ID NO: 38 - DNA, 21 bp, Artificial Sequence
  synthetic primer
catgctactcctgcttgctga

SEQ ID NO: 39 - DNA, 19 bp, Artificial Sequence
  synthetic primer
gtcggtgtgaacggatttg

SEQ ID NO: 40 - DNA, 20 bp, Artificial Sequence
  synthetic primer
caatgaaggggtcgttgatg

SEQ ID NO: 41 - DNA, 20 bp, Artificial Sequence
  synthetic primer
tgacctcaggcttcctccta

SEQ ID NO: 42 - DNA, 20 bp, Artificial Sequence
  synthetic primer
atcagcatctgggagattgg

SEQ ID NO: 43 - DNA, 17 bp, Artificial Sequence
  synthetic primer
tgaacacccccaatctc

SEQ ID NO: 44 - DNA, 10 bp, Artificial Sequence
  synthetic primer
gaacaccccc

SEQ ID NO: 45 - DNA, 11 bp, Artificial Sequence
  synthetic primer
gaacaccccca

SEQ ID NO: 46 - DNA, 14 bp, Artificial Sequence
  synthetic primer
gaacacccccaatc

SEQ ID NO: 47 - DNA, 10 bp, Artificial Sequence
  synthetic primer
gaacaccccc

SEQ ID NO: 48 - DNA, 9 bp, Artificial Sequence
  synthetic primer
gaacacccc

SEQ ID NO: 49 - DNA, 10 bp, Artificial Sequence
  synthetic primer
gaacaccccc

SEQ ID NO: 50 - DNA, 9 bp, Artificial Sequence
  synthetic primer
aacaccccc

SEQ ID NO: 51 - DNA, 10 bp, Artificial Sequence
  synthetic primer
acacccccaa

SEQ ID NO: 52 - DNA, 8 bp, Artificial Sequence
  synthetic primer
acaccccc

SEQ ID NO: 53 - DNA, 7 bp, Artificial Sequence
  synthetic primer
caccccc

SEQ ID NO: 54 - DNA, 7 bp, Artificial Sequence
  synthetic primer
caccccc

SEQ ID NO: 55 - DNA, 6 bp, Artificial Sequence
  synthetic primer
cacccc

SEQ ID NO: 56 - DNA, 7 bp, Artificial Sequence
  synthetic primer
caccccc

SEQ ID NO: 57 - DNA, 7 bp, Artificial Sequence
  synthetic primer
accccca

SEQ ID NO: 58 - DNA, 6 bp, Artificial Sequence
  synthetic primer
accccc

SEQ ID NO: 59 - DNA, 11 bp, Artificial Sequence
  synthetic primer
acccccaatct

SEQ ID NO: 60 - DNA, 6 bp, Artificial Sequence
  synthetic primer
ccccca

SEQ ID NO: 61 - DNA, 7 bp, Artificial Sequence
  synthetic primer
cccccaa

SEQ ID NO: 62 - DNA, 7 bp, Artificial Sequence
  synthetic primer
cccccaa

SEQ ID NO: 63 - DNA, 8 bp, Artificial Sequence
  synthetic primer
ccccaatc

SEQ ID NO: 64 - DNA, 8 bp, Artificial Sequence
  synthetic primer
ccccaatc

SEQ ID NO: 65 - DNA, 8 bp, Artificial Sequence
  synthetic primer
ccccaatc

SEQ ID NO: 66 - DNA, 10 bp, Artificial Sequence
  synthetic primer
cccaatctcc

SEQ ID NO: 67 - DNA, 10 bp, Artificial Sequence
  synthetic primer
cccaatctcc

SEQ ID NO: 68 - DNA, 9 bp, Artificial Sequence
  synthetic primer
cccaatctc

SEQ ID NO: 69 - DNA, 10 bp, Artificial Sequence
  synthetic primer
cccaatctcc

SEQ ID NO: 70 - DNA, 10 bp, Artificial Sequence
  synthetic primer
cccaatctcc

SEQ ID NO: 71 - DNA, 7 bp, Artificial Sequence
  synthetic primer
ccaatct

SEQ ID NO: 72 - DNA, 12 bp, Artificial Sequence
  synthetic primer
aatctcccagat

SEQ ID NO: 73 - DNA, 11 bp, Artificial Sequence
  synthetic primer
aatctcccaga

SEQ ID NO: 74 - DNA, 10 bp, Artificial Sequence
  synthetic primer
atctcccaga

SEQ ID NO: 75 - DNA, 16 bp, Artificial Sequence
  synthetic primer
atctcccagatgctga

SEQ ID NO: 76 - DNA, 7 bp, Artificial Sequence
  synthetic primer
tctccca

SEQ ID NO: 77 - DNA, 7 bp, Artificial Sequence
  synthetic primer
ctcccag

SEQ ID NO: 78 - DNA, 6 bp, Artificial Sequence
  synthetic primer
ctccca

SEQ ID NO: 79 - DNA, 13 bp, Artificial Sequence
  synthetic primer
ctcccagatgctg

SEQ ID NO: 80 - DNA, 9 bp, Artificial Sequence
  synthetic primer
tcccagatg

SEQ ID NO: 81 - DNA, 8 bp, Artificial Sequence
  synthetic primer
cccagatg

SEQ ID NO: 82 - DNA, 10 bp, Artificial Sequence
  synthetic primer
ccagatgctg

SEQ ID NO: 83 - DNA, 10 bp, Artificial Sequence
  synthetic primer
ccagatgctg

SEQ ID NO: 84 - DNA, 10 bp, Artificial Sequence
  synthetic primer
ccagatgctg

SEQ ID NO: 85 - DNA, 12 bp, Artificial Sequence
  synthetic primer
ccagatgctgat

SEQ ID NO: 86 - DNA, 7 bp, Artificial Sequence
  synthetic primer
ccagatg

SEQ ID NO: 87 - DNA, 11 bp, Artificial Sequence
  synthetic primer
cagatgctgat

SEQ ID NO: 88 - DNA, 6 bp, Artificial Sequence
  synthetic primer
cagatg

SEQ ID NO: 89 - DNA, 8 bp, Artificial Sequence
  synthetic primer
cagatgct

SEQ ID NO: 90 - DNA, 7 bp, Artificial Sequence
  synthetic primer
cagatgc

SEQ ID NO: 91 - DNA, 6 bp, Artificial Sequence
  synthetic primer
cagatg

SEQ ID NO: 92 - DNA, 7 bp, Artificial Sequence
  synthetic primer
atgctga

SEQ ID NO: 93 - DNA, 11 bp, Artificial Sequence
  synthetic primer
atgctgattca

SEQ ID NO: 94 - DNA, 7 bp, Artificial Sequence
  synthetic primer
tgctgat

SEQ ID NO: 95 - DNA, 10 bp, Artificial Sequence
  synthetic primer
tgctgattca

SEQ ID NO: 96 - DNA, 17 bp, Artificial Sequence
  synthetic primer
gacagtgctctccttcc

SEQ ID NO: 97 - DNA, 19 bp, Artificial Sequence
  synthetic primer
gtgctctccttcctctttg

SEQ ID NO: 98 - DNA, 17 bp, Artificial Sequence
  synthetic primer
gctctccttcctctttg

SEQ ID NO: 99 - DNA, 8 bp, Artificial Sequence
  synthetic primer
cttcctct

SEQ ID NO: 100 - DNA, 6 bp, Artificial Sequence
  synthetic primer
cttcct

SEQ ID NO: 101 - DNA, 17 bp, Artificial Sequence
  synthetic primer
ttcctctttgatgcctc

SEQ ID NO: 102 - DNA, 15 bp, Artificial Sequence
  synthetic primer
ttcctctttgatgcc

SEQ ID NO: 103 - DNA, 6 bp, Artificial Sequence
  synthetic primer
ttcctc

SEQ ID NO: 104 - DNA, 10 bp, Artificial Sequence
  synthetic primer
cctctttgat

SEQ ID NO: 105 - DNA, 13 bp, Artificial Sequence
  synthetic primer
ctctttgatgcct

SEQ ID NO: 106 - DNA, 10 bp, Artificial Sequence
  synthetic primer
ctctttgatg

SEQ ID NO: 107 - DNA, 9 bp, Artificial Sequence
  synthetic primer
tctttgatg

SEQ ID NO: 108 - DNA, 10 bp, Artificial Sequence
  synthetic primer
tctttgatgc

SEQ ID NO: 109 - DNA, 11 bp, Artificial Sequence
  synthetic primer
tctttgatgcc

SEQ ID NO: 110 - DNA, 8 bp, Artificial Sequence
  synthetic primer
ctttgatg

SEQ ID NO: 111 - DNA, 6 bp, Artificial Sequence
  synthetic primer
ctttga

SEQ ID NO: 112 - DNA, 10 bp, Artificial Sequence
  synthetic primer
atgcctctct

SEQ ID NO: 113 - DNA, 9 bp, Artificial Sequence
  synthetic primer
agacaccag

SEQ ID NO: 114 - DNA, 13 bp, Artificial Sequence
  synthetic primer
ttagacctaagga

SEQ ID NO: 115 - DNA, 22 bp, Artificial Sequence
  synthetic primer
aaggaaggacttccctgaggag

SEQ ID NO: 116 - DNA, 6 bp, Artificial Sequence
  synthetic primer
aggaag

SEQ ID NO: 117 - DNA, 12 bp, Artificial Sequence
  synthetic primer
aaggacttccct

SEQ ID NO: 118 - DNA, 6 bp, Artificial Sequence
  synthetic primer
acttcc

SEQ ID NO: 119 - DNA, 18 bp, Artificial Sequence
  synthetic primer
atggtcaccggcaggagc

SEQ ID NO: 120 - DNA, 7 bp, Artificial Sequence
  synthetic primer
ccggcag

SEQ ID NO: 121 - DNA, 7 bp, Artificial Sequence
  synthetic primer
ctggggc

SEQ ID NO: 122 - DNA, 7 bp, Artificial Sequence
  synthetic primer
cctccct

SEQ ID NO: 123 - DNA, 9 bp, Artificial Sequence
  synthetic primer
tcccttccc

SEQ ID NO: 124 - DNA, 6 bp, Artificial Sequence
  synthetic primer
ttcccc

SEQ ID NO: 125 - DNA, 10 bp, Artificial Sequence
  synthetic primer
ccccatcagc

SEQ ID NO: 126 - DNA, 16 bp, Artificial Sequence
  synthetic primer
cagccctaattgccaa

SEQ ID NO: 127 - DNA, 17 bp, Artificial Sequence
  synthetic primer
agccctaattgccaaga

SEQ ID NO: 128 - DNA, 9 bp, Artificial Sequence
  synthetic primer
ccctaattg

SEQ ID NO: 129 - DNA, 11 bp, Artificial Sequence
  synthetic primer
ccctaattgcc

SEQ ID NO: 130 - DNA, 11 bp, Artificial Sequence
  synthetic primer
cctaattgcca

SEQ ID NO: 131 - DNA, 10 bp, Artificial Sequence
  synthetic primer
cctaattgcc

SEQ ID NO: 132 - DNA, 8 bp, Artificial Sequence
  synthetic primer
ctaattgc

SEQ ID NO: 133 - DNA, 9 bp, Artificial Sequence
  synthetic primer
ctaattgcc

SEQ ID NO: 134 - DNA, 6 bp, Artificial Sequence
  synthetic primer
ctaatt

SEQ ID NO: 135 - DNA, 7 bp, Artificial Sequence
  synthetic primer
ctaattg

SEQ ID NO: 136 - DNA, 6 bp, Artificial Sequence
  synthetic primer
taattg

SEQ ID NO: 137 - DNA, 5 bp, Artificial Sequence
  synthetic primer
taatt

SEQ ID NO: 138 - DNA, 14 bp, Artificial Sequence
  synthetic primer
ttgccaagatgtca

SEQ ID NO: 139 - DNA, 7 bp, Artificial Sequence
  synthetic primer
tgccaag

SEQ ID NO: 140 - DNA, 17 bp, Artificial Sequence
  synthetic primer
tgtcatggggggaagag

SEQ ID NO: 141 - DNA, 7 bp, Artificial Sequence
  synthetic primer
atggggg

SEQ ID NO: 142 - DNA, 6 bp, Artificial Sequence
  synthetic primer
ggggaa

SEQ ID NO: 143 - DNA, 6 bp, Artificial Sequence
  synthetic primer
ggaaga

SEQ ID NO: 144 - DNA, 17 bp, Artificial Sequence
  synthetic primer
aggaggggattaagcag

SEQ ID NO: 145 - DNA, 14 bp, Artificial Sequence
  synthetic primer
aggaggggattaag

SEQ ID NO: 146 - DNA, 16 bp, Artificial Sequence
  synthetic primer
aggaggggattaagca

SEQ ID NO: 147 - DNA, 17 bp, Artificial Sequence
  synthetic primer
ggaggggattaagcaga

SEQ ID NO: 148 - DNA, 12 bp, Artificial Sequence
  synthetic primer
ggaggggattaa

SEQ ID NO: 149 - DNA, 11 bp, Artificial Sequence
  synthetic primer
gaggggattaa

SEQ ID NO: 150 - DNA, 13 bp, Artificial Sequence
  synthetic primer
aggggattaagca

SEQ ID NO: 151 - DNA, 10 bp, Artificial Sequence
  synthetic primer
ggggattaag

SEQ ID NO: 152 - DNA, 9 bp, Artificial Sequence
  synthetic primer
gggattaag

SEQ ID NO: 153 - DNA, 8 bp, Artificial Sequence
  synthetic primer
gggattaa

SEQ ID NO: 154 - DNA, 6 bp, Artificial Sequence
  synthetic primer
gattaa

SEQ ID NO: 155 - DNA, 18 bp, Artificial Sequence
  synthetic primer
gacgggtgcccctccccc

SEQ ID NO: 156 - DNA, 17 bp, Artificial Sequence
  synthetic primer
gggtgcccctccccctc

SEQ ID NO: 157 - DNA, 16 bp, Artificial Sequence
  synthetic primer
gtgcccctccccctcc

SEQ ID NO: 158 - DNA, 14 bp, Artificial Sequence
  synthetic primer
gtgcccctccccct

SEQ ID NO: 159 - DNA, 13 bp, Artificial Sequence
  synthetic primer
gtgcccctccccc

SEQ ID NO: 160 - DNA, 13 bp, Artificial Sequence
  synthetic primer
tgcccctccccct

SEQ ID NO: 161 - DNA, 14 bp, Artificial Sequence
  synthetic primer
gcccctccccctcc

SEQ ID NO: 162 - DNA, 10 bp, Artificial Sequence
  synthetic primer
gcccctcccc

SEQ ID NO: 163 - DNA, 13 bp, Artificial Sequence
  synthetic primer
gcccctccccctc

SEQ ID NO: 164 - DNA, 11 bp, Artificial Sequence
  synthetic primer
gcccctccccc

SEQ ID NO: 165 - DNA, 7 bp, Artificial Sequence
  synthetic primer
gcccctc

SEQ ID NO: 166 - DNA, 12 bp, Artificial Sequence
  synthetic primer
cccctccccctc

SEQ ID NO: 167 - DNA, 14 bp, Artificial Sequence
  synthetic primer
cccctccccctccc

SEQ ID NO: 168 - DNA, 11 bp, Artificial Sequence
  synthetic primer
cccctccccct

SEQ ID NO: 169 - DNA, 10 bp, Artificial Sequence
  synthetic primer
cccctccccc

SEQ ID NO: 170 - DNA, 13 bp, Artificial Sequence
  synthetic primer
cccctccccctcc

SEQ ID NO: 171 - DNA, 9 bp, Artificial Sequence
  synthetic primer
cccctcccc

SEQ ID NO: 172 - DNA, 9 bp, Artificial Sequence
  synthetic primer
ccctccccc

SEQ ID NO: 173 - DNA, 8 bp, Artificial Sequence
  synthetic primer
ccctcccc

SEQ ID NO: 174 - DNA, 14 bp, Artificial Sequence
  synthetic primer
cctccccctcccag

SEQ ID NO: 175 - DNA, 13 bp, Artificial Sequence
  synthetic primer
tccccctcccagc

SEQ ID NO: 176 - DNA, 7 bp, Artificial Sequence
  synthetic primer
ccccctc

SEQ ID NO: 177 - DNA, 12 bp, Artificial Sequence
  synthetic primer
cctcccagccaa

SEQ ID NO: 178 - DNA, 7 bp, Artificial Sequence
  synthetic primer
cctccca

SEQ ID NO: 179 - DNA, 10 bp, Artificial Sequence
  synthetic primer
tcccagccaa

SEQ ID NO: 180 - DNA, 11 bp, Artificial Sequence
  synthetic primer
ccagccaatgt

SEQ ID NO: 181 - DNA, 21 bp, Artificial Sequence
  synthetic primer
ccagccaatgtcacctcctgg

SEQ ID NO: 182 - DNA, 7 bp, Artificial Sequence
  synthetic primer
cagccaa

SEQ ID NO: 183 - DNA, 7 bp, Artificial Sequence
  synthetic primer
caatgtc

SEQ ID NO: 184 - DNA, 10 bp, Artificial Sequence
  synthetic primer
tcacctcctg

SEQ ID NO: 185 - DNA, 6 bp, Artificial Sequence
  synthetic primer
agacag

SEQ ID NO: 186 - DNA, 12 bp, Artificial Sequence
  synthetic primer
gatttcaccatg

SEQ ID NO: 187 - DNA, 14 bp, Artificial Sequence
  synthetic primer
ctggtctcgaactc

SEQ ID NO: 188 - DNA, 16 bp, Artificial Sequence
  synthetic primer
ggtctcgaactcctga

SEQ ID NO: 189 - DNA, 18 bp, Artificial Sequence
  synthetic primer
tctcgaactcctgacctt

SEQ ID NO: 190 - DNA, 20 bp, Artificial Sequence
  synthetic primer
aactcctgaccttgtgatcc

SEQ ID NO: 191 - DNA, 18 bp, Artificial Sequence
  synthetic primer
tcctgaccttgtgatcca

SEQ ID NO: 192 - DNA, 8 bp, Artificial Sequence
  synthetic primer
ctgacctt

SEQ ID NO: 193 - DNA, 12 bp, Artificial Sequence
  synthetic primer
ctgaccttgtga

SEQ ID NO: 194 - DNA, 8 bp, Artificial Sequence
  synthetic primer
tgaccttg

SEQ ID NO: 195 - DNA, 7 bp, Artificial Sequence
  synthetic primer
tgacctt

SEQ ID NO: 196 - DNA, 11 bp, Artificial Sequence
  synthetic primer
tgaccttgtga

SEQ ID NO: 197 - DNA, 5 bp, Artificial Sequence
  synthetic primer
ccacc

SEQ ID NO: 198 - DNA, 6 bp, Artificial Sequence
  synthetic primer
acccac

SEQ ID NO: 199 - DNA, 7 bp, Artificial Sequence
  synthetic primer
cctccca

SEQ ID NO: 200 - DNA, 7 bp, Artificial Sequence
  synthetic primer
caaagtg

SEQ ID NO: 201 - DNA, 10 bp, Artificial Sequence
  synthetic primer
tgggattaca

SEQ ID NO: 202 - DNA, 9 bp, Artificial Sequence
  synthetic primer
gggattaca

SEQ ID NO: 203 - DNA, 12 bp, Artificial Sequence
  synthetic primer
gagccaccgcac

SEQ ID NO: 204 - DNA, 12 bp, Artificial Sequence
  synthetic primer
accgcacccggc

SEQ ID NO: 205 - DNA, 13 bp, Artificial Sequence
  synthetic primer
ggccacctgggga

SEQ ID NO: 206 - DNA, 10 bp, Artificial Sequence
  synthetic primer
gccacctggg

SEQ ID NO: 207 - DNA, 8 bp, Artificial Sequence
  synthetic primer
gccacctg

SEQ ID NO: 208 - DNA, 7 bp, Artificial Sequence
  synthetic primer
cacctgg

SEQ ID NO: 209 - DNA, 6 bp, Artificial Sequence
  synthetic primer
ggggaa

SEQ ID NO: 210 - DNA, 16 bp, Artificial Sequence
  synthetic primer
gggaatcttctaatag

SEQ ID NO: 211 - DNA, 10 bp, Artificial Sequence
  synthetic primer
ggaatcttct

SEQ ID NO: 212 - DNA, 6 bp, Artificial Sequence
  synthetic primer
atcttc

SEQ ID NO: 213 - DNA, 9 bp, Artificial Sequence
  synthetic primer
ttagggcag

SEQ ID NO: 214 - DNA, 14 bp, Artificial Sequence
  synthetic primer
ctggcctggaccca

SEQ ID NO: 215 - DNA, 12 bp, Artificial Sequence
  synthetic primer
tggcctggaccc

SEQ ID NO: 216 - DNA, 12 bp, Artificial Sequence
  synthetic primer
ggacccacctcc

SEQ ID NO: 217 - DNA, 14 bp, Artificial Sequence
  synthetic primer
acccacctcccttg

SEQ ID NO: 218 - DNA, 13 bp, Artificial Sequence
  synthetic primer
ccacctcccttgc

SEQ ID NO: 219 - DNA, 11 bp, Artificial Sequence
  synthetic primer
ccttgccacct

SEQ ID NO: 220 - DNA, 12 bp, Artificial Sequence
  synthetic primer
ttgccacctgcc

SEQ ID NO: 221 - DNA, 11 bp, Artificial Sequence
  synthetic primer
tgccacctgcc

SEQ ID NO: 222 - DNA, 12 bp, Artificial Sequence
  synthetic primer
tgccacctgccc

SEQ ID NO: 223 - DNA, 14 bp, Artificial Sequence
  synthetic primer
gccacctgccccct

SEQ ID NO: 224 - DNA, 10 bp, Artificial Sequence
  synthetic primer
ccacctgccc

SEQ ID NO: 225 - DNA, 8 bp, Artificial Sequence
  synthetic primer
cacctgcc

SEQ ID NO: 226 - DNA, 7 bp, Artificial Sequence
  synthetic primer
cacctgc

SEQ ID NO: 227 - DNA, 16 bp, Artificial Sequence
  synthetic primer
cacctgccccctgcac

SEQ ID NO: 228 - DNA, 10 bp, Artificial Sequence
  synthetic primer
cacctgcccc

SEQ ID NO: 229 - DNA, 12 bp, Artificial Sequence
  synthetic primer
cctgccccctgc

SEQ ID NO: 230 - DNA, 9 bp, Artificial Sequence
  synthetic primer
tgccccctg

SEQ ID NO: 231 - DNA, 7 bp, Artificial Sequence
  synthetic primer
ccccctg

SEQ ID NO: 232 - DNA, 17 bp, Artificial Sequence
  synthetic primer
accgactgatcatgttc

SEQ ID NO: 233 - DNA, 22 bp, Artificial Sequence
  synthetic primer
tgatcatgttcagtcacccagg

SEQ ID NO: 234 - DNA, 14 bp, Artificial Sequence
  synthetic primer
catgttcagtcacc

SEQ ID NO: 235 - DNA, 13 bp, Artificial Sequence
  synthetic primer
atgttcagtcacc

SEQ ID NO: 236 - DNA, 11 bp, Artificial Sequence
  synthetic primer
gttcagtcacc

SEQ ID NO: 237 - DNA, 15 bp, Artificial Sequence
  synthetic primer
ggagaggctgatcag

SEQ ID NO: 238 - DNA, 17 bp, Artificial Sequence
  synthetic primer
agaggctgatcaggcct

SEQ ID NO: 239 - DNA, 9 bp, Artificial Sequence
  synthetic primer
gccagacat

SEQ ID NO: 240 - DNA, 14 bp, Artificial Sequence
  synthetic primer
gccagacatccaga

SEQ ID NO: 241 - DNA, 13 bp, Artificial Sequence
  synthetic primer
cagacatccagat

SEQ ID NO: 242 - DNA, 9 bp, Artificial Sequence
  synthetic primer
cagatacag

SEQ ID NO: 243 - DNA, 6 bp, Artificial Sequence
  synthetic primer
cagata

SEQ ID NO: 244 - DNA, 9 bp, Artificial Sequence
  synthetic primer
tacagccca

SEQ ID NO: 245 - DNA, 14 bp, Artificial Sequence
  synthetic primer
acagcccattcttc

SEQ ID NO: 246 - DNA, 6 bp, Artificial Sequence
  synthetic primer
ttcttc

SEQ ID NO: 247 - DNA, 15 bp, Artificial Sequence
  synthetic primer
tcttgccctttgctc

SEQ ID NO: 248 - DNA, 19 bp, Artificial Sequence
  synthetic primer
tcttgccctttgctcctga

SEQ ID NO: 249 - DNA, 6 bp, Artificial Sequence
  synthetic primer
tcttgc

SEQ ID NO: 250 - DNA, 15 bp, Artificial Sequence
  synthetic primer
ttgccctttgctcct

SEQ ID NO: 251 - DNA, 14 bp, Artificial Sequence
  synthetic primer
ttgccctttgctcc

SEQ ID NO: 252 - DNA, 20 bp, Artificial Sequence
  synthetic primer
ttgccctttgctcctgattc

SEQ ID NO: 253 - DNA, 15 bp, Artificial Sequence
  synthetic primer
tgccctttgctcctg

SEQ ID NO: 254 - DNA, 13 bp, Artificial Sequence
  synthetic primer
tgccctttgctcc

SEQ ID NO: 255 - DNA, 7 bp, Artificial Sequence
  synthetic primer
ccctttg

SEQ ID NO: 256 - DNA, 6 bp, Artificial Sequence
  synthetic primer
ttctcc

SEQ ID NO: 257 - DNA, 12 bp, Artificial Sequence
  synthetic primer
caagctgtaccc

SEQ ID NO: 258 - DNA, 15 bp, Artificial Sequence
  synthetic primer
caagctgtacccaga

SEQ ID NO: 259 - DNA, 11 bp, Artificial Sequence
  synthetic primer
gctgtacccag

SEQ ID NO: 260 - DNA, 12 bp, Artificial Sequence
  synthetic primer
gagctttctggt

SEQ ID NO: 261 - DNA, 14 bp, Artificial Sequence
  synthetic primer
gagctttctggttc

SEQ ID NO: 262 - DNA, 9 bp, Artificial Sequence
  synthetic primer
tttctggtt

SEQ ID NO: 263 - DNA, 21 bp, Artificial Sequence
  synthetic primer
ttctggttcagcatgtacctg

SEQ ID NO: 264 - DNA, 11 bp, Artificial Sequence
  synthetic primer
ctggttcagca

SEQ ID NO: 265 - DNA, 15 bp, Artificial Sequence
  synthetic primer
ggttcagcatgtacc

SEQ ID NO: 266 - DNA, 17 bp, Artificial Sequence
  synthetic primer
ggttcagcatgtacctg

SEQ ID NO: 267 - DNA, 14 bp, Artificial Sequence
  synthetic primer
ggttcagcatgtac

SEQ ID NO: 268 - DNA, 15 bp, Artificial Sequence
  synthetic primer
gttcagcatgtacct

SEQ ID NO: 269 - DNA, 7 bp, Artificial Sequence
  synthetic primer
ttcagca

SEQ ID NO: 270 - DNA, 16 bp, Artificial Sequence
  synthetic primer
tacctggaggctgctt

SEQ ID NO: 271 - DNA, 17 bp, Artificial Sequence
  synthetic primer
tttgaccatgttttgag

SEQ ID NO: 272 - DNA, 9 bp, Artificial Sequence
  synthetic primer
accatgttt

SEQ ID NO: 273 - DNA, 7 bp, Artificial Sequence
  synthetic primer
tttatct

SEQ ID NO: 274 - DNA, 6 bp, Artificial Sequence
  synthetic primer
ttatct

SEQ ID NO: 275 - DNA, 7 bp, Artificial Sequence
  synthetic primer
ctaatgg

SEQ ID NO: 276 - DNA, 6 bp, Artificial Sequence
  synthetic primer
gattag

SEQ ID NO: 277 - DNA, 10 bp, Artificial Sequence
  synthetic primer
attagcaaaa

SEQ ID NO: 278 - DNA, 15 bp, Artificial Sequence
  synthetic primer
atgtcttgtggatcc

SEQ ID NO: 279 - DNA, 12 bp, Artificial Sequence
  synthetic primer
tcttgtggatcc

SEQ ID NO: 280 - DNA, 16 bp, Artificial Sequence
  synthetic primer
gatcccacttttaatc

SEQ ID NO: 281 - DNA, 6 bp, Artificial Sequence
  synthetic primer
tttaat

SEQ ID NO: 282 - DNA, 22 bp, Artificial Sequence
  synthetic primer
tttaatctgcagcgttcaggct

SEQ ID NO: 283 - DNA, 6 bp, Artificial Sequence
  synthetic primer
ttaatc

SEQ ID NO: 284 - DNA, 8 bp, Artificial Sequence
  synthetic primer
cagcgttc

SEQ ID NO: 285 - DNA, 7 bp, Artificial Sequence
  synthetic primer
gcccctt

SEQ ID NO: 286 - DNA, 12 bp, Artificial Sequence
  synthetic primer
ttggctgggaag

SEQ ID NO: 287 - DNA, 10 bp, Artificial Sequence
  synthetic primer
ttggctggga

SEQ ID NO: 288 - DNA, 7 bp, Artificial Sequence
  synthetic primer
ttggctg

SEQ ID NO: 289 - DNA, 8 bp, Artificial Sequence
  synthetic primer
gggcgctg

SEQ ID NO: 290 - DNA, 11 bp, Artificial Sequence
  synthetic primer
cgctgaagaaa

SEQ ID NO: 291 - DNA, 6 bp, Artificial Sequence
  synthetic primer
gaagaa

SEQ ID NO: 292 - DNA, 6 bp, Artificial Sequence
  synthetic primer
aagaaa

SEQ ID NO: 293 - DNA, 11 bp, Artificial Sequence
  synthetic primer
cccatgtgtat

SEQ ID NO: 294 - DNA, 10 bp, Artificial Sequence
  synthetic primer
ctatttcttg

SEQ ID NO: 295 - DNA, 6 bp, Artificial Sequence
  synthetic primer
tttctt

SEQ ID NO: 296 - DNA, 27 bp, Artificial Sequence
  synthetic primer
cttgcctttccattctgttctattgat

SEQ ID NO: 297 - DNA, 19 bp, Artificial Sequence
  synthetic primer
cctttccattctgttctat

SEQ ID NO: 298 - DNA, 16 bp, Artificial Sequence
  synthetic primer
tttccattctgttcta

SEQ ID NO: 299 - DNA, 15 bp, Artificial Sequence
  synthetic primer
ttccattctgttcta

SEQ ID NO: 300 - DNA, 11 bp, Artificial Sequence
  synthetic primer
cattctgttct

SEQ ID NO: 301 - DNA, 13 bp, Artificial Sequence
  synthetic primer
cattctgttctat

SEQ ID NO: 302 - DNA, 7 bp, Artificial Sequence
  synthetic primer
ctgttct

SEQ ID NO: 303 - DNA, 12 bp, Artificial Sequence
  synthetic primer
ctattgatttgt

SEQ ID NO: 304 - DNA, 6 bp, Artificial Sequence
  synthetic primer
tattga

SEQ ID NO: 305 - DNA, 8 bp, Artificial Sequence
  synthetic primer
tattgatt

SEQ ID NO: 306 - DNA, 11 bp, Artificial Sequence
  synthetic primer
ttgatttgtct

SEQ ID NO: 307 - DNA, 10 bp, Artificial Sequence
  synthetic primer
ttgtctgcct

SEQ ID NO: 308 - DNA, 11 bp, Artificial Sequence
  synthetic primer
ttgtctgccta

SEQ ID NO: 309 - DNA, 9 bp, Artificial Sequence
  synthetic primer
tgtctgcct

SEQ ID NO: 310 - DNA, 7 bp, Artificial Sequence
  synthetic primer
tgtctgc

SEQ ID NO: 311 - DNA, 9 bp, Artificial Sequence
  synthetic primer
tgtctgcct

SEQ ID NO: 312 - DNA, 8 bp, Artificial Sequence
  synthetic primer
tgcctatt

SEQ ID NO: 313 - DNA, 16 bp, Artificial Sequence
  synthetic primer
ctattcacctacctgt

SEQ ID NO: 314 - DNA, 13 bp, Artificial Sequence
  synthetic primer
cacctacctgtac

SEQ ID NO: 315 - DNA, 7 bp, Artificial Sequence
  synthetic primer
agcattt

SEQ ID NO: 316 - DNA, 15 bp, Artificial Sequence
  synthetic primer
caacttatcaatgat

SEQ ID NO: 317 - DNA, 15 bp, Artificial Sequence
  synthetic primer
caacttatcaatgat

SEQ ID NO: 318 - DNA, 10 bp, Artificial Sequence
  synthetic primer
ttatcaatga

SEQ ID NO: 319 - DNA, 12 bp, Artificial Sequence
  synthetic primer
atcaatgatatg

SEQ ID NO: 320 - DNA, 9 bp, Artificial Sequence
  synthetic primer
tgatggtgg

SEQ ID NO: 321 - DNA, 6 bp, Artificial Sequence
  synthetic primer
tggtgg

SEQ ID NO: 322 - DNA, 5 bp, Artificial Sequence
  synthetic primer
ggtgg

SEQ ID NO: 323 - DNA, 7 bp, Artificial Sequence
  synthetic primer
tggcctt

SEQ ID NO: 324 - DNA, 7 bp, Artificial Sequence
  synthetic primer
gcctttg

SEQ ID NO: 325 - DNA, 16 bp, Artificial Sequence
  synthetic primer
ttggattataatattt

SEQ ID NO: 326 - DNA, 9 bp, Artificial Sequence
  synthetic primer
tggattata

SEQ ID NO: 327 - DNA, 5 bp, Artificial Sequence
  synthetic primer
attat

SEQ ID NO: 328 - DNA, 14 bp, Artificial Sequence
  synthetic primer
attataatatttaa

SEQ ID NO: 329 - DNA, 14 bp, Artificial Sequence
  synthetic primer
tataatatttaaca

SEQ ID NO: 330 - DNA, 12 bp, Artificial Sequence
  synthetic primer
ataatatttaac

SEQ ID NO: 331 - DNA, 5 bp, Artificial Sequence
  synthetic primer
ataat

SEQ ID NO: 332 - DNA, 11 bp, Artificial Sequence
  synthetic primer
attttatttta

SEQ ID NO: 333 - DNA, 6 bp, Artificial Sequence
  synthetic primer
ttttat

SEQ ID NO: 334 - DNA, 12 bp, Artificial Sequence
  synthetic primer
ttttattttatt

SEQ ID NO: 335 - DNA, 12 bp, Artificial Sequence
  synthetic primer
ttattttagcaa

SEQ ID NO: 336 - DNA, 14 bp, Artificial Sequence
  synthetic primer
tattttagcaaagt

SEQ ID NO: 337 - DNA, 7 bp, Artificial Sequence
  synthetic primer
caaagtg

SEQ ID NO: 338 - DNA, 23 bp, Artificial Sequence
  synthetic primer
tttgtgtattttcataattctag

SEQ ID NO: 339 - DNA, 14 bp, Artificial Sequence
  synthetic primer
tgtattttcataat

SEQ ID NO: 340 - DNA, 13 bp, Artificial Sequence
  synthetic primer
tgtattttcataa

SEQ ID NO: 341 - DNA, 11 bp, Artificial Sequence
  synthetic primer
gtattttcata

SEQ ID NO: 342 - DNA, 7 bp, Artificial Sequence
  synthetic primer
tattttc

SEQ ID NO: 343 - DNA, 12 bp, Artificial Sequence
  synthetic primer
tattttcataat

SEQ ID NO: 344 - DNA, 11 bp, Artificial Sequence
  synthetic primer
tattttcataa

SEQ ID NO: 345 - DNA, 9 bp, Artificial Sequence
  synthetic primer
attttcata

SEQ ID NO: 346 - DNA, 18 bp, Artificial Sequence
  synthetic primer
ttttcataattctagatg

SEQ ID NO: 347 - DNA, 5 bp, Artificial Sequence
  synthetic primer
taatt

SEQ ID NO: 348 - DNA, 22 bp, Artificial Sequence
  synthetic primer
taattctagatgccttatggtc

SEQ ID NO: 349 - DNA, 12 bp, Artificial Sequence
  synthetic primer
ccttatggtcct

SEQ ID NO: 350 - DNA, 7 bp, Artificial Sequence
  synthetic primer
ggtcctt

SEQ ID NO: 351 - DNA, 6 bp, Artificial Sequence
  synthetic primer
atcttc

SEQ ID NO: 352 - DNA, 7 bp, Artificial Sequence
  synthetic primer
cataaat

SEQ ID NO: 353 - DNA, 6 bp, Artificial Sequence
  synthetic primer
ataaat

SEQ ID NO: 354 - DNA, 7 bp, Artificial Sequence
  synthetic primer
ataaata

SEQ ID NO: 355 - DNA, 6 bp, Artificial Sequence
  synthetic primer
aataaa

SEQ ID NO: 356 - DNA, 6 bp, Artificial Sequence
  synthetic primer
ataaat

SEQ ID NO: 357 - DNA, 8 bp, Artificial Sequence
  synthetic primer
aaatgagg

SEQ ID NO: 358 - DNA, 17 bp, Artificial Sequence
  synthetic primer
tgagggttaaaagttgt

SEQ ID NO: 359 - DNA, 12 bp, Artificial Sequence
  synthetic primer
aaagttgtctgg

SEQ ID NO: 360 - DNA, 17 bp, Artificial Sequence
  synthetic primer
aaagttgtctgggagag

SEQ ID NO: 361 - DNA, 12 bp, Artificial Sequence
  synthetic primer
ttgtctgggaga

SEQ ID NO: 362 - DNA, 10 bp, Artificial Sequence
  synthetic primer
ttgtctggga

SEQ ID NO: 363 - DNA, 7 bp, Artificial Sequence
  synthetic primer
aaatgct

SEQ ID NO: 364 - DNA, 17 bp, Artificial Sequence
  synthetic primer
tggtgaagtactttttc

SEQ ID NO: 365 - DNA, 17 bp, Artificial Sequence
  synthetic primer
ggtgaagtactttttct

SEQ ID NO: 366 - DNA, 10 bp, Artificial Sequence
  synthetic primer
gaggataaca

SEQ ID NO: 367 - DNA, 17 bp, Artificial Sequence
  synthetic primer
ggcaagaaagaagatgc

SEQ ID NO: 368 - DNA, 6 bp, Artificial Sequence
  synthetic primer
gcaaga

SEQ ID NO: 369 - DNA, 6 bp, Artificial Sequence
  synthetic primer
gaagat

SEQ ID NO: 370 - DNA, 18 bp, Artificial Sequence
  synthetic primer
aagatgcaagggaaacct

SEQ ID NO: 371 - DNA, 14 bp, Artificial Sequence
  synthetic primer
agatgcaagggaaa

SEQ ID NO: 372 - DNA, 16 bp, Artificial Sequence
  synthetic primer
agggaaaccttcatga

SEQ ID NO: 373 - DNA, 6 bp, Artificial Sequence
  synthetic primer
gaggaa

SEQ ID NO: 374 - DNA, 14 bp, Artificial Sequence
  synthetic primer
gaagatttcacaac

SEQ ID NO: 375 - DNA, 13 bp, Artificial Sequence
  synthetic primer
agatttcacaact

SEQ ID NO: 376 - DNA, 14 bp, Artificial Sequence
  synthetic primer
agatttcacaactg

SEQ ID NO: 377 - DNA, 12 bp, Artificial Sequence
  synthetic primer
gatttcacaact

SEQ ID NO: 378 - DNA, 11 bp, Artificial Sequence
  synthetic primer
atttcacaact

SEQ ID NO: 379 - DNA, 16 bp, Artificial Sequence
  synthetic primer
atgctaggaactggtt

SEQ ID NO: 380 - DNA, 10 bp, Artificial Sequence
  synthetic primer
tgctaggaac

SEQ ID NO: 381 - DNA, 24 bp, Artificial Sequence
  synthetic primer
tgctaggaactggtttgcttctgg

SEQ ID NO: 382 - DNA, 18 bp, Artificial Sequence
  synthetic primer
gcttctggctgttgtctc

SEQ ID NO: 383 - DNA, 17 bp, Artificial Sequence
  synthetic primer
tctccttagggtgagct

SEQ ID NO: 384 - DNA, 11 bp, Artificial Sequence
  synthetic primer
cttagggtgag

SEQ ID NO: 385 - DNA, 12 bp, Artificial Sequence
  synthetic primer
tagggtgagctc

SEQ ID NO: 386 - DNA, 12 bp, Artificial Sequence
  synthetic primer
agggtgagctct

SEQ ID NO: 387 - DNA, 17 bp, Artificial Sequence
  synthetic primer
agctctgtctggtgatt

SEQ ID NO: 388 - DNA, 7 bp, Artificial Sequence
  synthetic primer
ctgtctg

SEQ ID NO: 389 - DNA, 8 bp, Artificial Sequence
  synthetic primer
ctgtctgg

SEQ ID NO: 390 - DNA, 6 bp, Artificial Sequence
  synthetic primer
ctgtct

SEQ ID NO: 391 - DNA, 18 bp, Artificial Sequence
  synthetic primer
tggtgattagcatcacca

SEQ ID NO: 392 - DNA, 13 bp, Artificial Sequence
  synthetic primer
gtgattagcatca

SEQ ID NO: 393 - DNA, 6 bp, Artificial Sequence
  synthetic primer
tgatta

SEQ ID NO: 394 - DNA, 15 bp, Artificial Sequence
  synthetic primer
gattagcatcaccat

SEQ ID NO: 395 - DNA, 6 bp, Artificial Sequence
  synthetic primer
gattag

SEQ ID NO: 396 - DNA, 10 bp, Artificial Sequence
  synthetic primer
attagcatca

SEQ ID NO: 397 - DNA, 17 bp, Artificial Sequence
  synthetic primer
attagcatcaccatgga

SEQ ID NO: 398 - DNA, 16 bp, Artificial Sequence
  synthetic primer
gcatcaccatggatta

SEQ ID NO: 399 - DNA, 14 bp, Artificial Sequence
  synthetic primer
catcaccatggatt

SEQ ID NO: 400 - DNA, 15 bp, Artificial Sequence
  synthetic primer
accatggattaaatt

SEQ ID NO: 401 - DNA, 10 bp, Artificial Sequence
  synthetic primer
ccatggatta

SEQ ID NO: 402 - DNA, 10 bp, Artificial Sequence
  synthetic primer
atggattaaa

SEQ ID NO: 403 - DNA, 9 bp, Artificial Sequence
  synthetic primer
tggattaaa

SEQ ID NO: 404 - DNA, 6 bp, Artificial Sequence
  synthetic primer
gattaa

SEQ ID NO: 405 - DNA, 17 bp, Artificial Sequence
  synthetic primer
gattaaattaattggct

SEQ ID NO: 406 - DNA, 6 bp, Artificial Sequence
  synthetic primer
attaaa

SEQ ID NO: 407 - DNA, 17 bp, Artificial Sequence
  synthetic primer
attaaattaattggctg

SEQ ID NO: 408 - DNA, 10 bp, Artificial Sequence
  synthetic primer
ttaaattaat

SEQ ID NO: 409 - DNA, 9 bp, Artificial Sequence
  synthetic primer
ttaaattaa

SEQ ID NO: 410 - DNA, 10 bp, Artificial Sequence
  synthetic primer
taaattaatt

SEQ ID NO: 411 - DNA, 17 bp, Artificial Sequence
  synthetic primer
taaattaattggctgta

SEQ ID NO: 412 - DNA, 16 bp, Artificial Sequence
  synthetic primer
aaattaattggctgta

SEQ ID NO: 413 - DNA, 11 bp, Artificial Sequence
  synthetic primer
aattaattggc

SEQ ID NO: 414 - DNA, 6 bp, Artificial Sequence
  synthetic primer
aattaa

SEQ ID NO: 415 - DNA, 5 bp, Artificial Sequence
  synthetic primer
aatta

SEQ ID NO: 416 - DNA, 11 bp, Artificial Sequence
  synthetic primer
attaattggct

SEQ ID NO: 417 - DNA, 10 bp, Artificial Sequence
  synthetic primer
attaattggc

SEQ ID NO: 418 - DNA, 8 bp, Artificial Sequence
  synthetic primer
ttaattgg

SEQ ID NO: 419 - DNA, 9 bp, Artificial Sequence
  synthetic primer
ttaattggc

SEQ ID NO: 420 - DNA, 6 bp, Artificial Sequence
  synthetic primer
ttaatt

SEQ ID NO: 421 - DNA, 6 bp, Artificial Sequence
  synthetic primer
taattg

SEQ ID NO: 422 - DNA, 5 bp, Artificial Sequence
  synthetic primer
taatt

SEQ ID NO: 423 - DNA, 11 bp, Artificial Sequence
  synthetic primer
taattggctgt

SEQ ID NO: 424 - DNA, 9 bp, Artificial Sequence
  synthetic primer
ctcaatccc

SEQ ID NO: 425 - DNA, 10 bp, Artificial Sequence
  synthetic primer
tcacctcctg

SEQ ID NO: 426 - DNA, 9 bp, Artificial Sequence
  synthetic primer
ctgcccaca

SEQ ID NO: 427 - DNA, 10 bp, Artificial Sequence
  synthetic primer
ttgtgggaag

SEQ ID NO: 428 - DNA, 15 bp, Artificial Sequence
  synthetic primer
tgtgggaagagggaa

SEQ ID NO: 429 - DNA, 9 bp, Artificial Sequence
  synthetic primer
gggccagtg

SEQ ID NO: 430 - DNA, 9 bp, Artificial Sequence
  synthetic primer
gtcaaggtt

SEQ ID NO: 431 - DNA, 11 bp, Artificial Sequence
  synthetic primer
cctgaaacgtc

SEQ ID NO: 432 - DNA, 14 bp, Artificial Sequence
  synthetic primer
ctgaaacgtcagtc

SEQ ID NO: 433 - DNA, 12 bp, Artificial Sequence
  synthetic primer
ctgaaacgtcag

SEQ ID NO: 434 - DNA, 12 bp, Artificial Sequence
  synthetic primer
tgaaacgtcagt

SEQ ID NO: 435 - DNA, 14 bp, Artificial Sequence
  synthetic primer
tgaaacgtcagtcc

SEQ ID NO: 436 - DNA, 9 bp, Artificial Sequence
  synthetic primer
aaacgtcag

SEQ ID NO: 437 - DNA, 11 bp, Artificial Sequence
  synthetic primer
aaacgtcagtc

SEQ ID NO: 438 - DNA, 9 bp, Artificial Sequence
  synthetic primer
aaacgtcag

SEQ ID NO: 439 - DNA, 11 bp, Artificial Sequence
  synthetic primer
aaacgtcagtc

SEQ ID NO: 440 - DNA, 8 bp, Artificial Sequence
  synthetic primer
aaacgtca

SEQ ID NO: 441 - DNA, 20 bp, Artificial Sequence
  synthetic primer
aaacgtcagtccccagccct

SEQ ID NO: 442 - DNA, 8 bp, Artificial Sequence
  synthetic primer
cgtcagtc

SEQ ID NO: 443 - DNA, 11 bp, Artificial Sequence
  synthetic primer
cagtccccagc

SEQ ID NO: 444 - DNA, 17 bp, Artificial Sequence
  synthetic primer
ctggcctcatgtctcct

SEQ ID NO: 445 - DNA, 12 bp, Artificial Sequence
  synthetic primer
ccttgggaagac

SEQ ID NO: 446 - DNA, 17 bp, Artificial Sequence
  synthetic primer
gaaggaattgtgttgta

SEQ ID NO: 447 - DNA, 22 bp, Artificial Sequence
  synthetic primer
gaaggaattgtgttgtaaagag

SEQ ID NO: 448 - DNA, 17 bp, Artificial Sequence
  synthetic primer
ggaattgtgttgtaaag

SEQ ID NO: 449 - DNA, 15 bp, Artificial Sequence
  synthetic primer
attgtgttgtaaaga

SEQ ID NO: 450 - DNA, 15 bp, Artificial Sequence
  synthetic primer
attgtgttgtaaaga

SEQ ID NO: 451 - DNA, 8 bp, Artificial Sequence
  synthetic primer
ttgtgttg

SEQ ID NO: 452 - DNA, 10 bp, Artificial Sequence
  synthetic primer
gttgtaaaga

SEQ ID NO: 453 - DNA, 14 bp, Artificial Sequence
  synthetic primer
gtaaagaggtgtca

SEQ ID NO: 454 - DNA, 13 bp, Artificial Sequence
  synthetic primer
taaagaggtgtca

SEQ ID NO: 455 - DNA, 17 bp, Artificial Sequence
  synthetic primer
aaagaggtgtcacaatg

SEQ ID NO: 456 - DNA, 12 bp, Artificial Sequence
  synthetic primer
agaggtgtcaca

SEQ ID NO: 457 - DNA, 18 bp, Artificial Sequence
  synthetic primer
aggtgtcacaatgccccc

SEQ ID NO: 458 - DNA, 18 bp, Artificial Sequence
  synthetic primer
aggtgtcacaatgccccc

SEQ ID NO: 459 - DNA, 8 bp, Artificial Sequence
  synthetic primer
aggtgtca

SEQ ID NO: 460 - DNA, 20 bp, Artificial Sequence
  synthetic primer
gtgtcacaatgccccctgcc

SEQ ID NO: 461 - DNA, 14 bp, Artificial Sequence
  synthetic primer
gtgtcacaatgccc

SEQ ID NO: 462 - DNA, 7 bp, Artificial Sequence
  synthetic primer
cacaatg

SEQ ID NO: 463 - DNA, 8 bp, Artificial Sequence
  synthetic primer
acaatgcc

SEQ ID NO: 464 - DNA, 13 bp, Artificial Sequence
  synthetic primer
atgccccctgccc

SEQ ID NO: 465 - DNA, 9 bp, Artificial Sequence
  synthetic primer
tgccccctg

SEQ ID NO: 466 - DNA, 15 bp, Artificial Sequence
  synthetic primer
tgccccctgccccat

SEQ ID NO: 467 - DNA, 11 bp, Artificial Sequence
  synthetic primer
tgccccctgcc

SEQ ID NO: 468 - DNA, 16 bp, Artificial Sequence
  synthetic primer
gccccctgccccatac

SEQ ID NO: 469 - DNA, 9 bp, Artificial Sequence
  synthetic primer
ccctgcccc

SEQ ID NO: 470 - DNA, 7 bp, Artificial Sequence
  synthetic primer
gccccat

SEQ ID NO: 471 - DNA, 11 bp, Artificial Sequence
  synthetic primer
gttctccccac

SEQ ID NO: 472 - DNA, 7 bp, Artificial Sequence
  synthetic primer
ctcccca

SEQ ID NO: 473 - DNA, 7 bp, Artificial Sequence
  synthetic primer
cttggca

SEQ ID NO: 474 - DNA, 15 bp, Artificial Sequence
  synthetic primer
ggcactctcagtatc

SEQ ID NO: 475 - DNA, 10 bp, Artificial Sequence
  synthetic primer
atcctgccaa

SEQ ID NO: 476 - DNA, 7 bp, Artificial Sequence
  synthetic primer
ctgccaa

SEQ ID NO: 477 - DNA, 7 bp, Artificial Sequence
  synthetic primer
tgccaag

SEQ ID NO: 478 - DNA, 31 bp, Artificial Sequence
  synthetic
tgaacacccccaatctcccagatgctgattc

SEQ ID NO: 479 - DNA, 46 bp, Artificial Sequence
  synthetic
gattaatatgattatgaacacccccaatctcccagatgctgattca

SEQ ID NO: 480 - DNA, 38 bp, Artificial Sequence
  synthetic
gattagcgttagtatgatatctcgcggatgctgaatca

SEQ ID NO: 481 - DNA, 45 bp, Artificial Sequence
  synthetic
attaatatgattaataacgcccccaatctcccaggtgctgattca

SEQ ID NO: 482 - DNA, 249 bp, Artificial Sequence
  synthetic
gccagacatccagatacagcccattcttctggccagtcttgccctttgctcctgattctccaagctgtacccagagctttctggttcagcatgtacctggaggctgcttttgaccatgttttgagagtggcatggctttatctaatgggatgaacctggctaaagagattagcaaaatgtattgtggatcccacttttaatctgcagcgttcaggctgccccttcttggctgggaagggcgctgaagaa

SEQ ID NO: 483 - DNA, 249 bp, Artificial Sequence
  synthetic
ctgacggtgctctccttcctctttgatgcctctctcttgggatactagctgaagctggagagacaccagttagacctaaggaaggacttccctgaggagtaggggcttatggtcaccggcaggagctggggcctcccttccccatcagccctaattgccaagatgtcatggggggaagaggaggggattaagcagacgggtgcccctccccctcccagccaatgtcacctcctggtgcccagtcgagtc

SEQ ID NO: 484 - DNA, 249 bp, Artificial Sequence
  synthetic
taacataaataaatgagggttaaaagttgtctgggagagaaatgcttgtgtactggtgaagtactttttctggaggataacactaggcaagaaagaagatgcaagggaaaccttcatgaggaagatttcacaactgcagagcatgctaggaactggtttgcttctggctgttgtctccttagggtgagctctgtctggtgattagcatcaccatggattaaattaattggctgtactcaatcccttgct

SEQ ID NO: 485 - DNA, 249 bp, Artificial Sequence
  synthetic
agtagagacaggatttcaccatgttggccaggctggtctcgaactcctgaccttgtgatccacccacctcggcctcccaaagtgctgggattacaggcatgagccaccgcacccggccacctggggaatcttctaatagtcttagggcagcttagctggcctggacccacctcccttgccacctgccccctgcaccgactgatcatgttcagtcacccaggagatgatggagaggctgatcaggcctcc

SEQ ID NO: 486 - DNA, 249 bp, Artificial Sequence
  synthetic
ttgaaatggcattttcatcatatctaaatgccaatgtgtatttgggtctatttcttgcctttccattctgttctattgatttgtctgcctattcacctacctgtacttagtagtatggtttagcattttggtacaacttatcaatgatatgatggtggcctttggattataatatttaacattttattttagcaaagtgtttgtgtattttcataattctagatgccttatggtccttctaatatcttc

SEQ ID NO: 487 - DNA, 248 bp, Artificial Sequence
  synthetic
ccattccctcctgcccacagttttagggttctatgggtttgtgggaagagggaatagcatagtgggccagtgtcaaggttaaaagacccttccaggtcctgaaacgtcagtccccagccctggcctcatgtctccttgggaagacagaaggaattgtgttgtaaagaggtgtcacaatgccccctgccccatacaagttctccccaccttggcactctcagtatcctgccaagagtcctccctgacca

SEQ ID NO: 488 - DNA, 9 bp, Artificial Sequence
  synthetic
ggcacttgt

SEQ ID NO: 489 - DNA, 7 bp, Artificial Sequence
  synthetic
tactttc

SEQ ID NO: 490 - DNA, 9 bp, Artificial Sequence
  synthetic
ctttctggc

SEQ ID NO: 491 - DNA, 15 bp, Artificial Sequence
  synthetic
tttctggctgagcag

SEQ ID NO: 492 - DNA, 28 bp, Artificial Sequence
  synthetic
tggctgagcagggcagtgtggccgacgg

SEQ ID NO: 493 - DNA, 11 bp, Artificial Sequence
  synthetic
ggctgagcagg

SEQ ID NO: 494 - DNA, 20 bp, Artificial Sequence
  synthetic
tgagcagggcagtgtggccg

SEQ ID NO: 495 - DNA, 23 bp, Artificial Sequence
  synthetic
gagcagggcagtgtggccgacgg

SEQ ID NO: 496 - DNA, 23 bp, Artificial Sequence
  synthetic
gagcagggcagtgtggccgacgg

SEQ ID NO: 497 - DNA, 18 bp, Artificial Sequence
  synthetic
gcagggcagtgtggccga

SEQ ID NO: 498 - DNA, 18 bp, Artificial Sequence
  synthetic
cagggcagtgtggccgac

SEQ ID NO: 499 - DNA, 18 bp, Artificial Sequence
  synthetic
cagggcagtgtggccgac

SEQ ID NO: 500 - DNA, 20 bp, Artificial Sequence
  synthetic
agggcagtgtggccgacggc

SEQ ID NO: 501 - DNA, 14 bp, Artificial Sequence
  synthetic
agggcagtgtggcc

SEQ ID NO: 502 - DNA, 15 bp, Artificial Sequence
  synthetic
agtgtggccgacggc

SEQ ID NO: 503 - DNA, 15 bp, Artificial Sequence
  synthetic
cggctgaaaggggaa

SEQ ID NO: 504 - DNA, 10 bp, Artificial Sequence
  synthetic
tgaaagggga

SEQ ID NO: 505 - DNA, 19 bp, Artificial Sequence
  synthetic
gaaaggggaagctgcggct

SEQ ID NO: 506 - DNA, 112 bp, Artificial Sequence
  synthetic
aggcacttgttactttctggctgagcagggcagtgtggccgacggctgaaaggggaagctgcggctgcttttgtgcaggggtggtggtgatgagggtgatgtggggggctgg

SEQ ID NO: 507 - DNA, 17 bp, Artificial Sequence
  synthetic
ggctgcttttgtgcagg

SEQ ID NO: 508 - DNA, 16 bp, Artificial Sequence
  synthetic
gtgcaggggtggtggt

SEQ ID NO: 509 - DNA, 9 bp, Artificial Sequence
  synthetic
caggggtgg

SEQ ID NO: 510 - DNA, 7 bp, Artificial Sequence
  synthetic
caggggt

SEQ ID NO: 511 - DNA, 9 bp, Artificial Sequence
  synthetic
ggggtggtg

SEQ ID NO: 512 - DNA, 10 bp, Artificial Sequence
  synthetic
ggggtggtgg

SEQ ID NO: 513 - DNA, 12 bp, Artificial Sequence
  synthetic
ggggtggtggtg

SEQ ID NO: 514 - DNA, 6 bp, Artificial Sequence
  synthetic
tggtgg

SEQ ID NO: 515 - DNA, 19 bp, Artificial Sequence
  synthetic
ggtggtgatgagggtgatg

SEQ ID NO: 516 - DNA, 14 bp, Artificial Sequence
  synthetic
gtggtgatgagggt

SEQ ID NO: 517 - DNA, 6 bp, Artificial Sequence
  synthetic
gatgag

SEQ ID NO: 518 - DNA, 14 bp, Artificial Sequence
  synthetic
agggtgatgtgggg

SEQ ID NO: 519 - DNA, 7 bp, Artificial Sequence
  synthetic
gtggggg

SEQ ID NO: 520 - DNA, 13 bp, Artificial Sequence
  synthetic
catggaggggaaa

SEQ ID NO: 521 - DNA, 14 bp, Artificial Sequence
  synthetic
ggggaaaggatctg

SEQ ID NO: 522 - DNA, 15 bp, Artificial Sequence
  synthetic
atctggctgactacc

SEQ ID NO: 523 - DNA, 22 bp, Artificial Sequence
  synthetic
tctggctgactacctggaagcc

SEQ ID NO: 524 - DNA, 6 bp, Artificial Sequence
  synthetic
gctgac

SEQ ID NO: 525 - DNA, 10 bp, Artificial Sequence
  synthetic
gctgactacc

SEQ ID NO: 526 - DNA, 16 bp, Artificial Sequence
  synthetic
actacctggaagccag

SEQ ID NO: 527 - DNA, 21 bp, Artificial Sequence
  synthetic
ctggaagccaggacagatccc

SEQ ID NO: 528 - DNA, 22 bp, Artificial Sequence
  synthetic
ggaagccaggacagatcccacc

SEQ ID NO: 529 - DNA, 19 bp, Artificial Sequence
  synthetic
ccaggacagatcccacccc

SEQ ID NO: 530 - DNA, 12 bp, Artificial Sequence
  synthetic
agatcccacccc

SEQ ID NO: 531 - DNA, 16 bp, Artificial Sequence
  synthetic
gatcccaccccagaaa

SEQ ID NO: 532 - DNA, 7 bp, Artificial Sequence
  synthetic
cccaccc

SEQ ID NO: 533 - DNA, 9 bp, Artificial Sequence
  synthetic
ccaccccag

SEQ ID NO: 534 - DNA, 7 bp, Artificial Sequence
  synthetic
cacccca

SEQ ID NO: 535 - DNA, 17 bp, Artificial Sequence
  synthetic
accccagaaaggcgcag

SEQ ID NO: 536 - DNA, 7 bp, Artificial Sequence
  synthetic
accccag

SEQ ID NO: 537 - DNA, 14 bp, Artificial Sequence
  synthetic
ccagaaaggcgcag

SEQ ID NO: 538 - DNA, 15 bp, Artificial Sequence
  synthetic
aggcgcagtaggggc

SEQ ID NO: 539 - DNA, 6 bp, Artificial Sequence
  synthetic
ctcatc

SEQ ID NO: 540 - DNA, 13 bp, Artificial Sequence
  synthetic
tagcccgcccctc

SEQ ID NO: 541 - DNA, 16 bp, Artificial Sequence
  synthetic
tagcccgcccctccct

SEQ ID NO: 542 - DNA, 10 bp, Artificial Sequence
  synthetic
agcccgcccc

SEQ ID NO: 543 - DNA, 11 bp, Artificial Sequence
  synthetic
gcccgcccctc

SEQ ID NO: 544 - DNA, 14 bp, Artificial Sequence
  synthetic
cccgcccctcccta

SEQ ID NO: 545 - DNA, 14 bp, Artificial Sequence
  synthetic
ccgcccctccctac

SEQ ID NO: 546 - DNA, 13 bp, Artificial Sequence
  synthetic
ctccctacctaat

SEQ ID NO: 547 - DNA, 17 bp, Artificial Sequence
  synthetic
cctacctaattaaggac

SEQ ID NO: 548 - DNA, 16 bp, Artificial Sequence
  synthetic
cctacctaattaagga

SEQ ID NO: 549 - DNA, 17 bp, Artificial Sequence
  synthetic
ctacctaattaaggacc

SEQ ID NO: 550 - DNA, 16 bp, Artificial Sequence
  synthetic
tacctaattaaggacc

SEQ ID NO: 551 - DNA, 12 bp, Artificial Sequence
  synthetic
tacctaattaag

SEQ ID NO: 552 - DNA, 11 bp, Artificial Sequence
  synthetic
acctaattaag

SEQ ID NO: 553 - DNA, 10 bp, Artificial Sequence
  synthetic
acctaattaa

SEQ ID NO: 554 - DNA, 10 bp, Artificial Sequence
  synthetic
cctaattaag

SEQ ID NO: 555 - DNA, 8 bp, Artificial Sequence
  synthetic
ctaattaa

SEQ ID NO: 556 - DNA, 16 bp, Artificial Sequence
  synthetic
ctaattaaggacccta

SEQ ID NO: 557 - DNA, 6 bp, Artificial Sequence
  synthetic
ctaatt

SEQ ID NO: 558 - DNA, 10 bp, Artificial Sequence
  synthetic
ctaattaagg

SEQ ID NO: 559 - DNA, 6 bp, Artificial Sequence
  synthetic
taatta

SEQ ID NO: 560 - DNA, 7 bp, Artificial Sequence
  synthetic
taattaa

SEQ ID NO: 561 - DNA, 5 bp, Artificial Sequence
  synthetic
taatt

SEQ ID NO: 562 - DNA, 7 bp, Artificial Sequence
  synthetic
aaggacc

SEQ ID NO: 563 - DNA, 17 bp, Artificial Sequence
  synthetic
gaccctaatcagcttgg

SEQ ID NO: 564 - DNA, 11 bp, Artificial Sequence
  synthetic
gaccctaatca

SEQ ID NO: 565 - DNA, 17 bp, Artificial Sequence
  synthetic
gaccctaatcagcttgg

SEQ ID NO: 566 - DNA, 10 bp, Artificial Sequence
  synthetic
ccctaatcag

SEQ ID NO: 567 - DNA, 6 bp, Artificial Sequence
  synthetic
ctaatc

SEQ ID NO: 568 - DNA, 6 bp, Artificial Sequence
  synthetic
taatca

SEQ ID NO: 569 - DNA, 13 bp, Artificial Sequence
  synthetic
caagatgtggcat

SEQ ID NO: 570 - DNA, 13 bp, Artificial Sequence
  synthetic
gtgaacagctgag

SEQ ID NO: 571 - DNA, 10 bp, Artificial Sequence
  synthetic
aacagctgag

SEQ ID NO: 572 - DNA, 6 bp, Artificial Sequence
  synthetic
cagctg

SEQ ID NO: 573 - DNA, 19 bp, Artificial Sequence
  synthetic
tgagcacacagggcaggag

SEQ ID NO: 574 - DNA, 14 bp, Artificial Sequence
  synthetic
gagcacacagggca

SEQ ID NO: 575 - DNA, 15 bp, Artificial Sequence
  synthetic
agggccccgggggac

SEQ ID NO: 576 - DNA, 14 bp, Artificial Sequence
  synthetic
gggccccgggggac

SEQ ID NO: 577 - DNA, 15 bp, Artificial Sequence
  synthetic
gggccccgggggacc

SEQ ID NO: 578 - DNA, 9 bp, Artificial Sequence
  synthetic
gccccgggg

SEQ ID NO: 579 - DNA, 7 bp, Artificial Sequence
  synthetic
gccccgg

SEQ ID NO: 580 - DNA, 7 bp, Artificial Sequence
  synthetic
ccggggg

SEQ ID NO: 581 - DNA, 6 bp, Artificial Sequence
  synthetic
cggggg

SEQ ID NO: 582 - DNA, 21 bp, Artificial Sequence
  synthetic
gggggaccttgggcagcccgg

SEQ ID NO: 583 - DNA, 8 bp, Artificial Sequence
  synthetic
gggacctt

SEQ ID NO: 584 - DNA, 17 bp, Artificial Sequence
  synthetic
gggcagcccgggaacca

SEQ ID NO: 585 - DNA, 9 bp, Artificial Sequence
  synthetic
aaccagcat

SEQ ID NO: 586 - DNA, 10 bp, Artificial Sequence
  synthetic
gtagcaggac

SEQ ID NO: 587 - DNA, 11 bp, Artificial Sequence
  synthetic
aggactgaccg

SEQ ID NO: 588 - DNA, 15 bp, Artificial Sequence
  synthetic
ccggctcccggggca

SEQ ID NO: 589 - DNA, 15 bp, Artificial Sequence
  synthetic
cccggggcaccttgg

SEQ ID NO: 590 - DNA, 7 bp, Artificial Sequence
  synthetic
ccggggc

SEQ ID NO: 591 - DNA, 7 bp, Artificial Sequence
  synthetic
taatgct

SEQ ID NO: 592 - DNA, 15 bp, Artificial Sequence
  synthetic
gctgcaggtgtggcc

SEQ ID NO: 593 - DNA, 9 bp, Artificial Sequence
  synthetic
ctgcaggtg

SEQ ID NO: 594 - DNA, 7 bp, Artificial Sequence
  synthetic
ctgcagg

SEQ ID NO: 595 - DNA, 10 bp, Artificial Sequence
  synthetic
tgcaggtgtg

SEQ ID NO: 596 - DNA, 10 bp, Artificial Sequence
  synthetic
gcaggtgtgg

SEQ ID NO: 597 - DNA, 12 bp, Artificial Sequence
  synthetic
gcaggtgtggcc

SEQ ID NO: 598 - DNA, 7 bp, Artificial Sequence
  synthetic
gcaggtg

SEQ ID NO: 599 - DNA, 8 bp, Artificial Sequence
  synthetic
gcaggtgt

SEQ ID NO: 600 - DNA, 12 bp, Artificial Sequence
  synthetic
tggccagttgat

SEQ ID NO: 601 - DNA, 9 bp, Artificial Sequence
  synthetic
ggccagttg

SEQ ID NO: 602 - DNA, 11 bp, Artificial Sequence
  synthetic
ggccagttgat

SEQ ID NO: 603 - DNA, 10 bp, Artificial Sequence
  synthetic
gccagttgat

SEQ ID NO: 604 - DNA, 10 bp, Artificial Sequence
  synthetic
gtggagacag

SEQ ID NO: 605 - DNA, 6 bp, Artificial Sequence
  synthetic
gattaa

SEQ ID NO: 606 - DNA, 8 bp, Artificial Sequence
  synthetic
cataaact

SEQ ID NO: 607 - DNA, 8 bp, Artificial Sequence
  synthetic
cagcggct

SEQ ID NO: 608 - DNA, 15 bp, Artificial Sequence
  synthetic
agcggctgccccggg

SEQ ID NO: 609 - DNA, 249 bp, Artificial Sequence
  synthetic
aagatttaaagtccttgggagaggtggagtccacacctctcttcaagatgtggcatgaaatggtgaacagctgagcacacagggcaggagggccccgggggaccttgggcaccccgggaaccagcatggggtagcaggactgaccggctcccggggcaccttggtaatgctgcaggtgtggccagttgatcccctgtggagacagtaaagattaagaggatcataaactgcgtccagcggctgccccgg

SEQ ID NO: 610 - DNA, 7 bp, Artificial Sequence
  synthetic
tgggcgg

SEQ ID NO: 611 - DNA, 7 bp, Artificial Sequence
  synthetic
gggcggt

SEQ ID NO: 612 - DNA, 14 bp, Artificial Sequence
  synthetic
gttggatttccagg

SEQ ID NO: 613 - DNA, 21 bp, Artificial Sequence
  synthetic
ttggatttccaggtaacctct

SEQ ID NO: 614 - DNA, 15 bp, Artificial Sequence
  synthetic
ttggatttccaggta

SEQ ID NO: 615 - DNA, 16 bp, Artificial Sequence
  synthetic
tggatttccaggtaac

SEQ ID NO: 616 - DNA, 13 bp, Artificial Sequence
  synthetic
gatttccaggtaa

SEQ ID NO: 617 - DNA, 8 bp, Artificial Sequence
  synthetic
gatttcca

SEQ ID NO: 618 - DNA, 19 bp, Artificial Sequence
  synthetic
ggtaacctctctgaccgac

SEQ ID NO: 619 - DNA, 16 bp, Artificial Sequence
  synthetic
taacctctctgaccga

SEQ ID NO: 620 - DNA, 11 bp, Artificial Sequence
  synthetic
ccgaccaatcg

SEQ ID NO: 621 - DNA, 10 bp, Artificial Sequence
  synthetic
ccaatcgaaa

SEQ ID NO: 622 - DNA, 9 bp, Artificial Sequence
  synthetic
cgaaatccc

SEQ ID NO: 623 - DNA, 15 bp, Artificial Sequence
  synthetic
atccctctcggaaga

SEQ ID NO: 624 - DNA, 10 bp, Artificial Sequence
  synthetic
ctcggaagaa

SEQ ID NO: 625 - DNA, 18 bp, Artificial Sequence
  synthetic
ctcggaagaaagcgcttc

SEQ ID NO: 626 - DNA, 6 bp, Artificial Sequence
  synthetic
cggaag

SEQ ID NO: 627 - DNA, 23 bp, Artificial Sequence
  synthetic
cggaagaaagcgcttcactagct

SEQ ID NO: 628 - DNA, 17 bp, Artificial Sequence
  synthetic
actagcttatctcatct

SEQ ID NO: 629 - DNA, 14 bp, Artificial Sequence
  synthetic
actagcttatctca

SEQ ID NO: 630 - DNA, 14 bp, Artificial Sequence
  synthetic
ctagcttatctcat

SEQ ID NO: 631 - DNA, 15 bp, Artificial Sequence
  synthetic
tagcttatctcatct

SEQ ID NO: 632 - DNA, 11 bp, Artificial Sequence
  synthetic
agcttatctca

SEQ ID NO: 633 - DNA, 11 bp, Artificial Sequence
  synthetic
agcttatctca

SEQ ID NO: 634 - DNA, 20 bp, Artificial Sequence
  synthetic
agcttatctcatctaaccaa

SEQ ID NO: 635 - DNA, 8 bp, Artificial Sequence
  synthetic
gcttatct

SEQ ID NO: 636 - DNA, 19 bp, Artificial Sequence
  synthetic
gcttatctcatctaaccaa

SEQ ID NO: 637 - DNA, 8 bp, Artificial Sequence
  synthetic
gcttatct

SEQ ID NO: 638 - DNA, 11 bp, Artificial Sequence
  synthetic
gcttatctcat

SEQ ID NO: 639 - DNA, 9 bp, Artificial Sequence
  synthetic
gcttatctc

SEQ ID NO: 640 - DNA, 8 bp, Artificial Sequence
  synthetic
gcttatct

SEQ ID NO: 641 - DNA, 7 bp, Artificial Sequence
  synthetic
gcttatc

SEQ ID NO: 642 - DNA, 7 bp, Artificial Sequence
  synthetic
cttatct

SEQ ID NO: 643 - DNA, 6 bp, Artificial Sequence
  synthetic
ctcatc

SEQ ID NO: 644 - DNA, 14 bp, Artificial Sequence
  synthetic
tctaaccaattaga

SEQ ID NO: 645 - DNA, 17 bp, Artificial Sequence
  synthetic
tctaaccaattagaagc

SEQ ID NO: 646 - DNA, 11 bp, Artificial Sequence
  synthetic
taaccaattag

SEQ ID NO: 647 - DNA, 11 bp, Artificial Sequence
  synthetic
aaccaattaga

SEQ ID NO: 648 - DNA, 9 bp, Artificial Sequence
  synthetic
accaattag

SEQ ID NO: 649 - DNA, 10 bp, Artificial Sequence
  synthetic
accaattaga

SEQ ID NO: 650 - DNA, 11 bp, Artificial Sequence
  synthetic
accaattagaa

SEQ ID NO: 651 - DNA, 8 bp, Artificial Sequence
  synthetic
ccaattag

SEQ ID NO: 652 - DNA, 10 bp, Artificial Sequence
  synthetic
ccaattagaa

SEQ ID NO: 653 - DNA, 6 bp, Artificial Sequence
  synthetic
caatta

SEQ ID NO: 654 - DNA, 7 bp, Artificial Sequence
  synthetic
caattag

SEQ ID NO: 655 - DNA, 6 bp, Artificial Sequence
  synthetic
aattag

SEQ ID NO: 656 - DNA, 6 bp, Artificial Sequence
  synthetic
tcaaag

SEQ ID NO: 657 - DNA, 22 bp, Artificial Sequence
  synthetic
accctcgacgcccccaccttat

SEQ ID NO: 658 - DNA, 17 bp, Artificial Sequence
  synthetic
accctcgacgcccccac

SEQ ID NO: 659 - DNA, 17 bp, Artificial Sequence
  synthetic
cctcgacgcccccacct

SEQ ID NO: 660 - DNA, 11 bp, Artificial Sequence
  synthetic
gacgcccccac

SEQ ID NO: 661 - DNA, 10 bp, Artificial Sequence
  synthetic
acgcccccac

SEQ ID NO: 662 - DNA, 14 bp, Artificial Sequence
  synthetic
acgcccccacctta

SEQ ID NO: 663 - DNA, 9 bp, Artificial Sequence
  synthetic
cgcccccac

SEQ ID NO: 664 - DNA, 11 bp, Artificial Sequence
  synthetic
cgcccccacct

SEQ ID NO: 665 - DNA, 10 bp, Artificial Sequence
  synthetic
cgcccccacc

SEQ ID NO: 666 - DNA, 7 bp, Artificial Sequence
  synthetic
cccccac

SEQ ID NO: 667 - DNA, 19 bp, Artificial Sequence
  synthetic
ccccaccttatcgaccaat

SEQ ID NO: 668 - DNA, 10 bp, Artificial Sequence
  synthetic
caccttatcg

SEQ ID NO: 669 - DNA, 15 bp, Artificial Sequence
  synthetic
ttatcgaccaatcag

SEQ ID NO: 670 - DNA, 13 bp, Artificial Sequence
  synthetic
tatcgaccaatca

SEQ ID NO: 671 - DNA, 16 bp, Artificial Sequence
  synthetic
atcgaccaatcagagc

SEQ ID NO: 672 - DNA, 14 bp, Artificial Sequence
  synthetic
atcgaccaatcaga

SEQ ID NO: 673 - DNA, 18 bp, Artificial Sequence
  synthetic
tcgaccaatcagagcgcc

SEQ ID NO: 674 - DNA, 14 bp, Artificial Sequence
  synthetic
tcgaccaatcagag

SEQ ID NO: 675 - DNA, 11 bp, Artificial Sequence
  synthetic
tcgaccaatca

SEQ ID NO: 676 - DNA, 14 bp, Artificial Sequence
  synthetic
cgaccaatcagagc

SEQ ID NO: 677 - DNA, 14 bp, Artificial Sequence
  synthetic
cgaccaatcagagc

SEQ ID NO: 678 - DNA, 11 bp, Artificial Sequence
  synthetic
gaccaatcaga

SEQ ID NO: 679 - DNA, 10 bp, Artificial Sequence
  synthetic
ccaatcagag

SEQ ID NO: 680 - skipped

SEQ ID NO: 681 - DNA, 17 bp, Artificial Sequence
  synthetic
aatcagagcgccccctt

SEQ ID NO: 682 - DNA, 13 bp, Artificial Sequence
  synthetic
cagagcgccccct

SEQ ID NO: 683 - DNA, 9 bp, Artificial Sequence
  synthetic
agcgccccc

SEQ ID NO: 684 - DNA, 12 bp, Artificial Sequence
  synthetic
cgcccccttaca

SEQ ID NO: 685 - DNA, 7 bp, Artificial Sequence
  synthetic
ccccctt

SEQ ID NO: 686 - DNA, 17 bp, Artificial Sequence
  synthetic
cccttacaaaggccggc

SEQ ID NO: 687 - DNA, 11 bp, Artificial Sequence
  synthetic
ttacaaaggcc

SEQ ID NO: 688 - DNA, 7 bp, Artificial Sequence
  synthetic
tacaaag

SEQ ID NO: 689 - DNA, 7 bp, Artificial Sequence
  synthetic
caaaggc

SEQ ID NO: 690 - DNA, 12 bp, Artificial Sequence
  synthetic
gccggcagcagt

SEQ ID NO: 691 - DNA, 7 bp, Artificial Sequence
  synthetic
ccggcag

SEQ ID NO: 692 - DNA, 8 bp, Artificial Sequence
  synthetic
cagtgaca

SEQ ID NO: 693 - DNA, 12 bp, Artificial Sequence
  synthetic
gacagccaatga

SEQ ID NO: 694 - DNA, 11 bp, Artificial Sequence
  synthetic
acagccaatga

SEQ ID NO: 695 - DNA, 7 bp, Artificial Sequence
  synthetic
cagccaa

SEQ ID NO: 696 - DNA, 11 bp, Artificial Sequence
  synthetic
cagccaatgaa

SEQ ID NO: 697 - DNA, 10 bp, Artificial Sequence
  synthetic
ccaatgaaaa

SEQ ID NO: 698 - DNA, 9 bp, Artificial Sequence
  synthetic
aatgaaaat

SEQ ID NO: 699 - DNA, 7 bp, Artificial Sequence
  synthetic
gaaaata

SEQ ID NO: 700 - DNA, 7 bp, Artificial Sequence
  synthetic
aaaatag

SEQ ID NO: 701 - DNA, 10 bp, Artificial Sequence
  synthetic
taacacccct

SEQ ID NO: 702 - DNA, 7 bp, Artificial Sequence
  synthetic
cctcctt

SEQ ID NO: 703 - DNA, 23 bp, Artificial Sequence
  synthetic
cctccttcctcgcctcacgccca

SEQ ID NO: 704 - DNA, 10 bp, Artificial Sequence
  synthetic
tccttcctcg

SEQ ID NO: 705 - DNA, 6 bp, Artificial Sequence
  synthetic
cttcct

SEQ ID NO: 706 - DNA, 6 bp, Artificial Sequence
  synthetic
ttcctc

SEQ ID NO: 707 - DNA, 7 bp, Artificial Sequence
  synthetic
gcctcac

SEQ ID NO: 708 - DNA, 11 bp, Artificial Sequence
  synthetic
cctcacgccca

SEQ ID NO: 709 - DNA, 15 bp, Artificial Sequence
  synthetic
ctcacgcccacgtgg

SEQ ID NO: 710 - DNA, 14 bp, Artificial Sequence
  synthetic
tcacgcccacgtgg

SEQ ID NO: 711 - DNA, 13 bp, Artificial Sequence
  synthetic
tcacgcccacgtg

SEQ ID NO: 712 - DNA, 12 bp, Artificial Sequence
  synthetic
acgcccacgtgg

SEQ ID NO: 713 - DNA, 11 bp, Artificial Sequence
  synthetic
acgcccacgtg

SEQ ID NO: 714 - DNA, 10 bp, Artificial Sequence
  synthetic
cgcccacgtg

SEQ ID NO: 715 - DNA, 12 bp, Artificial Sequence
  synthetic
gcccacgtggtg

SEQ ID NO: 716 - DNA, 11 bp, Artificial Sequence
  synthetic
gcccacgtggt

SEQ ID NO: 717 - DNA, 8 bp, Artificial Sequence
  synthetic
gcccacgt

SEQ ID NO: 718 - DNA, 10 bp, Artificial Sequence
  synthetic
cccacgtggt

SEQ ID NO: 719 - DNA, 11 bp, Artificial Sequence
  synthetic
cccacgtggtg

SEQ ID NO: 720 - DNA, 8 bp, Artificial Sequence
  synthetic
cccacgtg

SEQ ID NO: 721 - DNA, 7 bp, Artificial Sequence
  synthetic
ccacgtg

SEQ ID NO: 722 - DNA, 8 bp, Artificial Sequence
  synthetic
ccacgtgg

SEQ ID NO: 723 - DNA, 5 bp, Artificial Sequence
  synthetic
ccacg

SEQ ID NO: 724 - DNA, 6 bp, Artificial Sequence
  synthetic
cacgtg

SEQ ID NO: 725 - DNA, 7 bp, Artificial Sequence
  synthetic
cacgtgg

SEQ ID NO: 726 - DNA, 8 bp, Artificial Sequence
  synthetic
cacgtggt

SEQ ID NO: 727 - DNA, 5 bp, Artificial Sequence
  synthetic
cgtgg

SEQ ID NO: 728 - DNA, 14 bp, Artificial Sequence
  synthetic
attttaagcaaagc

SEQ ID NO: 729 - DNA, 13 bp, Artificial Sequence
  synthetic
aaagaacattctg

SEQ ID NO: 730 - DNA, 15 bp, Artificial Sequence
  synthetic
aagaacattctggta

SEQ ID NO: 731 - DNA, 15 bp, Artificial Sequence
  synthetic
agaacattctggtaa

SEQ ID NO: 732 - DNA, 10 bp, Artificial Sequence
  synthetic
agaacattct

SEQ ID NO: 733 - DNA, 12 bp, Artificial Sequence
  synthetic
gaacattctggt

SEQ ID NO: 734 - DNA, 14 bp, Artificial Sequence
  synthetic
gaacattctggtaa

SEQ ID NO: 735 - DNA, 17 bp, Artificial Sequence
  synthetic
gtcggaggcctggattt

SEQ ID NO: 736 - DNA, 6 bp, Artificial Sequence
  synthetic
tttgtt

SEQ ID NO: 737 - DNA, 7 bp, Artificial Sequence
  synthetic
ttgcctg

SEQ ID NO: 738 - DNA, 13 bp, Artificial Sequence
  synthetic
tcgtccccccgtg

SEQ ID NO: 739 - DNA, 7 bp, Artificial Sequence
  synthetic
tcgtccc

SEQ ID NO: 740 - DNA, 19 bp, Artificial Sequence
  synthetic
cgtccccccgtgcagcagc

SEQ ID NO: 741 - DNA, 10 bp, Artificial Sequence
  synthetic
cccccgtgca

SEQ ID NO: 742 - DNA, 6 bp, Artificial Sequence
  synthetic
cccccg

SEQ ID NO: 743 - DNA, 7 bp, Artificial Sequence
  synthetic
cccccgt

SEQ ID NO: 744 - DNA, 8 bp, Artificial Sequence
  synthetic
cagcggcc

SEQ ID NO: 745 - DNA, 17 bp, Artificial Sequence
  synthetic
ctgttttcctccccctg

SEQ ID NO: 746 - DNA, 6 bp, Artificial Sequence
  synthetic
ttttcc

SEQ ID NO: 747 - DNA, 6 bp, Artificial Sequence
  synthetic
ttcctc

SEQ ID NO: 748 - DNA, 15 bp, Artificial Sequence
  synthetic
cccctgttgtgtgtt

SEQ ID NO: 749 - DNA, 14 bp, Artificial Sequence
  synthetic
cccctgttgtgtgt

SEQ ID NO: 750 - DNA, 16 bp, Artificial Sequence
  synthetic
gttgtgtgtttttatt

SEQ ID NO: 751 - DNA, 9 bp, Artificial Sequence
  synthetic
ttgtgtgtt

SEQ ID NO: 752 - DNA, 14 bp, Artificial Sequence
  synthetic
tgtgtgtttttatt

SEQ ID NO: 753 - DNA, 9 bp, Artificial Sequence
  synthetic
gtgtgtttt

SEQ ID NO: 754 - DNA, 8 bp, Artificial Sequence
  synthetic
tttttatt

SEQ ID NO: 755 - DNA, 11 bp, Artificial Sequence
  synthetic
tttttattatt

SEQ ID NO: 756 - DNA, 8 bp, Artificial Sequence
  synthetic
ttttatta

SEQ ID NO: 757 - DNA, 10 bp, Artificial Sequence
  synthetic
ttttattatt

SEQ ID NO: 758 - DNA, 6 bp, Artificial Sequence
  synthetic
ttttat

SEQ ID NO: 759 - DNA, 6 bp, Artificial Sequence
  synthetic
ttatta

SEQ ID NO: 760 - DNA, 7 bp, Artificial Sequence
  synthetic
tattttc

SEQ ID NO: 761 - DNA, 8 bp, Artificial Sequence
  synthetic
attttccc

SEQ ID NO: 762 - DNA, 14 bp, Artificial Sequence
  synthetic
gcttagatgtgtca

SEQ ID NO: 763 - DNA, 12 bp, Artificial Sequence
  synthetic
gatgtgtcaatc

SEQ ID NO: 764 - DNA, 14 bp, Artificial Sequence
  synthetic
atgtgtcaatcatt

SEQ ID NO: 765 - DNA, 6 bp, Artificial Sequence
  synthetic
caatca

SEQ ID NO: 766 - DNA, 11 bp, Artificial Sequence
  synthetic
caatcattctc

SEQ ID NO: 767 - DNA, 13 bp, Artificial Sequence
  synthetic
ctgccattggttg

SEQ ID NO: 768 - DNA, 7 bp, Artificial Sequence
  synthetic
gccattg

SEQ ID NO: 769 - DNA, 11 bp, Artificial Sequence
  synthetic
ccattggttgg

SEQ ID NO: 770 - DNA, 15 bp, Artificial Sequence
  synthetic
gagagtttgcgtcaa

SEQ ID NO: 771 - DNA, 14 bp, Artificial Sequence
  synthetic
agtttgcgtcaaaa

SEQ ID NO: 772 - DNA, 14 bp, Artificial Sequence
  synthetic
agtttgcgtcaaaa

SEQ ID NO: 773 - DNA, 12 bp, Artificial Sequence
  synthetic
gtttgcgtcaaa

SEQ ID NO: 774 - DNA, 15 bp, Artificial Sequence
  synthetic
tttgcgtcaaaaagt

SEQ ID NO: 775 - DNA, 9 bp, Artificial Sequence
  synthetic
ttgcgtcaa

SEQ ID NO: 776 - DNA, 11 bp, Artificial Sequence
  synthetic
ttgcgtcaaaa

SEQ ID NO: 777 - DNA, 8 bp, Artificial Sequence
  synthetic
gcgtcaaa

SEQ ID NO: 778 - DNA, 23 bp, Artificial Sequence
  synthetic
tgccagagaggcgctttctcagc

SEQ ID NO: 779 - DNA, 9 bp, Artificial Sequence
  synthetic
gccagagag

SEQ ID NO: 780 - DNA, 15 bp, Artificial Sequence
  synthetic
cgctttctcagcaaa

SEQ ID NO: 781 - DNA, 11 bp, Artificial Sequence
  synthetic
ctttctcagca

SEQ ID NO: 782 - DNA, 7 bp, Artificial Sequence
  synthetic
ctcagca

SEQ ID NO: 783 - DNA, 28 bp, Artificial Sequence
  synthetic
caaatctccctgagagcgggaccggcct

SEQ ID NO: 784 - DNA, 21 bp, Artificial Sequence
  synthetic
cctgagagcgggaccggcctc

SEQ ID NO: 785 - DNA, 11 bp, Artificial Sequence
  synthetic
agagcgggacc

SEQ ID NO: 786 - DNA, 8 bp, Artificial Sequence
  synthetic
agccgctg

SEQ ID NO: 787 - DNA, 10 bp, Artificial Sequence
  synthetic
agccgctggc

SEQ ID NO: 788 - DNA, 9 bp, Artificial Sequence
  synthetic
cctcccgag

SEQ ID NO: 789 - DNA, 9 bp, Artificial Sequence
  synthetic
ctcccgagc

SEQ ID NO: 790 - DNA, 14 bp, Artificial Sequence
  synthetic
cccgagcagggccg

SEQ ID NO: 791 - DNA, 9 bp, Artificial Sequence
  synthetic
agcagggcc

SEQ ID NO: 792 - DNA, 11 bp, Artificial Sequence
  synthetic
ggccgtcacct

SEQ ID NO: 793 - DNA, 13 bp, Artificial Sequence
  synthetic
ccgtcacctggga

SEQ ID NO: 794 - DNA, 14 bp, Artificial Sequence
  synthetic
cgtcacctgggaaa

SEQ ID NO: 795 - DNA, 7 bp, Artificial Sequence
  synthetic
tcacctg

SEQ ID NO: 796 - DNA, 7 bp, Artificial Sequence
  synthetic
cacctgg

SEQ ID NO: 797 - DNA, 17 bp, Artificial Sequence
  synthetic
cacctgggaaaagggca

SEQ ID NO: 798 - DNA, 15 bp, Artificial Sequence
  synthetic
cctgggaaaagggca

SEQ ID NO: 799 - DNA, 15 bp, Artificial Sequence
  synthetic
ctgggaaaagggcaa

SEQ ID NO: 800 - DNA, 13 bp, Artificial Sequence
  synthetic
tgggaaaagggca

SEQ ID NO: 801 - DNA, 6 bp, Artificial Sequence
  synthetic
ggaaaa

SEQ ID NO: 802 - DNA, 15 bp, Artificial Sequence
  synthetic
aaagggcaagaggta

SEQ ID NO: 803 - DNA, 17 bp, Artificial Sequence
  synthetic
aagggcaagaggtactc

SEQ ID NO: 804 - DNA, 6 bp, Artificial Sequence
  synthetic
gcaaga

SEQ ID NO: 805 - DNA, 10 bp, Artificial Sequence
  synthetic
acccatcacc

SEQ ID NO: 806 - DNA, 13 bp, Artificial Sequence
  synthetic
ccatcacctgaag

SEQ ID NO: 807 - DNA, 11 bp, Artificial Sequence
  synthetic
catcacctgaa

SEQ ID NO: 808 - DNA, 7 bp, Artificial Sequence
  synthetic
tcacctg

SEQ ID NO: 809 - DNA, 9 bp, Artificial Sequence
  synthetic
gggattctg

SEQ ID NO: 810 - DNA, 11 bp, Artificial Sequence
  synthetic
tgtctccccac

SEQ ID NO: 811 - DNA, 7 bp, Artificial Sequence
  synthetic
ctcccca

SEQ ID NO: 812 - DNA, 10 bp, Artificial Sequence
  synthetic
actttacatg

SEQ ID NO: 813 - DNA, 10 bp, Artificial Sequence
  synthetic
tgtgtccggt

SEQ ID NO: 814 - DNA, 14 bp, Artificial Sequence
  synthetic
ctgcccctttcagg

SEQ ID NO: 815 - DNA, 7 bp, Artificial Sequence
  synthetic
gcccctt

SEQ ID NO: 816 - DNA, 15 bp, Artificial Sequence
  synthetic
gcagccccaggcttg

SEQ ID NO: 817 - DNA, 15 bp, Artificial Sequence
  synthetic
gcagccccaggcttg

SEQ ID NO: 818 - DNA, 11 bp, Artificial Sequence
  synthetic
agccccaggct

SEQ ID NO: 819 - DNA, 14 bp, Artificial Sequence
  synthetic
agccccaggcttga

SEQ ID NO: 820 - DNA, 11 bp, Artificial Sequence
  synthetic
agccccaggct

SEQ ID NO: 821 - DNA, 10 bp, Artificial Sequence
  synthetic
agccccaggc

SEQ ID NO: 822 - DNA, 7 bp, Artificial Sequence
  synthetic
gccccag

SEQ ID NO: 823 - DNA, 19 bp, Artificial Sequence
  synthetic
ccccaggcttgaatgctcg

SEQ ID NO: 824 - DNA, 11 bp, Artificial Sequence
  synthetic
gaggggaggag

SEQ ID NO: 825 - DNA, 11 bp, Artificial Sequence
  synthetic
cggtggtaaag

SEQ ID NO: 826 - DNA, 7 bp, Artificial Sequence
  synthetic
cggtggt

SEQ ID NO: 827 - DNA, 22 bp, Artificial Sequence
  synthetic
ggtggtaaaggatcaagggcct

SEQ ID NO: 828 - DNA, 21 bp, Artificial Sequence
  synthetic
ggatcaagggcctccttctgg

SEQ ID NO: 829 - DNA, 20 bp, Artificial Sequence
  synthetic
caagggcctccttctggcag

SEQ ID NO: 830 - DNA, 19 bp, Artificial Sequence
  synthetic
aagggcctccttctggcag

SEQ ID NO: 831 - DNA, 20 bp, Artificial Sequence
  synthetic
aagggcctccttctggcagg

SEQ ID NO: 832 - DNA, 17 bp, Artificial Sequence
  synthetic
agggcctccttctggca

SEQ ID NO: 833 - DNA, 20 bp, Artificial Sequence
  synthetic
gggcctccttctggcagggc

SEQ ID NO: 834 - DNA, 9 bp, Artificial Sequence
  synthetic
ctggcaggg

SEQ ID NO: 835 - DNA, 7 bp, Artificial Sequence
  synthetic
ctggcag

SEQ ID NO: 836 - DNA, 1251 bp, Artificial Sequence
  synthetic
atggtggaccacttacttccagtggacgagaacttctcgtcgccaaaatgcccagttgggtatctgggtgataggctggttggccggcgggcatatcacatgctgccctcacccgtctctgaagatgacagcgatgcctccagcccctgctcctgttccagtcccgactctcaagccctctgctcctgctatggtggaggcctgggcaccgagagccaggacagcatcttggacttcctattgtcccaggccacgctgggcagtggcgggggcagcggcagtagcattggggccagcagtggccccgtggcctgggggccctggcgaagggcagcggcccctgtgaagggggagcatttctgcttgcccgagtttcctttgggtgatcctgatgacgtcccacggcccttccagcctaccctggaggagattgaagagtttctggaggagaacatggagcctggagtcaaggaggtccctgagggcaacagcaaggacttggatgcctgcagccagctctcagctgggccacacaagagccacctccatcctgggtccagcgggagagagcgctgttcccctccaccaggtggtgccagtgcaggaggtgcccagggcccaggtgggggccccacgcctgatggccccatcccagtgttgctgcagatccagcccgtgcctgtgaagcaggaatcgggcacagggcctgcctcccctgggcaagccccagagaatgtcaaggttgcccagctcctggtcaacatccaggggcagaccttcgcactcgtgccccaggtggtaccctcctccaacttgaacctgccctccaagtttgtgcgcattgcccctgtgcccattgccgccaagcctgttggatcgggacccctggggcctggccctgccggtctcctcatgggccagaagttccccaagaacccagccgcagaactcatcaaaatgcacaaatgtactttccctggctgcagcaagatgtacaccaaaagcagccacctcaaggcccacctgcgccggcacacgggtgagaagcccttcgcctgcacctggccaggctgcggctggaggttctcgcgctctgacgagctgtcgcggcacaggcgctcgcactcaggtgtgaagccgtaccagtgtcctgtgtgcgagaagaagttcgcgcggagcgaccacctctccaagcacatcaaggtgcaccgcttcccgcggagcagccgctccgtgcgctccgtgaactga

SEQ ID NO: 837 - DNA, 1080 bp, Artificial Sequence
  synthetic
atggtcgatatggataaactcataaacaacttggaggtccaacttaattcagaaggtggctcaatgcaggtattcaagcaggtcactgcttctgttcggaacagagatccccctgagatagaatacagaagtaatatgacttctccaacactcctggatgccaaccccatggagaacccagcactgtttaatgacatcaagattgagcccccagaagaacttttggctagtgatttcagcctgccccaagtggaaccagttgacctctcctttcacaagcccaaggctcctctccagcctgctagcatgctacaagctccaatacgtccccccaagccacagtcttctccccagacccttgtggtgtccacgtcaacatctgacatgagcacttcagcaaacattcctactgttctgaccccaggctctgtcctgacctcctctcagagcactggtagccagcagatcttacatgtcattcacactatcccctcagtcagtctgccaaataagatgggtggcctgaagaccatcccagtggtagtgcagtctctgcccatggtgtatactactttgcctgcagatgggggccctgcagccattacagtcccactcattggaggagatggtaaaaatgctggatcagtgaaagttgaccccacctccatgtctccactggaaattccaagtgacagtgaggagagtacaattgagagtggatcctcagccttgcagagtctgcagggactacagcaagaaccagcagcaatggcccaaatgcagggagaagagtcgcttgacttgaagagaagacggattcaccaatgtgactttgcaggatgcagcaaagtgtacaccaaaagctctcacctgaaagctcaccgcagaatccatacaggagagaagccttataaatgcacctgggatggctgctcctggaaatttgctcgctcagatgagctcactcgccatttccgcaagcacacaggcatcaagccttttcggtgcacagactgcaaccgcagcttttctcgttctgaccacctgtccctgcatcgccgtcgccatgacaccatgtga

SEQ ID NO: 838 - protein, 359 aa, Artificial Sequence
  synthetic
MVDMDKLINNLEVQLNSEGGSMQVFKQVTASVRNRDPPEIEYRSNMTSPTLLDANPMENPALFNDIKIEPPEELLASDFSLPQVEPVDLSFHKPKAPLQPASMLQAPIRPPKPQSSPQTLVVSTSTSDMSTSANIPTVLTPGSVLTSSQSTGSQQILHVIHTIPSVSLPNKMGGLKTIPVVVQSLPMVYTTLPADGGPAAITVPLIGGDGKNAGSVKVDPTSMSPLEIPSDSEESTIESGSSALQSLQGLQQEPAAMAQMQGEESLDLKRRRIHQCDFAGCSKVYTKSSHLKAHRRIHTGEKPYKCTWDGCSWKFARSDELTRHFRKHTGIKPFRCTDCNRSFSRSDHLSLHRRRHDTM

SEQ ID NO: 839 - DNA, 1929 bp, Artificial Sequence
  synthetic
atggtccatggatcagtgacattcagggatgtggccattgacttctctcaggaggagtgggagtgcctgcagcctgatcagaggaccttgtacagggatgtgatgttggagaactacagccacctgatctcactggcaggaagttccatttctaaaccagatgtaattacgttactagagcaagagaaagagccctggatggttgtaaggaaagaaacaagcagacggtatccagatttggagttaaaatatggacctgagaaagtatctccagaaaatgatacctctgaagtaaatttacccaaacaggttataaagcaaataagtacaactcttggcattgaggccttttattttagaaatgactcagaatatagacaatttgagggactacagggatatcaagaaggaaatatcaatcaaaagatgatcagctatgaaaaactgcctactcatactcctcatgcttctcttatttgcaatacacataaaccgtatgaatgtaaggaatgtgggaaatactttagtcgtagtgcaaatcttattcagcatcagagtattcatactggagagaaaccctttgaatgtaaggagtgtgggaaagcctttcgacttcacatacaatttactcgacatcagaaatttcatactggtgagaaaccttttgaatgtaacgaatgtggaaaggcctttagtcttcttaccctgcttaatcgccataagaacattcacacaggtgagaaactgtttgaatgtaaggaatgtgggaagtcctttaatcgtagctcaaaccttgttcaacatcagagtattcattctggtgtaaaaccatatgaatgtaaggagtgtgggaaaggctttaatcgtggtgcacaccttattcagcatcagaaaattcattccaatgagaaaccctttgtatgtaaggaatgtgggatggcctttcgatatcattaccaacttattgaacattgccaaattcatactggtgagaaaccctttgaatgtaaagaatgtggaaaggcgtttactcttctgacaaagcttgttcgacatcagaagattcatactggtgagaaaccctttgaatgcagggaatgtgggaaggcctttagtcttctcaaccagcttaatcgccataagaacattcacacaggtgaaaaaccgtttgaatgtaaggaatgtgggaagtcctttaatcgtagctcaaaccttgttcaacatcagagtattcatgctggtataaaaccatatgaatgtaaggagtgtgggaaaggctttaatcgtggtgcacaccttattcagcatcagaaaattcattccaatgagaaaccttttgtatgtagggaatgtgagatggcctttagatatcattgccaacttattgaacattctcgaattcatactggtgacaagccatttgaatgtcaagactgtgggaaggccttcaatcgtggctcaagccttgttcaacatcagagtattcacactggtgagaagccctatgaatgtaaggagtgtgggaaggcttttagactttacctacaactttcccaacatcagaaaactcacacaggtgaaaaaccatttgaatgtaaggaatgtgggaaattctttcgtcgtggttcaaatcttaatcaacatcgaagtattcatactggaaagaaaccctttgaatgtaaggaatgtgggaaagcctttcgacttcatatgcaccttattcgacatcagaaattgcatactggtgagaaaccctttgaatgtaaggagtgtgggaaagcctttcgacttcatatgcaacttattcgacatcagaaattgcatactggtgagaaaccctttgaatgtaaggaatgtggaaaggtttttagtcttcccacccagcttaatcgccataagaacattcacacaggtgagaaggcatcttga

SEQ ID NO: 840 - protein, 642 aa, Artificial Sequence
  synthetic
MVHGSVTFRDVAIDFSQEEWECLQPDQRTLYRDVMLENYSHLISLAGSSISKPDVITLLEQEKEPWMVVRKETSRRYPDLELKYGPEKVSPENDTSEVNLPKQVIKQISTTLGIEAFYFRNDSEYRQFEGLQGYQEGNINQKMISYEKLPTHTPHASLICNTHKPYECKECGKYFSRSANLIQHQSIHTGEKPFECKECGKAFRLHIQFTRHQKFHTGEKPFECNECGKAFSLLTLLNRHKNIHTGEKLFECKECGKSFNRSSNLVQHQSIHSGVKPYECKECGKGFNRGAHLIQHQKIHSNEKPFVCKECGMAFRYHYQLIEHCQIHTGEKPFECKECGKAFTLLTKLVRHQKIHTGEKPFECRECGKAFSLLNQLNRHKNIHTGEKPFECKECGKSFNRSSNLVQHQSIHAGIKPYECKECGKGFNRGAHLIQHQKIHSNEKPFVCRECEMAFRYHCQLIEHSRIHTGDKPFECQDCGKAFNRGSSLVQHQSIHTGEKPYECKECGKAFRLYLQLSQHQKTHTGEKPFECKECGKFFRRGSNLNQHRSIHTGKKPFECKECGKAFRLHMHLIRHQKLHTGEKPFECKECGKAFRLHMQLIRHQKLHTGEKPFECKECGKVFSLPTQLNRHKNIHTGEKAS

SEQ ID NO: 841 - DNA, 1047 bp, Artificial Sequence
  synthetic
atgcctgacgagctgacggagcccgggcgcgccacgccggcccgcgcctcctccttcctcatcgagaacctgctggcggccgaggccaagggcgcagggcgcgcgacccagggcgacggcagccgggaggacgaggaggaggacgacgacgaccccgaagacgaggacgccgagcaggcgcggcggcgacggctacagcggcggcgacagttgctcgcgggcaccgggcccggcggggaggcgcgggcccgtgcgctgctcgggccgggcgcgctgggcctcggtcctcggccgccccccggtcccgggccgcccttcgctctgggctgcggaggcgcagcgcgctggtacccacgggcgcacggtggctatggaggcggcctcagtcctgacaccagcgaccgggactcaccggagacgggcgaggagatgggccgtgcggagggcgcctggccgcgaggccccgggccgggagcggtgcagcgggaggcagcggagctggcggcgcgtggcccggcggccggcacggaggaggcgtcggagctggccgaggtccctgcggcggctggggagacacgcggcggcgttggcgtgggcggcggccgaaagaagaagacgcgcacagtcttctcccgcagccaggtcttccagctggaatccaccttcgacctgaagcgctacctgagcagcgccgagcgcgccggcctggccgcctccctgcagctcaccgagacgcaggttaagatctggttccagaaccgccgcaacaagtggaagcggcagctggcagccgagctggaggcggccagcctgtccccgccgggagcgcagcgcctggtccgcgtgccggtgctctaccacgaaagccccccggccgcagccgccgctgggcccccggccaccctgcccttcccgctggcgcccgccgcgcccgcgccgcccccaccgctgctcggcttctccggggccctcgcctacccgctggccgccttcccggccgccgcctccgtgccctttctgcgggcgcagatgcctggcctggtgtga

SEQ ID NO: 842 - protein, 348 aa, Artificial Sequence
  synthetic
MPDELTEPGRATPARASSFLIENLLAAEAKGAGRATQGDGSREDEEEDDDDPEDEDAEQARRRRLQRRRQLLAGTGPGGEARARALLGPGALGLGPRPPPGPGPPFALGCGGAARWYPRAHGGYGGGLSPDTSDRDSPETGEEMGRAEGAWPRGPGPGAVQREAAELAARGPAAGTEEASELAEVPAAAGETRGGVGVGGGRKKKTRTVFSRSQVFQLESTFDLKRYLSSAERAGLAASLQLTETQVKIWFQNRRNKWKRQLAAELEAASLSPPGAQRLVRVPVLYHESPPAAAAAGPPATLPFPLAPAAPAPPPPLLGFSGALAYPLAAFPAAASVPFLRAQMPGLV

SEQ ID NO: 843 - DNA, 2204 bp, Artificial Sequence
  synthetic
tgaggcctgcggtgctgggctccccagaccgagcacccccagaagatgaggggcctgtcatggtgaagctagaggactctgaggaggagggtgaggctgccttatgggacccaggccctgaagctgcacgcctgcgtttccggtgcttccgctatgaggaggccacagggccccaagaggccctggcccagctccgagagctgtgtcgccagtggctgcgtccagaggtacgctccaaggagcagatgctggagctgttggtgctggagcagttcctgggcgcactgccccctgagatccaggcccgtgtgcaggggcagcggccaggcagccccgaggaggctgctgccctagtagatgggctgcgccgggagccgggcggaccccggagatgggtcacagtccaggtgcagggccaggaggtcctatcagagaagatggagccctccagtttccagcccctacctgaaactgagcctccaactccagagcctgggcccaagacacctcctaggactatgcaggaatcaccactgggcctgcaggtgaaagaggagtcagaggttacagaggactcagatttcctggagtctgggcctctagctgccacccaggagtctgtacccaccctcctgcctgaggaggcccagagatgtgggaccgtgctggaccagatctttccccacagcaagactgggcctgagggtccctcatggagggagcaccccagggccctgtggcatgaggaagctgggggcatcttctccccagggttcgcgctgcagctaggcagcatctccgcaggtccaggtagtgtaagccctcacctccacgtcccctgggacctcggcatggctggcctttctggccagatccaatcaccctcccgcgaaggtggctttgcgcatgcgcttctgctccccagcgatctgaggagtgaacaggaccccacggacgaggatccctgccggggtgtgggccctgctctgatcaccacccgctggcgctcccccaggggccggagccggggccgccccagcactgggggcggggtggttaggggcggccgttgcgatgtatgtggcaaggtgttcagccaacgcagcaacctgctgaggcaccagaagatccacacgggtgagcgaccattcgtgtgcagcgagtgcggccgcagcttcagccgcagctcgcacctgctgcgccaccagcttacgcacaccgaggagcggccgttcgtgtgcggcgactgtggccagggcttcgtgcgcagcgcgcgcctggaagagcatcggagagtgcacacgggcgaacagcctttccgttgcgctgagtgcggccagagcttccggcagcgctccaatctgctgcagcaccagcgcatccacggcgatcccccgggccctggcgctaagcccccggcccctcctggtgcgcccgagcctcccggcccctttccgtgcagcgagtgccgcgagagcttcgcgcggcgcgccgtgctgctggagcaccaggcggtacacacgggcgacaagtcctttggctgcgtcgagtgcggcgagcgcttcggccgccgctcagtgctgctgcagcaccggcgcgtgcacagtggcgagcggcccttcgcctgtgccgagtgcggccagagcttccggcagcgctccaacctgacgcagcaccggcgcatccacaccggggagcggcccttcgcctgcgccgagtgtggcaaggccttccgccagcggcctacgctcacgcagcatctccgcgtacacacgggcgagaaaccctttgcctgccccgagtgtggccagcgcttcagccagcgcctcaagctcacgcgtcatcagaggacacacaccggcgaaaagccctaccactgcggtgagtgcggcctgggcttcacgcaggtctcgcggctcaccgagcaccagcgcatccacacgggcgaacggcccttcgcctgccccgagtgcggccagagctttcggcagcacgccaacctcacccagcaccggcgcatccacacgggtgaacggccctacgcatgccctgagtgtggcaaggccttccgccagcggcccacgctcacgcagcatctgcgcacccaccgacgagagaagcccttcgcctgccaggactgtggccgccgcttccaccagagcaccaagctcattcagcaccagcgcgtccacagcgccgagtag

SEQ ID NO: 844 - protein, 734 aa, Artificial Sequence
  synthetic
MRPAVLGSPDRAPPEDEGPVMVKLEDSEEEGEAALWDPGPEAARLRFRCFRYEEATGPQEALAQLRELCRQWLRPEVRSKEQMLELLVLEQFLGALPPEIQARVQGQRPGSPEEAAALVDGLRREPGGPRRWVTVQVQGQEVLSEKMEPSSFQPLPETEPPTPEPGPKTPPRTMQESPLGLQVKEESEVTEDSDFLESGPLAATQESVPTLLPEEAQRCGTVLDQIFPHSKTGPEGPSWREHPRALWHEEAGGIFSPGFALQLGSISAGPGSVSPHLHVPWDLGMAGLSGQIQSPSREGGFAHALLLPSDLRSEQDPTDEDPCRGVGPALITTRWRSPRGRSRGRPSTGGGVVRGGRCDVCGKVFSQRSNLLRHQKIHTGERPFVCSECGRSFSRSSHLLRHQLTHTEERPFVCGDCGQGFVRSARLEEHRRVHTGEQPFRCAECGQSFRQRSNLLQHQRIHGDPPGPGAKPPAPPGAPEPPGPFPCSECRESFARRAVLLEHQAVHTGDKSFGCVECGERFGRRSVLLQHRRVHSGERPFACAECGQSFRQRSNLTQHRRIHTGERPFACAECGKAFRQRPTLTQHLRVHTGEKPFACPECGQRFSQRLKLTRHQRTHTGEKPYHCGECGLGFTQVSRLTEHQRIHTGERPFACPECGQSFRQHANLTQHRRIHTGERPYACPECGKAFRQRPTLTQHLRTHRREKPFACQDCGRRFHQSTKLIQHQRVHSAE

SEQ ID NO: 845 - DNA, 1929 bp, Artificial Sequence
  synthetic
atggactcagtctcctttgaggatgtggccgtgaacttcaccctggaggagtgggctttgctggattcttcacagaaaaagctctatgaagatgtgatgcaggagaccttcaaaaacctggtttgtctaggaaaaaagtgggaagaccaggacattgaagatgaccacagaaaccaggggaaaaatcgaagatgtcatatggttgagagactctgtgaaagtagaagaggtagcaaatgtggagaaaccactagccagatgccaaatgttaatatcaacaaggaaacttttactggagcaaaaccacatgaatgcagcttttgtggaagagacttcattcatcattcgtcccttaataggcacatgagatctcacactggacagaaaccaaatgagtatcaggaatatgaaaagcaaccatgtaaatgtaaagcagttgggaaaaccttcagttatcaccactgctttcgcaaacatgaaagaactcacactggagtgaagccctatgaatgtaaacagtgtgggaaagcctttatatattaccagccatttcaaagacatgaaaggactcatgctggacagaaaccctatgaatgtaagcaatgtggaaaaacctttatatattaccagtcttttcaaaaacatgctcatactggaaagaaaccctatgaatgtaaacagtgtgggaaagcctttatatgttaccaatcttttcaaagacacaaaaggactcacactggagagaaaccctatgaatgtaagcaatgtggtaaggctttcagttgtcccacatactttcgaactcatgaaagaactcacactggagaaaaaccctacaaatgtaaagaatgtggtaaagccttcagttttctcagttcttttcgaaggcataaaaggactcatagtggagagaaaccctatgaatgtaaagaatgtggaaaagccttcttttattctgcaagctttcgagcacatgtaataatacacactggggctcgaccttataaatgtaaagaatgtgggaaagccttcaactcttctaattcctgtcgagtgcatgaaagaactcatattggagaaaaaccatatgaatgtaaacgatgtggcaaatcattcagttggtccatttctcttcgattgcatgaaagaactcatactggagagaaaccttatgagtgtaaacagtgtcataaaaccttcagtttttcaagttcccttcgagaacacgaaacaactcacactggagagaaaccctatgaatgtaaacaatgtggtaaaaccttcagtttttcaagttcccttcaaagacatgaaaggactcacaatgcagagaaaccctatgaatgtaaacagtgtgggaaagccttcaggtgttcaagttattttcgaattcatgaaaggtcacacactggagagaaaccctatgaatgtaaacagtgtggaaaagttttcattcgttccagttcctttcgactgcatgaaagaacacacactggagagaaaccctatgaatgtaaactatgcggtaaaaccttcagtttttcaagttcccttcgagaacatgaaaaaattcacactggaaataagccttttgagtgtaagcaatgtggtaaggccttccttcgttccagtcaaattcgattgcatgaaaggactcacactggagagaaaccgtatcaatgtaaacaatgtggaaaagccttcatttcttccagtaaatttcgaatgcatgagagaactcacacgggagagaaaccctatcgatgtaaacaatgtgggaaagccttcagattttcaagttctgttcgaattcatgaaaggtctcacactggagagaaaccttatgaatgcaaacaatgtggaaaagccttcatttcttccagtcactttcgactgcatgaaaggactcatatgggagagaaagtctaa

SEQ ID NO: 846 - protein, 642 aa, Artificial Sequence
  synthetic
MDSVSFEDVAVNFTLEEWALLDSSQKKLYEDVMQETFKNLVCLGKKWEDQDIEDDHRNQGKNRRCHMVERLCESRRGSKCGETTSQMPNVNINKETFTGAKPHECSFCGRDFIHHSSLNRHMRSHTGQKPNEYQEYEKQPCKCKAVGKTFSYHHCFRKHERTHTGVKPYECKQCGKAFIYYQPFQRHERTHAGQKPYECKQCGKTFIYYQSFQKHAHTGKKPYECKQCGKAFICYQSFQRHKRTHTGEKPYECKQCGKAFSCPTYFRTHERTHTGEKPYKCKECGKAFSFLSSFRRHKRTHSGEKPYECKECGKAFFYSASFRAHVIIHTGARPYKCKECGKAFNSSNSCRVHERTHIGEKPYECKRCGKSFSWSISLRLHERTHTGEKPYECKQCHKTFSFSSSLREHETTHTGEKPYECKQCGKTFSFSSSLQRHERTHNAEKPYECKQCGKAFRCSSYFRIHERSHTGEKPYECKQCGKVFIRSSSFRLHERTHTGEKPYECKLCGKTFSFSSSLREHEKIHTGNKPFECKQCGKAFLRSSQIRLHERTHTGEKPYQCKQCGKAFISSSKFRMHERTHTGEKPYRCKQCGKAFRFSSSVRIHERSHTGEKPYECKQCGKAFISSSHFRLHERTHMGEKV

SEQ ID NO: 847 - DNA, 1998 bp, Artificial Sequence
  synthetic
atggaatccgtcacctttgaggatgtggccgtggagttcatccaggagtgggcattgctggacagcgcacggaggagcctgtgcaaatacaggatgcttgaccagtgcaggaccctggcctccaggggaactccaccatgcaaacccagttgtgtctcccagctggggcaaagagcagagccaaaggcaacagaacgagggattctccgtgccacaggtgttgcctgggaatctcaacttaaacccgaagagttgccttctatgcaggatcttttggaagaagcatcctccagggacatgcaaatggggccggggctgttcctgaggatgcagctggtgccctccatagaagagagggagacaccattgactcgagaggaccggccagctctccaggagccgccttggtctctgggatgcacgggactgaaggccgctatgcagattcagagggtggtgataccagtgcctactctgggccaccgcaacccatgggtggccagggattctgctgtgcctgcacgtgaccctgcctggcttcaggaggacaaagtggaggaagaagctatggctcctgggctgccaaccgcctgttcacaggaaccagtcacctttgcagatgtggctgtggtgttcaccccagaagaatgggtgtttctggactctactcagaggagcctgtatagagatgtgatgctggagaactacaggaacctggcctctgtggctgatcaactgtgcaaacccaatgcgttgtcttatttggaagaaagaggagagcagtggaccactgacaggggcgtcctctcagacacctgtgcagaacctcagtgtcaaccccaagaggcaattcctagccaagatacttttacagagatcctgtccattgatgtgaaaggggagcaacctcagcctggagaaaaactctataaatataatgaacttgagaaaccttttaacagcattgaaccacttttccagtaccagagaattcatgctggagaggcatcctgtgaatgtcaagagattagaaattccttcttccagagtgcccacctaattgtgcccgagaaaatccgtagtggggataaatcctatgcatgtaacaaatgtgaaaaatccttcagatacagctctgaccttatcaggcatgagaagactcatactgcagagaagtgctttgactgtcaagaatgtgggcaagccttcaaatattcctcgaatctccggcgacacatgagaacccataccggagagaagccatttgaatgtagtcagtgtgggaaaaccttcacgaggaactttaacctgattttgcaccagagaaaccacacaggagagaagccctacgagtgtaaagattgtgggaaagccttcaatcagccatcatccctcaggagccacgtgagaactcacactggagagaagccctttgaatgcagccagtgtgggaaagccttcagggaacactcttcactgaagacacatctgcgaacccataccagagagaaaccatatgaatgcaaccagtgtggcaagcccttccggacgagcactcatctgaacgtgcacaagaggatacacacaggggagaaactgtatgagtgcgcgacttgcggtcaggtcttgagtcgtctttcaaccctgaagagtcacatgcgaactcacactggagagaagccctatgtgtgccaggaatgtgggcgagccttcagtgagccctcatccctcaggaaacatgcaaggactcacagtggcaagaagccctatgcatgccaggaatgcgggcgagcctttggtcagtcttcacatcttattgtacatgtgagaacacacagtgccgggagaccctatcaatgtaatcagtgtgagaaagccttcaggcacagctcctcactcactgtacacaaaagaacccatgtgggaagagagaccattaggaatggcagcctgcctttatccatgtctcatccatactgtgggccccttgctaattaa

SEQ ID NO: 848 - protein, 665 aa, Artificial Sequence
  synthetic
MESVTFEDVAVEFIQEWALLDSARRSLCKYRMLDQCRTLASRGTPPCKPSCVSQLGQRAEPKATERGILRATGVAWESQLKPEELPSMQDLLEEASSRDMQMGPGLFLRMQLVPSIEERETPLTREDRPALQEPPWSLGCTGLKAAMQIQRVVIPVPTLGHRNPWVARDSAVPARDPAWLQEDKVEEEAMAPGLPTACSQEPVTFADVAVVFTPEEWVFLDSTQRSLYRDVMLENYRNLASVADQLCKPNALSYLEERGEQWTTDRGVLSDTCAEPQCQPQEAIPSQDTFTEILSIDVKGEQPQPGEKLYKYNELEKPFNSIEPLFQYQRIHAGEASCECQEIRNSFFQSAHLIVPEKIRSGDKSYACNKCEKSFRYSSDLIRHEKTHTAEKCFDCQECGQAFKYSSNLRRHMRTHTGEKPFECSQCGKTFTRNFNLILHQRNHTGEKPYECKDCGKAFNQPSSLRSHVRTHTGEKPFECSQCGKAFREHSSLKTHLRTHTREKPYECNQCGKPFRTSTHLNVHKRIHTGEKLYECATCGQVLSRLSTLKSHMRTHTGEKPYVCQECGRAFSEPSSLRKHARTHSGKKPYACQECGRAFGQSSHLIVHVRTHSAGRPYQCNQCEKAFRHSSSLTVHKRTHVGRETIRNGSLPLSMSHPYCGPLAN

SEQ ID NO: 849 - DNA, 1926 bp, Artificial Sequence
  synthetic
atggactcagtggtctttgaggatgtggctgtgaacttcacccaggaggagtgggctttgctgggtccctctcagaagaaactctacagagatgtgatgcaagaaacctttgttaacttggcctctataggggaaaactgggaggagaagaacattgaagatcacaaaaatcaggggagaaagctaagaagtcatatggtagagaggctctgtgaaaggaaagaaggtagtcagtttggagaaaccatcagtcagactccaaatcctaaaccaaacaagaaaacttttactagagtaaaaccatatgaatgtagtgtgtgtggaaaggactatatgtgtcattcatctcttaataggcacatgagatctcatactgaacatagatcatatgaatatcacaaatatggagagaaatcatatgaatgtaaggaatgtgggaaaagattcagctttcgaagttcatttcgaatacatgaaagaactcacactggagagaaaccctataaatgtaaacagtgtggtaaggctttcagttggcccagttcctttcaaatacatgaaagaactcatactggagagaaaccttatgaatgtaaggaatgtgggaaggccttcatttatcacacaacctttcgaggacacatgagaatgcacacaggggagaaaccctataaatgtaaagaatgcgggaaaacgttcagtcatcccagttcttttcgaaatcatgaaagaactcactctggagagaaaccctatgaatgtaaacaatgtggaaaagctttcagatattaccaaacttttcaaatacatgaaaggactcacactggggaaaaaccctatcagtgtaagcaatgtggtaaagctcttagttgtcccacatcctttcgaagtcatgaaaggattcacactggagaaaaaccctataaatgtaaaaaatgtgggaaagccttcagttttcctagttcctttagaaaacatgaaagaattcatacaggagagaaaccctatgattgtaaggaatgtgggaaagcattcatttctcttccaagctatcgaagacatatgataatgcacactggaaatggaccttataaatgcaaggaatgtgggaaagcctttgattgtcctagttcttttcaaatccatgaacgaactcacactggagagaaaccctatgaatgtaaacagtgtggtaaagccttcagttgttccagttcctttcgaatgcatgaaagaactcacactggagagaaaccccatgaatgtaaacaatgtggtaaagccttcagttgttccagttctgttcgaatacatgaaaggactcacactggagagaaaccctatgaatgtaaacagtgtggtaaagccttcagttgttccagttcctttcgaatgcatgaaagaattcacactggagagaaaccctatgaatgtaaacagtgtggtaaagcctttagtttttctagttcctttcggatgcatgaaaggactcacactggagagaaaccctatgaatgtaaacaatgtggtaaagccttcagttgttccagttcctttcgaatgcatgaaaggactcacactggggagaaaccctatgaatgtaaacagtgtggtaaggcgtttagttgttccagttccattcgaatacatgaaaggactcacactggagagaaaccttatgagtgtaaacaatgtggtaaggccttcagttgttctagttctgttcgaatgcatgaaaggactcacactggagtgaaaccctatgaatgtaaacaatgtgacaaagccttcagttgctcacgttcctttcgaatccatgaacgaactcacactggagagaaaccctatgcatgtcaacaatgtggtaaagccttcaagtgttcccgttcctttcgaatacatgaaagagttcatagtggagagtaa

SEQ ID NO: 850 - protein, 641 aa, Artificial Sequence
  synthetic
MDSVVFEDVAVNFTQEEWALLGPSQKKLYRDVMQETFVNLASIGENWEEKNIEDHKNQGRKLRSHMVERLCERKEGSQFGETISQTPNPKPNKKTFTRVKPYECSVCGKDYMCHSSLNRHMRSHTEHRSYEYHKYGEKSYECKECGKRFSFRSSFRIHERTHTGEKPYKCKQCGKAFSWPSSFQIHERTHTGEKPYECKECGKAFIYHTTFRGHMRMHTGEKPYKCKECGKTFSHPSSFRNHERTHSGEKPYECKQCGKAFRYYQTFQIHERTHTGEKPYQCKQCGKALSCPTSFRSHERIHTGEKPYKCKKCGKAFSFPSSFRKHERIHTGEKPYDCKECGKAFISLPSYRRHMIMHTGNGPYKCKECGKAFDCPSSFQIHERTHTGEKPYECKQCGKAFSCSSSFRMHERTHTGEKPHECKQCGKAFSCSSSVRIHERTHTGEKPYECKQCGKAFSCSSSFRMHERIHTGEKPYECKQCGKAFSFSSSFRMHERTHTGEKPYECKQCGKAFSCSSSFRMHERTHTGEKPYECKQCGKAFSCSSSIRIHERTHTGEKPYECKQCGKAFSCSSSVRMHERTHTGVKPYECKQCDKAFSCSRSFRIHERTHTGEKPYACQQCGKAFKCSRSFRIHERVHSGE

SEQ ID NO: 851 - DNA, 1584 bp, Artificial Sequence
  synthetic
atgactgcagaattgagagaagccatggccctagccccatggggcccagtgaaggtgaaaaaggaggaggaagaagaagaaaacttcccaggtcaggcatccagccaacaagtgcactccgagaacatcaaagtctgggccccagtgcagggtcttcagacaggccttgatggatcagaagaggaagaaaagggtcagaacatatcctgggatatggcggtagtcctgaaagcaactcaggaggcacctgctgcttcaacccttggcagctactcattaccagggactctggccaagagtgagatactggagactcatgggaccatgaactttctaggtgctgaaaccaagaacctacagttactggttccaaaaactgagatatgtgaggaagctgaaaaacccctcatcatatcagaaagaatccagaaagctgatcctcaaggacctgagttaggagaagcttgtgaaaagggaaacatgttaaagaggcagagaataaagagagaaaagaaagatttcagacaagtgatagtgaatgactgtcacttacctgaaagcttcaaagaagaggaaaaccagaaatgtaagaaatctggaggaaaatatagccttaattctggcgctgttaaaaatccaaaaacccagcttggacaaaagccttttacgtgtagcgtgtgtgggaaaggatttagtcagagtgcaaacctcgttgtgcatcagcgaatccacactggagagaaaccctttgaatgtcatgagtgtgggaaggccttcattcagagtgcaaacctcgttgtgcatcagagaatccacactggacagaaaccttatgtttgctcaaaatgtgggaaagccttcactcagagttcaaatctgactgtacatcaaaaaatccactccttagaaaaaacttttaagtgcaatgaatgtgagaaagcctttagttacagctcacaacttgctcggcaccagaaagtccacattacggaaaaatgctatgaatgtaatgaatgtgggaaaacatttactaggagctcaaacctcattgtccaccagaggatccacactggggagaagccctttgcctgtaacgactgtggcaaagcctttacccagagtgcaaatcttattgtacatcagcgaagccatactggtgagaagccatatgagtgtaaagagtgtgggaaagcctttagttgtttttcacaccttattgtgcaccagagaattcacactgcagagaaaccttacgactgcagcgaatgtgggaaagccttcagtcagctctcttgccttattgtccaccagagaattcacagtggagatcttccttacgtgtgtaatgaatgtgggaaggccttcacatgtagctcatacctacttattcatcagagaattcataatggagaaaaaccttacacatgtaatgagtgtgggaaggccttcagacagaggtcgagcctcaccgtgcaccagagaacccacactggggagaagccctatgaatgtgagaagtgtggtgcagctttcatttccaactcacacctcatgcgacaccatagaacccatcttgttgaataa

SEQ ID NO: 852 - protein, 527 aa, Artificial Sequence
  synthetic
MTAELREAMALAPWGPVKVKKEEEEEENFPGQASSQQVHSENIKVWAPVQGLQTGLDGSEEEEKGQNISWDMAVVLKATQEAPAASTLGSYSLPGTLAKSEILETHGTMNFLGAETKNLQLLVPKTEICEEAEKPLIISERIQKADPQGPELGEACEKGNMLKRQRIKREKKDFRQVIVNDCHLPESFKEEENQKCKKSGGKYSLNSGAVKNPKTQLGQKPFTCSVCGKGFSQSANLVVHQRIHTGEKPFECHECGKAFIQSANLVVHQRIHTGQKPYVCSKCGKAFTQSSNLTVHQKIHSLEKTFKCNECEKAFSYSSQLARHQKVHITEKCYECNECGKTFTRSSNLIVHQRIHTGEKPFACNDCGKAFTQSANLIVHQRSHTGEKPYECKECGKAFSCFSHLIVHQRIHTAEKPYDCSECGKAFSQLSCLIVHQRIHSGDLPYVCNECGKAFTCSSYLLIHQRIHNGEKPYTCNECGKAFRQRSSLTVHQRTHTGEKPYECEKCGAAFISNSHLMRHHRTHLVE

SEQ ID NO: 853 - DNA, 1047 bp, Artificial Sequence
  synthetic
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 854 - protein, 348 aa, Artificial Sequence [sequence omitted]

SEQ ID NO: 855 - DNA, 1041 bp, Artificial Sequence
  synthetic
atggcgctactgaaagtcaagtttgaccagaagaagcgggtcaagttggcccaagggctctggctcatgaactggttctccgtgttggctggcatcatcatcttcagcctaggactgttcctgaagattgaactccgaaagaggagcgatgtgatgaataattctgagagccattttgtgcccaactcattgatagggatgggggtgctatcctgtgtcttcaactcgctggctgggaagatctgctacgacgccctggacccagccaagtatgccagatggaagccctggctgaagccgtacctggctatctgtgttctcttcaacatcatcctcttccttgtggctctctgctgctttctgcttcggggctcgctggagaacaccctgggccaagggctcaagaacggcatgaagtactaccgggacacagacacccctggcaggtgtttcatgaagaagaccatcgacatgctgcagatcgagttcaaatgctgcggcaacaacggttttcgggactggtttgagattcagtggatcagcaatcgctacctggacttttcctccaaagaagtcaaagatcgaatcaagagcaacgtggatgggcggtacctggtggacggcgtccctttcagctgctgcaatcctagctcgccacggccctgcatccagtatcagatcaccaacaactcagcacactacagttacgaccaccagacggaggagctcaacctgtgggtgcgtggctgcagggctgccctgctgagctactacagcagcctcatgaactccatgggtgtcgtcacgctcctcatttggctcttcgaggtgaccattacaattgggctgcgctacctacagacgtcgctggatggtgtgtccaaccccgaggaatctgagagcgagagccagggctggctgctggagaggagcgtgccggagacctggaaggcctttctggagagtgtgaagaagctgggcaagggcaaccaggtggaagccgagggcgcagacgcaggccaggccccagaggctggctga

SEQ ID NO: 856 - protein, 346 aa, Artificial Sequence
  synthetic
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESQGWLLERSVPETWKAFLESVKKLGKGNQVEAEGADAGQAPEAG

SEQ ID NO: 857 - DNA, 6471 bp, Artificial Sequence
  synthetic
atgagtgataccccttctactggtttttccatcattcatcctacgtcttctgaaggtcaagttccaccccctcgccatttgagcctcactcatcctgttgtggccaagcgaatcagtttctacaagagcggagacccccaattcggcggggtcagggtggtggtcaaccctcgctcctttaagtcctttgatgctctgctggataacttgtccaggaaggtgcccctcccttttggagtgaggaacatcagcacccctcggggcaggcacagcatcacgcgcctggaggagctggaggacggcgagtcctacctatgttcccacggcaggaaggtgcagcctgtagacctggacaaagcccgtcggcgcccgcggccctggctcagcagccgggccattagcgcgcactcaccgccccaccccgtagccgtcgctgctcccggcatgccccgccccccacggagcctagtggtcttcaggaatggcgacccgaagacgaggcgtgcggttcttctgagcaggagggtcacccagagcttcgaggcatttctacagcacctgacagaggtcatgcagcgccctgtggtcaagctgtacgctacggacggaaggagggttcccagcctccaggcagtgatcctgagctctggagctgtggtggcggcaggaagggagccatttaaaccaggaaattatgacatccaaaaatacttgcttcctgctagattaccagggatctctcagcgtgtgtaccccaagggaaatgcaaagtcagaaagcagaaagataagcacacatatgtcttcaagctcaaggtcccagatttattctgtttcttctgagaaaacacataataatgattgctacttagactattcttttgttcctgaaaagtacttggccttagaaaagaatgattctcagaatttaccaatatatccttctgaagatgatattgagaaatcaattatttttaatcaagacggcactatgacagttgagatgaaagttcgattcagaataaaagaggaagaaaccataaaatggacaactactgtcagtaaaactggtccttctaataatgatgaaaagagtgagatgagttttccaggaagaacagaaagtcgatcatctggtttaaagcttgcagcatgttcattctctgcagatgtgtcacctatggagcgaagcagtaatcaagagggcagtttggcagaggagataaacattcaaatgacagatcaagtggctgaaacttgcagttctgctagttgggagaatgctactgtggacacagatatcatccagggaactcaagaccaagcaaagcatcgtttttataggccccctacacctggactaagaagagtgagacaaaagaaatctgtgattggcagtgtgaccttagtatctgaaactgaggttcaagagaaaatgattggacagttttcatatagtgaagaaagggaaagtggggaaaacaagtctgagtatcacatgtttacacattcttgcagtaaaatgtcatcagtatctaacaaaccagtacttgttcagatcaataacaatgatcaaatggaggagtcatcattagaaagaaaaaaggaaaacagtctgcttaagtcaagtgcaataagtgctggtgttatagaaattacaagtcagaagatgttagagatgtcacataataatggtttgccatcaactatatcaaataactcaattgtggaggaagatgtagttgattgtgtggtattggacaacaaaactggtatcaagaacttcaaaacttatggtaacaccaatgataggttcagtcctatttcagcagatgcaacccatttttcaagtaataactctggaactgacaaaaatatttctgaggctccagcttcagaagcatcctctactgtcactgcaagaattgacagactaattaatgaatttgctcagtgtggtttaacaaaacttccaaaaaatgaaaagaagattttgtcatctgttgccagcaaaaagaagaaaaaatctcgacagcaagcaataaattccaggtatcaagatggacagcttgcaaccaaaggaattcttaataagaatgagagaataaacacaaaaggtagaattacaaaggaaatgatagtgcaagattcagatagtccccttaaaggagggatactttgtgaggaagacctccagaaaagtgatactgtaattgaatcaaatactttttgttccaaaagtaatctcaattccacgatttccaagaatttccatagaaataaattaaatactactcaaaattccaaggttcaaggacttttaaccaaaagaaaatctagatcactaaataaaataagcttaggagcacctaaaaaaagagaaatcggtcaaagagataaagtgtttcctcacaatgaatctaaatattgcaaaagtacttttgaaaacaaaagtttatttcatgtatttaacatccttgagcaaaaacccaaagatttttatgcaccgcaatctcaagcagaagtggcatctgggtatttgagaggaatggcaaagaagagtttagtttcaaaagttactgattcacacataactttaaaaagccagaaaaaacgtaaaggggataaagtgaaagcaagtgctattttaagtaaacaacatgctacaaccagggcaaattctttagcttctttgaaaaaacctgattttcctgaggctattgctcatcattcaattcaaaattatatacagagttggttgcagaacataaatccatatccaactttaaagcctataaaatcagctccagtatgtagaaatgaaacgagtgtggtaaattgtagcaataatagtttttcagggaatgatccccatacaaattctggaaaaataagtaattttgttatggaaagtaataagcacataactaaaattgccggtttgacaggagataatctatgtaaagagggagataagtcttttattgccaatgacactggtgaagaagatctccatgagacacaggttggatctctgaatgatgcttatttggttcccctgcatgaacactgtactttgtcacagtcagctattaatgatcataatactaaaagtcatatagctgctgaaaaatcaggaccagagaaaaaacttgtttaccaggaaataaacctagctagaaaaaggcaaagtgtagaggctgccattcaagtagatcctatagaagaggaaactccaaaagacctcttaccagtcctgatgcttcaccaattgcaagcttcagttcctggtattcacaagactcagaatggagttgttcaaatgccaggttcacttgcaggtgttccctttcattctgcaatatgtaattcatccactaatctccttctagcttggctcttggtgctaaacctaaagggaagtatgaatagcttctgtcaagttgatgctcacaaggctaccaacaaatcttcagaaacacttgcattgttggagattctaaagcacatagctatcacagaggaagctgatgacttgaaagctgctgttgccaatttagtggagtcaactacaagccactttggactcagtgagaaagaacaagacatggttccaatagatctttctgcaaattgttccacggtcaacattcagagtgttcctaagtgcagtgaaaatgaaagaacacaaggaatctcctctttggatggaggttgctctgccagtgaggcatgtgcccctgaagtctgtgttttggaagtgacttgctctccatgtgagatgtgcactgtaaataaggcttattctccaaaagagacatgtaaccccagtgacactttttttcctagtgatggttatggtgtggatcagacttctatgaataaggcttgtttcctaggagaggtctgttcacttactgatactgtgttttctgataaggcttgtgctcaaaaggagaaccatacctatgagggagcttgcccaattgatgagacctacgttcctgtcaatgtctgcaataccattgactttttaaactccaaagaaaacacatatactgataacttggattcaactgaagagttagaaagaggtgatgacattcagaaagatctaaatattttgacagaccctgaatataaaaatggatttaatacattggtgtcacatcaaaatgtcagtaatttaagctcctgtggcctttgcctaagtgaaaaagaagcagaacttgataagaaacatagttctctagatgattttgaaaattgttcactaaggaagtttcaggatgaaaatgcatatacttcctttgatatggaagaaccacggacttctgaagaaccaggctcaataaccaacagcatgacatcaagtgaaagaaacatttcagaattggaatcttttgaagaattagaaaaccatgacactgatatctttaatacagtggtaaatggaggagagcaagccactgaagaattaatccaagaagaggtagaggctagtaaaactttagaattgatagacatctctagtaagaatattatggaagaaaaaagaatgaacggtataatttatgaaataatcagtaagaggctggcaacaccaccatctttagatttttgctatgattctaagcaaaatagtgaaaaggagaccaatgaaggagaaactaagatggtaaaaatgatggtgaaaactatggaaactggaagttattcagagtcctctcctgatttaaaaaaatgcatcaaaagtccagtgacttctgattggtcagactatcggcctgacagtgacagtgagcagccatataaaacatccagtgatgatcccaatgacagtggcgaacttacccaagagaaagaatataacataggatttgttaaaagggcaatagaaaaactgtacggtaaagcagatattatcaaaccatctttttttcctgggtctacccgcaaatctcaggtttgtccttataattctgtggaatttcagtgttccaggaaagcaagtctttatgattctgaagggcagtcatttggctcttctgaacaggtatctagtagttcatctatgttgcaggaattccaggaggaaagacaagataagtgtgatgttagtgctgtgagggacaattattgtaggggtgacattgtagaacctggtacaaaacaaaatgatgatagcagaatcctcacagacatagaggaaggagtactgattgacaaaggcaaatggcttctgaaagaaaatcatttgctaaggatgtcatctgaaaatcctggcatgtgtggcaatgcagacaccacatcagtggacaccctacttgataataacagcagtgaggtaccatattcacattttggtaatttggccccaggcccaacgatggatgaactctcctcttcagaactcgaggaactgactcaaccccttgaactaaaatgcaattactttaacatgcctcatggtagtgactcagaaccttttcatgaggacttgctggatgttcgcaatgaaacctgtgccaaggaaagaatagcaaatcatcatacagaggagaagggtagtcatcagtcagaaagagtatgcacatctgtcactcattcctttatttctgctggtaacaaagtctaccctgtctctgatgatgctattaaaaaccaaccattgcctggcagtaatatgattcatggtacacttcaggaagctgactctttggataaactgtatgctctttgtggtcaacattgcccaatactaactgttattatccaacccatgaatgaggaagaccgaggatttgcatatcgcaaagaatctgatattgaaaatttcttgggtttttatttatggatgaaaatacacccatatttacttcagacagacaaaaatgtgttcagggaagagaacaataaagcaagtatgagacaaaatcttattgataatgccattggtgatatatttgatcagttttatttcagtaacacatttgacttgatgggtaaaagaagaaaacaaaaaagaattaacttcttggggttagaggaagaaggtaatttaaagaaatttcaaccagatttgaaggaaaggttttgtatgaatttcttgcacacatcattgttagttgtgggtaatgtggattcaaatacacaagacctcagcggtcagacaaatgaaatctttaaagcagtcgatgagaataacaacttattaaataacagattccagggctcaagaacaaatctcaaccaagtagtaagagaaaatatcaactgtcattacttctttgaaatgcttggtcaagcttgcctcttagatatttgccaagttgagacctccttaaatattagcaacagaaatattttagaactttgtatgtttgagggtgaaaatcttttcatttgggaagaggaagacatattaaatttaactgatcttgaaagcagtagagaacaagaagatttataa

SEQ ID NO: 858 - protein, 2156 aa, Artificial Sequence
  synthetic
MSDTPSTGFSIIHPTSSEGQVPPPRHLSLTHPVVAKRISFYKSGDPQFGGVRVVVNPRSFKSFDALLDNLSRKVPLPFGVRNISTPRGRHSITRLEELEDGESYLCSHGRKVQPVDLDKARRRPRPWLSSRAISAHSPPHPVAVAAPGMPRPPRSLVVFRNGDPKTRRAVLLSRRVTQSFEAFLQHLTEVMQRPVVKLYATDGRRVPSLQAVILSSGAVVAAGREPFKPGNYDIQKYLLPARLPGISQRVYPKGNAKSESRKISTHMSSSSRSQIYSVSSEKTHNNDCYLDYSFVPEKYLALEKNDSQNLPIYPSEDDIEKSIIFNQDGTMTVEMKVRFRIKEEETIKWTTTVSKTGPSNNDEKSEMSFPGRTESRSSGLKLAACSFSADVSPMERSSNQEGSLAEEINIQMTDQVAETCSSASWENATVDTDIIQGTQDQAKHRFYRPPTPGLRRVRQKKSVIGSVTLVSETEVQEKMIGQFSYSEERESGENKSEYHMFTHSCSKMSSVSNKPVLVQINNNDQMEESSLERKKENSLLKSSAISAGVIEITSQKMLEMSHNNGLPSTISNNSIVEEDVVDCVVLDNKTGIKNFKTYGNTNDRFSPISADATHFSSNNSGTDKNISEAPASEASSTVTARIDRLINEFAQCGLTKLPKNEKKILSSVASKKKKKSRQQAINSRYQDGQLATKGILNKNERINTKGRITKEMIVQDSDSPLKGGILCEEDLQKSDTVIESNTFCSKSNLNSTISKNFHRNKLNTTQNSKVQGLLTKRKSRSLNKISLGAPKKREIGQRDKVFPHNESKYCKSTFENKSLFHVFNILEQKPKDFYAPQSQAEVASGYLRGMAKKSLVSKVTDSHITLKSQKKRKGDKVKASAILSKQHATTRANSLASLKKPDFPEAIAHHSIQNYIQSWLQNINPYPTLKPIKSAPVCRNETSVVNCSNNSFSGNDPHTNSGKISNFVMESNKHITKIAGLTGDNLCKEGDKSFIANDTGEEDLHETQVGSLNDAYLVPLHEHCTLSQSAINDHNTKSHIAAEKSGPEKKLVYQEINLARKRQSVEAAIQVDPIEEETPKDLLPVLMLHQLQASVPGIHKTQNGVVQMPGSLAGVPFHSAICNSSTNLLLAWLLVLNLKGSMNSFCQVDAHKATNKSSETLALLEILKHIAITEEADDLKAAVANLVESTTSHFGLSEKEQDMVPIDLSANCSTVNIQSVPKCSENERTQGISSLDGGCSASEACAPEVCVLEVTCSPCEMCTVNKAYSPKETCNPSDTFFPSDGYGVDQTSMNKACFLGEVCSLTDTVFSDKACAQKENHTYEGACPIDETYVPVNVCNTIDFLNSKENTYTDNLDSTEELERGDDIQKDLNILTDPEYKNGFNTLVSHQNVSNLSSCGLCLSEKEAELDKKHSSLDDFENCSLRKFQDENAYTSFDMEEPRTSEEPGSITNSMTSSERNISELESFEELENHDTDIFNTVVNGGEQATEELIQEEVEASKTLELIDISSKNIMEEKRMNGIIYEIISKRLATPPSLDFCYDSKQNSEKETNEGETKMVKMMVKTMETGSYSESSPDLKKCIKSPVTSDWSDYRPDSDSEQPYKTSSDDPNDSGELTQEKEYNIGFVKRAIEKLYGKADIIKPSFFPGSTRKSQVCPYNSVEFQCSRKASLYDSEGQSFGSSEQVSSSSSMLQEFQEERQDKCDVSAVRDNYCRGDIVEPGTKQNDDSRILTDIEEGVLIDKGKWLLKENHLLRMSSENPGMCGNADTTSVDTLLDNNSSEVPYSHFGNLAPGPTMDELSSSELEELTQPLELKCNYFNMPHGSDSEPFHEDLLDVRNETCAKERIANHHTEEKGSHQSERVCTSVTHSFISAGNKVYPVSDDAIKNQPLPGSNMIHGTLQEADSLDKLYALCGQHCPILTVIIQPMNEEDRGFAYRKESDIENFLGFYLWMKIHPYLLQTDKNVFREENNKASMRQNLIDNAIGDIFDQFYFSNTFDLMGKRRKQKRINFLGLEEEGNLKKFQPDLKERFCMNFLHTSLLVVGNVDSNTQDLSGQTNEIFKAVDENNNLLNNRFQGSRTNLNQVVRENINCHYFFEMLGQACLLDICQVETSLNISNRNILELCMFEGENLFIWEEEDILNLTDLESSREQEDL

SEQ ID NO: 859 - DNA, 900 bp, Artificial Sequence
  synthetic
atgatggcgtatatgaacccggggccccactattctgtcaacgccttggccctaagtggccccagtgtggatctgatgcaccaggctgtgccctacccaagcgcccccaggaagcagcggcgggagcgcaccaccttcacccggagccaactggaggagctggaggcactgtttgccaagacccagtacccagacgtctatgcccgtgaggaggtggctctgaagatcaatctgcctgagtccagggttcaggtttggttcaagaaccggagggctaaatgcaggcagcagcgacagcagcagaaacagcagcagcagcccccagggggccaggccaaggcccggcctgccaagaggaaggcgggcacgtccccaagaccctccacagatgtgtgtccagaccctctgggcatctcagattcctacagtccccctctgcccggcccctcaggctccccaaccacggcagtggccactgtgtccatctggagcccagcctcagagtcccctttgcctgaggcgcagcgggctgggctggtggcctcagggccgtctctgacctccgccccctatgccatgacctacgccccggcctccgctttctgctcttccccctccgcctatgggtctccgagctcctatttcagcggcctagacccctacctttctcccatggtgccccagctagggggcccggctcttagccccctctctggcccctccgtgggaccttccctggcccagtcccccacctccctatcaggccagagctatggcgcctacagccccgtggatagcttggaattcaaggaccccacgggcacctggaaattcacctacaatcccatggaccctctggactacaaggatcagagtgcctggaagtttcagatcttgtag

SEQ ID NO: 860 - protein, 299 aa, Artificial Sequence [sequence omitted]

SEQ ID NO: 861 - DNA, 603 bp, Artificial Sequence
  synthetic
atggggcaggagtttagctgggaggaggcggaggcagctggcgagatagatgtggcggagctccaggagtggtacaagaagtttgtgatggagtgccccagcggcacactctttatgcatgagtttaagcgcttcttcaaggtcacagacgatgaggaggcctcccagtatgtagagggcatgttccgagccttcgacaagaatggggacaacaccatcgacttcctggagtacgtggcagctctgaatctcgtgctgaggggcaccctggagcacaagctgaagtggacattcaagatctatgataaggatggcaatggctgcatcgaccgcctggagctactcaacattgtggagggaatttaccagctgaagaaagcctgccggcgagagctacaaactgagcaaggccagctgctcacacccgaggaggtcgtggacaggatcttcctcctggtggatgagaatggagatggccagctgtctctgaacgagtttgttgaaggtgcccgtcgggacaagtgggtgatgaagatgctgcagatggacatgaatcccagcagctggctcgctcagcagagacggaaaagtgccatgttctga

SEQ ID NO: 862 - protein, 200 aa, Artificial Sequence
  synthetic
MGQEFSWEEAEAAGEIDVAELQEWYKKFVMECPSGTLFMHEFKRFFKVTDDEEASQYVEGMFRAFDKNGDNTIDFLEYVAALNLVLRGTLEHKLKWTFKIYDKDGNGCIDRLELLNIVEGIYQLKKACRRELQTEQGQLLTPEEVVDRIFLLVDENGDGQLSLNEFVEGARRDKWVMKMLQMDMNPSSWLAQQRRKSAMF

SEQ ID NO: 863 - DNA, 951 bp, Artificial Sequence
  synthetic
atgctggtcaccttgggactgctcacctccttcttctcgttcctgtatatggtagctccatccatcaggaagttctttgctggtggagtgtgtagaacaaatgtgcagcttcctggcaaggtagtggtgatcactggcgccaacacgggcattggcaaggagacggccagagagctcgctagccgaggagcccgagtctatattgcctgcagagatgtactgaagggggagtctgctgccagtgaaatccgagtggatacaaagaactcccaggtgctggtgcggaaattggacctatccgacaccaaatctatccgagcctttgctgagggctttctggcagaggaaaagcagctccatattctgatcaacaatgcgggagtaatgatgtgtccatattccaagacagctgatggctttgaaacccacctgggagtcaaccacctgggccacttcctcctcacctacctgctcctggagcggctaaaggtgtctgcccctgcacgggtggttaatgtgtcctcggtggctcaccacattggcaagattcccttccacgacctccagagcgagaagcgctacagcaggggttttgcctattgccacagcaagctggccaatgtgctttttactcgtgagctggccaagaggctccaaggcaccggggtcaccacctacgcagtgcacccaggcgtcgtccgctctgagctggtccggcactcctccctgctctgcctgctctggcggctcttctccccctttgtcaagacggcacgggagggggcgcagaccagcctgcactgcgccctggctgagggcctggagcccctgagtggcaagtacttcagtgactgcaagaggacctgggtgtctccaagggcccgaaataacaaaacagctgagcgcctatggaatgtcagctgtgagcttctaggaatccggtgggagtag

SEQ ID NO: 864 - protein, 316 aa, Artificial Sequence
  synthetic
MLVTLGLLTSFFSFLYMVAPSIRKFFAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFSDCKRTWVSPRARNNKTAERLWNVSCELLGIRWE

SEQ ID NO: 865 - DNA, 1104 bp, Artificial Sequence
  synthetic
atggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtga

SEQ ID NO: 866 - protein, 367 aa, Artificial Sequence
  synthetic
METRPTALMSSTVAAAAPAAGAASRKESPGRWGLGEDPTGVSPSLQCRVCGDSSSGKHYGIYACNGCSGFFKRSVRRRLIYRCQVGAGMCPVDKAHRNQCQACRLKKCLQAGMNQDAVQNERQPRSTAQVHLDSMESNTESRPESLVAPPAPAGRSPRGPTPMSAARALGHHFMASLITAETCAKLEPEDADENIDVTSNDPEFPSSPYSSSSPCGLDSIHETSARLLFMAVKWAKNLPVFSSLPFRDQVILLEEAWSELFLLGAIQWSLPLDSCPLLAPPEASAAGGAQGRLTLASMETRVLQETISRFRALAVDPTEFACMKALVLFKPETRGLKDPEHVEALQDQSQVMLSQHSKAHHPSQPVR

SEQ ID NO: 867 - DNA, 714 bp, Artificial Sequence
  synthetic
atggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctga

SEQ ID NO: 868 - protein, 237 aa, Artificial Sequence
  synthetic
MALPPSPLAMEYVNDFDLMKFEVKREPSEGRPGPPTASLGSTPYSSVPPSPTFSEPGMVGATEGTRPGLEELYWLATLQQQLGAGEALGLSPEEAMELLQGQGPVPVDGPHGYYPGSPEETGAQHVQLAERFSDAALVSMSVRELNRQLRGCGRDEALRLKQRRRTLKNRGYAQACRSKRLQQRRGLEAERARLAAQLDALRAEVARLARERDLYKARCDRLTSSGPGSGDPSHLFL

SEQ ID NO: 869 - DNA, 1056 bp, Artificial Sequence
  synthetic
atggcgccggtgttgcccctggtgctgcccctgcagccccgcatccgcctggcacaagggctctggctcctctcctggctgctggcgctggctggtggcgtcatcctcctctgtagtgggcacctcctggtccagctaaggcaccttggcaccttcctggctccctcctgtcagttccctgtcctgccccaggctgccctggcagcgggcgcggtggctctgggcacaggactagtgggtgtaggagccagccgggcaagtctgaatgcagctctataccctccctggcgaggggtcctgggcccgctgctggtggctggcacggctggtgggggggggctcctggtcgtcggcctcgggctagccctggctttgcctgggagtctggatgaggcgctggaggagggcctggtgactgccttggctcactacaaggacacagaggtgcctgggcactgtcaggccaaaaggctggtggatgagctgcaactgaggtaccactgctgcgggcgccacgggtacaaggattggtttggggtccagtgggtcagcagccgttacctggatcccggtgaccgggatgtggctgaccggatccagagcaatgtagaaggcctatacctgactgatggggtccctttctcctgttgcaacccccactcaccccggccttgcctgcaaaaccgtctttcagactcctacgcccaccccctgttcgatccccgacaacccaaccaaaacctctgggcccaagggtgccatgaggtgctgctggagcacttgcaggacttggcaggcacactgggtagcatgctggctgtcaccttcctactgcaggctctggtgctccttggcctgcggtacctgcaaacagcactggaggggcttggaggggtcattgatgcgggaggagagacccagggctatctctttcccagtgggctgaaagatatgctgaaaacagcatggctacagggaggggttgcctgcaggccagcacctgaggaggccccaccaggagaagcacctcccaaggaggatctatctgaggcctag

SEQ ID NO: 870 - protein, 351 aa, Artificial Sequence
  synthetic
MAPVLPLVLPLQPRIRLAQGLWLLSWLLALAGGVILLCSGHLLVQLRHLGTFLAPSCQFPVLPQAALAAGAVALGTGLVGVGASRASLNAALYPPWRGVLGPLLVAGTAGGGGLLVVGLGLALALPGSLDEALEEGLVTALAHYKDTEVPGHCQAKRLVDELQLRYHCCGRHGYKDWFGVQWVSSRYLDPGDRDVADRIQSNVEGLYLTDGVPFSCCNPHSPRPCLQNRLSDSYAHPLFDPRQPNQNLWAQGCHEVLLEHLQDLAGTLGSMLAVTFLLQALVLLGLRYLQTALEGLGGVIDAGGETQGYLFPSGLKDMLKTAWLQGGVACRPAPEEAPPGEAPPKEDLSEA

SEQ ID NO: 871 - DNA, 894 bp, Artificial Sequence
  synthetic
atgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttgtga

SEQ ID NO: 872 - protein, 297 aa, Artificial Sequence
  synthetic
MMSYLKQPPYAVNGLSLTTSGMDLLHPSVGYPGPWASCPAATPRKQRRERTTFTRAQLDVLEALFAKTRYPDIFMREEVALKINLPESRVQVWFKNRRAKCRQQQQQQQNGGQNKVRPAKKKTSPAREVSSESGTSGQFTPPSSTSVPTIASSSAPVSIWSPASISPLSDPLSTSSSCMQRSYPMTYTQASGYSQGYAGSTSYFGGMDCGSYLTPMHHQLPGPGATLSPMGTNAVTSHLNQSPASLSTQGYGASSLGFNSTTDCLDYKDQTASWKLNFNADCLDYKDQTSSWKFQVL

SEQ ID NO: 873 - DNA, 606 bp, Artificial Sequence
  synthetic
atgggcaacgtgatggagggaaagtcagtggaggagctgagcagcaccgagtgccaccagtggtacaagaagttcatgactgagtgcccctctggccaactcaccctctatgagttccgccagttcttcggcctcaagaacctgagcccgtcggccagccagtacgtggaacagatgtttgagacttttgacttcaacaaggacggctacattgatttcatggagtacgtggcagcgctcagcttggtcctcaaggggaaggtggaacagaagctccgctggtacttcaagctctatgatgtagatggcaacggctgcattgaccgcgatgagctgctcaccatcatccaggccattcgcgccattaacccctgcagcgataccaccatgactgcagaggagttcaccgatacagtgttctccaagattgacgtcaacggggatggggaactctccctggaagagtttatagagggcgtccagaaggaccagatgctcctggacacactgacacgaagcctggaccttacccgcatcgtgcgcaggctccagaatggcgagcaagacgaggagggggctgacgaggccgctgaggcagccggctga

SEQ ID NO: 874 - protein, 201 aa, Artificial Sequence
  synthetic
MGNVMEGKSVEELSSTECHQWYKKFMTECPSGQLTLYEFRQFFGLKNLSPSASQYVEQMFETFDFNKDGYIDFMEYVAALSLVLKGKVEQKLRWYFKLYDVDGNGCIDRDELLTIIQAIRAINPCSDTTMTAEEFTDTVFSKIDVNGDGELSLEEFIEGVQKDQMLLDTLTRSLDLTRIVRRLQNGEQDEEGADEAAEAAG

SEQ ID NO: 875 - DNA, 3312 bp, Artificial Sequence
  synthetic
atgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttga

SEQ ID NO: 876 - protein, 1103 aa, Artificial Sequence
  synthetic
MTACARRAGGLPDPGLCGPAWWAPSLPRLPRALPRLPLLLLLLLLQPPALSAVFTVGVLGPWACDPIFSRARPDLAARLAAARLNRDPGLAGGPRFEVALLPEPCRTPGSLGAVSSALARVSGLVGPVNPAACRPAELLAEEAGIALVPWGCPWTQAEGTTAPAVTPAADALYALLRAFGWARVALVTAPQDLWVEAGRSLSTALRARGLPVASVTSMEPLDLSGAREALRKVRDGPRVTAVIMVMHSVLLGGEEQRYLLEAAEELGLTDGSLVFLPFDTIHYALSPGPEALAALANSSQLRRAHDAVLTLTRHCPSEGSVLDSLRRAQERRELPSDLNLQQVSPLFGTIYDAVFLLARGVAEARAAAGGRWVSGAAVARHIRDAQVPGFCGDLGGDEEPPFVLLDTDAAGDRLFATYMLDPARGSFLSAGTRMHFPRGGSAPGPDPSCWFDPNNICGGGLEPGLVFLGFLLVVGMGLAGAFLAHYVRHRLLHMQMVSGPNKIILTVDDITFLHPHGGTSRKVAQGSRSSLGARSMSDIRSGPSQHLDSPNIGVYEGDRVWLKKFPGDQHIAIRPATKTAFSKLQELRHENVALYLGLFLARGAEGPAALWEGNLAVVSEHCTRGSLQDLLAQREIKLDWMFKSSLLLDLIKGIRYLHHRGVAHGRLKSRNCIVDGRFVLKITDHGHGRLLEAQKVLPEPPRAEDQLWTAPELLRDPALERRGTLAGDVFSLAIIMQEVVCRSAPYAMLELTPEEVVQRVRSPPPLCRPLVSMDQAPVECILLMKQCWAEQPELRPSMDHTFDLFKNINKGRKTNIIDSMLRMLEQYSSNLEDLIRERTEELELEKQKTDRLLTQMLPPSVAEALKTGTPVEPEYFEQVTLYFSDIVGFTTISAMSEPIEVVDLLNDLYTLFDAIIGSHDVYKVETIGDAYMVASGLPQRNGQRHAAEIANMSLDILSAVGTFRMRHMPEVPVRIRIGLHSGPCVAGVVGLTMPRYCLFGDTVNTASRMESTGLPYRIHVNLSTVGILRALDSGYQVELRGRTELKGKGAEDTFWLVGRRGFNKPIPKPPDLQPGSSNHGISLQEIPPERRRKLEKARPGQFS

SEQ ID NO: 877 - DNA, 6038 bp, Artificial Sequence
  synthetic
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctccctgcaggtcataaaatcccagtccagagtcaccagcccttcttaaccacttcctactgtgtgaccctttcagcctttacttcctcatcagtaaaatgaggctgatgatatgggcatccatactccagggccagtgtgagcttacaacaagataaggagtggtgctgagcctggtgccgggcaggcagcaggcatgtttctcccaattatgccctctcactgccagccccacctccattgtcctcacccccagggctcaaggttctgccttcccctttctcagccctgaccctactgaacatgtctccccactcccaggcagtgccagggcctctcctggagggttgcggggacagaaggacagccggagtgcagagtcagcggttgagggattggggctatgccagctaatccgaagggttgggggggctgagctggattcacctgtccttgtctctgattggctcttggacacccctagcccccaaatcccactaagcagccccaccagggattgcacaggtccgtagagagccagttgattgcaggtcctcctggggccagaagggtgcctgggaggccaggttctggggatcccctccatccagaagaaccacctgctcactctgtcccttcgcctgctgctgggaccgcggccgcatggaggtccacactaatcaagaccccctggatgccgaggtgcacaccaaccaggaccctctggaccatatggtggaccacttacttccagtggacgagaacttctcgtcgccaaaatgcccagttgggtatctgggtgataggctggttggccggcgggcatatcacatgctgccctcacccgtctctgaagatgacagcgatgcctccagcccctgctcctgttccagtcccgactctcaagccctctgctcctgctatggtggaggcctgggcaccgagagccaggacagcatcttggacttcctattgtcccaggccacgctgggcagtggcgggggcagcggcagtagcattggggccagcagtggccccgtggcctgggggccctggcgaagggcagcggcccctgtgaagggggagcatttctgcttgcccgagtttcctttgggtgatcctgatgacgtcccacggcccttccagcctaccctggaggagattgaagagtttctggaggagaacatggagcctggagtcaaggaggtccctgagggcaacagcaaggacttggatgcctgcagccagctctcagctgggccacacaagagccacctccatcctgggtccagcgggagagagcgctgttcccctccaccaggtggtgccagtgcaggaggtgcccagggcccaggtgggggccccacgcctgatggccccatcccagtgttgctgcagatccagcccgtgcctgtgaagcaggaatcgggcacagggcctgcctcccctgggcaagccccagagaatgtcaaggttgcccagctcctggtcaacatccaggggcagaccttcgcactcgtgccccaggtggtaccctcctccaacttgaacctgccctccaagtttgtgcgcattgcccctgtgcccattgccgccaagcctgttggatcgggacccctggggcctggccctgccggtctcctcatgggccagaagttccccaagaacccagccgcagaactcatcaaaatgcacaaatgtactttccctggctgcagcaagatgtacaccaaaagcagccacctcaaggcccacctgcgccggcacacgggtgagaagcccttcgcctgcacctggccaggctgcggctggaggttctcgcgctctgacgagctgtcgcggcacaggcgctcgcactcaggtgtgaagccgtaccagtgtcctgtgtgcgagaagaagttcgcgcggagcgaccacctctccaagcacatcaaggtgcaccgcttcccgcggagcagccgctccgtgcgctccgtgaactctagatacccgtacgacgttccagactatgcatcttgatagaagcaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 878 - DNA, 5722 bp, Artificial Sequence
  synthetic
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctccctgcaggtcataaaatcccagtccagagtcaccagcccttcttaaccacttcctactgtgtgaccctttcagcctttacttcctcatcagtaaaatgaggctgatgatatgggcatccatactccagggccagtgtgagcttacaacaagataaggagtggtgctgagcctggtgccgggcaggcagcaggcatgtttctcccaattatgccctctcactgccagccccacctccattgtcctcacccccagggctcaaggttctgccttcccctttctcagccctgaccctactgaacatgtctccccactcccaggcagtgccagggcctctcctggagggttgcggggacagaaggacagccggagtgcagagtcagcggttgagggattggggctatgccagctaatccgaagggttgggggggctgagctggattcacctgtccttgtctctgattggctcttggacacccctagcccccaaatcccactaagcagccccaccagggattgcacaggtccgtagagagccagttgattgcaggtcctcctggggccagaagggtgcctgggaggccaggttctggggatcccctccatccagaagaaccacctgctcactctgtcccttcgcctgctgctgggaccgcggccgcatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 879 - DNA, 7647 bp, Artificial Sequence
  synthetic
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctcctcctagtgtcaccttggcccctcttagaagccaattaggccctcagtttctgcagcggggattaatatgattatgaaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcccaattgatggaggtccacactaatcaagaccccctggatgccgaggtgcacaccaaccaggaccctctggaccatatggtggaccacttacttccagtggacgagaacttctcgtcgccaaaatgcccagttgggtatctgggtgataggctggttggccggcgggcatatcacatgctgccctcacccgtctctgaagatgacagcgatgcctccagcccctgctcctgttccagtcccgactctcaagccctctgctcctgctatggtggaggcctgggcaccgagagccaggacagcatcttggacttcctattgtcccaggccacgctgggcagtggcgggggcagcggcagtagcattggggccagcagtggccccgtggcctgggggccctggcgaagggcagcggcccctgtgaagggggagcatttctgcttgcccgagtttcctttgggtgatcctgatgacgtcccacggcccttccagcctaccctggaggagattgaagagtttctggaggagaacatggagcctggagtcaaggaggtccctgagggcaacagcaaggacttggatgcctgcagccagctctcagctgggccacacaagagccacctccatcctgggtccagcgggagagagcgctgttcccctccaccaggtggtgccagtgcaggaggtgcccagggcccaggtgggggccccacgcctgatggccccatcccagtgttgctgcagatccagcccgtgcctgtgaagcaggaatcgggcacagggcctgcctcccctgggcaagccccagagaatgtcaaggttgcccagctcctggtcaacatccaggggcagaccttcgcactcgtgccccaggtggtaccctcctccaacttgaacctgccctccaagtttgtgcgcattgcccctgtgcccattgccgccaagcctgttggatcgggacccctggggcctggccctgccggtctcctcatgggccagaagttccccaagaacccagccgcagaactcatcaaaatgcacaaatgtactttccctggctgcagcaagatgtacaccaaaagcagccacctcaaggcccacctgcgccggcacacgggtgagaagcccttcgcctgcacctggccaggctgcggctggaggttctcgcgctctgacgagctgtcgcggcacaggcgctcgcactcaggtgtgaagccgtaccagtgtcctgtgtgcgagaagaagttcgcgcggagcgaccacctctccaagcacatcaaggtgcaccgcttcccgcggagcagccgctccgtgcgctccgtgaactctagatacccgtacgacgttccagactatgcatcttgacatatggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagttgtagttaatgattaacccgccatgctacttatctacgtagccatgctctaggaagatcggaattcgcccttaagttacgctagctccctgcaggtcataaaatcccagtccagagtcaccagcccttcttaaccacttcctactgtgtgaccctttcagcctttacttcctcatcagtaaaatgaggctgatgatatgggcatccatactccagggccagtgtgagcttacaacaagataaggagtggtgctgagcctggtgccgggcaggcagcaggcatgtttctcccaattatgccctctcactgccagccccacctccattgtcctcacccccagggctcaaggttctgccttcccctttctcagccctgaccctactgaacatgtctccccactcccaggcagtgccagggcctctcctggagggttgcggggacagaaggacagccggagtgcagagtcagcggttgagggattggggctatgccagctaatccgaagggttgggggggctgagctggattcacctgtccttgtctctgattggctcttggacacccctagcccccaaatcccactaagcagccccaccagggattgcacaggtccgtagagagccagttgattgcaggtcctcctggggccagaagggtgcctgggaggccaggttctggggatcccctccatccagaagaaccacctgctcactctgtcccttcgcctgctgctgggaccgcggccgcatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 880 - DNA, 7175 bp, Artificial Sequence
  synthetic
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctcctcctagtgtcaccttggcccctcttagaagccaattaggccctcagtttctgcagcggggattaatatgattatgaaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcccaattgatggaggtccacactaatcaagaccccctggatgccgaggtgcacaccaaccaggaccctctggaccatatggtcgatatggataaactcataaacaacttggaggtccaacttaattcagaaggtggctcaatgcaggtattcaagcaggtcactgcttctgttcggaacagagatccccctgagatagaatacagaagtaatatgacttctccaacactcctggatgccaaccccatggagaacccagcactgtttaatgacatcaagattgagcccccagaagaacttttggctagtgatttcagcctgccccaagtggaaccagttgacctctcctttcacaagcccaaggctcctctccagcctgctagcatgctacaagctccaatacgtccccccaagccacagtcttctccccagacccttgtggtgtccacgtcaacatctgacatgagcacttcagcaaacattcctactgttctgaccccaggctctgtcctgacctcctctcagagcactggtagccagcagatcttacatgtcattcacactatcccctcagtcagtctgccaaataagatgggtggcctgaagaccatcccagtggtagtgcagtctctgcccatggtgtatactactttgcctgcagatgggggccctgcagccattacagtcccactcattggaggagatggtaaaaatgctggatcagtgaaagttgaccccacctccatgtctccactggaaattccaagtgacagtgaggagagtacaattgagagtggatcctcagccttgcagagtctgcagggactacagcaagagagagaagccttataaactctagatacccgtacgacgttccagactatgcatcttgacatatggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagttgtagttaatgattaacccgccatgctacttatctacgtagccatgctctaggaagatcggaattcgcccttaagttacgctagctccctgcaggtcataaaatcccagtccagagtcaccagcccttcttaaccacttcctactgtgtgaccctttcagcctttacttcctcatcagtaaaatgaggctgatgatatgggcatccatactccagggccagtgtgagcttacaacaagataaggagtggtgctgagcctggtgccgggcaggcagcaggcatgtttctcccaattatgccctctcactgccagccccacctccattgtcctcacccccagggctcaaggttctgccttcccctttctcagccctgaccctactgaacatgtctccccactcccaggcagtgccagggcctctcctggagggttgcggggacagaaggacagccggagtgcagagtcagcggttgagggattggggctatgccagctaatccgaagggttgggggggctgagctggattcacctgtccttgtctctgattggctcttggacacccctagcccccaaatcccactaagcagccccaccagggattgcacaggtccgtagagagccagttgattgcaggtcctcctggggccagaagggtgcctgggaggccaggttctggggatcccctccatccagaagaaccacctgctcactctgtcccttcgcctgctgctgggaccgcggccgcatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 881 - DNA, 249 bp, Artificial Sequence
  synthetic
aggggtgttgggcggtgttggatttccaggtaacctctctgaccgaccaatcgaaatccctctcggaagaaagcgcttcactagcttatctcatctaaccaattagaagccttaggactcaaagaccctcgacgcccccaccttatcgaccaatcagagcgcccccttacaaaggccggcagcagtgacagccaatgaaaatagaatttggcctaacacccctccttcctcgcctcacgcccacgtggt

SEQ ID NO: 882 - DNA, 249 bp, Artificial Sequence
  synthetic
gtatattttaagcaaagcccagtgtctttaaagaacattctggtaatgtcggaggcctggatttgttgcctgctcgtccccccgtgcagcagcggcctgttttcctccccctgttgtgtgtttttattattttccctctggcttagatgtgtcaatcattctctccctgccattggttggagagtttgcgtcaaaaagttgccagagaggcgctttctcagcaaatctccctgagagcgggaccggcct

SEQ ID NO: 883 - DNA, 249 bp, Artificial Sequence
  synthetic
tgctcctgcacccgaagccgttggctcagagactggagggcctcccgagcagggccgtcacctgggaaaagggcaagaggtactcagaatgtacccatcacctgaagaaagcgtagaggggattctgtctccccacaactttacatgtgtccggtaggactgcccctttcaggcagccccaggcttgaatgctcgtccagaggggaggagacggtggtaaaggatcaagggcctccttctggcagggca